(19) United States
(12) Patent Application Publication
(10) Pub. No.: US 2019/0252037 A1
(43) Pub. Date: Aug. 15, 2019

(54) Viral Polypeptide Fragments That Bind Cellular POL II C-Terminal Domain (CTD) and Their Uses

(71) Applicant: THE EUROPEAN MOLECULAR BIOLOGY LABORATORY, Heidelberg (DE)

(72) Inventors: Stephen CUSACK, Seyssinet-Pariset (FR); Alexander PFLUG, Cambridge (GB); Maria LUKARSKA, Grenoble (FR)

(21) Appl. No.: 16/315,882

(22) PCT Filed: Jul. 7, 2017
(86) PCT No.: PCT/EP2017/067144

§ 371 Date: Jan. 7, 2019

(30) Foreign Application Priority Data
Jul. 7, 2016 (EP) 16178396.4

(51) Int. Cl. G16B 15/30; G16B 15/20; C12N 9/12; G16C 20/30; G16C 20/50; C12N 15/85

(52) U.S. Cl. CPC G16B 15/30; G16B 15/20; C12N 15/85; G16C 20/30; G16C 20/50; C12N 9/127

(57) ABSTRACT

The present invention relates to in silico methods for identifying compounds which decrease or prevent the binding of the viral RNA-dependent RNA polymerase from the Orthomyxoviridae family or variant thereof, to its ligand, (preferably to cellular Pol II, more preferably to CTD), as well as methods of producing the identified compounds. The present invention also relates to a compounds identifiable and/or producible by said methods. The present invention also relates to antibodies directed against the binding site of the RNA-dependent RNA polymerase, to its ligand (in particular to cellular Pol II, in particular to CTD of Pol II) as well as nucleic acids encoding said antibodies and vectors comprising the nucleic acid. The present invention relates to a pharmaceutical composition producible according to said method, and/or comprising said compound, said antibody, said nucleic acid, or said vector. The present invention also relates to the use of said compound, said antibody, said nucleic acid, said vector or said pharmaceutical in treating, ameliorating, or preventing disease conditions caused by viral infections with viruses of the Orthomyxoviridae family.

### TECHNICAL FIELD OF INVENTION

The present invention relates to in silico methods for identifying compounds which decrease or prevent the binding of the viral RNA-dependent RNA polymerase from the Orthomyxoviridae family or variant thereof, to its ligand, (preferably to cellular Pol II, more preferably to the Pol II C-terminal domain, CTD), as well as methods of producing the identified compounds. The present invention also relates to compounds identifiable and/or producible by said methods. The present invention also relates to antibodies directed against the binding site of the RNA-dependent RNA polymerase, to its ligand (in particular to cellular Pol II, in particular to CTD of Pol II) as well as nucleic acids encoding said antibodies and vectors comprising the nucleic acid. The present invention relates to a pharmaceutical composition producible according to said method, and/or comprising said compound, said antibody, said nucleic acid, or said vector. The present invention also relates to the use of said compound, said antibody, said nucleic acid, said vector or said pharmaceutical in treating, ameliorating, or preventing disease conditions caused by viral infections with viruses of the Orthomyxoviridae family.

### BACKGROUND OF THE INVENTION

Influenza is responsible for much morbidity and mortality in the world and is considered by many as belonging to the most significant viral threats to humans Annual Influenza epidemics swipe the globe and occasional new virulent strains cause pandemics of great destructive power. At present the primary means of controlling Influenza virus epidemics is vaccination. However, mutant Influenza viruses are rapidly generated which escape the effects of vaccination. In the light of the fact that it takes approximately 6 months to generate a new Influenza vaccine, alternative therapeutic means, i.e., antiviral medication, are required especially as the first line of defence against a rapidly spreading pandemic.

An excellent starting point for the development of antiviral medication is structural data of essential viral proteins. Thus, the crystal structure determination of the Influenza virus surface antigen neuraminidase (von Itzstein et al., 1993, Nature 363:418-423) led directly to the development of neuraminidase inhibitors with anti-viral activity preventing the release of virus from the cells, however, not the virus production. These and their derivatives have subsequently developed into the anti-Influenza drugs, zanamivir (Glaxo) and oseltamivir (Roche), which are currently being stockpiled by many countries as a first line of defence against an eventual pandemic. However, these medicaments provide only a reduction in the duration of the clinical disease. Alternatively, other anti-Influenza compounds such as amantadine and rimantadine target an ion channel protein, i.e., the M2 protein, in the viral membrane interfering with the uncoating of the virus inside the cell. However, they have not been extensively used due to their side effects and the rapid development of resistant virus mutants (Magden et al., 2005, Appl. Microbiol. Biotechnol. 66:612-621). In addition, more unspecific viral drugs, such as ribavirin, have been shown to work for treatment of Influenza infections (Eriksson et al., 1977, Antimicrob. Agents Chemother. 11:946-951). However, ribavirin is only approved in a few countries, probably due to severe side effects (Furuta et al., 2005, Antimicrob. Agents Chemother. 49:981-986). Clearly, new antiviral compounds are needed, preferably directed against different targets.

Influenza virus A, B, C and Isavirus as well as Thogotovirus belong to the family of Orthomyxoviridae which, as well as the family of the Bunyaviridae, including the Hantavirus, Nairovirus, Orthobunyavirus, Phlebovirus, and Tospovirus, are negative stranded RNA viruses. Their genome is segmented and comes in ribonucleoprotein particles that include the RNA dependent RNA polymerase which carries out (i) the initial copying of the single-stranded virion RNA (vRNA) into viral mRNAs and (ii) the vRNA replication. The polymerase complex seems to be an appropriate antiviral drug target since it is essential for synthesis of viral mRNA and viral replication and contains several functional active sites likely to be significantly different from those found in host cell proteins (Magden et al., supra). Thus, for example, there have been attempts to interfere with the assembly of polymerase subunits by a 25-amino-acid peptide resembling the PA-binding domain within PB1 (Ghanem et al., 2007, J. Virol. 81:7801-7804). Moreover, there have been attempts to interfere with viral transcription by nucleoside analogs, such as 2′-deoxy-2′-fluoroguanosine (Tisdale et al., 1995, Antimicrob. Agents Chemother. 39:2454-2458) and it has been shown that T-705, a substituted pyrazine compound may function as a specific inhibitor of Influenza virus RNA polymerase (Furuta et al., supra). Furthermore, the endonuclease activity of the polymerase has been targeted and a series of 4-substituted 2,4-dioxobutanoic acid compounds has been identified as selective inhibitors of this activity in Influenza viruses (Tomassini et al., 1994, Antimicrob. Agents Chemother. 38:2827-2837). In addition, flutimide, a substituted 2,6-diketopiperazine, identified in extracts of Delitschia confertaspora, a fungal species, has been shown to inhibit the endonuclease of Influenza virus (Tomassini et al., 1996, Antimicrob. Agents Chemother. 40:1189-1193).

The PA subunit of the polymerase was the least well-characterised functionally, being implicated in both cap-binding and endonuclease activity, vRNA replication, and a controversial protease activity. PA (716 residues in influenza A) is separable by trypsination at residue 213. The crystal structure of the C-terminal two-thirds of PA bound to a PB1 N-terminal peptide provided the first structural insight into both a large part of the PA subunit and the exact nature of one of the critical inter-subunit interactions (He et al., 2008, Nature 454:1123-1126; Obayashi et al., 2008, Nature 454:1127-1131). Systematic mutation of conserved residues in the PA amino-terminal domain have identified residues important for protein stability, promoter binding, cap-binding and endonuclease activity of the polymerase complex (Hara et al., 2006, J. Virol. 80:7789-7798). Subsequently it was shown that the cap-snatching endonuclease constituted the N-terminal part of PA (˜residues 1-200) (Dias et al Nature 2009, PMID: 19194459, Yuan et al, Nature PMID: 19194459) and this has greatly aided drug development targeting the endonuclease (e.g. Kowalinski et al, PLoS Pathog. 2012, PMID 22876177). Finally in 2014, the crystal structures of the complete bat influenza A (Pflug et al, Nature 2014) and human influenza B (Reich et al, 2014 Nature) showed how PA is integrated into the full heterotrimer and has additional roles in stabilising the PB1 subunit and, together with PB1, binding the 5′ end of the vRNA promoter.

Viral replication requires actively transcribing cellular RNA polymerase II (Pol II) (Mahy et al. 1972, PNAS 69:1421-1424) and a physical association of FluPol with the C-terminal domain (CTD) of Pol II has been shown (Engelhardt et al. 2005, Journal of virology 79:5812-5818; Loucaides et al. 2009, Virology 394:154-163). This close coupling of viral and cellular transcription is thought to enable ‘cap-snatching’, the unique mechanism by which the viral polymerase pirates short capped oligomers, derived from nascent Pol II transcripts, for transcription priming (Plotch et al. 1981, Cell 23:847-858; Reich et al. 2014, Nature 516:361-366). Pol II CTD in mammalian cells consists of 52 heptad repeats with consensus sequence Y₁S₂P₃T₄S₅P₆S₇ (Palancade et al. 2003, Eur J Biochem 270:3859-3870). Most of these residues can be reversibly phosphorylated and the temporal phosphorylation pattern, defined by the regulated interplay of several kinases and phosphatases, correlates with distinct phases of transcription (Lidschreiber et al. 2013, Mol Cell Biol 33:3805-3816; Hsin et al. 2014, Mol Cell Biol 34:2488-2498; Martinez-Rucobo et al. 2015, Mol Cell 58:1079-1089). It has been shown that Flu Pol associates with initiating Pol II, when the CTD is Ser5-phosphorylated, but not to the Ser2-phosphorylated form, the hallmark of elongation (Engelhardt et al. 2005, Journal of virology 79:5812-5818; Loucaides et al. 2009, Virology 394:154-163; Chan et al. 2006, Virology 351:210-217). Indeed viral polymerase inhibits elongation as well as inducing Pol II degradation (Rodriguez et al. 2007, Journal of virology 81:5315-5324; Vreede et al. 2010, Virology 396, 125-134). This contributes to the inhibition of cellular transcription (‘host shut-off’), which is thought to be of importance in countering the anti-viral response and in the switch from viral transcription to replication (Vreede et al. 2010, Virology 396, 125-134). Despite the well-established functional coupling between viral and cellular transcription, the exact nature of the structural interaction between the two polymerases remains unclear. There is thus a need in understanding exactly how the viral RNS polymerase and cellular Pol II interact in order to be able to identify mechanisms and compounds targeting the binding sites of the viral polymerase and thereby interfere with the binding to the cellular polymerase.

The inventors have achieved to structurally characterize the interaction between the two polymerases by co-crystallisation of the entire viral polymerase with a Pol II CTD peptidomimetic comprising four repeats of the Ser5 phosphorylated CTD and thereby identified the binding sites between the two. Thus, the present invention provides the unique opportunity to study the interaction site between the two polymerases which will considerably simplify the development of new anti-viral compounds targeting viral replication as well as optimising previously identified compounds.

The surprising achievement of the present inventors to identify a system that allows for performing in vitro high-throughput screening for inhibitors of the interaction site on the viral polymerase using easily obtainable material. Furthermore, the structural data of the Pol II CTD bound to the complete influenza polymerase and notably to the PA subunit allows for directed design of inhibitors and in silico screening for potentially therapeutic compounds targeting the CTD binding site on the polymerase. Finally the same system can be used for structurally characterising the interaction of eventual inhibitors with the viral polymerase and using this data to optimise further their inhibitory properties.

### SUMMARY OF THE INVENTION

In a first aspect, the present invention relates to an in silico method for identifying compounds which decrease or prevent the binding of the viral RNA-dependent RNA polymerase from the Orthomyxoviridae family or variant thereof, to its ligand, (in particular to cellular Pol II, in particular to CTD) comprising the steps of

(a) constructing a computer model based on the structure coordinates of the one or more binding site(s) of the viral RNA-dependent RNA polymerase to its ligand;

(b) selecting a potential modulating compound by a method selected from the group consisting of:
(i) modifying the co-crystallised ligand inside the binding site,
(ii) filtering and selecting compounds from small molecule databases based on the interaction profile of the co-crystallised ligand with the one or more binding site(s) of the viral RNA-dependent RNA polymerase, and/or based on 3D similarity to the co-crystallised ligand, and
(iii) de novo ligand design of said compound based on the interaction profile of the co-crystallised ligand with the one or more binding site(s) of the viral RNA-dependent RNA polymerase and/or based on 3D similarity to the co-crystallised ligand;

(c) employing computational means to perform a fitting program operation between computer models of the said compound and said one or more binding site(s) in order to provide an energy-minimized configuration of the said compound in the active site; and/or employing computational docking methods to position and place said compounds into said one or more binding site(s) in order to provide reasonable 3D-arrangements of the chemical entities, said compounds; and

(d) evaluating the results of said fitting operation and/or said docking methods to quantify the association between the said compound and the binding site model, thereby evaluating the ability of said compound to associate with the said active site.

In a second aspect, the present invention relates to a method of producing a compound which decreases or prevents the binding of the viral RNA-dependent RNA polymerase from the Orthomyxoviridae family or variant thereof, to its ligand, (in particular to cellular Pol II, in particular to CTD) comprising the steps of

(a) identifying said compound via the method of the first aspect, and

(b) synthesizing said compound and optionally formulating said compound or a pharmaceutically acceptable salt thereof with one or more pharmaceutically acceptable excipient(s) and/or carrier(s).

In a third aspect, the present invention relates to a compound identifiable by the method of the first aspect and/or producible by the method of the second aspect, wherein said compound is able to decrease or prevent the binding of the viral RNA-dependent RNA polymerase or variant thereof, to its ligand (in particular to cellular Pol II, in particular to CTD of Pol II).

In a fourth aspect, the present invention relates to an antibody directed against the one or more binding site(s) of the RNA-dependent RNA polymerase from a virus belonging to the Orthomyxoviridae family, or variant thereof, to its ligand (in particular to cellular Pol II, in particular to CTD of Pol II).

In a fifth aspect, the present invention relates to a nucleic acid encoding the antibody of the fourth aspect of the present invention.

In a sixth aspect, the present invention relates to a vector comprising the nucleic acid of the fifth aspect of the present invention.

In a seventh aspect, the present invention relates to a recombinant host cell comprising the nucleic acid of the fifth aspect or the vector of sixth aspect of the present invention.

In an eighth aspect, the present invention relates to a pharmaceutical composition producible according to the method of the second aspect of the present invention.

In a ninth aspect, the present invention relates to a pharmaceutical composition comprising the compound of the third aspect of the present invention, an antibody of the fourth aspect of the present invention, a nucleic acid of the fifth aspect of the present invention, or a vector of the sixth aspect of the present invention.

In a tenth aspect, the present invention relates to a compound according to the third aspect of the present invention, an antibody of the fourth aspect of the present invention, a nucleic acid of the fifth aspect of the present invention, a vector of the sixth aspect of the present invention, or a pharmaceutical composition of the seventh or eighth aspect of the present invention, for use in treating, ameliorating, or preventing disease conditions caused by viral infections with viruses of the Orthomyxoviridae family, in particular disease conditions caused by viral infections with Influenza virus.

In an eleventh aspect, the present invention relates to a method of treating ameliorating, or preventing disease conditions caused by viral infections with viruses of the Orthomyxoviridae family, in particular disease conditions caused by viral infections with Influenza virus, comprising administering a therapeutically effective amount of the compound according to the third aspect of the present invention, an antibody of the fourth aspect of the present invention, a nucleic acid of the fifth aspect of the present invention, a vector of the sixth aspect of the present invention, or a pharmaceutical composition of the seventh or eighth aspect of the present invention.

### BRIEF DESCRIPTION OF THE DRAWINGS

FIG. 1: Conformations of CTD peptides bound to FluA and other Pol II interacting factors.
Structures of proteins bound to SeP5 or SeP2-SeP5 CTD peptides. The structures of all proteins are shown in grey cartoon, the peptides are coloured to highlight their position and zoomed in next to their corresponding structure. CTD peptides are in stick representation. A.,B. Binding sites 1 (A) and 2 (B) of FluA polymerase with the corresponding parts of CTD SeP5 peptides. C. Ser2-Ser5-phosphorylated CTD bound to the peptidyl-prolyl isomerase Pin1 (PDB: 1F8A). D. Ser5-phosphorylated CTD bound to C. albicans capping enzyme Cgt1 (PDB: 1P16). E. Ser2-Ser5 phosphorylated CTD bound to mammalian capping enzyme Mce1 (PDB: 3RTX). F. Ser2-Ser5-phosphorylated CTD bound to S. pombe capping enzyme Pce1 (PDB: 4PZ6). G. Ser5-phosphorylated CTD bound to human Ssu72 (PDB: 302Q).

FIG. 2: Structure of influenza A polymerase bound to Ser5-phosphorylated CTD peptide.
A. Surface representation of the bat influenza A polymerase structure with bound Ser5-phosphorylated CTD peptide (blue sticks). The two peptide binding sites are located on the C-terminal domain of PA (PA-C).
B. Superposition of the bat FluA polymerase PA-C domain with (green) or without (yellow) CTD-bound peptide showing the movement of the 550 loop. Assuming only one 4-repeat peptide is bound, CTD residues from consecutive repeats are coloured (from the N-terminus) blue, white, magenta and cyan and the dashed line illustrates the missing connection (most of the second repeat).
C., D. Details of the interactions of PA residues (green) in binding site 1 (C) and site 2 (D) with the CTD peptide (blue). Putative hydrogen bonds are drawn as dashed lines.

FIG. 3: CTD peptide binding to FluB polymerase.
A. Sequence alignment of representative influenza strains A-D, showing that residues forming site 1 are conserved in FluA and FluB strains but that site 2 residues are only conserved in FluA strains.
B. Top: Omit difference electron density (Fo-Fc, 3.0 σ, blue mesh) observed on the surface of full-length FluB polymerase co-crystallised with the Ser5-phosphorylated CTD peptide. Bottom: Close-up of red boxed region showing interactions of the CTD peptide bound to site 1 of FluB polymerase in the same orientation as the corresponding site 1 in FluA (FIG. 1C). The additional extra density extending over PB2-627 domain cannot be modelled.
C., D. Fluorescence anisotropy data comparing the binding of two- and four-repeat fluorescently labelled Ser5-phosphorylated CTD peptides to FluA (C) and FluB (D). Error bars show SD of three experiments, KD are shown±the error of the fit.

FIG. 4: Sequence alignment of the PA subunit of various influenza strains (bat A, human A, avian A, B, C, D). Only the PA-C region from residue 220 is shown. Absolutely conserved residues are white on a red background and highly conserved residues are red letters boxed in blue. The amino acid numbering and secondary structure is for bat FluA polymerase. CTD binding site 1 residues are indicated with a cyan triangle (conserved in Flu A and Flu B strains) and site 2 with a yellow triangle (key residues are only conserved in Flu A strains). Residues referred to in the discussion (site 1: C/R448 (H1N1 453), site 2: I/L545 (550), T/5547 (552)) are shown with a black triangle.

FIG. 5: Fluorescence anisotropy data for Flu A polymerase. A. Displacement assay: Fluorescently labelled Ser5-phosphorylated peptide (4-repeat) bound to bat polymerase: vRNA is titrated with the same non-labelled peptide. The apparent K_{D} (K_{D}′) is shown. B. Binding of Ser5-phosphorylated peptide in the absence of the vRNA promotor. C. Interaction with non-phosphorylated 4-repeat CTD peptide (Y₁S₂P₃T₄S₅P₆S₇). The binding curve can be extrapolated to an estimated KD in the range of >10 μM. Error bars represent SD of three independent experiments, K_{D}s are shown±the error of fit.

FIG. 6: Mutational analysis of the SeP5 binding pocket. A. Table showing the measured KD for recombinant bat FluA mutants to Ser5-phosphorylated 4-repeat CTD peptide. Each double mutation affects only one of the two CTD binding sites on FluA (K289A and R449A for site 1, and, K630A and R633A for site 2, bat numbering). Also shown is the fold change compared to wild type protein (FluA or FluB for the corresponding mutants). B. Mini-genome assay comparing the activity of single and double mutants with decreased binding to phosphorylated Ser5 CTD. Error bars represent SD of three independent experiments, performed in triplicate. C. In vitro cap-dependent transcription activity assay, comparing wild type FluA to the corresponding double mutants in site 1 and site 2. Error bars show SD from three different experiments. Rate constants are shown±the error of the fit.

FIG. 7: Fluorescence anisotropy data of the interaction of mutant polymerase proteins to 4-repeat Ser5-phosphorylated CTD peptide. (A) Bat FluA site 1 double mutant K630A/R633A (left), site 2 double mutant K289A_R449A (right); (B) Influenza B site 1 double mutant K631A/R634A. Error bars represent SD of three independent experiments, K_{D}s are shown±the error of fit. C. Time-courses of unprimed replication reactions in vitro with vRNA and cRNA as template, comparing the site 1 and 2 double mutants with the wild type bat FluA. Error bars show the SD from three different reactions. The tables show the measured rate constants±the error of the fit.

FIG. 8: Mutant virus rescue experiments. A. Plaque assay showing the titers and plaque phenotype of the recombinant A/WSN/33 viruses in reverse genetics supernatants. Crystal violet staining of cell monolayers infected with the indicated viral dilutions is shown. WT, Mock: control performed with wild-type PA or no PA plasmid, respectively. B. Growth curves of recombinant A/WSN/33 viruses on MDCK cells. At the indicated time points, viral infectious titers were determined by plaque assay in MDCK cells. The X-axis was set at the limit of detection of the assay (25 pfu/mL). Error bars show SD of triplicates. Viral titers were possibly under-estimated for the R454A mutant because of the very small size of the plaques. C. Left: Wild type bat FluA structure bound to CTD peptide, showing the R633-SeP5 interaction. Right: C448R/R633A double mutant modelled on the CTD-bound bat FluA structure (corresponding to C453R/R638A in avian/human FluA) showing that R448 can compensate for R633. Residues involved in the double mutant are coloured magenta. D. View of the CTD-FluA interaction showing the position of the I545 and T547 residues. I545 (L550 in human/avian strains) makes contacts with Y1c and P6c; T547 (S/T552 in human/avian influenza) is close to S7b. FluA PA-C is coloured in green, I545 and T547 in magenta and the CTD peptide in blue.

FIG. 9: Table 1. Data collection and refinement statistics of FluA and FluB polymerases bound to 28 amino-acid SeP5 CTD peptide.

FIG. 10: Table 2. Reported binding affinities to CTD peptides with SeP2/SeP5 or SeP5 phosphorylation of various CTD-interacting proteins.

FIGS. 11 to 12: (common annotation): Refined atomic structure coordinates of the C-terminal domain of the PA subunit (chain A) as set forth in SEQ ID NO: 1 or SEQ ID NO: 2, with bound CTD (chain X). The file header gives information about the structure refinement of the complete heterotrimeric structure with bound vRNA promoter and CTD. “Atom” refers to the element whose coordinates are measured. The first letter in the column defines the element. The 3-letter code of the respective amino acid is given and the amino acid sequence position. The first 3 values in the line “Atom” define the atomic position of the element as measured. The fourth value corresponds to the occupancy and the fifth (last) value is the temperature factor (B factor). The occupancy factor refers to the fraction of the molecules in which each atom occupies the position specified by the coordinates. A value of “1” indicates that each atom has the same conformation, i.e., the same position, in all equivalent molecules of the crystal. B is a thermal factor that measures movement of the atom around its atomic center. This nomenclature corresponds to the Protein Data Bank (PDB) format.

FIG. 11: Structural co-ordinates of the C-terminal domain of the PA subunit (residues 258-713) of the RNA-dependent RNA polymerase of Bat Influenza A/H17N10 with bound CTD in standard Protein Data Bank (PDB) format.

FIG. 12: Structural co-ordinates of the C-terminal domain of the PA subunit (residues 258-722) of RNA-dependent RNA polymerase of human Influenza B/Memphis/13/03 with bound CTD in standard Protein Data Bank (PDB) format.

FIG. 13: Comparison of PA-CTD crystal structure for complete bat FluA and A/H7N9 core polymerases. Binding at site 1 is not observed in the H7N9 structure possibly due to the conformational changes in PA in this region. FluA PA is coloured in green, H7N9 PA in cyan, and the CTD peptide in blue (bat FluA site 1 and site 2) or magenta (H7N9 site 2).

FIG. 14: Conservation of site 2 CTD binding mode and phosphoserine interacting basic residues in PA of A/H7N9 avian influenza polymerase. H7N9 PA is coloured in cyan, and the CTD peptide in magenta. Putative hydrogen bonds are shown as yellow dotted lines.

FIG. 15: Table 3. Corresponding amino acid positions of SEQ ID NO: 1, SEQ ID NO: 2 and SEQ ID NO: 44

### LIST OF SEQUENCES

SEQ ID NO: 1: amino acid sequence of RNA-dependent RNA polymerase PA subunit A/little yellow-shouldered bat/Guatemala/060/2010(H17N10); GenBank: AFC35437.1

SEQ ID NO: 2: amino acid sequence of RNA-dependent RNA polymerase PA subunit of Human Influenza B/Memphis/13/03

SEQ ID NO: 3: peptide linker sequence GMGSGMA

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence. Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “nucleic acid” and “nucleic acid molecule” are used synonymously herein and are understood as single or double-stranded oligo- or polymers of deoxyribonucleotide or ribonucleotide bases or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers, In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single- or double-stranded molecules and are composed of naturally occurring nucleotides. The depiction of a single strand of a nucleic acid also defines (at least partially) the sequence of the complementary strand. The nucleic acid may be single or double stranded, or may contain portions of both double and single stranded sequences. Exemplified, double-stranded nucleic acid molecules can have 3′ or 5′ overhangs and as such are not required or assumed to be completely double-stranded over their entire length. The nucleic acid may be obtained by biological, biochemical or chemical synthesis methods or any of the methods known in the art, including but not limited to methods of amplification, and reverse transcription of RNA. The term nucleic acid comprises chromosomes or chromosomal segments, vectors (e.g., expression vectors), expression cassettes, naked DNA or RNA polymer, primers, probes, cDNA, genomic DNA, recombinant DNA, cRNA, mRNA, tRNA, microRNA (miRNA) or small interfering RNA (siRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

Nucleic acids may be degraded by endonucleases or exonucleases, in particular by DNases and RNases which can be found in the cell. It may, therefore, be advantageous to modify the nucleic acids in order to stabilize them against degradation, thereby ensuring that a high concentration of the nucleic acid is maintained in the cell over a long period of time. Typically, such stabilization can be obtained by introducing one or more internucleotide phosphorus groups or by introducing one or more non-phosphorus internucleotides. Accordingly, nucleic acids can be composed of non-naturally occurring nucleotides and/or modifications to naturally occurring nucleotides, and/or changes to the backbone of the molecule. Modified internucleotide phosphate radicals and/or non-phosphorus bridges in a nucleic acid include but are not limited to methyl phosphonate, phosphorothioate, phosphoramidate, phosphorodithioate and/or phosphate esters, whereas non-phosphorus internucleotide analogues include but are not limited to, siloxane bridges, carbonate bridges, carboxymethyl esters, acetamidate bridges and/or thioether bridges. Further examples of nucleotide modifications include but are not limited to: phosphorylation of 5′ or 3′ nucleotides to allow for ligation or prevention of exonuclease degradation/polymerase extension, respectively; amino, thiol, alkyne, or biotinyl modifications for covalent and near covalent attachments; fluorphores and quenchers; and modified bases such as deoxyInosine (dI), 5-Bromo-deoxyuridine (5-Bromo-dU), deoxyUridine, 2-Aminopurine, 2,6-Diaminopurine, inverted dT, inverted Dideoxy-T, dideoxyCytidine (ddC 5-Methyl deoxyCytidine (5-Methyl dC), locked nucleic acids (LNA's), 5-Nitroindole, Iso-dC and -dG bases, 2′-O-Methyl RNA bases, Hydroxmethyl dC, 5-hydroxybutynl-2′-deoxyuridine, 8-aza-7-deazaguanosineand Fluorine Modified Bases. Thus, the nucleic acid can also be an artificial nucleic acid which includes but is not limited to polyamide or peptide nucleic acid (PNA), morpholino and locked nucleic acid (LNA), as well as glycol nucleic acid (GNA) and threose nucleic acid (TNA).

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

In the context of the present invention, the term “oligonucleotide” refers to a nucleic acid sequence of up to about 50 nucleotides, e.g. 2 to about 50 nucleotides in length.

The term “polynucleotide” when used in the context of the present invention, refers to a nucleic acid of more than about 50 nucleotides in length, e.g. 51 or more nucleotides in length.

Oligonucleotides and polypeptides are prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (Meth. Enzymol. 68:90-99, 1979); the phosphodiester method of Brown et al. (Meth. Enzymol. 68:109-151, 1979); the diethylphosphoramidite method of Beaucage et al. (Tetrahedron Lett. 22:1859-1862, 1981); the triester method of Matteucci et al. (J. Am. Chem. Soc. 103:3185-3191, 1981); automated synthesis methods; or the solid support method of U.S. Pat. No. 4,458,066, or other methods known to those skilled in the art.

As used herein, the term “vector” refers to a protein or a polynucleotide or a mixture thereof which is capable of being introduced or of introducing proteins and/or nucleic acids comprised therein into a cell. Examples of vectors include but are not limited to plasmids, cosmids, phages, viruses or artificial chromosomes. In particular, a vector is used to transport a gene product of interest, such as e.g. foreign or heterologous DNA into a suitable host cell. Vectors may contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Vectors may further encompass “expression control sequences” that regulate the expression of the gene of interest. Typically, expression control sequences are polypeptides or polynucleotides such as but not limited to promoters, enhancers, silencers, insulators, or repressors. In a vector comprising more than one polynucleotide encoding for one or more gene products of interest, the expression may be controlled together or separately by one or more expression control sequences. More specifically, each polynucleotide comprised on the vector may be control by a separate expression control sequence or all polynucleotides comprised on the vector may be controlled by a single expression control sequence. Polynucleotides comprised on a single vector controlled by a single expression control sequence may form an open reading frame. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid.” The structures of these twenty natural amino acids are shown in, e.g., Stryer et al., Biochemistry, 5th ed., Freeman and Company (2002). Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12(13):R464-R466). The term “amino acid” also includes unnatural amino acids, modified amino acids (e.g., having modified side chains and/or backbones), and amino acid analogs. See, e.g., Zhang et al. (2004) “Selective incorporation of 5-hydroxytryptophan into proteins in mammalian cells,” Proc. Natl. Acad. Sci. U.S.A. 101(24):8882-8887, Anderson et al. (2004) “An expanded genetic code with a functional quadruplet codon” Proc. Natl. Acad. Sci. U.S.A. 101(20):7566-7571, Ikeda et al. (2003) “Synthesis of a novel histidine analogue and its efficient incorporation into a protein in vivo,” Protein Eng. Des. Sel. 16(9):699-706, Chin et al. (2003) “An Expanded Eukaryotic Genetic Code,” Science 301(5635):964-967, James et al. (2001) “Kinetic characterization of ribonuclease S mutants containing photoisomerizable phenylazophenylalanine residues,” Protein Eng. Des. Sel. 14(12):983-991, Kohrer et al. (2001) “Import of amber and ochre suppressor tRNAs into mammalian cells: A general approach to site-specific insertion of amino acid analogues into proteins,” Proc. Natl. Acad. Sci. U.S.A. 98(25):14310-14315, Bacher et al. (2001) “Selection and Characterization of Escherichia coli Variants Capable of Growth on an Otherwise Toxic Tryptophan Analogue,” J. Bacteriol. 183(18):5414-5425, Hamano-Takaku et al. (2000) “A Mutant Escherichia coli Tyrosyl-tRNA Synthetase Utilizes the Unnatural Amino Acid Azatyrosine More Efficiently than Tyrosine,” J. Biol. Chem. 275(51):40324-40328, and Budisa et al. (2001) “Proteins with {beta}-(thienopyrrolyl) alanines as alternative chromophores and pharmaceutically active amino acids,” Protein Sci. 10(7):1281-1292 Amino acids can be merged into peptides, polypeptides, or proteins.

In the context of the present invention, the term “peptide” refers to a short polymer of amino acids linked by peptide bonds. It has the same chemical (peptide) bonds as proteins, but is commonly shorter in length. The shortest peptide is a dipeptide, consisting of two amino acids joined by a single peptide bond. There can also be a tripeptide, tetrapeptide, pentapeptide, etc. Typically, a peptide has a length of up to 8, 10, 12, 15, 18 or 20 amino acids. A peptide has an amino end and a carboxyl end, unless it is a cyclic peptide.

In the context of the present invention, the term “polypeptide” refers to a single linear chain of amino acids bonded together by peptide bonds and typically comprises at least about 21 amino acids. A polypeptide can be one chain of a protein that is composed of more than one chain or it can be the protein itself if the protein is composed of one chain.

The term “stretch of amino acids” refers to a part of a peptide, polypeptide or protein having a particular amino acid sequence. The stretch of amino acids is thus defined firstly by the amino acids present in said stretch and secondly by the particular sequence of the amino acids present in that stretch. For example, in case a polypeptide comprises a certain stretch of amino acids, it is understood that the polypeptide comprises the amino acids specified to be present in that stretch in the particular order in which they are arranged in that stretch. However, a polypeptide not comprising a particular stretch of amino acids, may comprise the individual amino acids of that stretch but does not comprise the specified amino acids in that particular order in which they are arranged in that stretch.

In the context of present invention, the “primary structure” of a protein or polypeptide is the sequence of amino acids in the polypeptide chain. The “secondary structure” in a protein is the general three-dimensional form of local segments of the protein. It does not, however, describe specific atomic positions in three-dimensional space, which are considered to be tertiary structure. In proteins, the secondary structure is defined by patterns of hydrogen bonds between backbone amide and carboxyl groups. The “tertiary structure” of a protein is the three-dimensional structure of the protein determined by the atomic coordinates. The “quaternary structure” is the arrangement of multiple folded or coiled protein or polypeptide molecules in a multi-subunit complex.

The term “folding” or “protein folding” as used herein refers to the process by which a protein assumes its three-dimensional shape or conformation, i.e. whereby the protein is directed to form a specific three-dimensional shape through non-covalent interactions, such as but not limited to hydrogen bonding, metal coordination, hydrophobic forces, van der Waals forces, pi-pi interactions, and/or electrostatic effects. The term “folded protein” thus, refers to a protein its three-dimensional shape, such as its secondary, tertiary, or quaternary structure.

The term “fragment” used herein refers to naturally occurring fragments (e.g. splice variants) as well as artificially constructed fragments, in particular to those obtained by gene-technological means. Typically, a fragment has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130 , 140, 150, 160, 170, 180, 190, 200, 210, 220, 230, 240, 250, 260, 270, 280, 290, or 300 amino acids at its N-terminus and/or at its C-terminus and/or internally as compared to the parent polypeptide, preferably at its N-terminus, at its N- and C-terminus, or at its C-terminus.

The term “subunit” refers to any part of a macromolecule (e.g. a polypeptide, protein or polyprotein) into which this macromolecule can be divided. A macromolecule may consist of one or more subunits. Such division may exist due to functional (e.g. having certain binding or interaction functions) or structural (e.g. nucleotide or amino acid sequence, or secondary or tertiary structure) properties of the macromolecule and/or the individual subunit. In the context of the present invention it is preferred that the term “subunit” refers to a part of a protein or polyprotein. It is particularly preferred that such subunit folds and/or functions independently of the rest of the protein or polyprotein. In particular, in the context of the present invention, subunits of the RNA dependent RNA polymerase refers to the PA subunit, PB1 subunit, and PB2 subunit. The term subunit also encompasses variants, such as fragments, derivatives, or codon-optimized variants, of the native subunit. Preferably such variant of a subunit still exhibits the same function as the native subunit.

The term “carboxy-terminal fragment of the PA subunit” refers to a fragment of the PA subunit which is derived from the carboxy-terminal part of the PA subunit. The term “carboxy-terminal fragment of the PA subunit” does not require that the C-terminus of the PA subunit is present in the fragment, but refers to the fact that the fragment is derived from that part of the PA subunit which is positioned at the C-terminal two-thirds of the PA subunit, i.e. C-terminal of amino acid residue 258 (of the 713 amino acid residues in influenza A or the 726 amino acid residues in influenza B) at which the PA subunit is separable by trypsination. Accordingly, the term “carboxy-terminal fragment of the PA subunit” refers to a fragment which is derived from amino acids 258-713 of the PA subunit of Influenza A, or amino acids 258-726 of the PA subunit of Influenza B, or amino acids corresponding thereto, i.e. amino acids having an analogous position in a PA subunit aligned thereto.

The term “CTD” or “Pol II CTD” or “CTD of the cellular Pol II” refers to the c-terminal domain of the DNA-dependent RNA polymerase II present in eukaryotic cells. Typically, Pol II CTD in mammalian cells, in particular in human cells, consists of 52 heptad repeats with consensus sequence Y₁S₂P₃T₄S₅P₆S₇.

As used herein, the term “variant” is to be understood as a polypeptide or polynucleotide which differs in comparison to the polypeptide or polynucleotide from which it is derived by one or more changes in its length or sequence. The polypeptide or polynucleotide from which a polypeptide or polynucleotide variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed are modified molecules such as but not limited to post-translationally modified proteins (e.g. glycosylated, biotinylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Typically, a variant is constructed artificially, preferably by gene-technological means, whilst the parent protein or polynucleotide is a wild-type protein or polynucleotide, or a consensus sequence thereof. However, also naturally occurring variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, i.e. is functionally active.

In particular, the term “peptide variant”, “polypeptide variant”, “protein variant” is to be understood as a peptide, polypeptide, or protein which differs in comparison to the peptide, polypeptide, or protein from which it is derived by one or more changes in the amino acid sequence. The peptide, polypeptide, or protein, from which a peptide, polypeptide, or protein variant is derived, is also known as the parent peptide, polypeptide, or protein. Further, the variants usable in the present invention may also be derived from homologs, orthologs, or paralogs of the parent peptide, polypeptide, or protein or from artificially constructed variant, provided that the variant exhibits at least one biological activity of the parent peptide, polypeptide, or protein. The changes in the amino acid sequence may be amino acid exchanges, insertions, deletions, N-terminal truncations, or C-terminal truncations, or any combination of these changes, which may occur at one or several sites. A peptide, polypeptide, or protein variant may exhibit a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200) changes in the amino acid sequence (i.e. exchanges, insertions, deletions, N-terminal truncations, and/or C-terminal truncations). The amino acid exchanges may be conservative and/or non-conservative. Alternatively or additionally, a “variant” as used herein, can be characterized by a certain degree of sequence identity to the parent peptide, polypeptide, or protein from which it is derived. More precisely, a peptide, polypeptide, or protein variant in the context of the present invention exhibits at least 80% sequence identity to its parent peptide, polypeptide, or protein. The sequence identity of peptide, polypeptide, or protein variants is over a continuous stretch of 20, 30, 40, 45, 50, 60, 70, 80, 90, 100 or more amino acids.

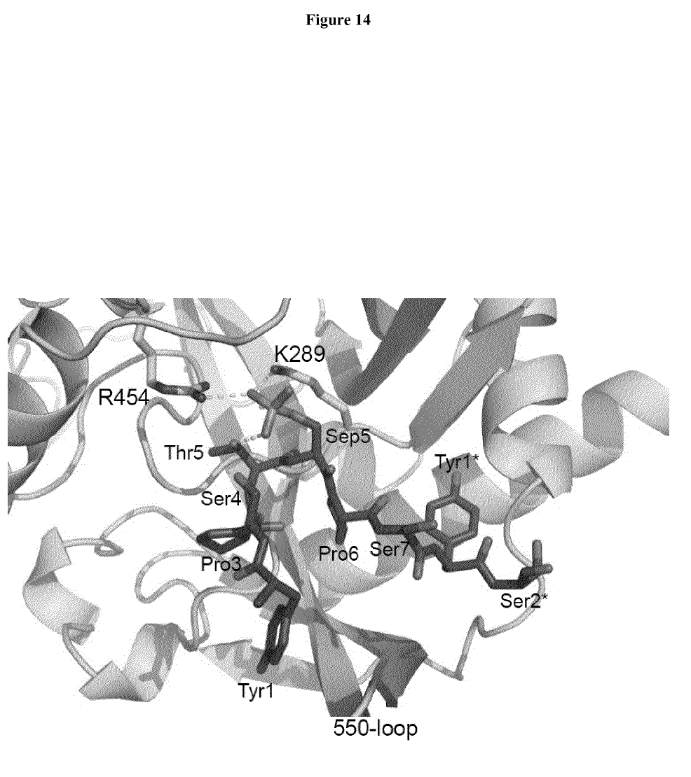

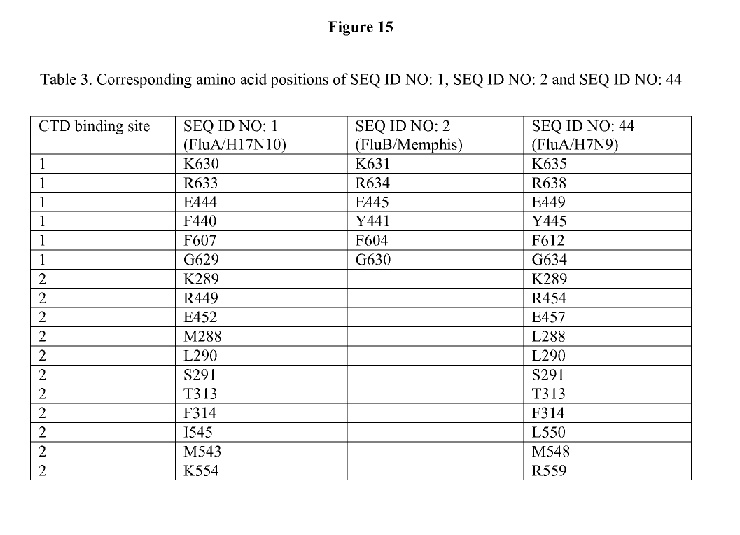

Residues in two or more polypeptides are said to “correspond” to each other if the residues occupy an analogous position in the polypeptide structures. As is well known in the art, analogous positions in two or more polypeptides can be determined by aligning the polypeptide sequences based on amino acid sequence or structural similarities. The term “correspondence” to another sequence (e.g., regions, fragments, nucleotide or amino acid positions, or the like) refers to the convention of numbering of nucleotide or amino acid positions and the alignment of the sequences in a manner that maximizes the percentage of sequence identity. Because not all positions within a given “corresponding region” need be identical, non-matching positions within a corresponding region may be regarded as “corresponding positions.” Accordingly, as used herein, referral to an “amino acid position corresponding to amino acid position [X]” of a specified nucleotide or amino acid sequence refers to equivalent positions, based on alignment, in another nucleotide or amino acid sequence aligned thereto, and structural homologues and families. In some embodiments of the present invention, “correspondence” of amino acid positions are determined with respect to a region of interest comprising one or more motifs of SEQ ID NO: 1, SEQ ID NO: 2, or SEQ ID NO: 44. For example, amino acid arginine at position 449 (R449) of SEQ ID NO: 1 corresponds to amino acid arginine at position 454 (R454) of SEQ ID NO: 44. In particular, corresponding amino acid positions for CTD binding site 1 and 2 of SEQ ID NO: 1, SEQ ID NO: 2, and SEQ ID NO: 44 are shown in FIG. 15. When a polypeptide sequence differs from a specified sequence e.g., by changes in amino acids or addition or deletion of amino acids, it may be that a particular mutation associated with improved activity will not be in the same position number as it is in said specified sequence. Alignment tools allowing the skilled person to analyse the sequences and to identify corresponding positions are very-well known in the art and can be, for example, obtained on the World Wide Web, e.g., ClustalW (www.ebi.ac.uk/clustalw) or Align (http://www.ebi.ac.uk/emboss/align/index.html) using standard settings, preferably for Align EMBOSS:needle, Matrix: Blosum62, Gap Open 10.0, Gap Extend 0.5. Those skilled in the art understand that it may be necessary to introduce gaps in either sequence to produce a satisfactory alignment. Residues in two or more PA subunits are said to “correspond” if the residues are aligned in the best sequence alignment. The “best sequence alignment” between two polypeptides is defined as the alignment that produces the largest number of aligned identical residues. The “region of best sequence alignment” ends and, thus, determines the metes and bounds of the length of the comparison sequence for the purpose of the determination of the similarity score, if the sequence similarity, preferably identity, between two aligned sequences drops to less than 30%, preferably less than 20%, more preferably less than 10% over a length of 10, 20 or 30 amino acids.

The term “associate” as used in the context of identifying compounds with the methods of the present invention refers to a condition of proximity between a moiety (i.e., chemical entity or compound or portions or fragments thereof), and an endonuclease active site of the PA subunit. The association may be non-covalent, i.e., where the juxtaposition is energetically favoured by, for example, hydrogen-bonding, van der Waals, electrostatic, or hydrophobic interactions, or it may be covalent.

The term “recombinant” refers to an amino acid sequence or a nucleotide sequence that is intentionally modified by recombinant methods. The term “recombinant nucleic acid” as used herein refers to a nucleic acid which is formed in vitro, and optionally further manipulated by endonucleases to form a nucleic acid molecule not normally found in nature. Exemplified, recombinant nucleic acids include cDNA, in a linear form, as well as vectors formed in vitro by ligating DNA molecules that are not normally joined. It is understood that once a recombinant nucleic acid is made and introduced into a host cell, it will replicate non-recombinantly, i.e. using the in vivo cellular machinery of the host cell rather than in vitro manipulations. Accordingly, nucleic acids which were produced recombinantly, may be replicated subsequently non-recombinantly. A “recombinant protein” is a protein made using recombinant techniques, e.g. through the expression of a recombinant nucleic acid as depicted above. The term “recombinant vector” as used herein includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

The term “host cell” refers to a cell that harbours a vector (e.g. a plasmid or virus). Such host cell may either be a prokaryotic (e.g. a bacterial cell) or a eukaryotic cell (e.g. a fungal, plant or animal cell). Host cells include both single-cellular prokaryote and eukaryote organisms (e.g., bacteria, yeast, and actinomycetes) as well as single cells from higher order plants or animals when being grown in cell culture. “Recombinant host cell”, as used herein, refers to a host cell that comprises a polynucleotide that codes for a polypeptide fragment of interest, i.e., the fragment of the viral PA subunit or variants thereof according to the invention. This polynucleotide may be found inside the host cell (i) freely dispersed as such, (ii) incorporated in a recombinant vector, or (iii) integrated into the host cell genome or mitochondrial DNA. The recombinant cell can be used for expression of a polynucleotide of interest or for amplification of the polynucleotide or the recombinant vector of the invention. The term “recombinant host cell” includes the progeny of the original cell which has been transformed, transfected, or infected with the polynucleotide or the recombinant vector of the invention. A recombinant host cell may be a bacterial cell such as an E. coli cell, a yeast cell such as Saccharomyces cerevisiae or Pichia pastoris, a plant cell, an insect cell such as SF9 or High Five cells, or a mammalian cell. Preferred examples of mammalian cells are Chinese hamster ovary (CHO) cells, green African monkey kidney (COS) cells, human embryonic kidney (HEK293) cells, HELA cells, and the like.

The “percentage of sequences identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” in the context of two or more nucleic acids or polypeptide sequences, refers to two or more sequences or subsequences that are the same, i.e. comprise the same sequence of nucleotides or amino acids. Sequences are “substantially identical” to each other if they have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over a specified region), when compared and aligned for maximum correspondence over a comparison window, or designated region as measured using one of the following sequence comparison algorithms or by manual alignment and visual inspection. These definitions also refer to the complement of a test sequence. Optionally, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide. Optionally, the polynucleotide in question and the reference polynucleotide exhibit the indicated sequence identity over a continuous stretch of 60, 90, 120, 135, 150, 180, 210, 240, 270, 300, 400, 500, 600, 700, 800, 900, 1000 or more nucleotides or over the entire length of the reference polynucleotide.

For term “sequence comparison” refers to the process wherein one sequence acts as a reference sequence, to which test sequences are compared. When using a sequence comparison algorithm, test and reference sequences are entered into a computer, if necessary subsequence coordinates are designated, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise.

In a sequence alignment, the term “comparison window” refers to those stretches of contiguous positions of a sequence which are compared to a reference stretch of contiguous positions of a sequence having the same number of positions. The number of contiguous positions selected may range from 10 to 1000, i.e. may comprise 20, 30, 40, 50, 60, 70, 80, 90, 100, 150, 200, 250, 300, 350, 400, 450, 500, 550, 600, 650, 700, 750, 800, 850, 900, 950, or 1000 contiguous positions. Typically, the number of contiguous positions ranges from about 20 to 800 contiguous positions, from about 20 to 600 contiguous positions, from about 50 to 400 contiguous positions, from about 50 to about 200 contiguous positions, from about 100 to about 150 contiguous positions.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=-4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=-4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

As used herein, the term “consensus” refers to an amino acid or nucleotide sequence that represents the results of a multiple sequence alignment, wherein related sequences were compared to each other. Such consensus sequence is composed of the amino acids or nucleotides most commonly observed at each position. In the context of the present invention it is preferred that the sequences used in the sequence alignment to obtain the consensus sequence are sequences of different viral subtypes strains isolated in various different disease outbreaks worldwide. Each individual sequence used in the sequence alignment is referred to as the sequence of a particular virus “isolate”.

Semi-conservative and especially conservative amino acid substitutions, wherein an amino acid is substituted with a chemically related amino acid are preferred. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are:

| Amino acid | Conservative | Semi-conservative substitution |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure.

A tag (or marker or label) is any kind of substance which is able to indicate the presence of another substance or complex of substances. The marker can be a substance that is linked to or introduced in the substance to be detected. Detectable markers are used in molecular biology and biotechnology to detect e.g. a protein, a product of an enzymatic reaction, a second messenger, DNA, interactions of molecules etc. Examples of suitable tags or labels include fluorophores, chromophores, radiolabels, metal colloids, enzymes, or chemiluminescent or bioluminescent molecules. In the context of the present invention suitable tags are preferably protein tags whose peptide sequences is genetically grafted into or onto a recombinant protein. Protein tags may e.g. encompass affinity tags, solubilization tags, chromatography tags, epitope tags, or Fluorescence tags.

“Affinity tags” are appended to proteins so that the protein can be purified from its crude biological source using an affinity technique. These include chitin binding protein (CBP), maltose binding protein (MBP), and glutathione-S-transferase (GST). The poly(His) tag is a widely used protein tag which binds to metal matrices.

“Solubilization tags” are used, especially for recombinant proteins expressed in chaperone-deficient species to assist in the proper folding in proteins and keep them from precipitating. These include thioredoxin (TRX) and poly(NANP). Some affinity tags have a dual role as a solubilization agent, such as MBP, and GST.

“Chromatography tags” are used to alter chromatographic properties of the protein to afford different resolution across a particular separation technique. Often, these consist of polyanionic amino acids, such as FLAG-tag.

“Epitope tags” are short peptide sequences which are chosen because high-affinity antibodies can be reliably produced in many different species. These are usually derived from viral genes, which explain their high immunoreactivity. Epitope tags include V5-tag, Myc-tag, and HA-tag. These tags are particularly useful for western blotting, immunofluorescence and immunoprecipitation experiments, although they also find use in antibody purification.

“Fluorescence tags” are used to give visual readout on a protein. GFP and its variants are the most commonly used fluorescence tags. More advanced applications of GFP include using it as a folding reporter (fluorescent if folded, colourless if not). Further examples of fluorophores include fluorescein, rhodamine, and sulfoindocyanine dye Cy5.

Examples of such tag include but are not limited to AviTag (a peptide allowing biotinylation by the enzyme BirA and isolation by streptavidin (SEQ ID NO: 4, GLNDIFEAQKIEWHE)), Calmodulin-tag (a peptide bound by the protein calmodulin (SEQ ID NO: 5, KRRWKKNFIAVSAANRFKKISSSGAL)), polyglutamate tag (a peptide binding efficiently to anion-exchange resin such as Mono-Q (SEQ ID NO: 6, EEEEEE)), E-tag (a peptide recognized by an antibody (SEQ ID NO: 7, GAPVPYPDPLEPR)), FLAG-tag (a peptide recognized by an antibody (SEQ ID NO: 8, DYKDDDDK)), HA-tag (a peptide recognized by an antibody (SEQ ID NO: 9, YPYDVPDYA)), His-tag (5-10 histidines bound by a nickel or cobalt chelate (SEQ ID NO: 10, HHHHHH)), Myc-tag (a short peptide recognized by an antibody (SEQ ID NO: 11, EQKLISEEDL)), S-tag (SEQ ID NO: 12, KETAAAKFERQHMDS), SBP-tag (a peptide which binds to streptavidin (SEQ ID NO: 13, MDEKTTGWRGGHVVEGLAGELEQLRARLEHHPQGQREP)), Softag 1 (for mammalian expression (SEQ ID NO: 14, SLAELLNAGLGGS)), Softag 3 (for prokaryotic expression (SEQ ID NO: 15, TQDPSRVG)), Strep-tag (a peptide which binds to streptavidin or the modified streptavidin called streptactin (Strep-tag II: SEQ ID NO: 16, WSHPQFEK)), TC tag (a tetracysteine tag that is recognized by FlAsH and ReAsH biarsenical compounds (SEQ ID NO: 17, CCPGCC)), V5 tag (a peptide recognized by an antibody (SEQ ID NO: 18, GKPIPNPLLGLDST)), VSV-tag (a peptide recognized by an antibody (SEQ ID NO: 19, YTDIEMNRLGK)), Xpress tag (SEQ ID NO: 20, DLYDDDDK), Isopeptag (a peptide which binds covalently to pilin-C protein (SEQ ID NO: 22, TDKDMTITFTNKKDAE)), SpyTag (a peptide which binds covalently to SpyCatcher protein (SEQ ID NO: 23, AHIVMVDAYKPTK)), BCCP (Biotin Carboxyl Carrier Protein, a protein domain biotinylated by BirA enabling recognition by streptavidin), Glutathione-S-transferase-tag (a protein which binds to immobilized glutathione), Green fluorescent protein-tag (a protein which is spontaneously fluorescent and can be bound by nanobodies), Maltose binding protein-tag (a protein which binds to amylose agarose), Nus-tag, Thioredoxin-tag, Fc-tag (derived from immunoglobulin Fc domain), Ty tag, Designed Intrinsically Disordered tags containing disorder promoting amino acids (P,E,S,T,A,Q,G, . . . ) Minde, David P; Els F Halff; Sander J Tans (2013 Sep. 1). “Designing disorder: Tales of the unexpected tails”. Intrinsically Disordered Proteins 1 (2): e26790.

As used herein, the term “crystal” or “crystalline” means a structure (such as a three-dimensional solid aggregate) in which the plane faces intersect at definite angles and in which there is a regular structure (such as internal structure) of the constituent chemical species. The term “crystal” can include any one of: a solid physical crystal form such as an experimentally prepared crystal, a crystal structure derivable from the crystal (including secondary and/or tertiary and/or quaternary structural elements), a 2D and/or 3D model based on the crystal structure, a representation thereof such as a schematic representation thereof or a diagrammatic representation thereof, or a data set thereof for a computer. In one aspect, the crystal is usable in X-ray crystallography techniques. Here, the crystals used can withstand exposure to X-ray beams and are used to produce diffraction pattern data necessary to solve the X-ray crystallographic structure. A crystal may be characterized as being capable of diffracting X-rays in a pattern defined by one of the crystal forms depicted in T. L. Blundell and L. N. Johnson, “Protein Crystallography”, Academic Press, New York (1976).

The term “unit cell” refers to a basic cubic or parallelepiped shaped block. The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal. In a space group designation the capital letter indicates the lattice type and the other symbols represent symmetry operations that can be carried out on the contents of the asymmetric unit without changing its appearance.

The term “structure coordinates” refers to a set of values that define the position of one or more amino acid residues with reference to a system of axes. The term refers to a data set that defines the three-dimensional structure of a molecule or molecules (e.g., Cartesian coordinates, temperature factors, and occupancies). Structural coordinates can be slightly modified and still render nearly identical three-dimensional structures. A measure of a unique set of structural coordinates is the root mean square deviation of the resulting structure. Structural coordinates that render three-dimensional structures (in particular, a three-dimensional structure of an enzymatically active center) that deviate from one another by a root mean square deviation of less than 3 Å, 2 Å, 1.5 Å, 1.0 Å, or 0.5 Å may be viewed by a person of ordinary skill in the art as very similar.

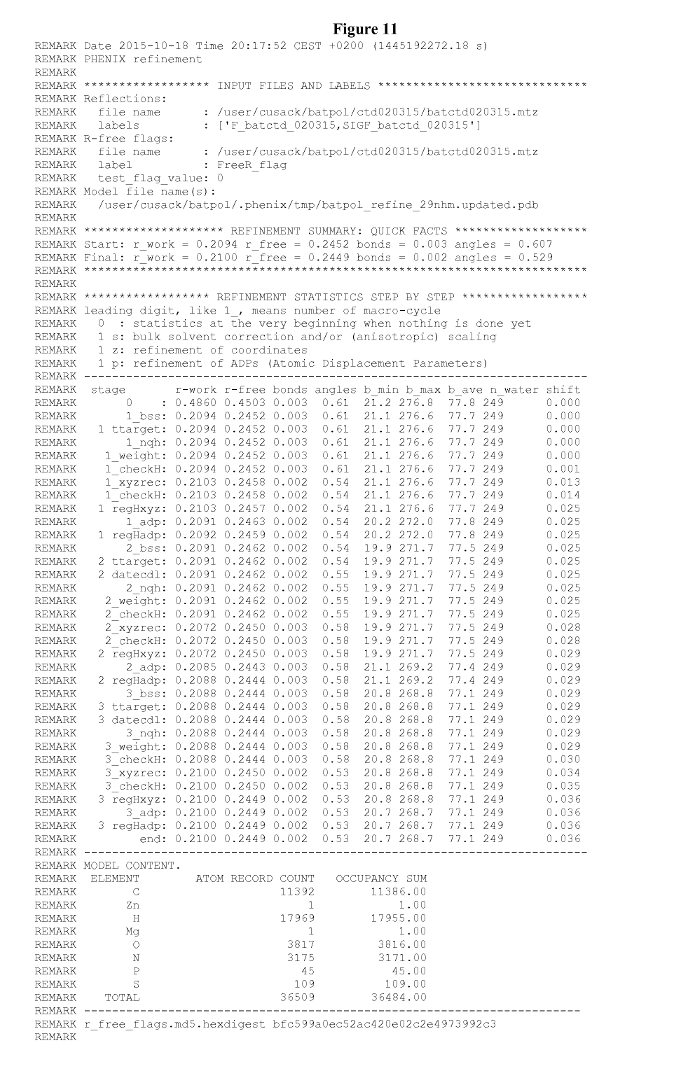

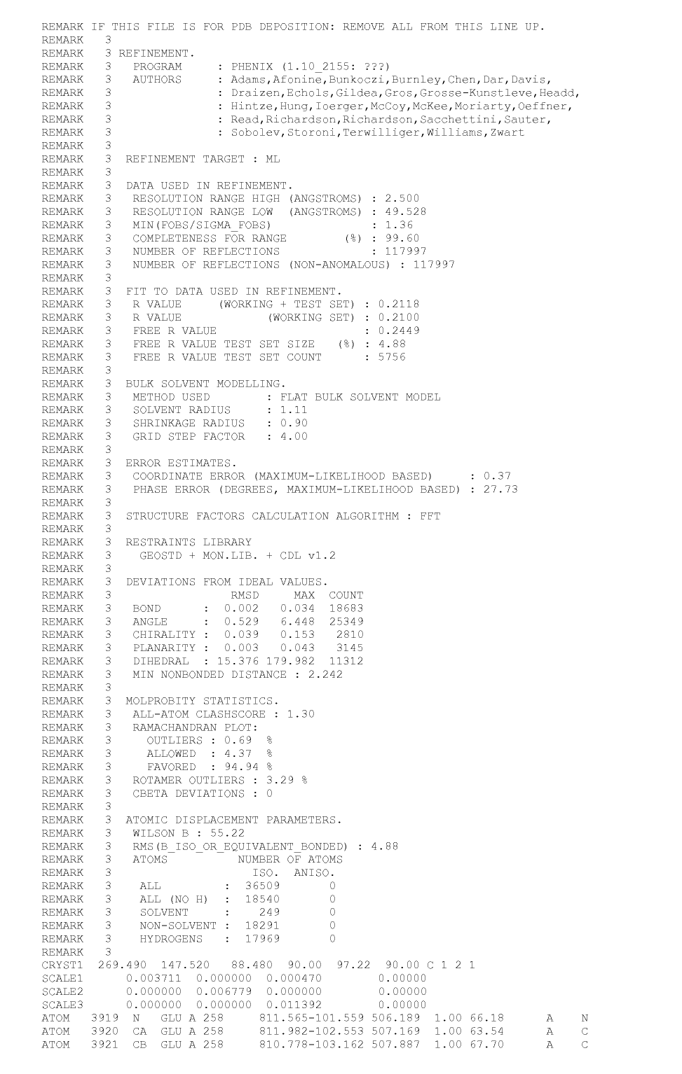

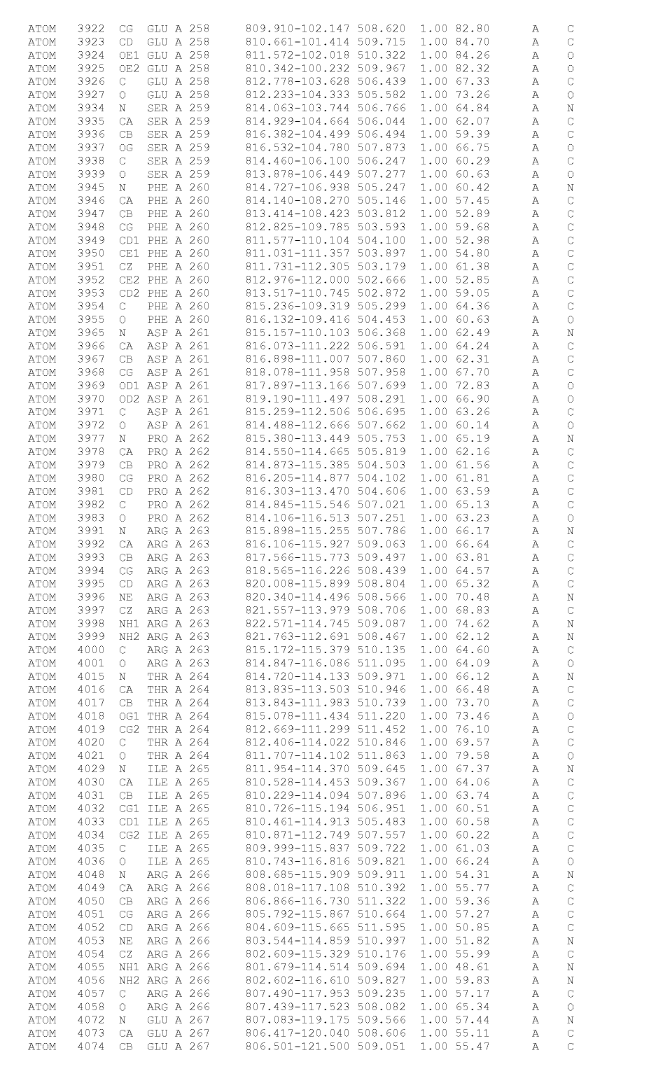

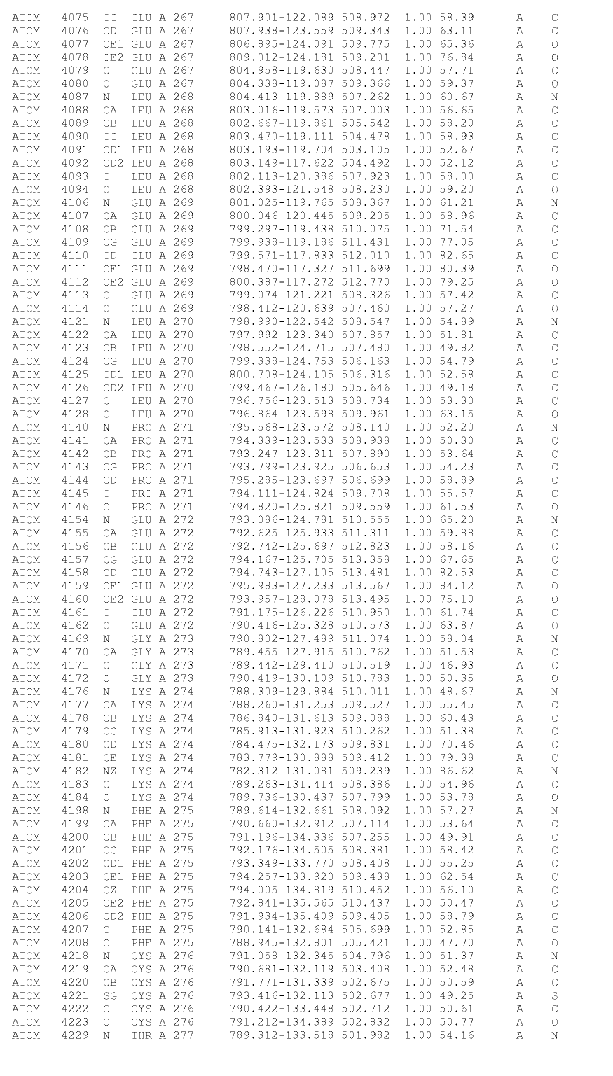

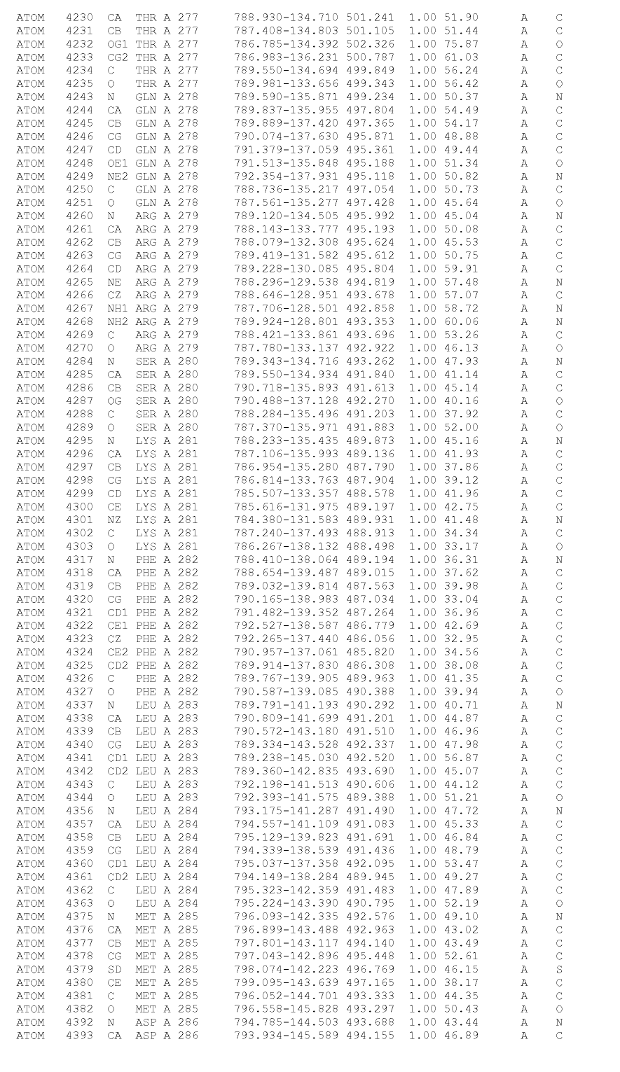

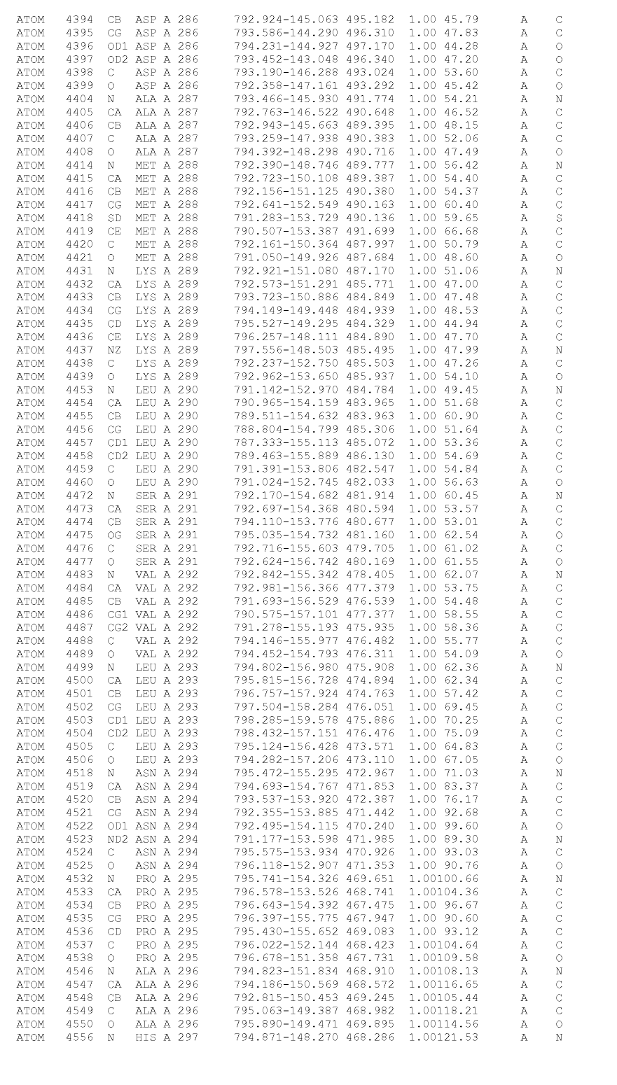

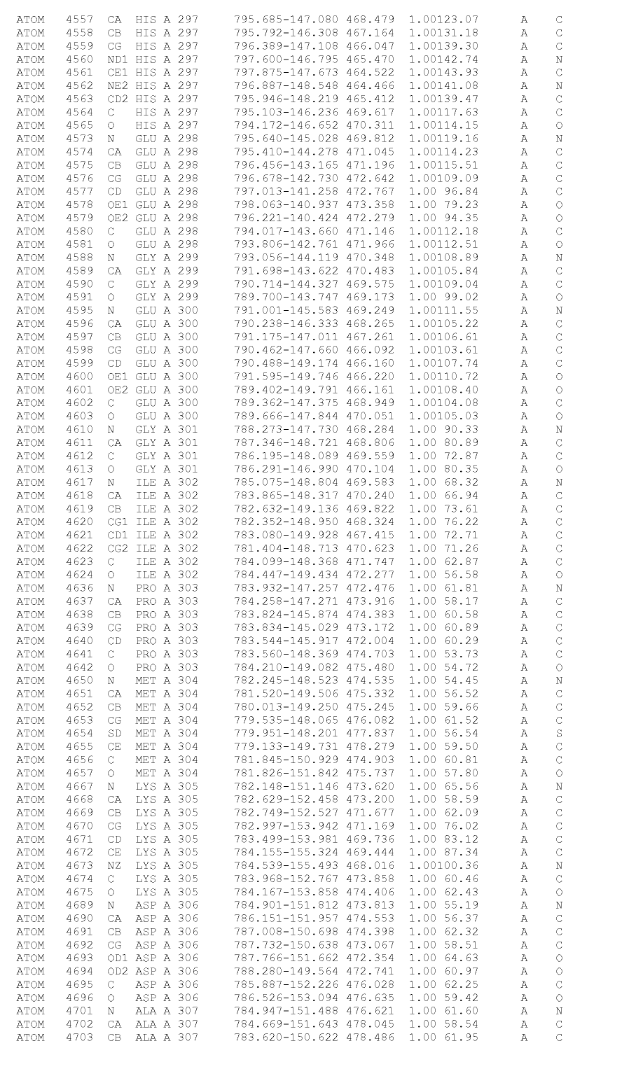

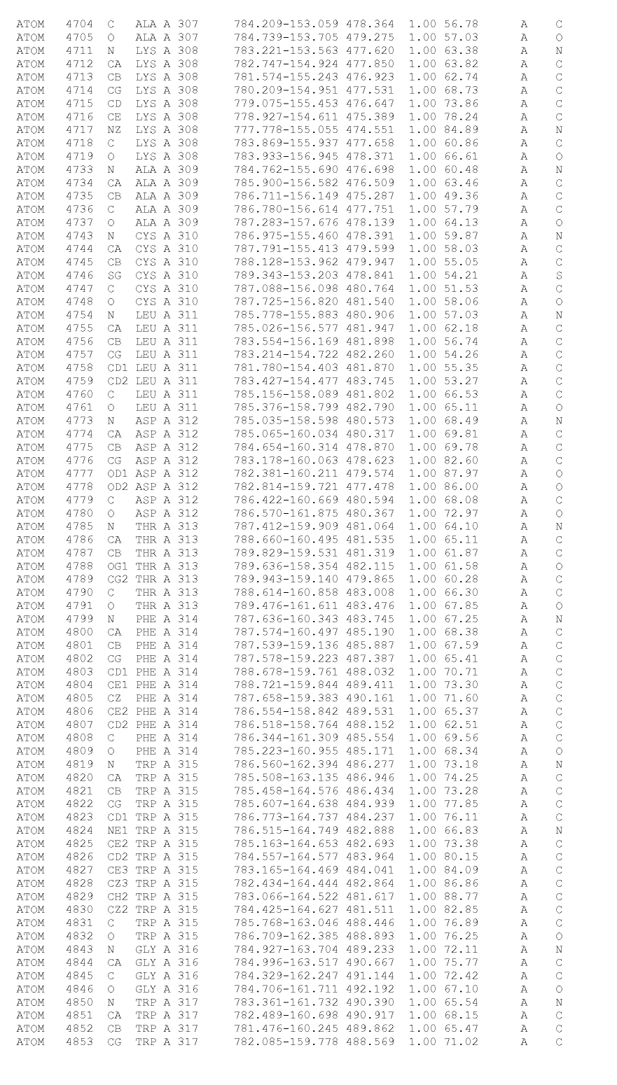

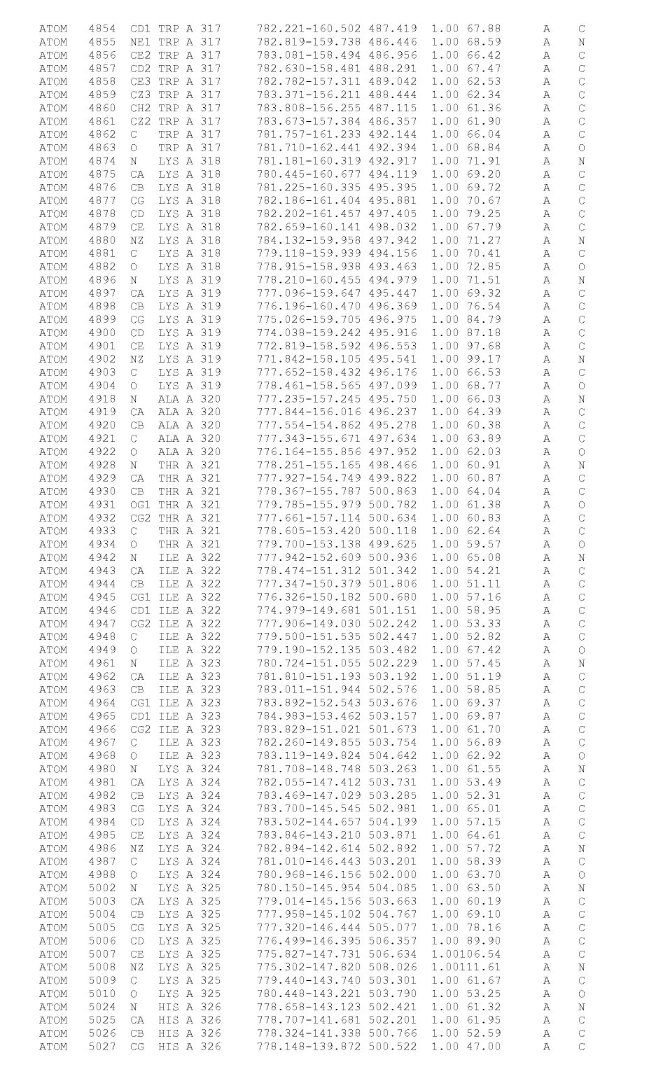

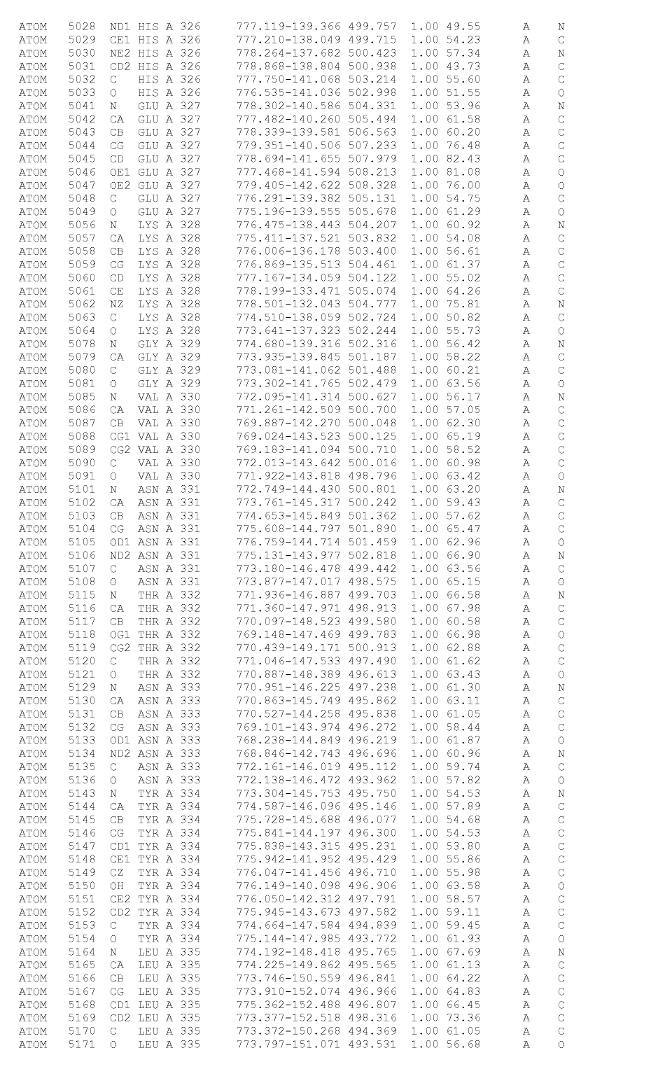

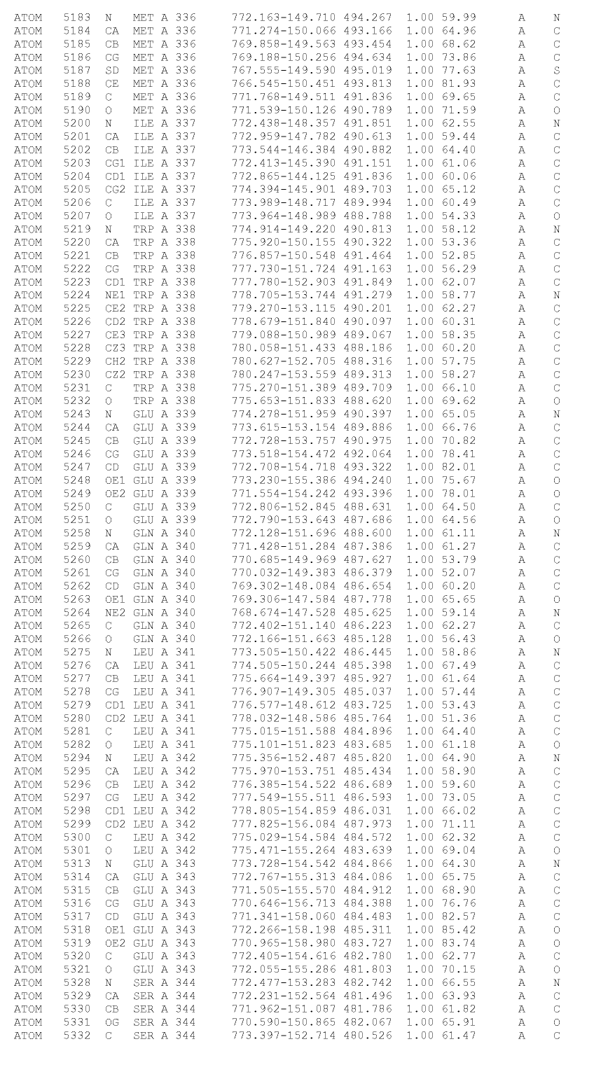

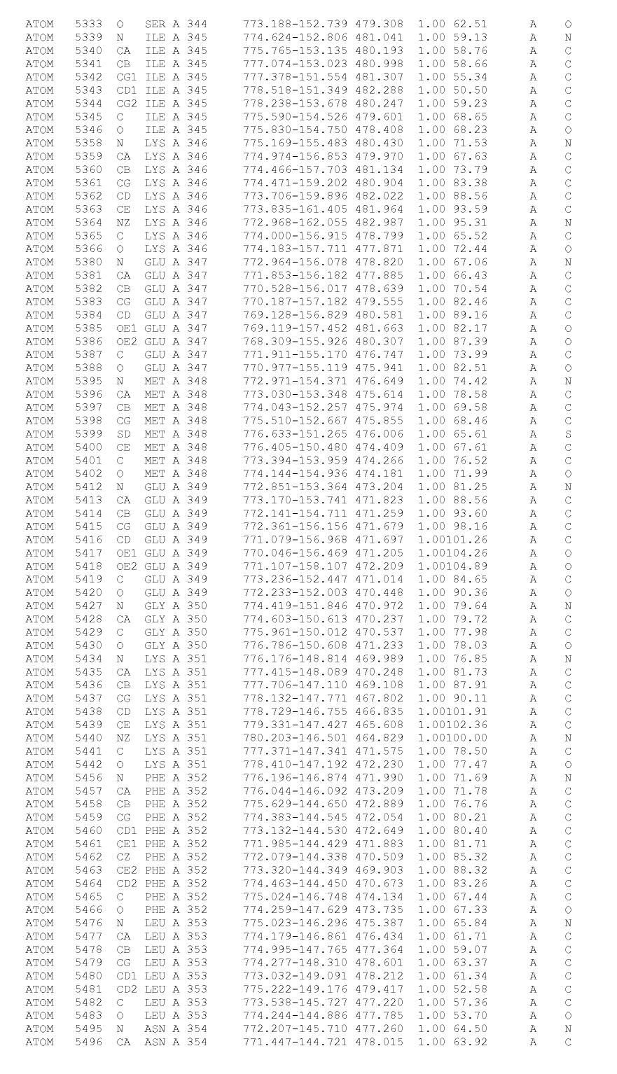

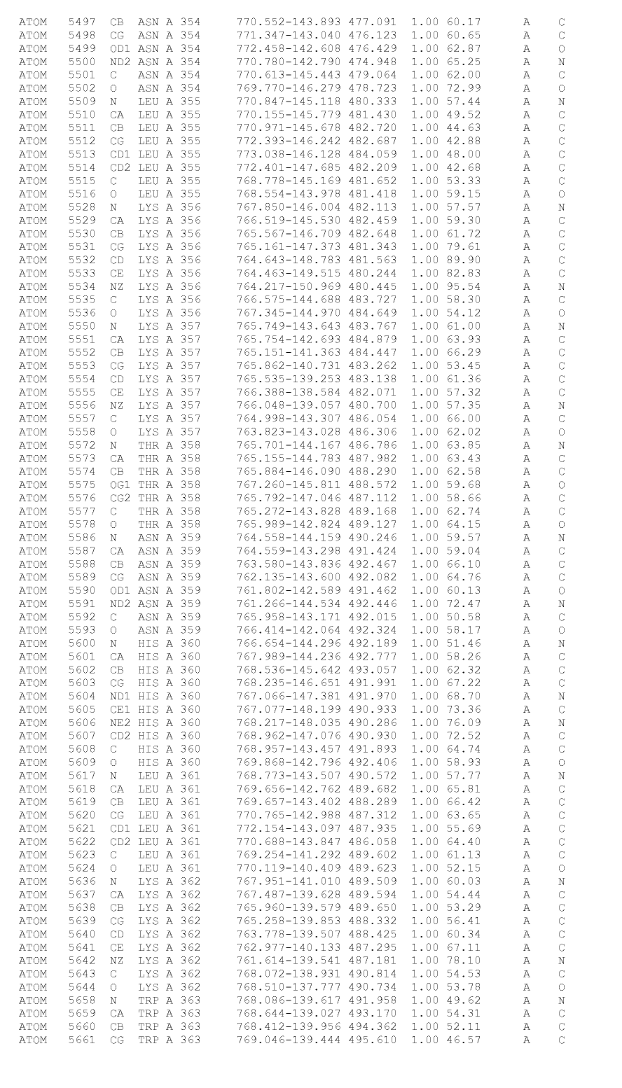

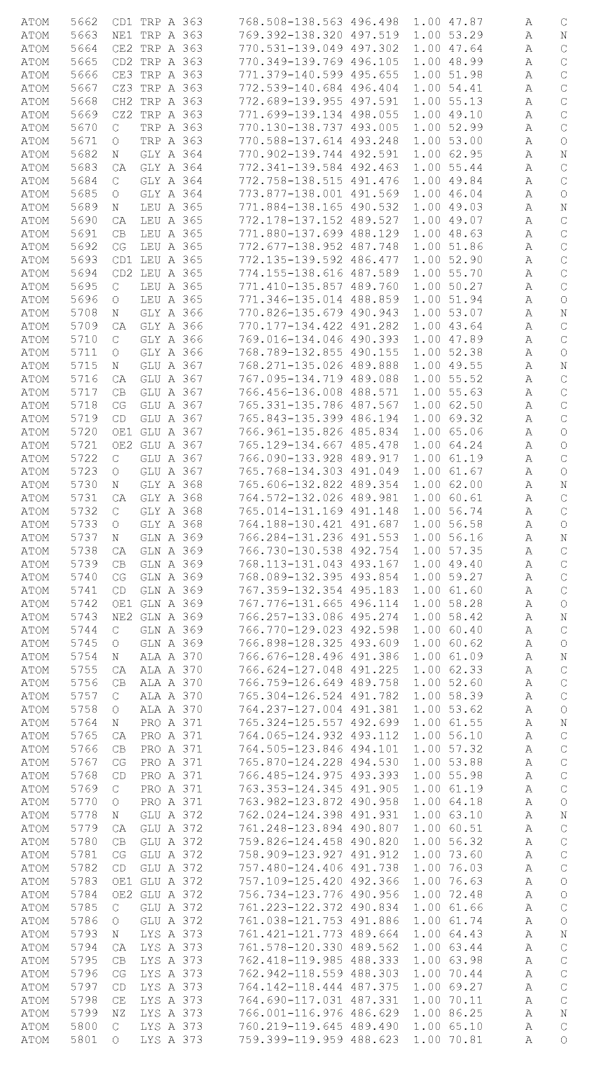

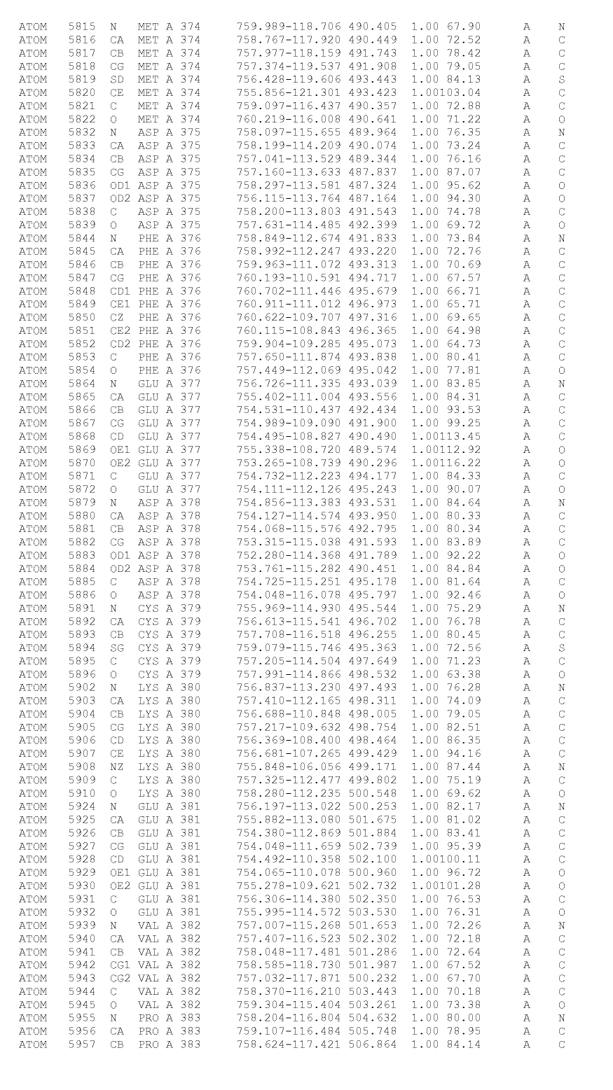

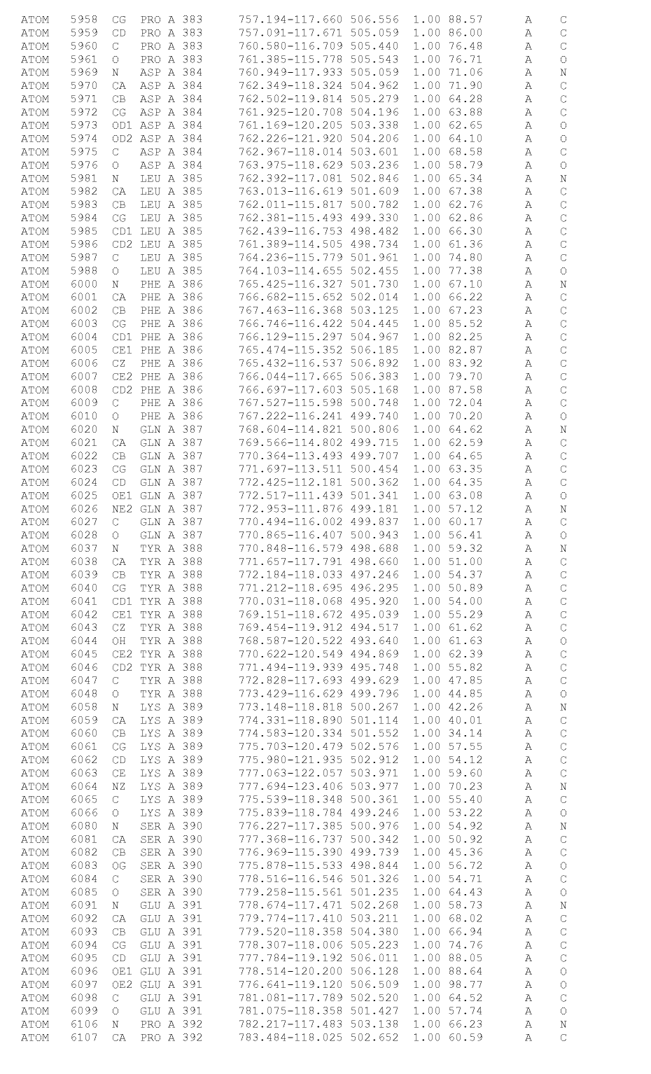

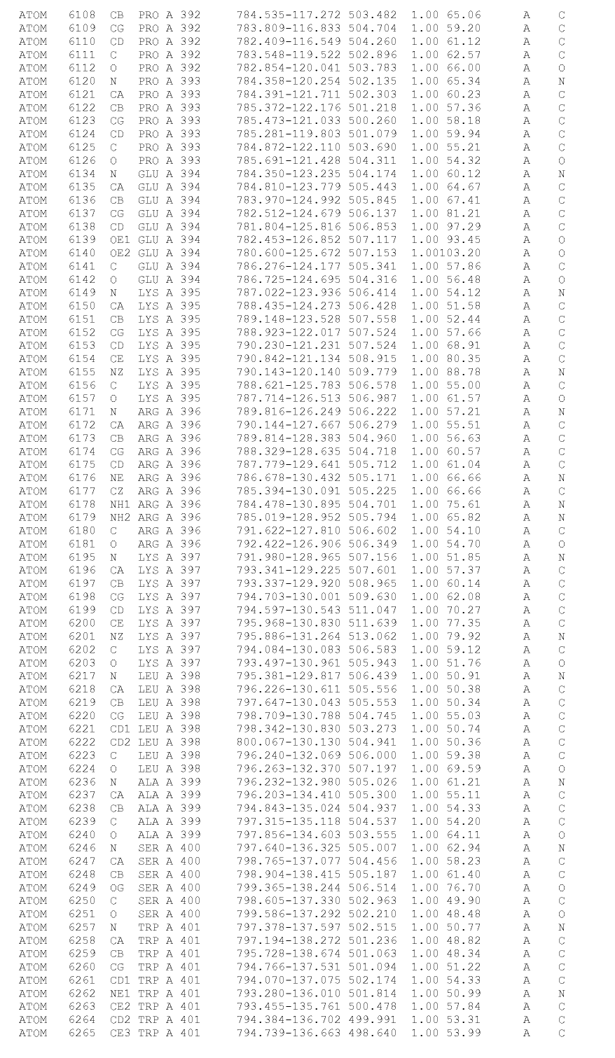

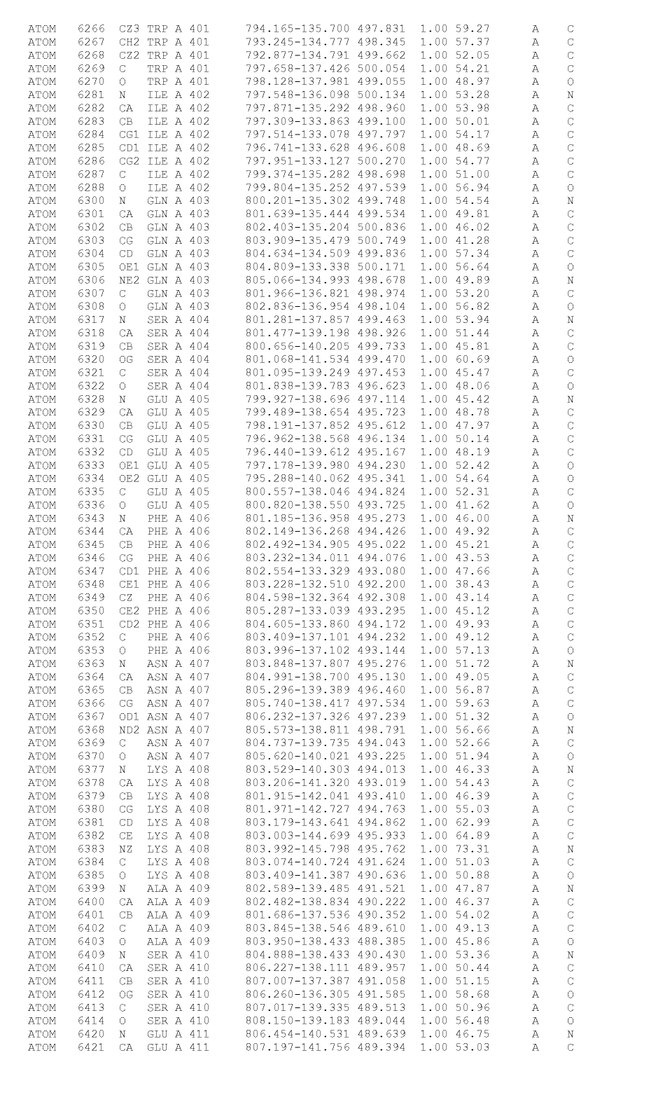

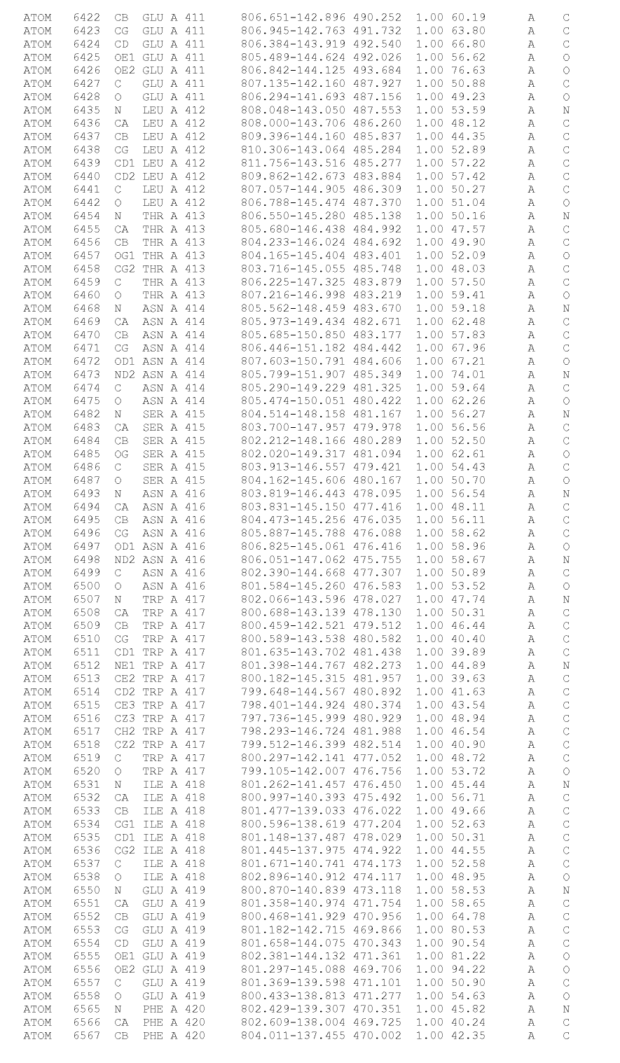

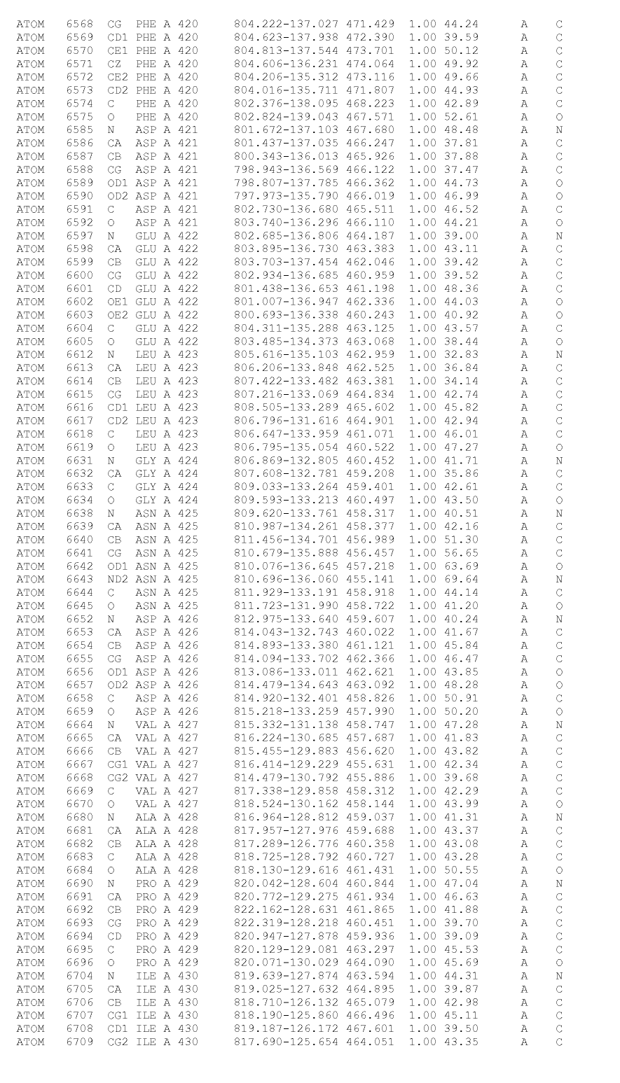

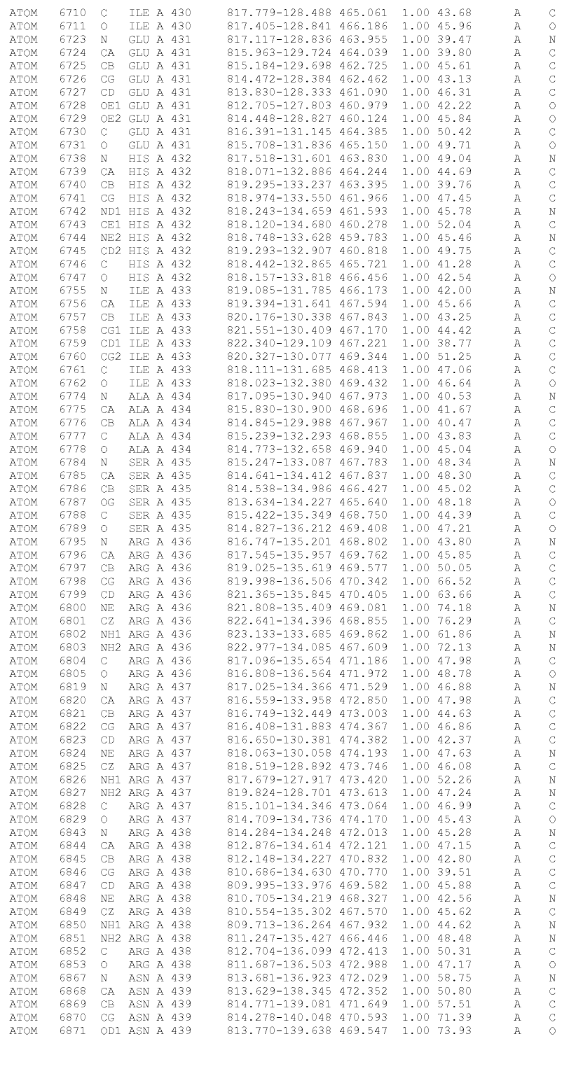

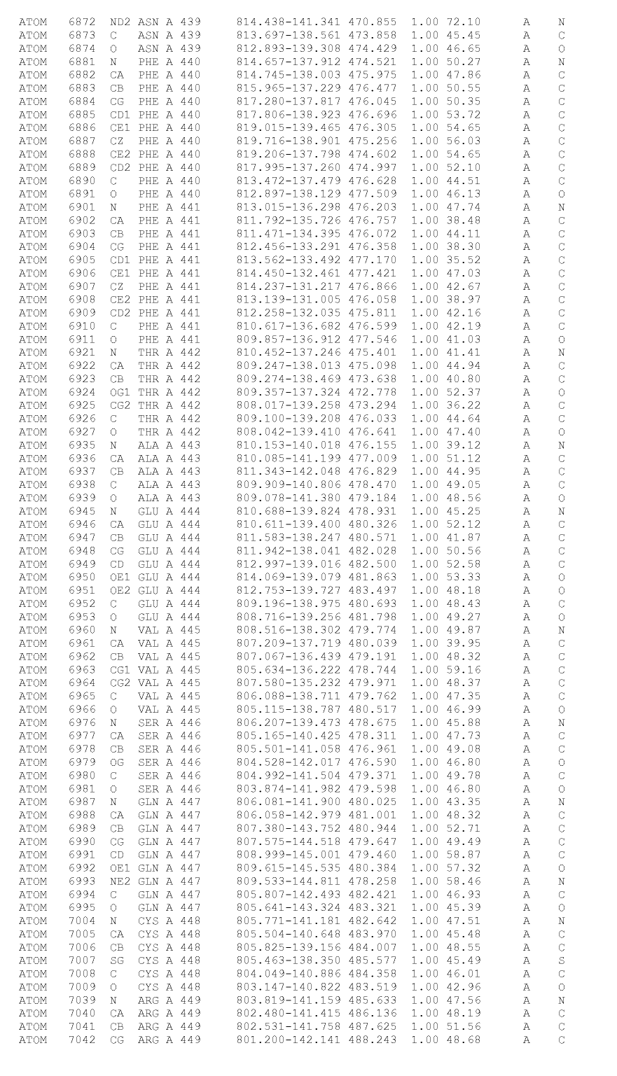

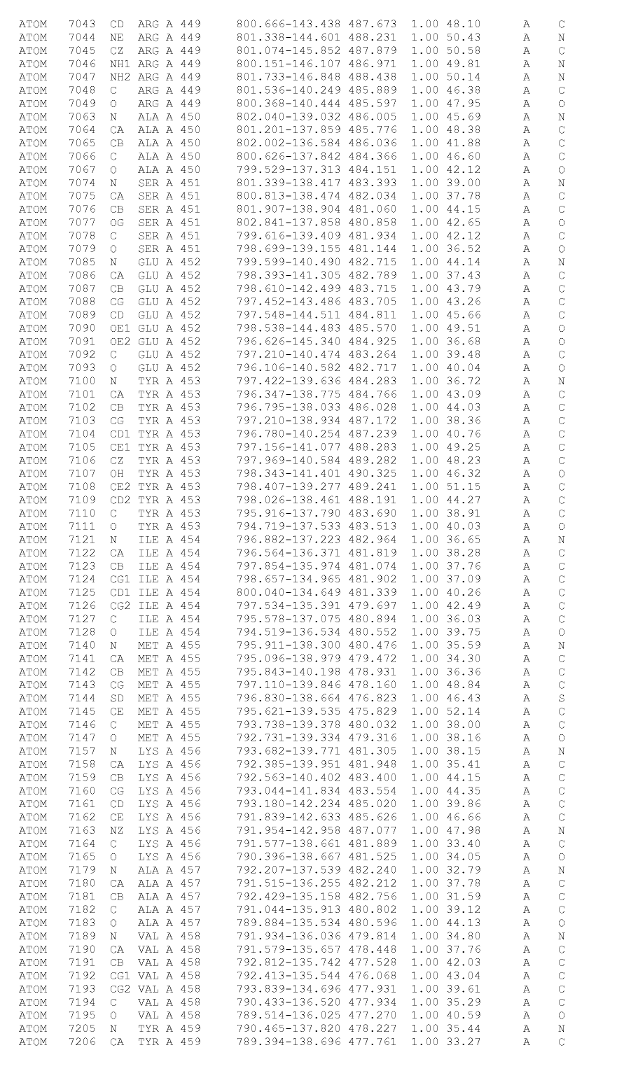

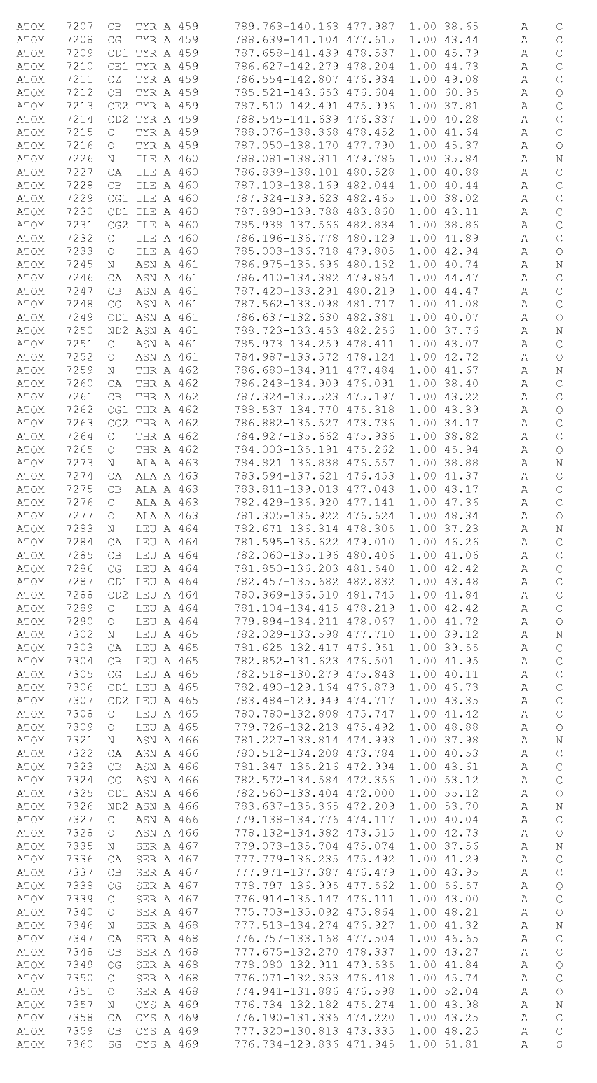

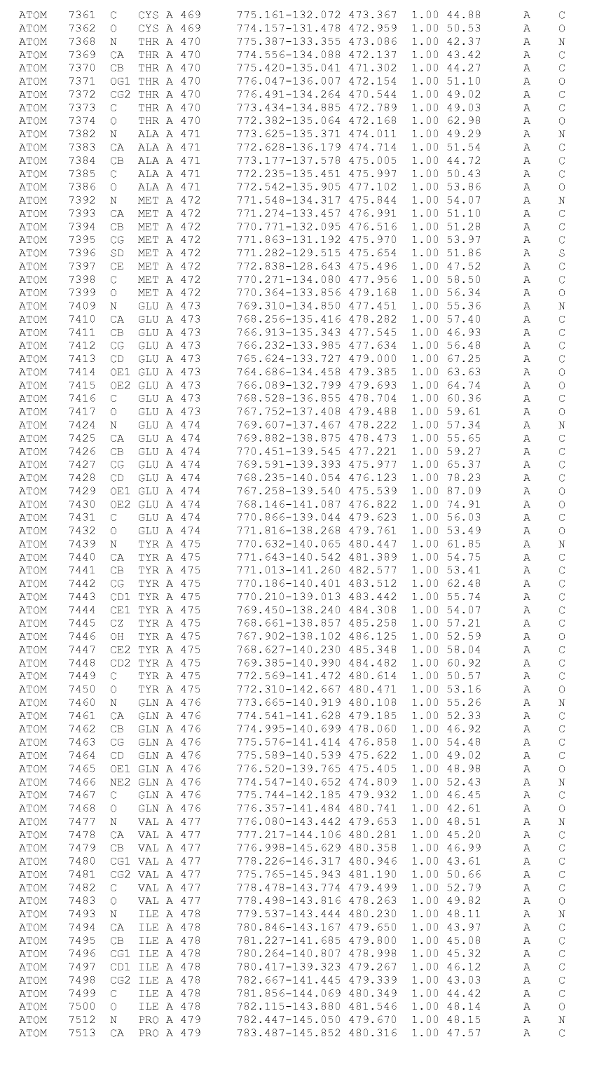

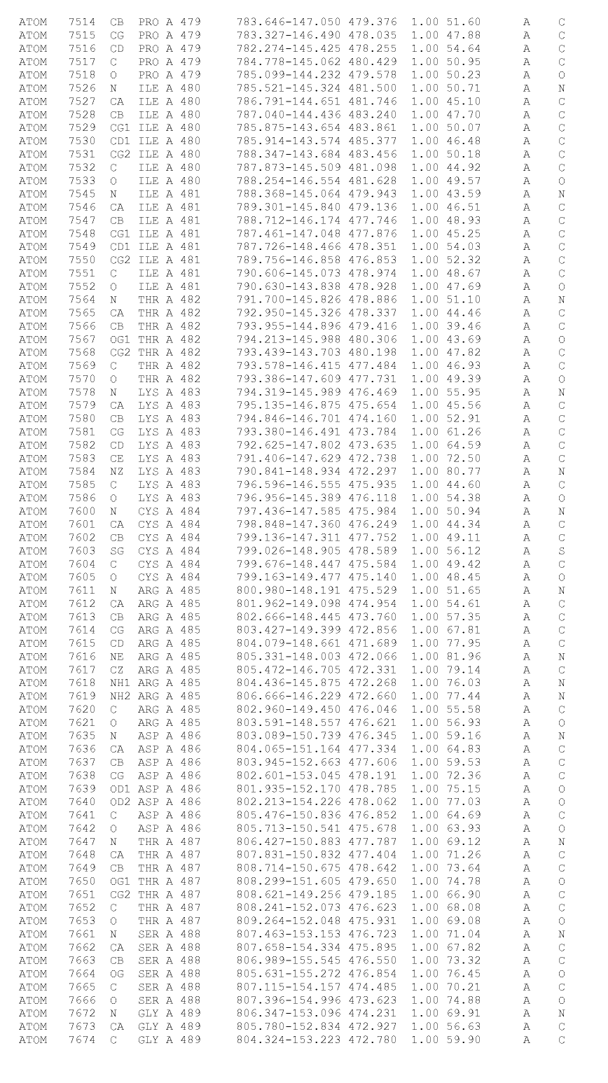

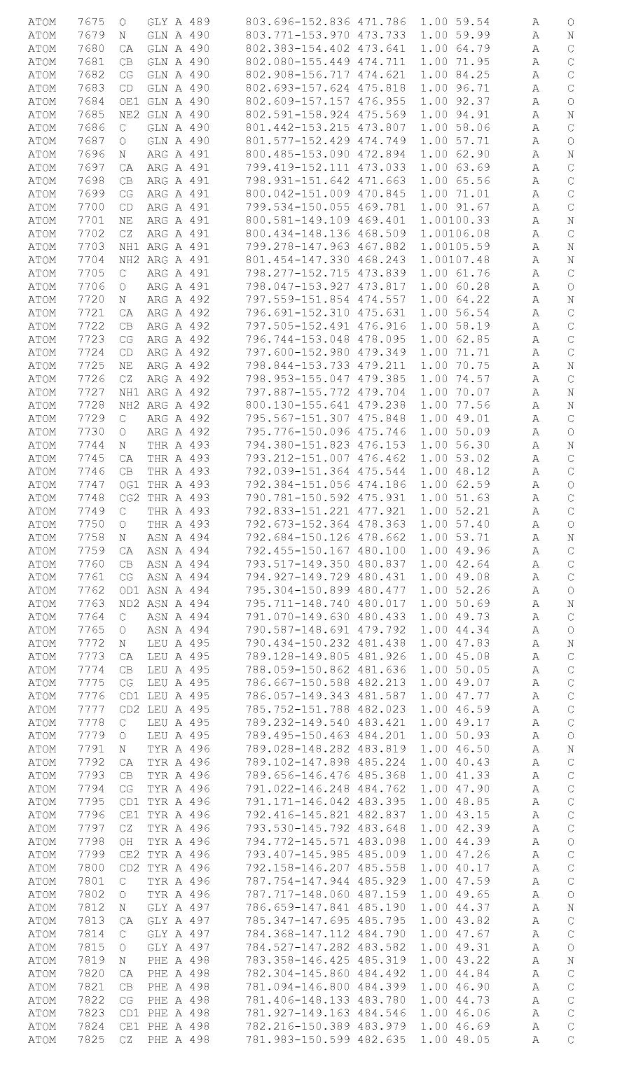

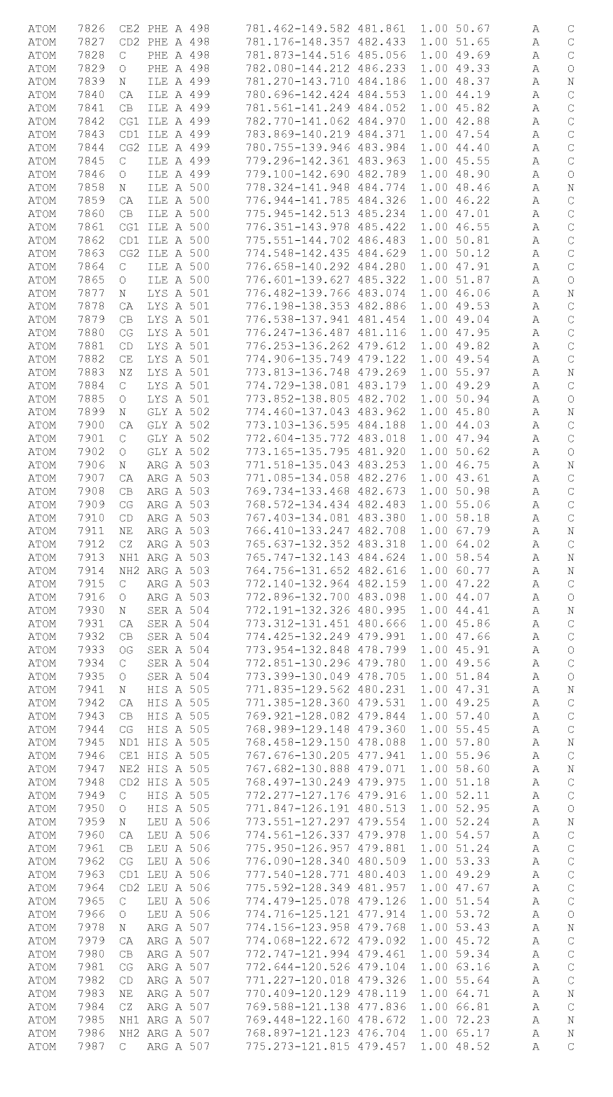

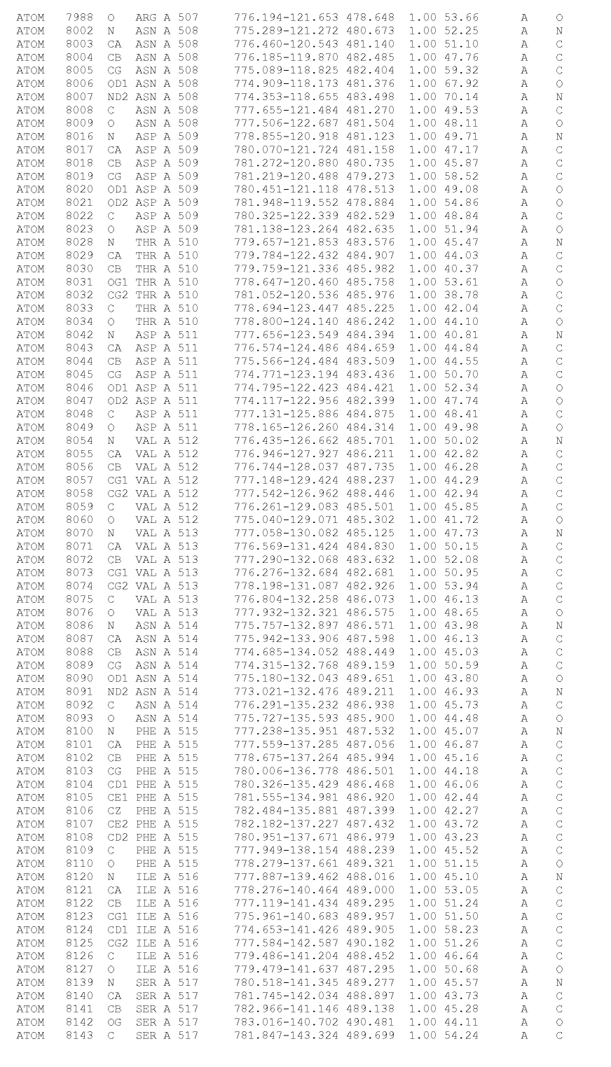

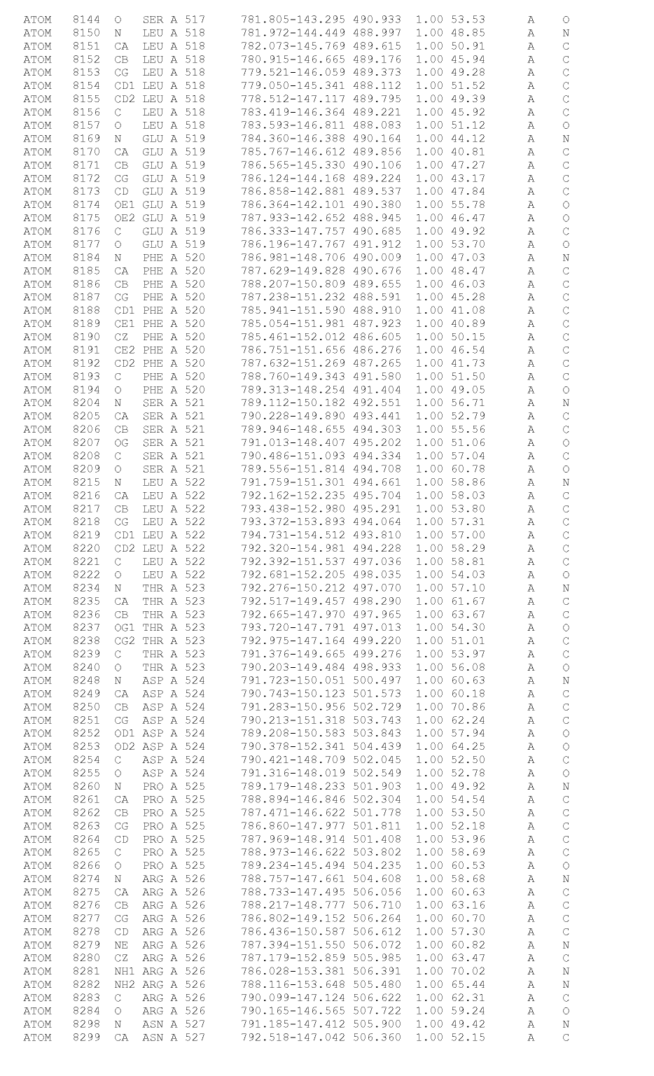

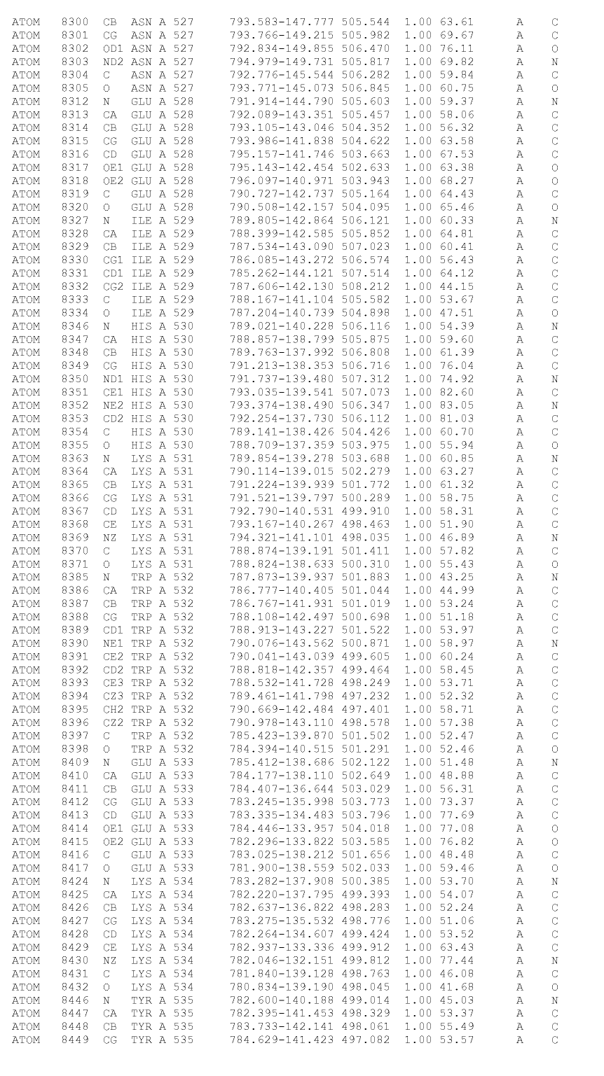

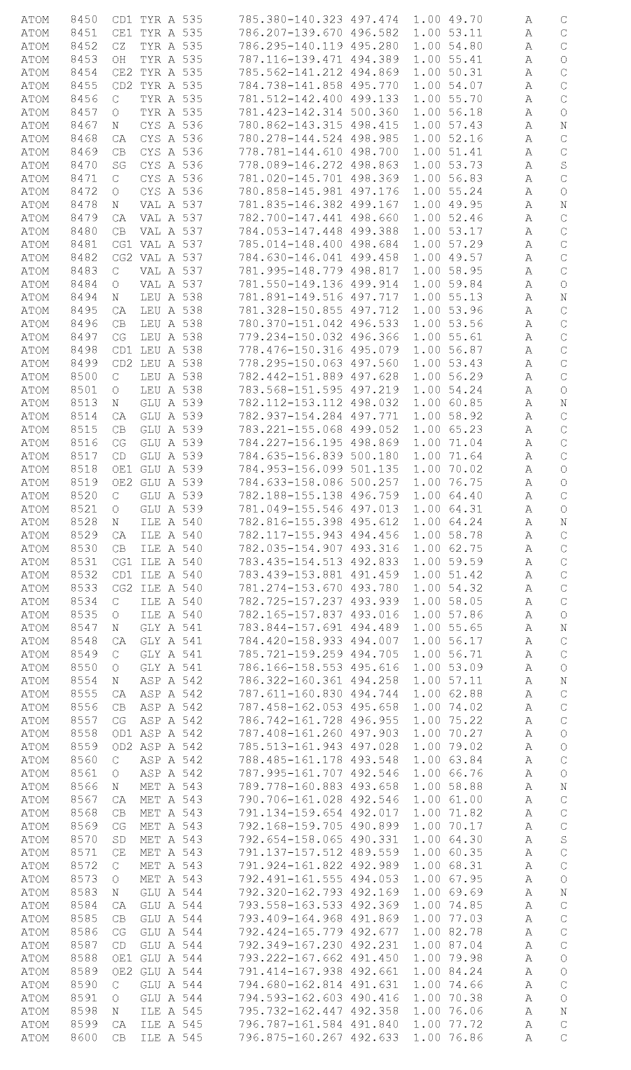

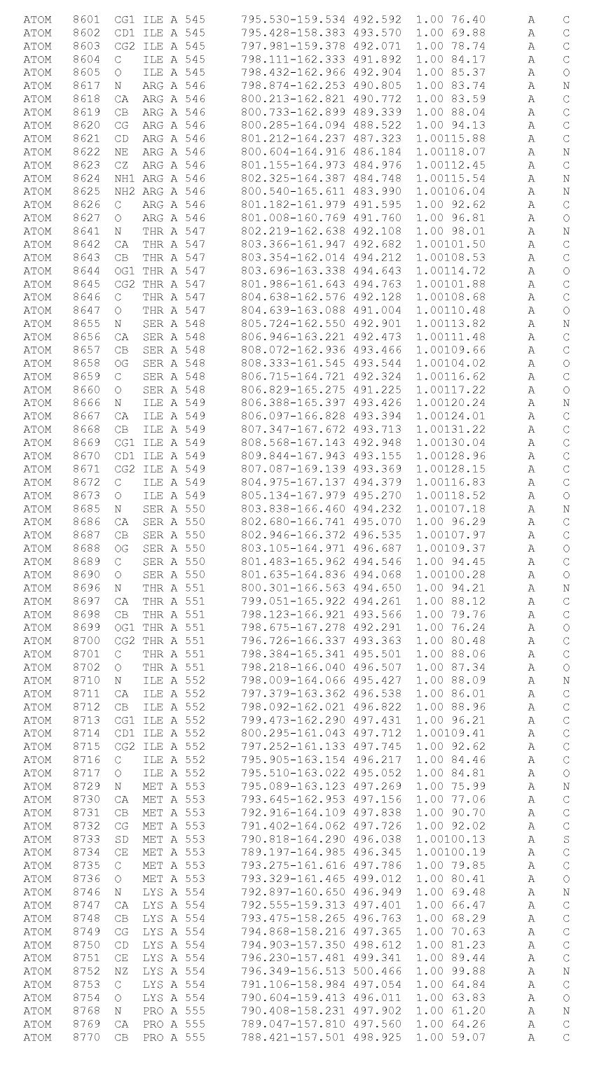

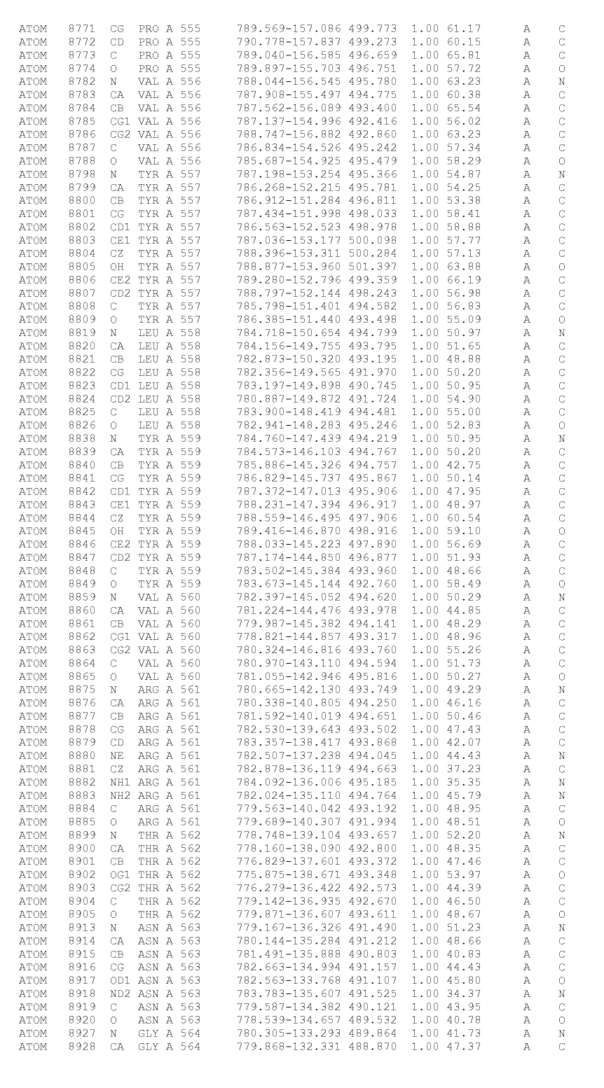

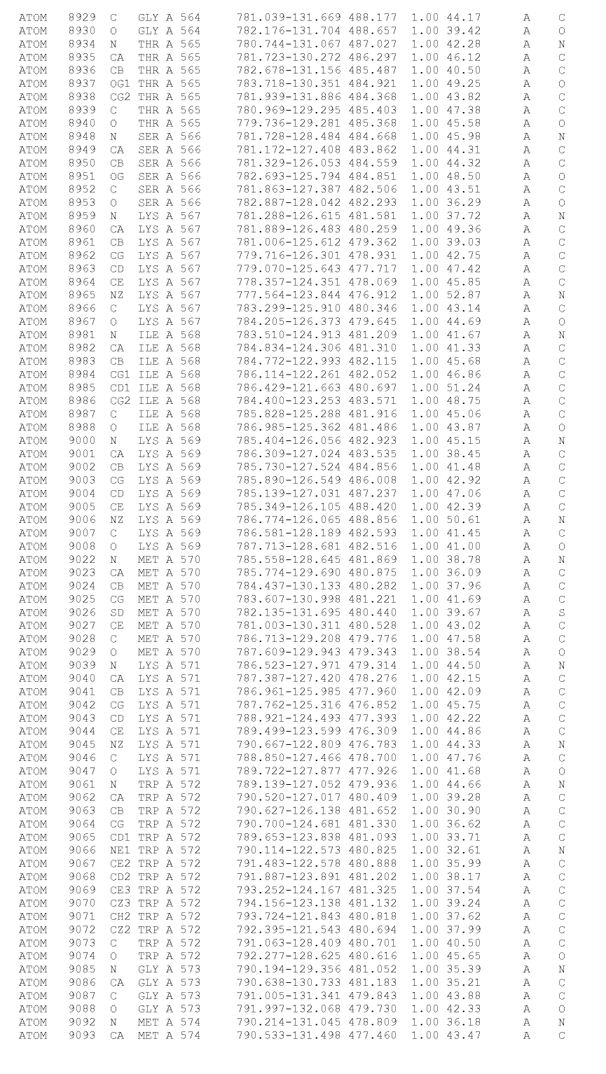

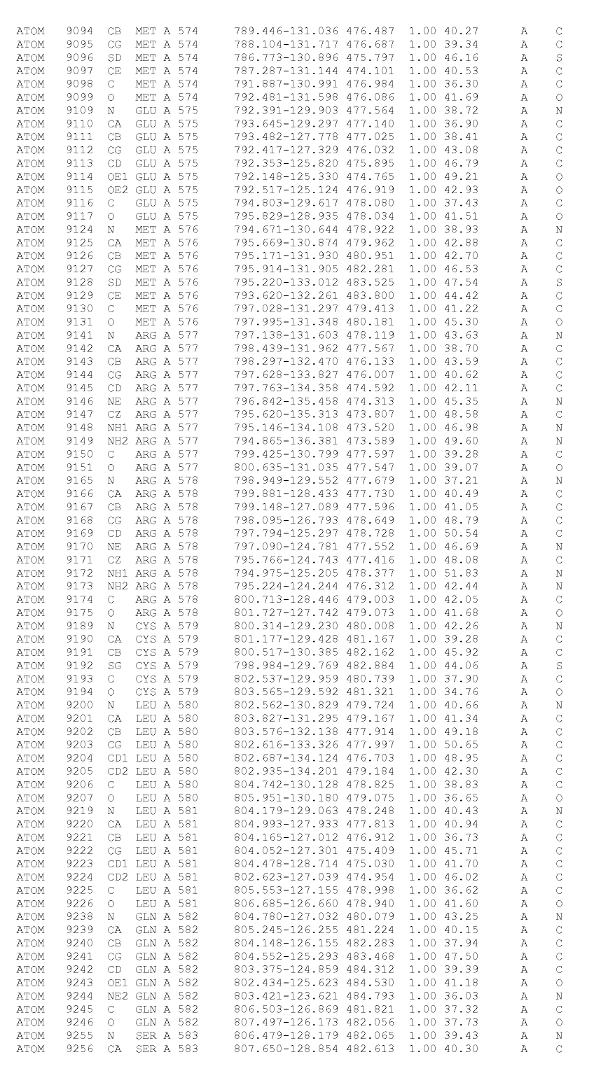

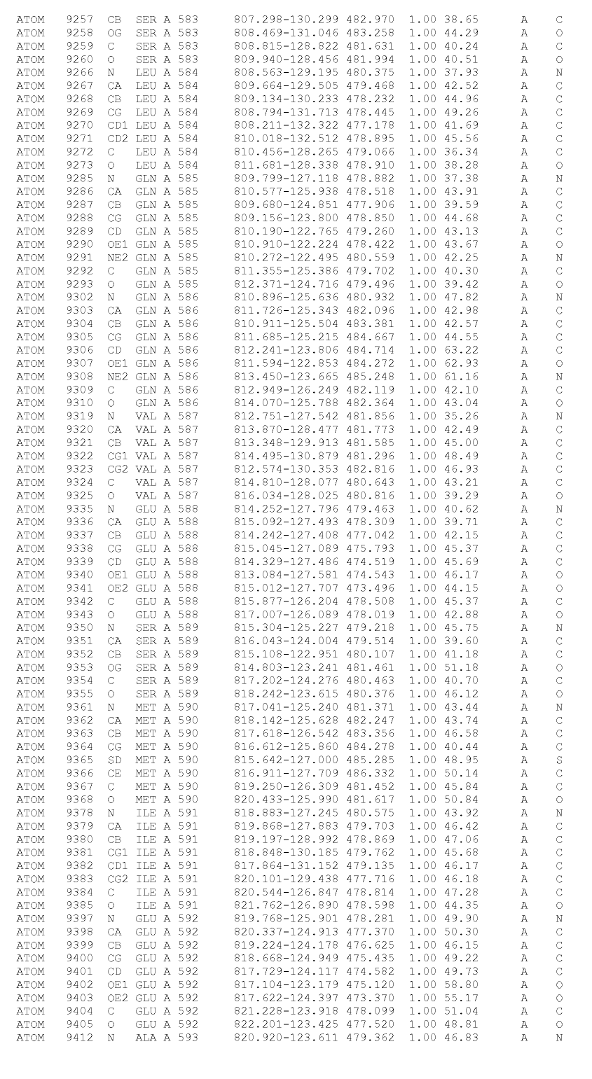

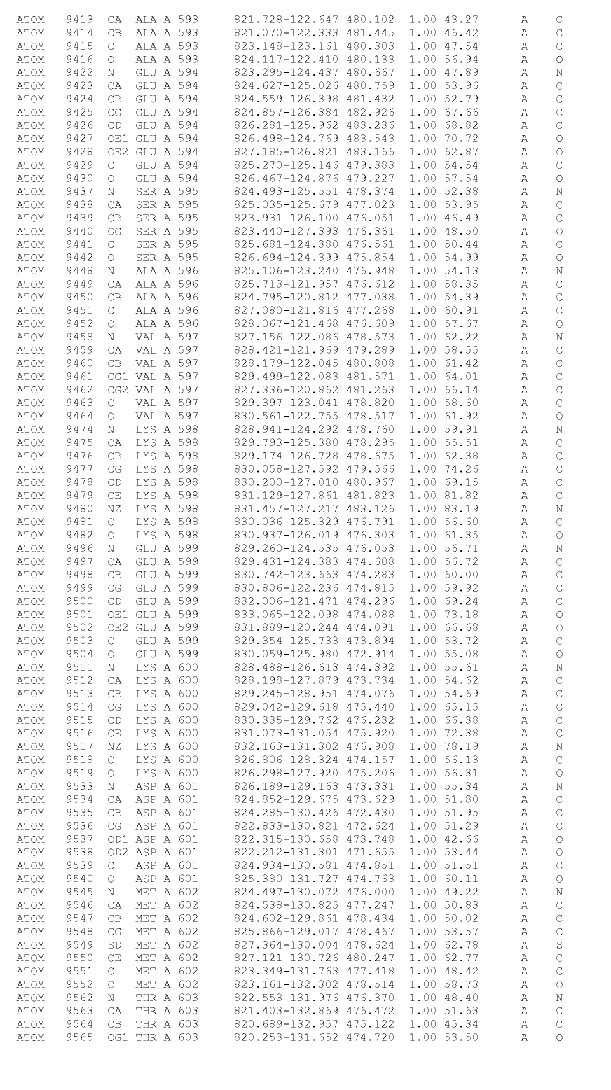

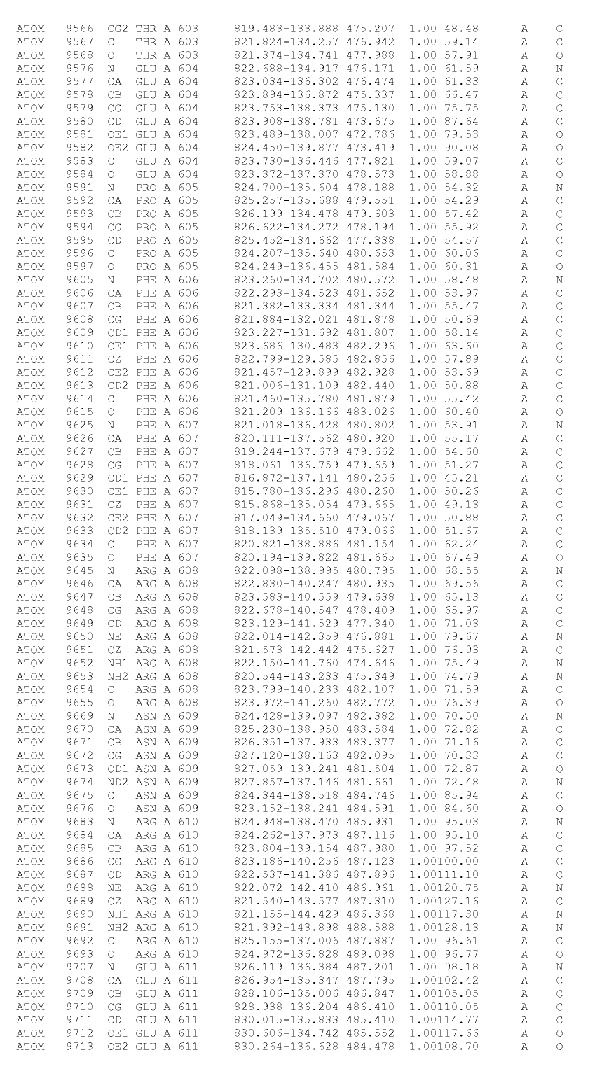

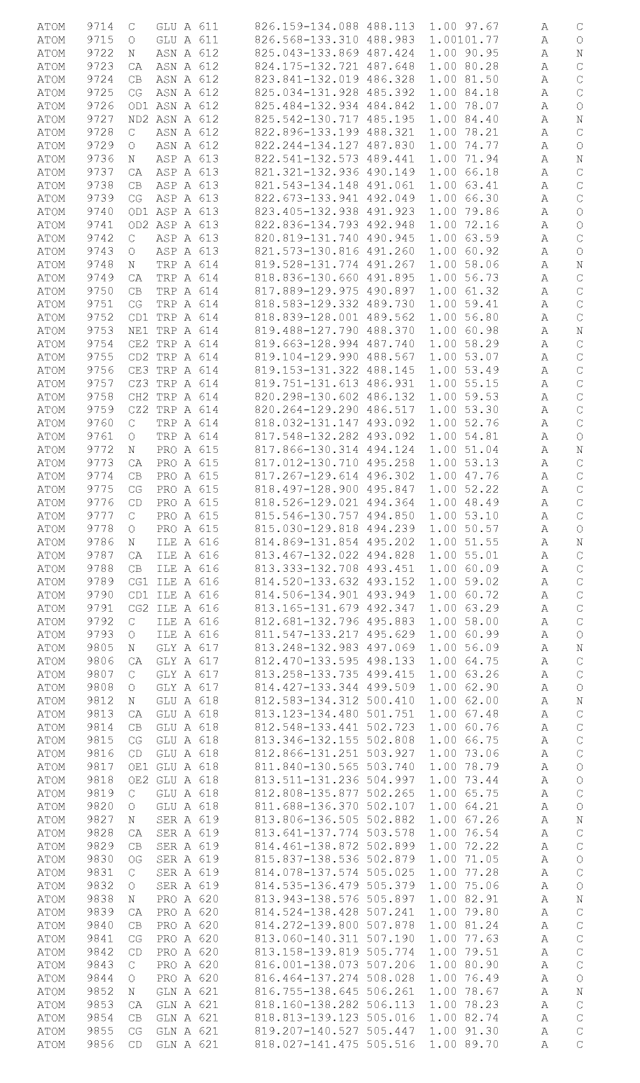

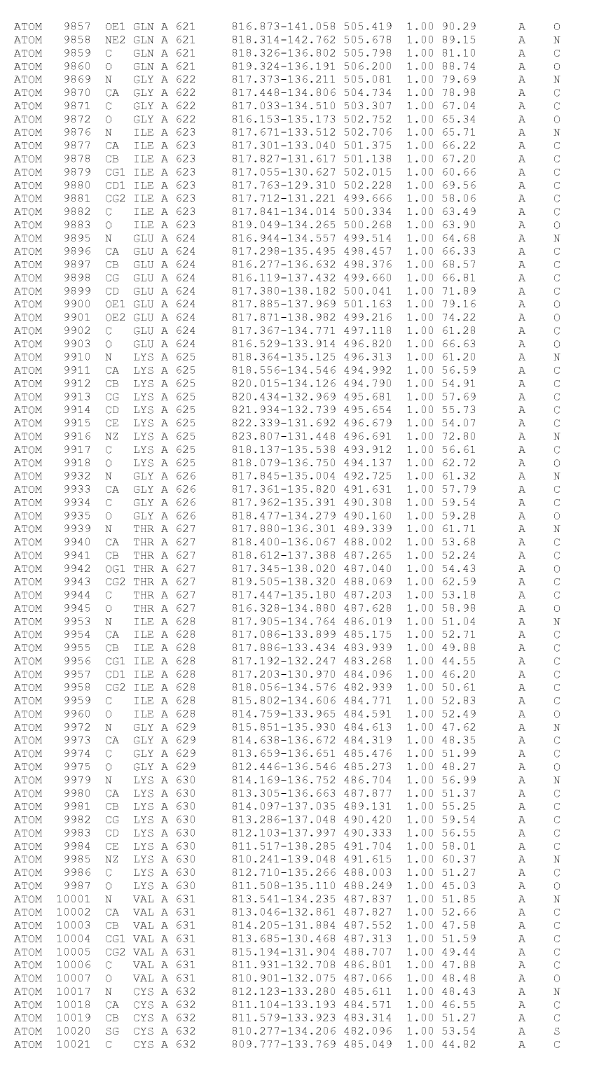

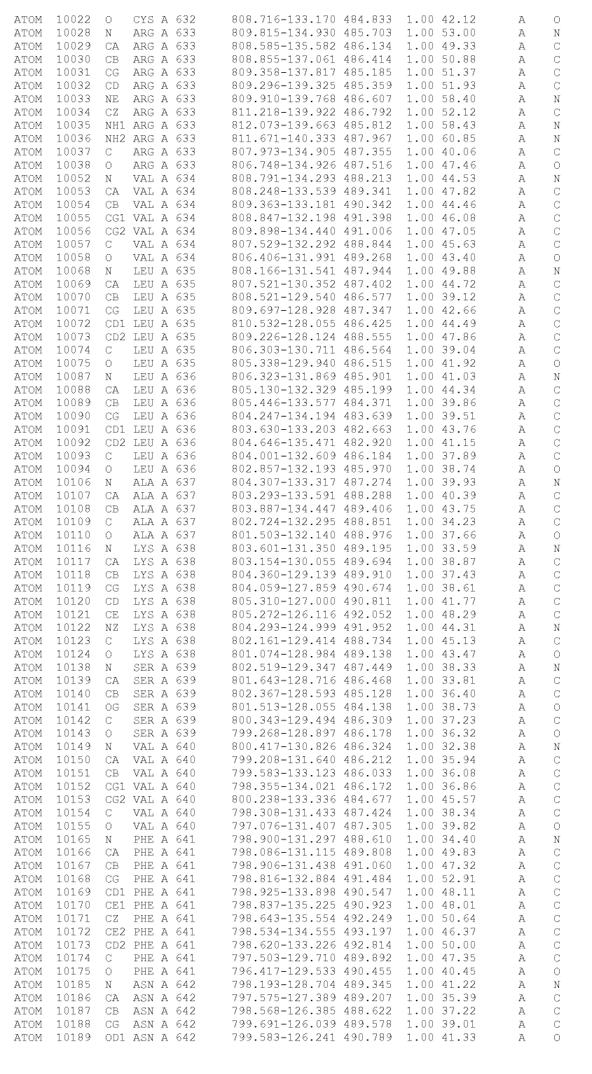

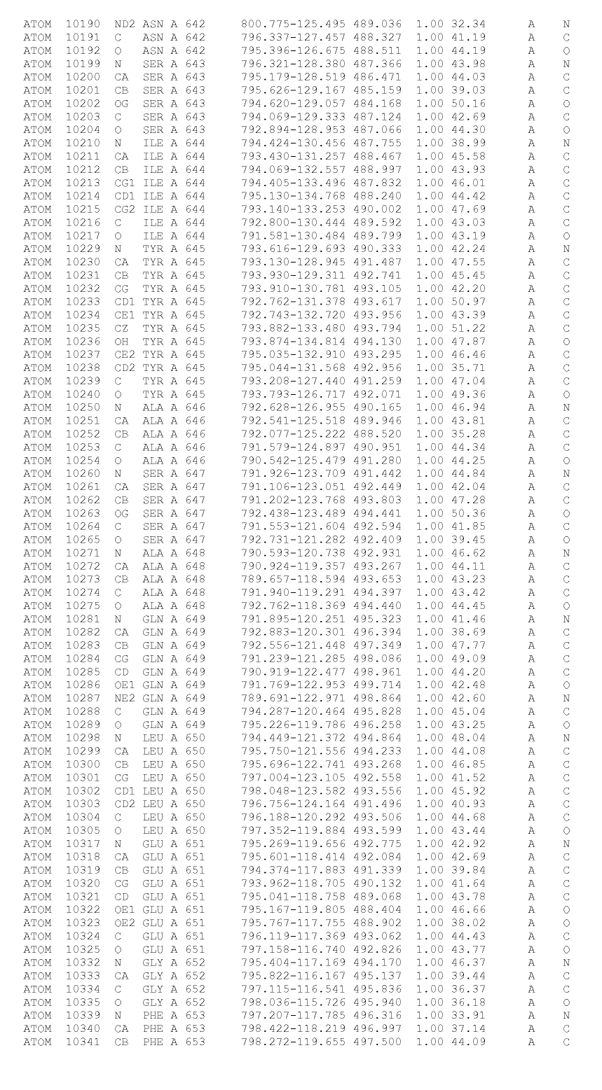

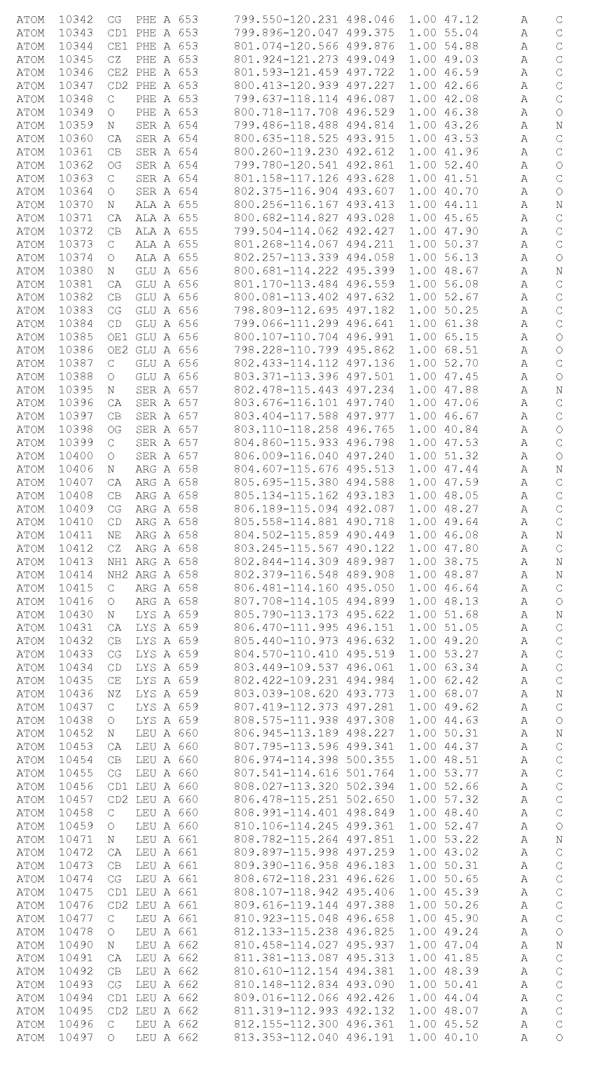

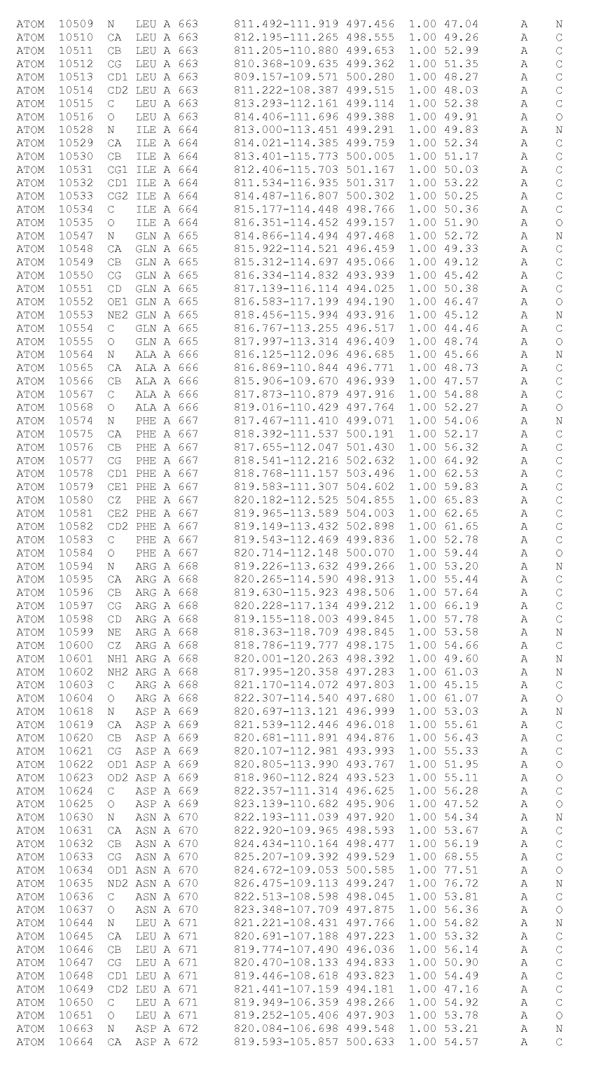

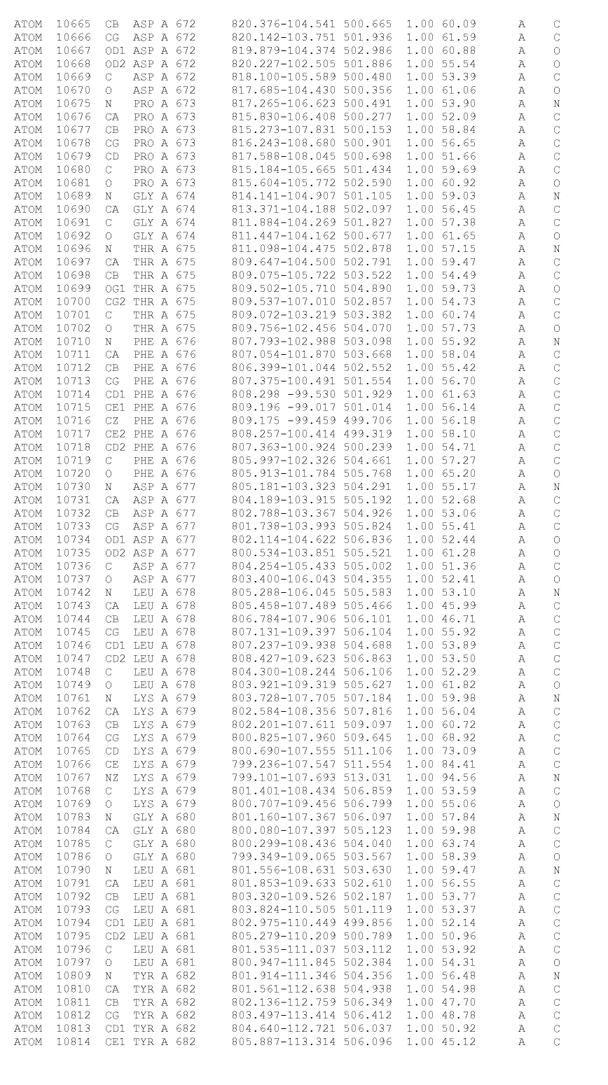

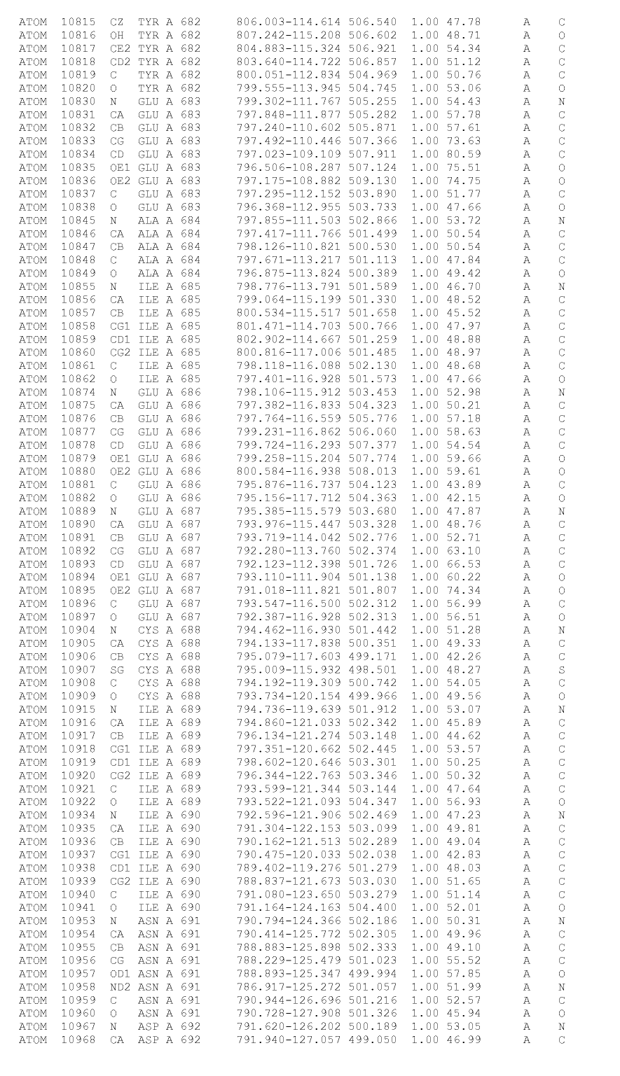

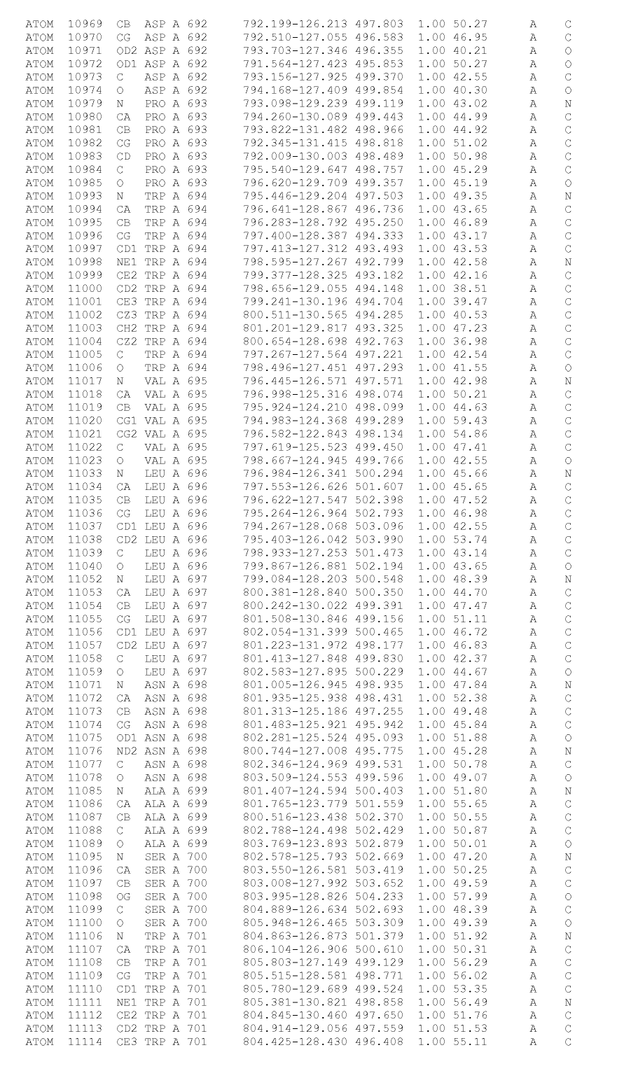

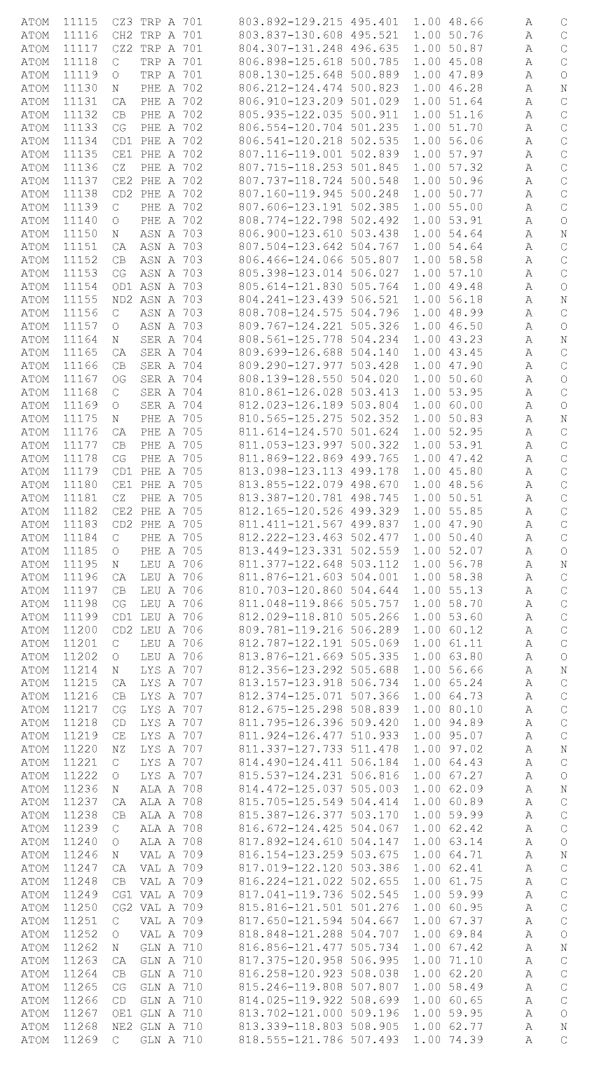

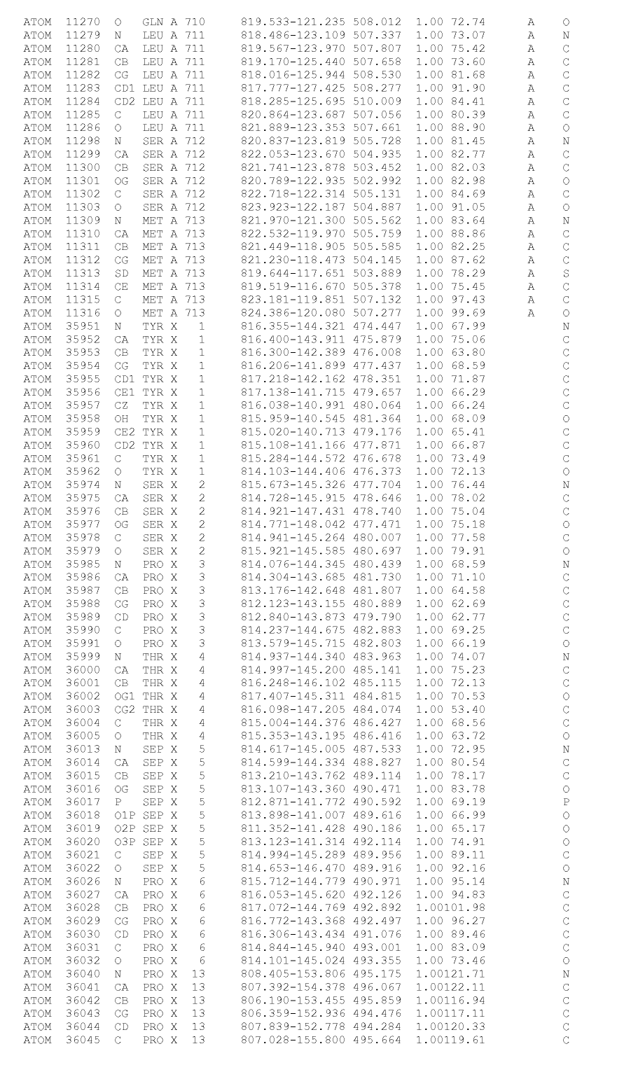

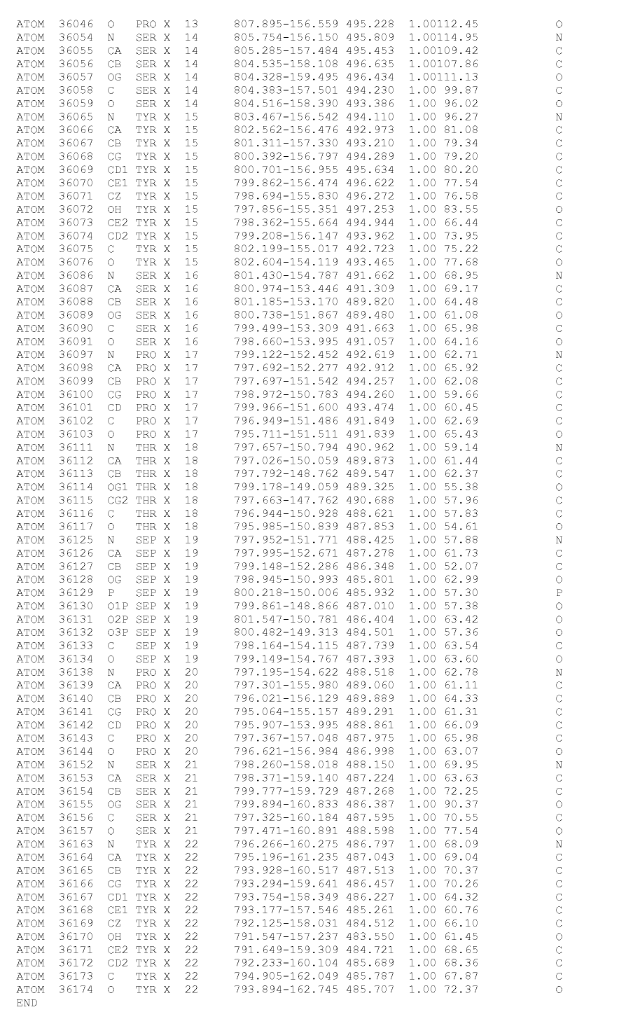

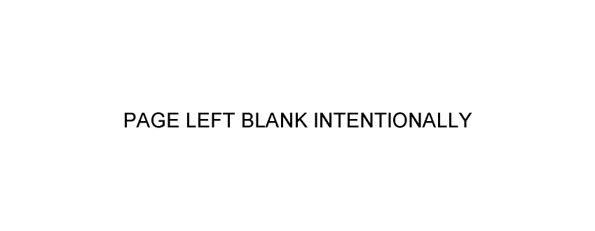

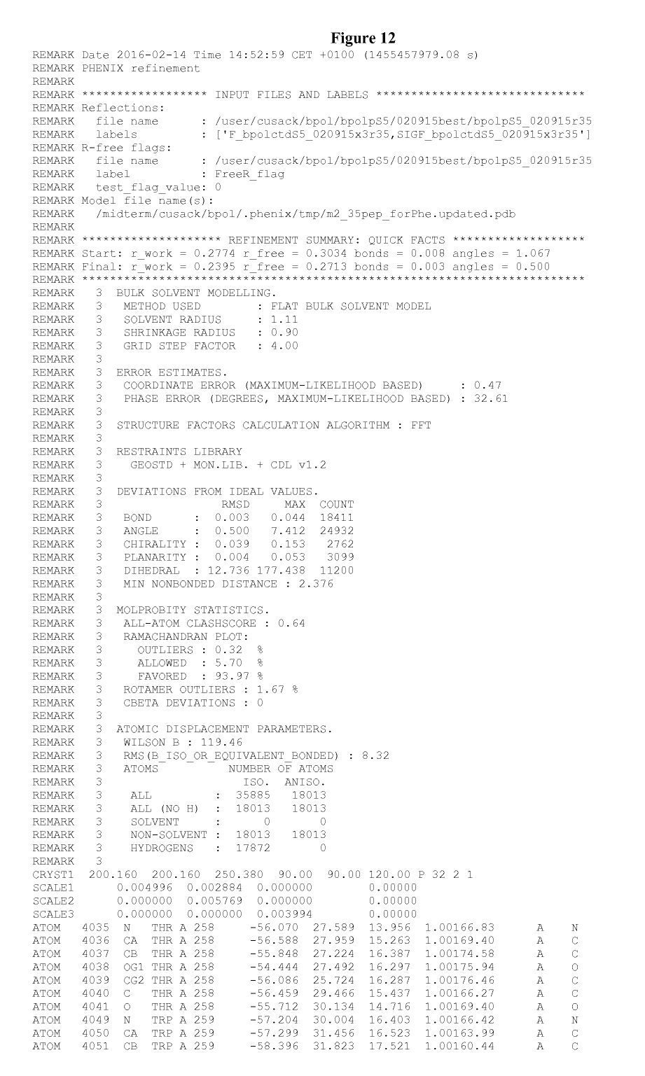

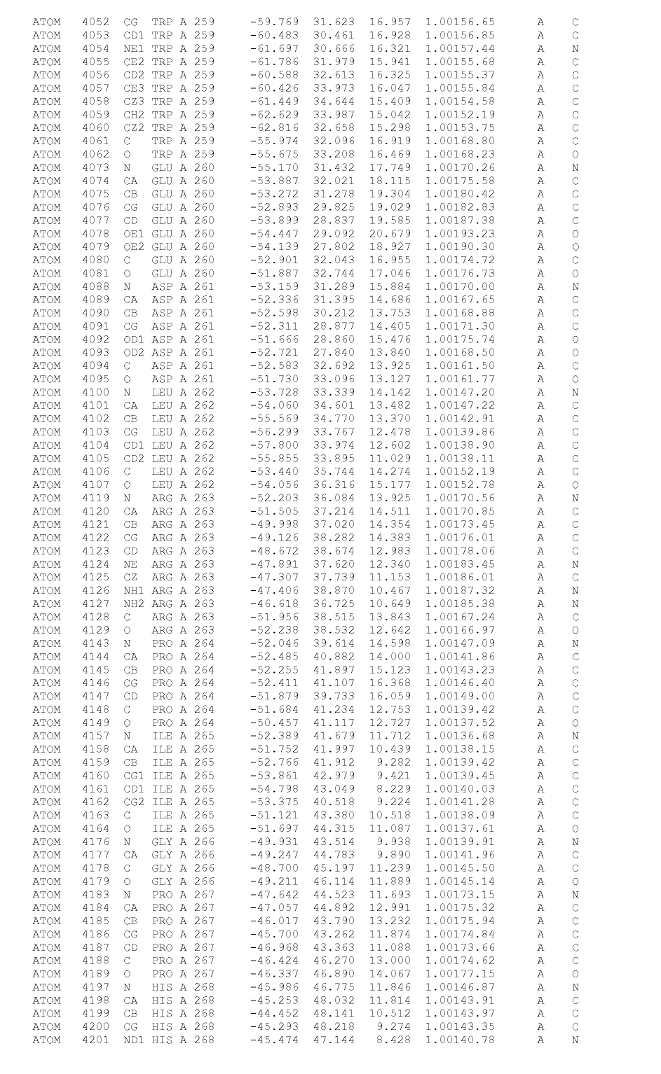

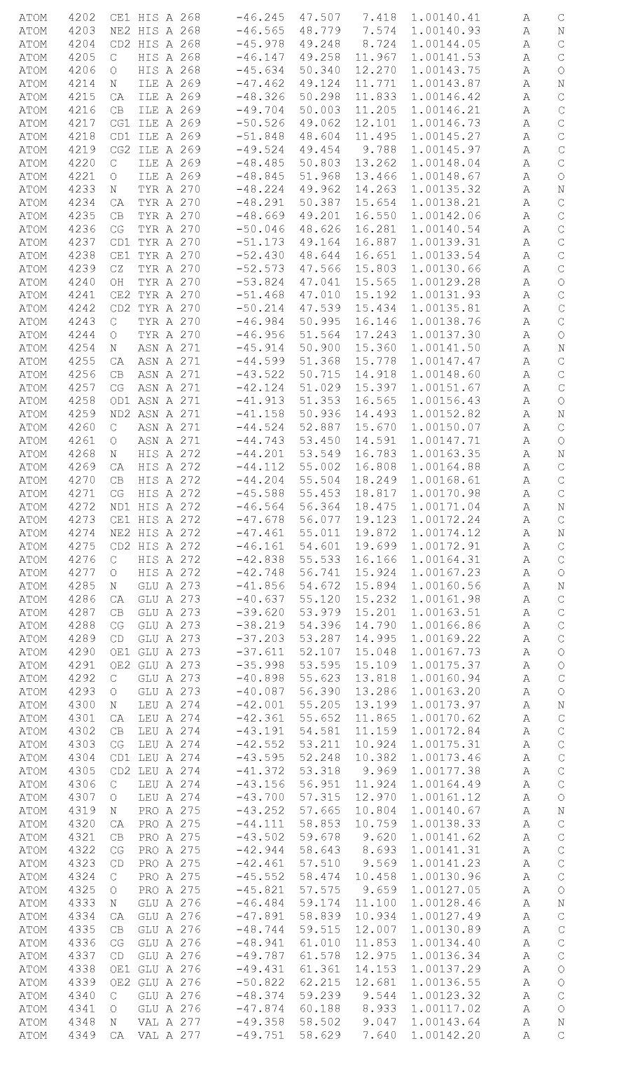

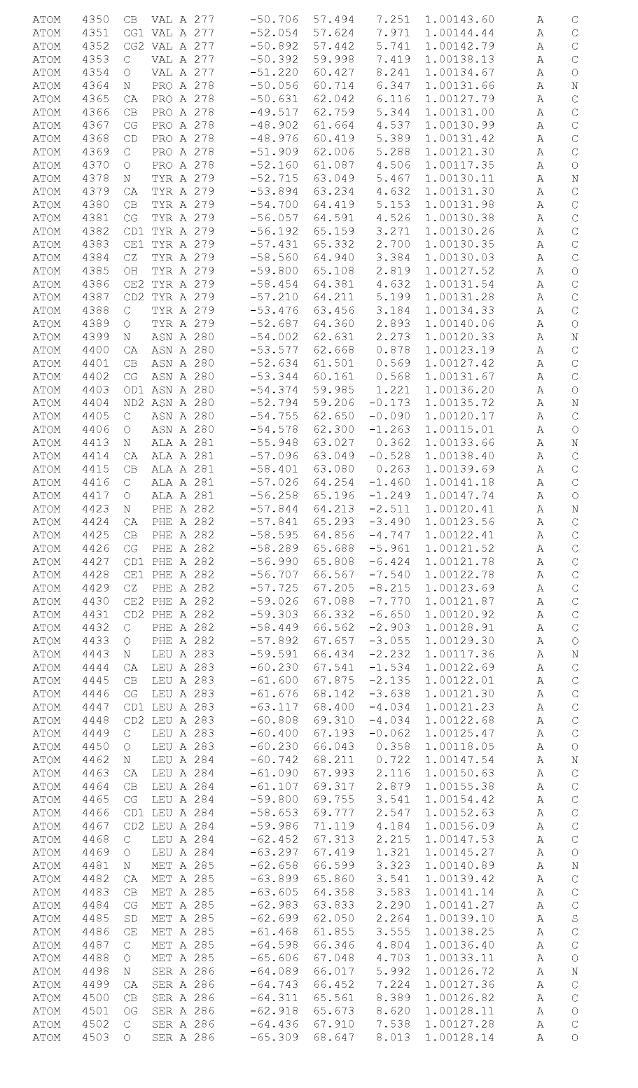

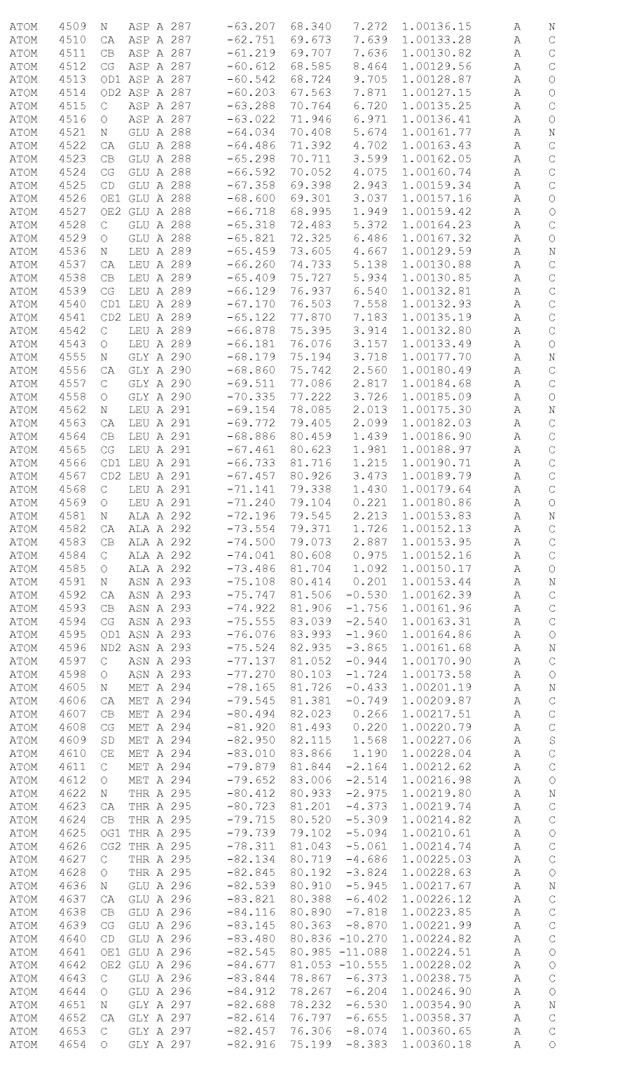

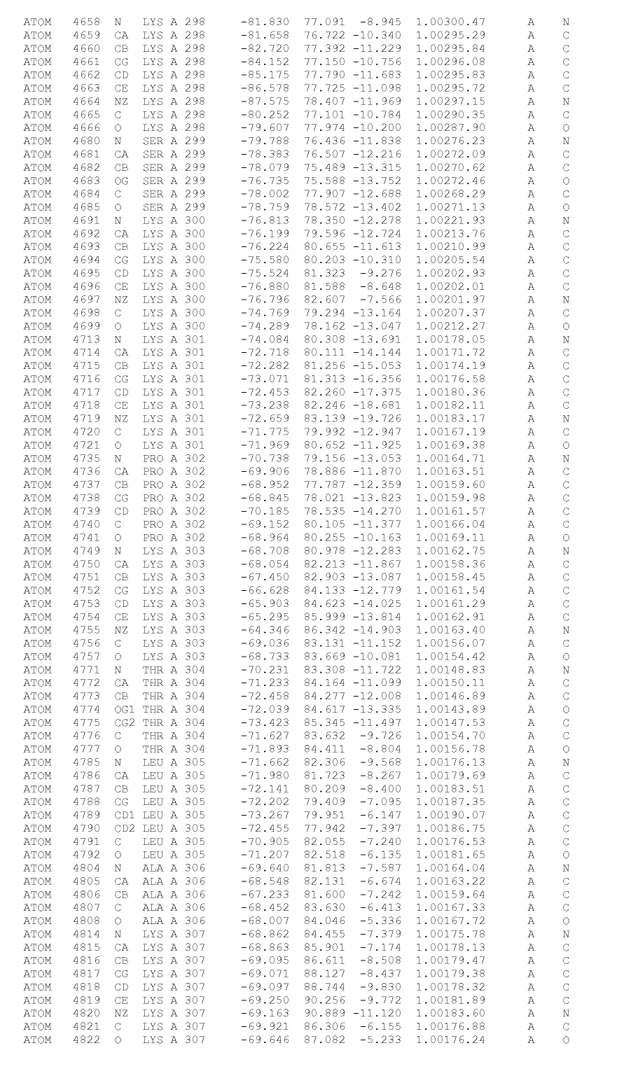

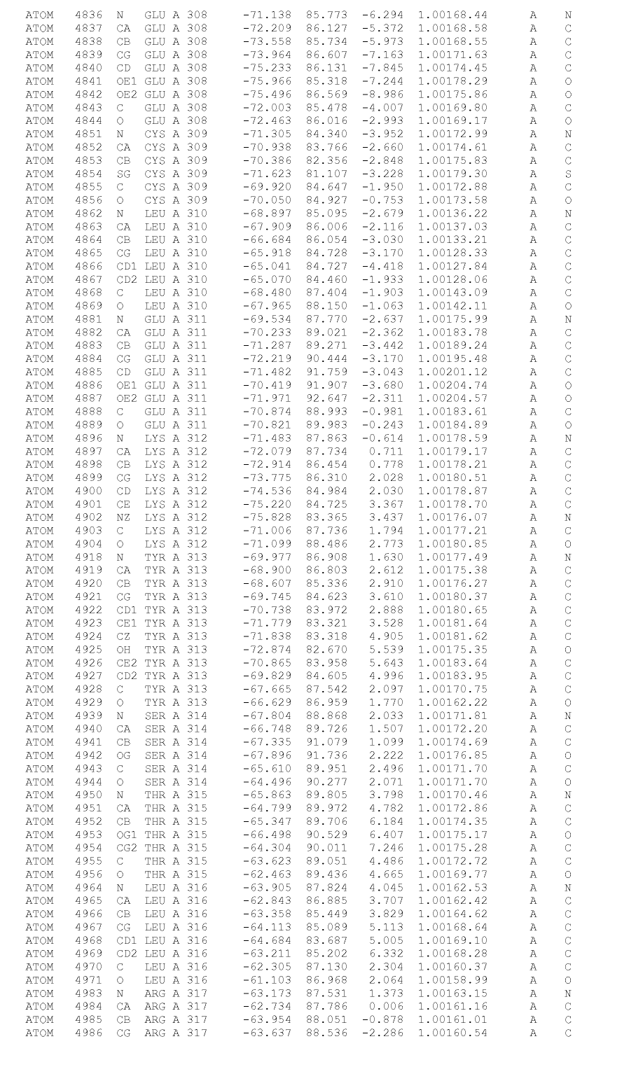

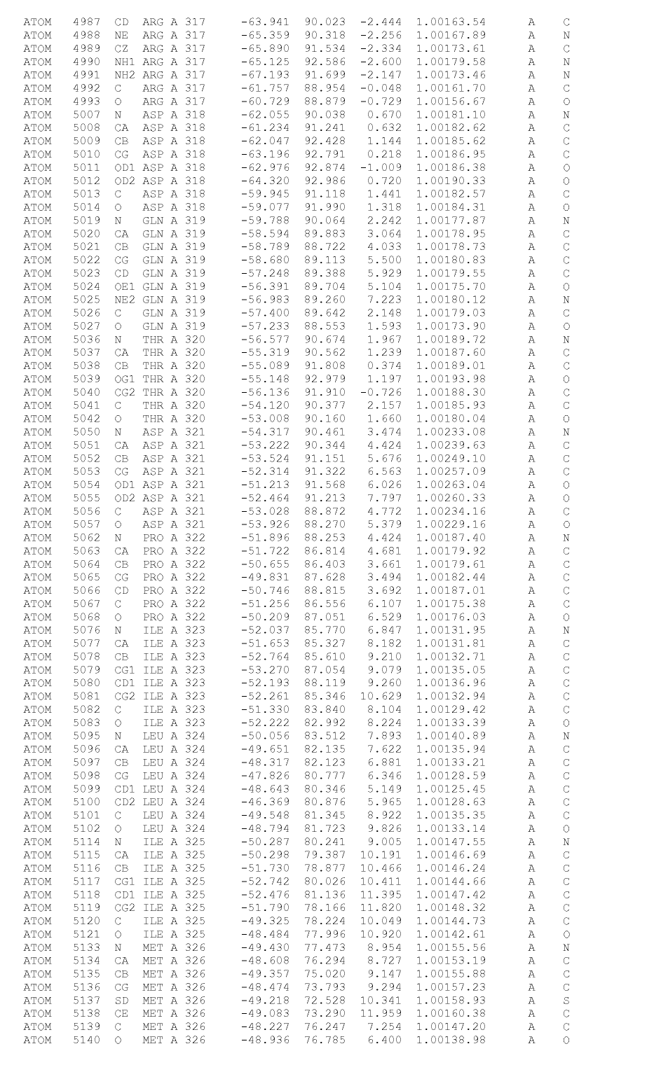

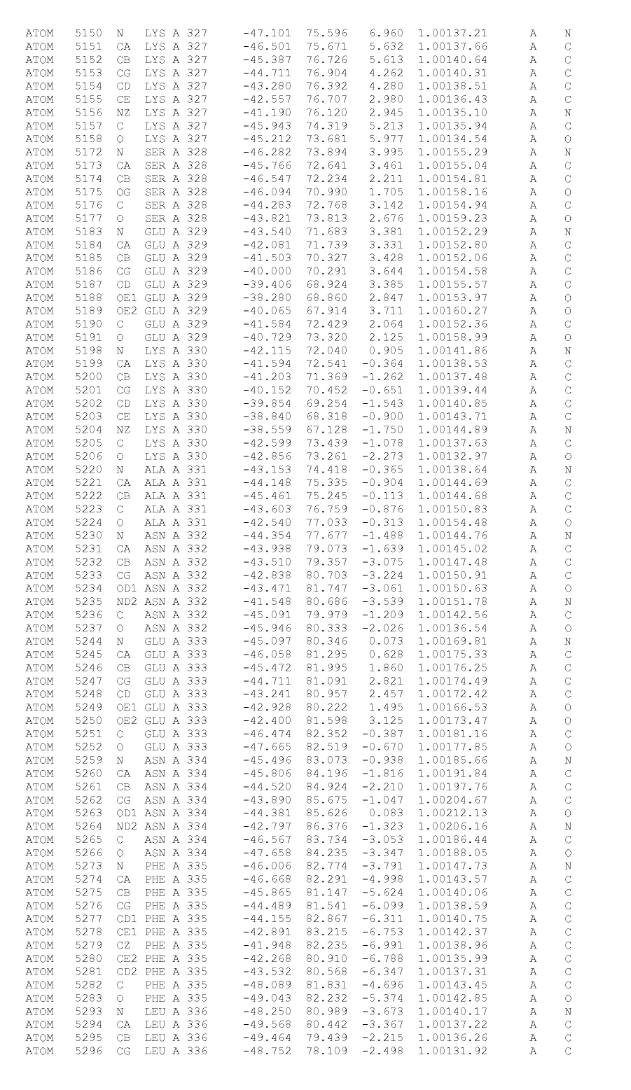

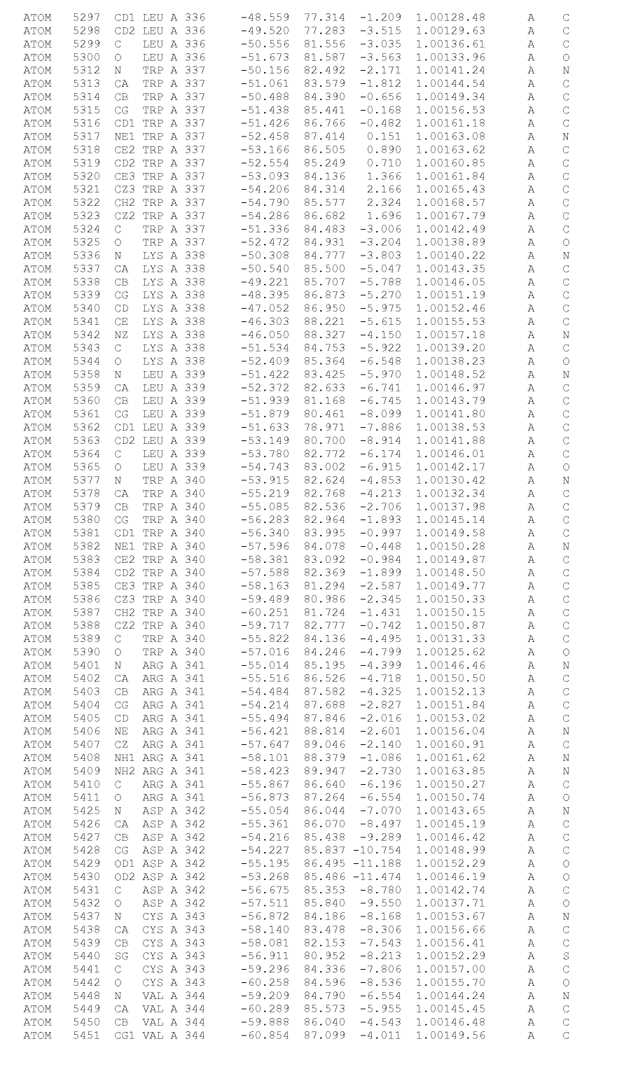

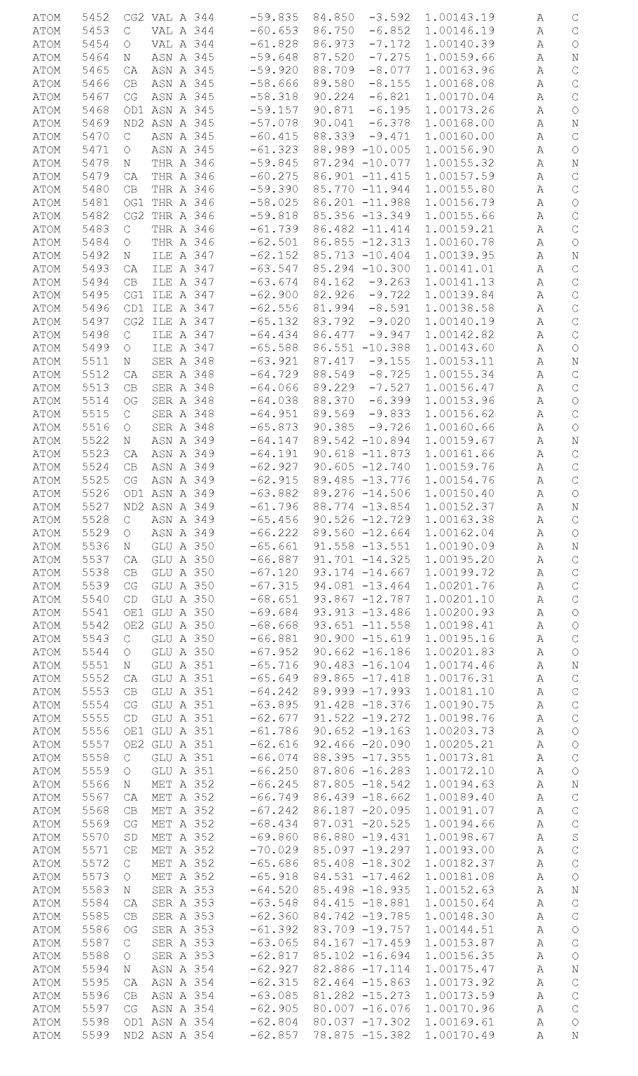

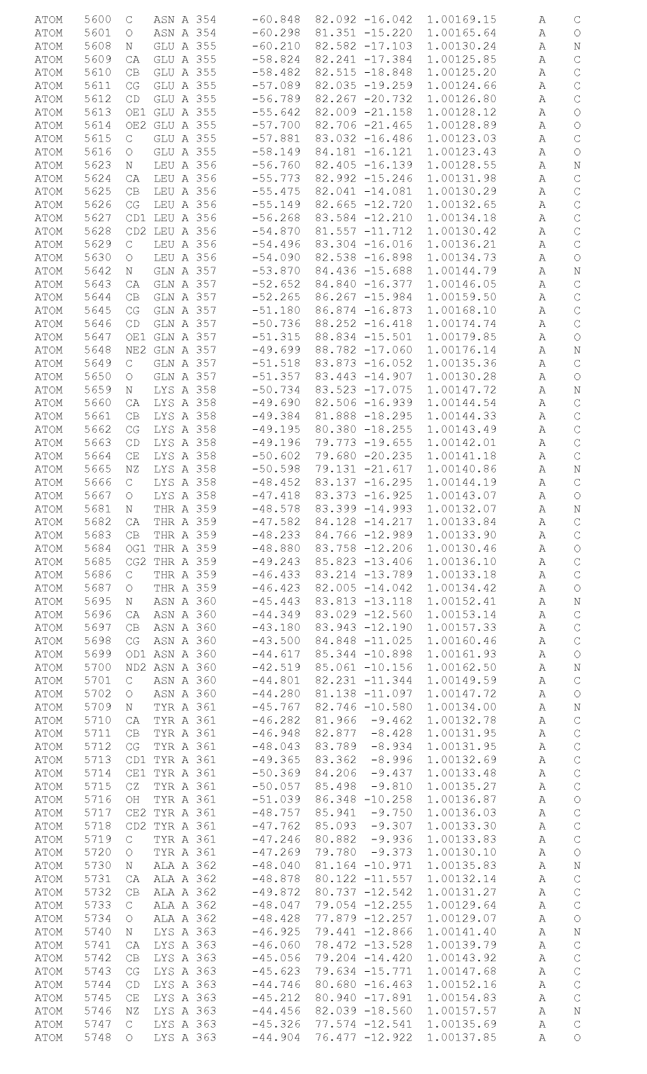

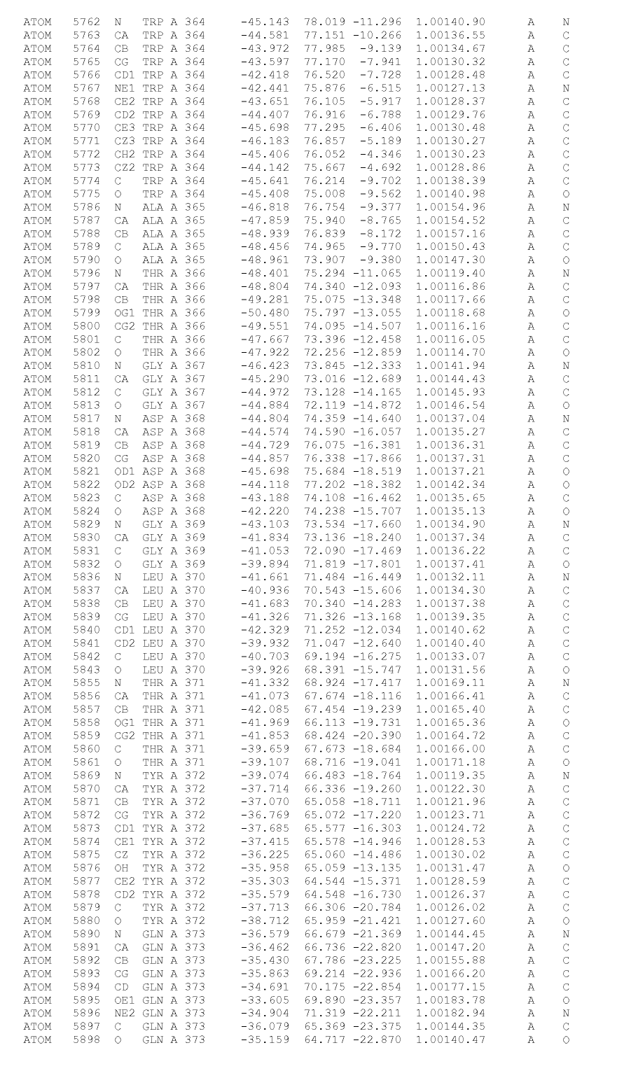

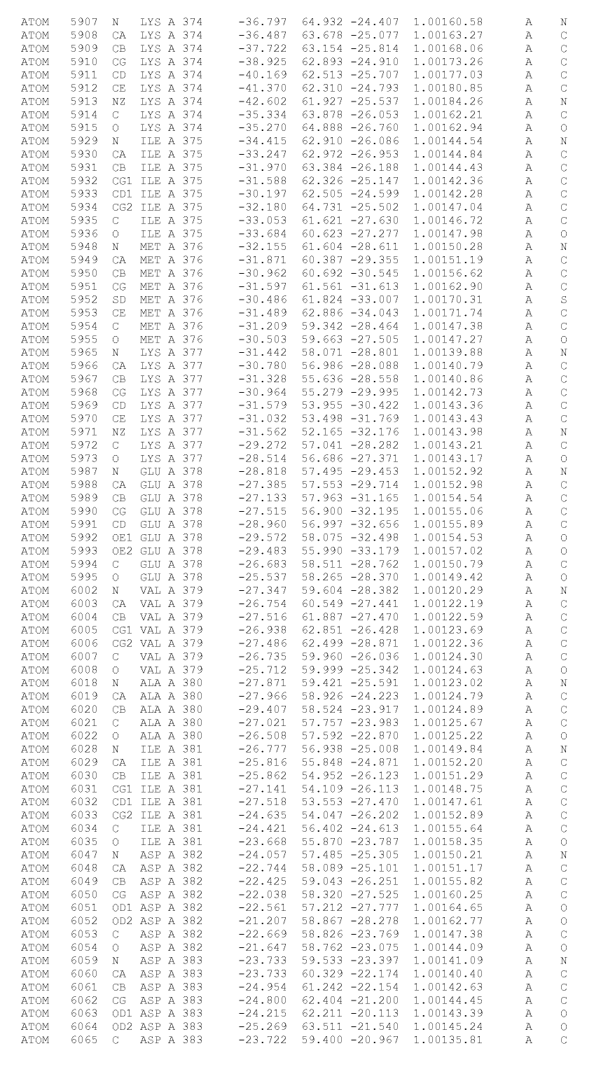

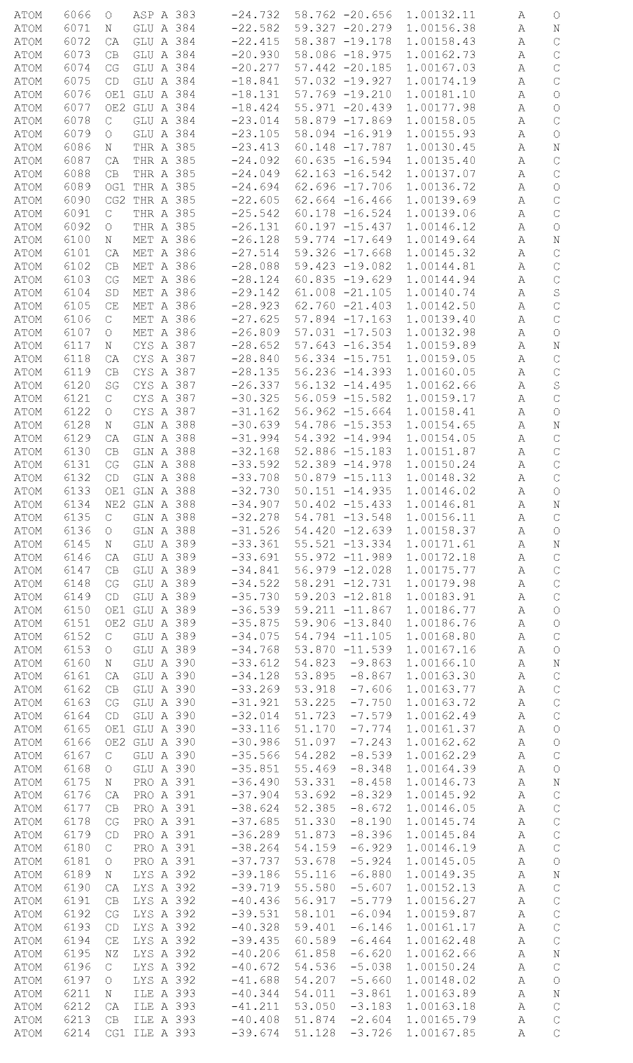

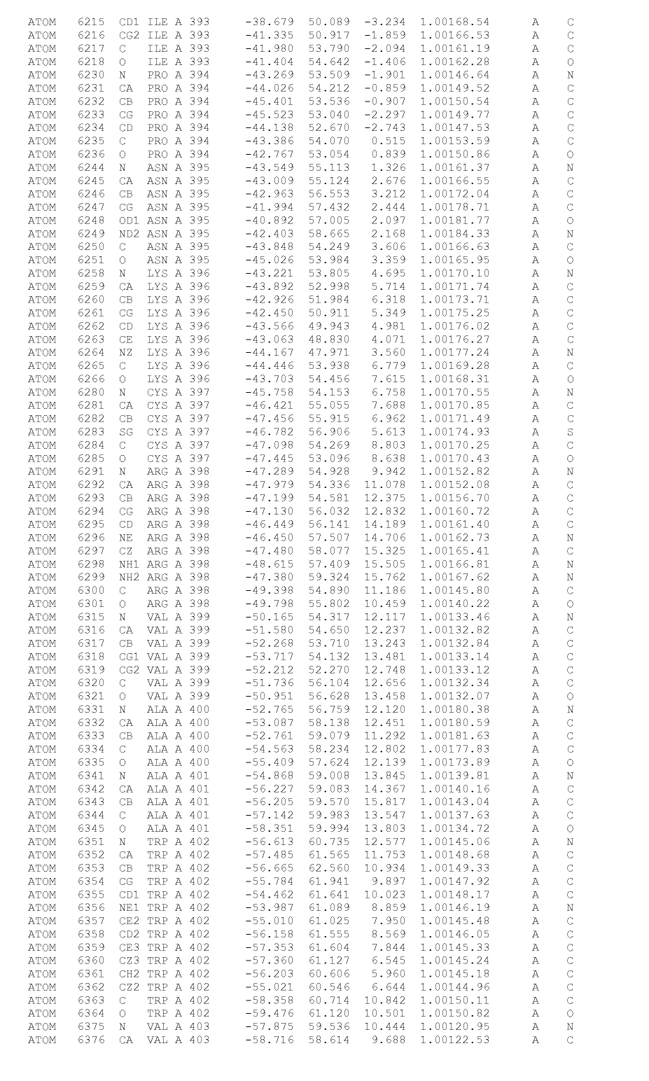

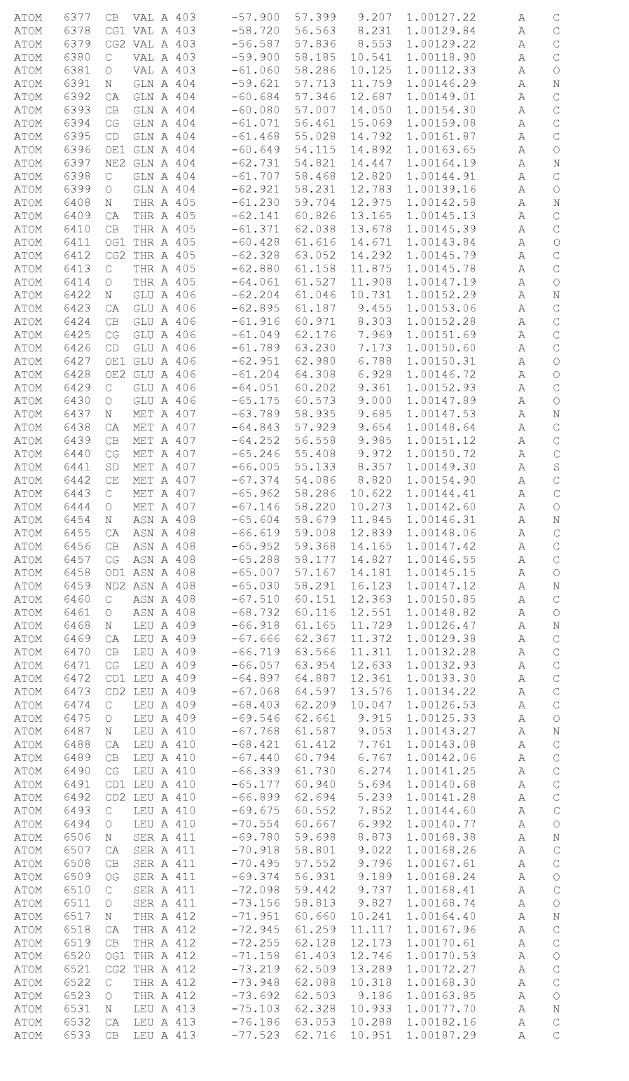

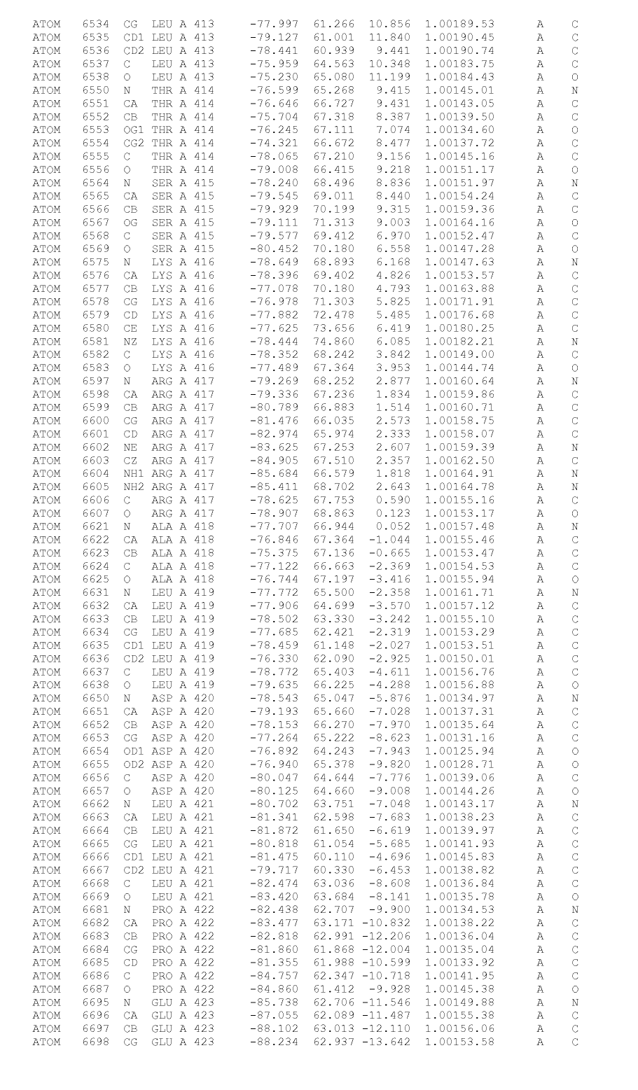

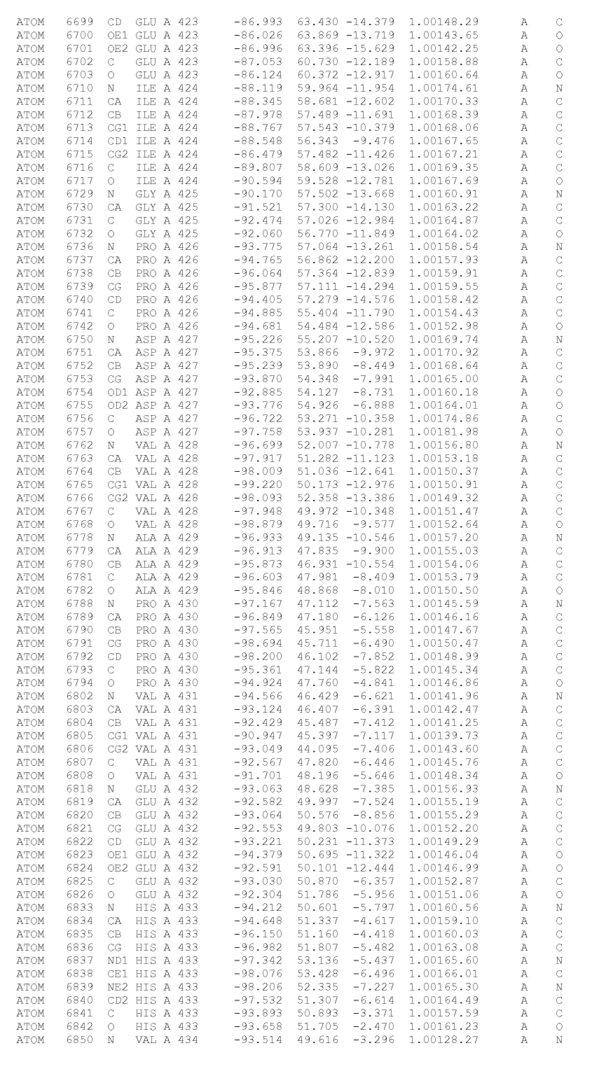

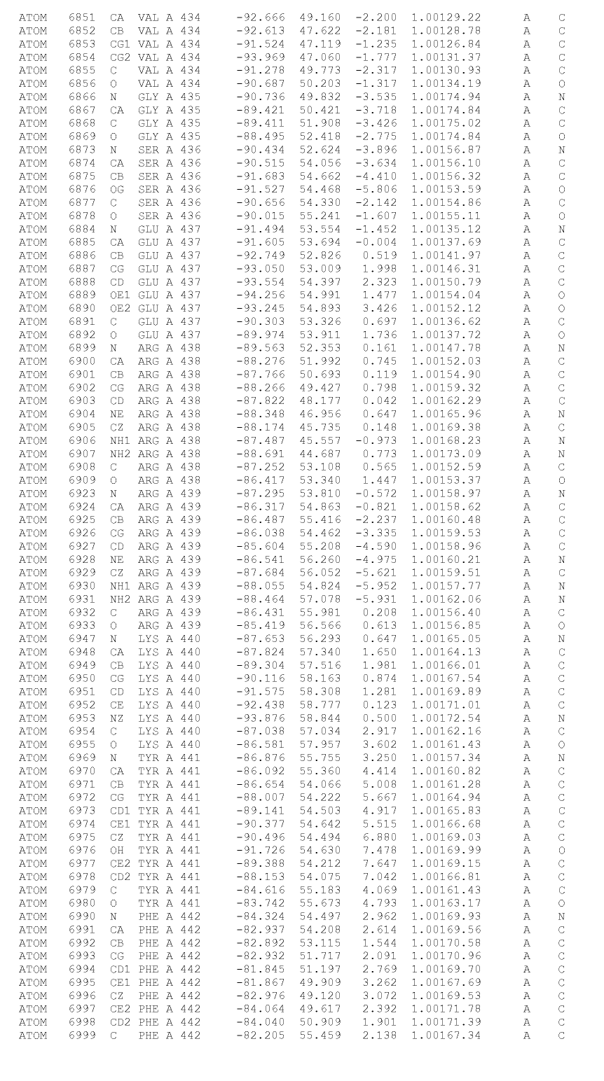

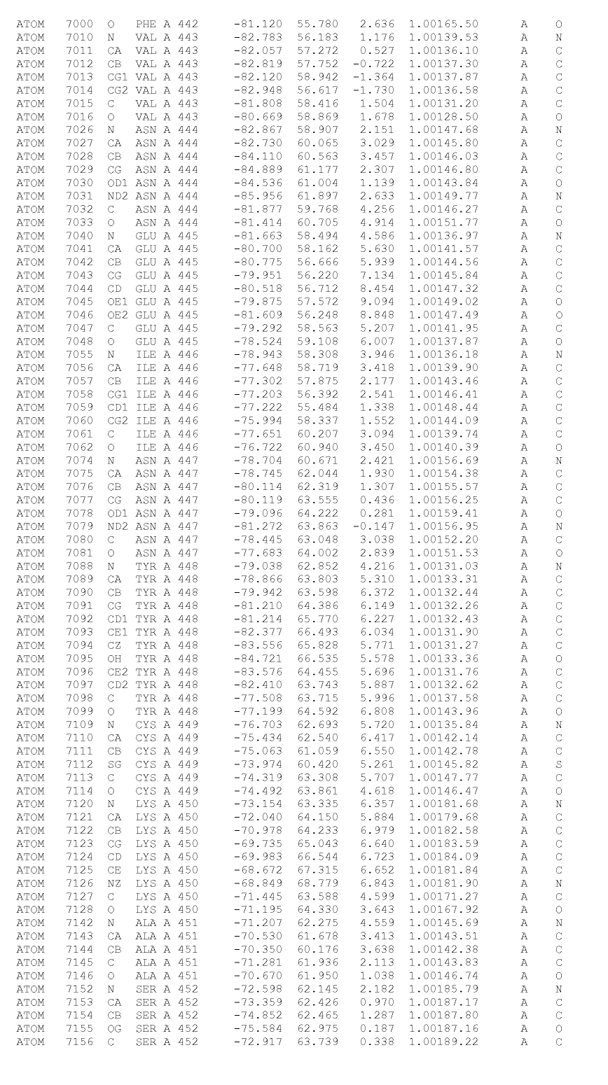

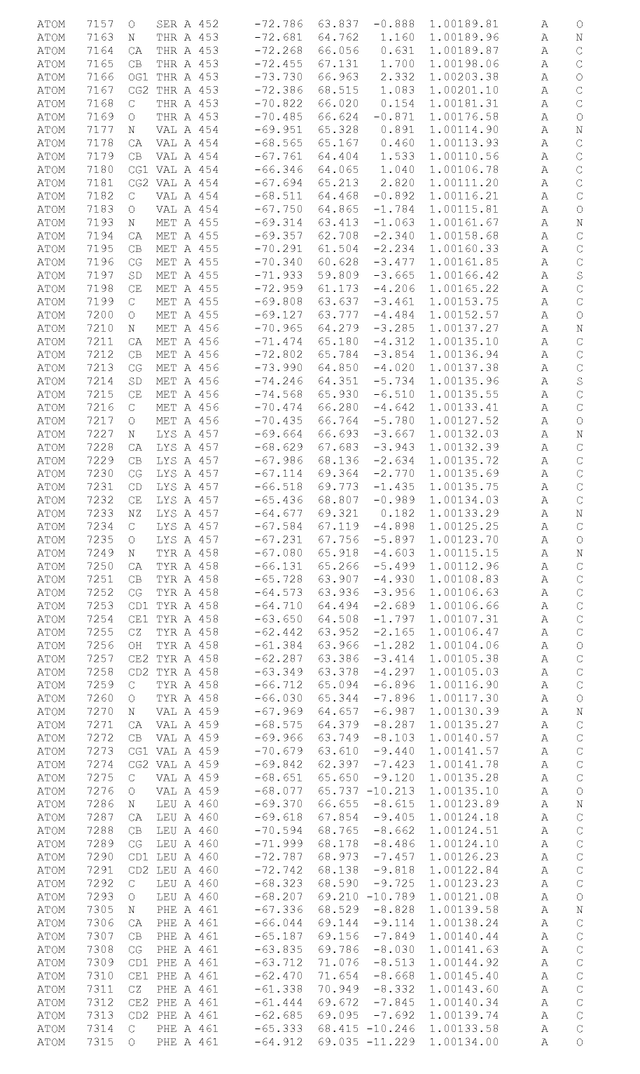

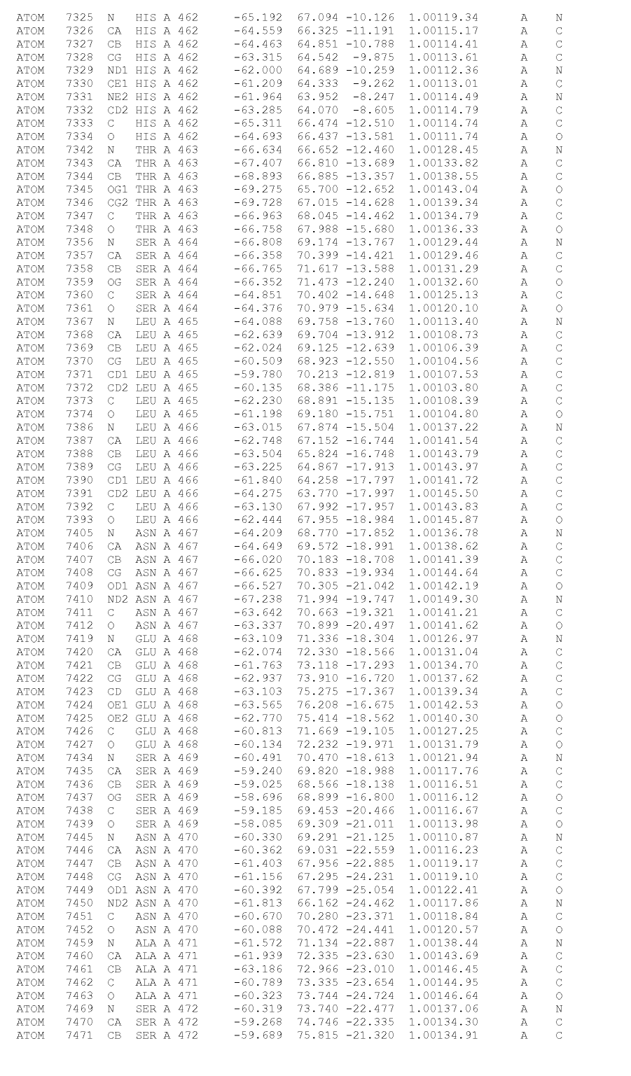

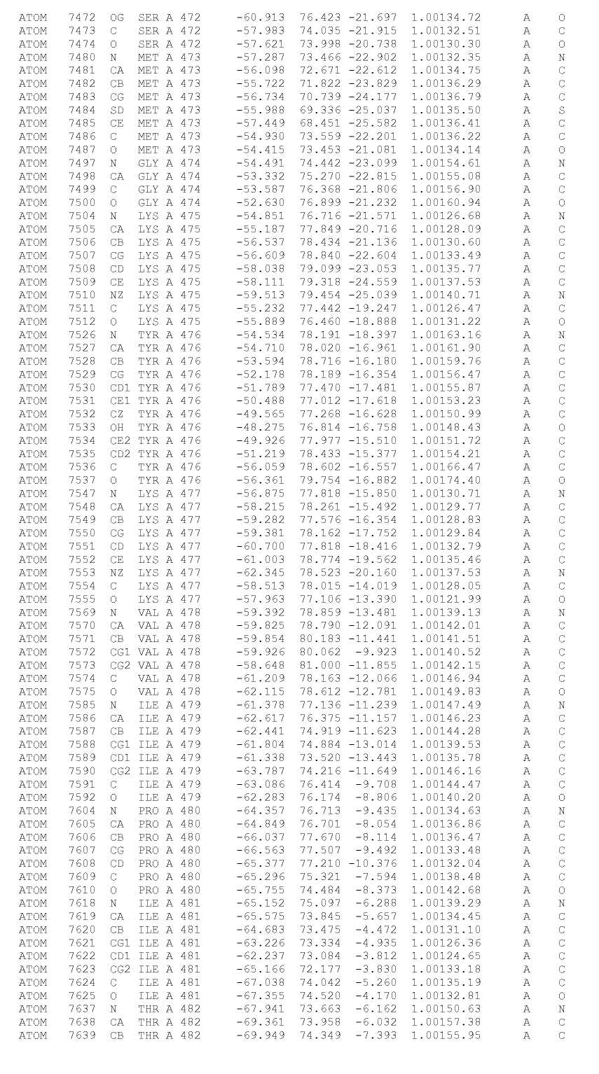

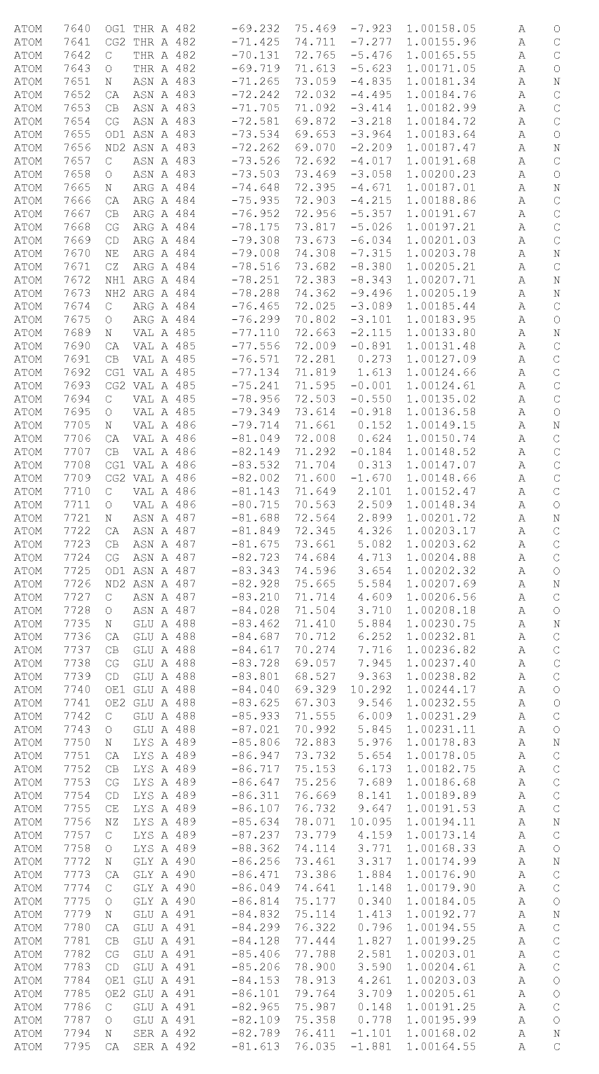

The term “root mean square deviation” means the square root of the arithmetic mean of the squares of the deviations from the mean. It is a way to express the deviation or variation from a trend or object. For purposes of this invention, the “root mean square deviation” defines the variation in the backbone of a variant of the PA subunit or the binding site(s) therein from the backbone of the PA subunit or the binding site(s) therein as defined by the structure coordinates of the PA subunit according to FIG. 11 or 12.

As used herein, the term “constructing a computer model” includes the quantitative and qualitative analysis of molecular structure and/or function based on atomic structural information and interaction models. The term “modeling” includes conventional numeric-based molecular dynamic and energy minimization models, interactive computer graphic models, modified molecular mechanics models, distance geometry, and other structure-based constraint models.

The term “fitting program operation” refers to an operation that utilizes the structure coordinates of a chemical entity, an enzymatically active center, a binding pocket, molecule or molecular complex, or portion thereof, to associate the chemical entity with the enzymatically active center, the binding pocket, molecule or molecular complex, or portion thereof. This may be achieved by positioning, rotating or translating the chemical entity in the enzymatically active center to match the shape and electrostatic complementarity of the enzymatically active center or binding site. Covalent interactions, non-covalent interactions such as hydrogen bond, electrostatic, hydrophobic, van der Waals interactions may be optimized, and non-complementary electrostatic interactions such as repulsive charge-charge, dipole-dipole and charge-dipole interactions may be reduced. Alternatively, one may minimize the deformation energy of binding of the chemical entity to the enzymatically active center or binding site.

The term “computational docking methods” refers to an operation that utilizes the structure coordinates of a chemical entity and of an enzymatically active center, a binding pocket, molecule or molecular complex, or portion thereof, to associate the chemical entity with the enzymatically active center, the binding pocket, molecule or molecular complex, or portion thereof. This may be achieved by positioning, rotating or translating the chemical entity and changing its torsional bonds in a chemical reasonable manner inside the enzymatically active center, binding site, molecule, or molecular complex, or portion thereof, to match the shape and electrostatic complementarity of the enzymatically active center, binding site, molecule, or molecular complex. Covalent interactions, non-covalent interactions such as hydrogen bond, electrostatic, hydrophobic, van der Waals interactions may be optimized, and non-complementary electrostatic interactions such as repulsive charge-charge, dipole-dipole and charge-dipole interactions may be reduced.

As used herein, the term “test compound” refers to an agent comprising a compound, molecule, or complex that is being tested for its ability to decrease or prevent the binding of the polypeptide fragment of interest, e.g., to the PA subunit of the viral RNA-dependent RNA polymerase from the Orthomyxoviridae family or variants thereof to its ligand, in particular to CTD of the cellular Pol II. Test compounds can be any agents including, but not restricted to, peptides, peptoids, polypeptides, proteins (including antibodies), lipids, metals, nucleotides, nucleotide analogs, nucleosides, nucleic acids, small organic or inorganic molecules, chemical compounds, elements, saccharides, isotopes, carbohydrates, imaging agents, lipoproteins, glycoproteins, enzymes, analytical probes, polyamines, and combinations and derivatives thereof.

The term “small molecules” refers to molecules that have a molecular weight between 50 and about 2,500 Daltons, preferably in the range of 200-800 Daltons. In addition, a test compound according to the present invention may optionally comprise a detectable label. Such labels include, but are not limited to, enzymatic labels, radioisotope or radioactive compounds or elements, fluorescent compounds or metals, chemiluminescent compounds and bioluminescent compounds. Well known methods may be used for attaching such a detectable label to a test compound. The test compound of the invention may also comprise complex mixtures of substances, such as extracts containing natural products, or the products of mixed combinatorial syntheses. These can also be tested and the component that inhibits the binding of the carboxy-terminal domain of the PA subunit can be purified from the mixture in a subsequent step. Test compounds can be derived or selected from libraries of synthetic or natural compounds. For instance, synthetic compound libraries are commercially available from Enamine Ltd (Kiev, Ukraine), ChemBridge Corporation (San Diego, Calif.), or LabNetwork Inc. (South Portland, Me., USA). A natural compound library is, for example, available from TimTec LLC (Newark, Del., USA). Alternatively, libraries of natural compounds in the form of bacterial, fungal, plant and animal cell and tissue extracts can be used. Additionally, test compounds can be synthetically produced using combinatorial chemistry either as individual compounds or as mixtures. A collection of compounds made using combinatorial chemistry is referred to herein as a combinatorial library.

In the context of the present invention, “a compound which decrease the binding of the viral RNA-dependent RNA polymerase from the Orthomyxoviridae family or variant thereof, to its ligand” decreases the binding of the viral RNA-dependent RNA polymerase to its cellular ligand, in particular to the CTD of the cellular polymerase Pol II. Such a compound may be specific for the carboxy-terminal fragment of the PA subunit of the viral RNA-dependent RNA polymerase or variant thereof and does not modulate other polymerases, preferably does not modulate the activity of other polymerases, in particular mammalian polymerases. Typically, the activity is decreased by 80-100%, in particular by 90-100%, compared to the activity without the compound.

In the context of the present invention, “a compound which prevents the binding of the viral RNA-dependent RNA polymerase from the Orthomyxoviridae family or variant thereof, to its ligand” completely abolishes the binding of the viral RNA-dependent RNA polymerase to its cellular ligand, in particular to the CTD of the cellular polymerase Pol II. Such a compound may be specific for the carboxy-terminal fragment of the PA subunit of the viral RNA-dependent RNA polymerase or variant thereof and does not modulate, preferably modulate the activity of other polymerases, in particular mammalian polymerases.

The term “in a high-throughput setting” refers to high-throughput screening assays and techniques of various types which are used to screen libraries of test compounds for their ability to decrease or prevent the binding of the PA subunit of the viral RNA-dependent RNA polymerase to its ligand. Typically, the high-throughput assays are performed in a multi-well format and include cell-free as well as cell-based assays.

The term “purified” in reference to a polypeptide, does not require absolute purity such as a homogenous preparation, rather it represents an indication that the polypeptide is relatively purer than in the natural environment. Generally, a purified polypeptide is substantially free of other proteins, lipids, carbohydrates, or other materials with which it is naturally associated, preferably at a functionally significant level, for example, at least 85% pure, more preferably at least 90% or 95% pure, most preferably at least 99% pure. The expression “purified to an extent to be suitable for crystallization” refers to a polypeptide that is 85% to 100%, preferably 90% to 100%, more preferably 95% to 100% or 98% to 100% pure and can be concentrated to higher than 3 mg/ml, preferably higher than 10 mg/ml, more preferably higher than 18 mg/ml without precipitation. A skilled artisan can purify a polypeptide using standard techniques for protein purification. A substantially pure polypeptide will yield a single major band on a non-reducing polyacrylamide gel.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, γ, δ, ε, and μ. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol 4^{.}389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜—strands arranged in two ˜—sheets. Typically, the heavy chain of an antibody comprises four Ig domains with three of them being constant (CH domains: CHI. CH2. CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCy1-Cy2-Cy3), whereas the human IgG light chain is composed of two immunoglobulin domains linked from N- to C-terminus in the order VL-CL, being either of the kappa or lambda type (VK-CK or VA.-CA.). Exemplified, the constant chain of human IgG comprises 447 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5th ed. U.S. Department of Health and Human Service, National Institutes of Health, Bethesda, Md. The “EU index as in Kabat” refers to the residue numbering of the human IgG lEU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat. Papain digestion of antibodies produces two identical antigen binding fragments, called “Fab fragments” (also referred to as “Fab portion” or “Fab region”) each with a single antigen binding site, and a residual “Fe fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG, IgA and IgD isotypes, the Fe region is composed of two identical protein fragments, derived from the CH2 and CH3 domains of the antibody's two heavy chains; in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a V H and V L domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA). “Bi-specific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

The “Kd” or “Kd-value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

The Kd or Kd-value may also be measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately ten response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min Association rates (kon) and dissociation rates (koff) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M-1s-1 by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kon” can also be determined as described above using a BIACORE®-2000 or a BIACORE®-3000 system (BIAcore, Inc., Piscataway, N.J.).

Typically, antibodies bind with a sufficient binding affinity to their target, for example, with a Kd value of between 500 nM-1 pM, i.e. 500 nM, 450 nM, 400 nM, 350 nM, 300 nM, 250 nM, 200 nM, 150 nM, 100 nM, 50 nM, 1 nM, 900 pM, 800 pM, 700 pM, 600 pM, 500 pM, 400 pM, 300 pM, 200 pM, 100 pM, 50 pM, 1 pM.

The term “pharmaceutically acceptable salt” refers to a salt of a compound identifiable by the methods of the present invention or a compound of the present invention. Suitable pharmaceutically acceptable salts include acid addition salts which may, for example, be formed by mixing a solution of compounds of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, S. M. Berge et al., “Pharmaceutical Salts”, J. Pharm. Sci. 66:1-19 (1977)).

The term “excipient” when used herein is intended to indicate all substances in a pharmaceutical formulation which are not active ingredients such as, e.g., carriers, binders, lubricants, thickeners, surface active agents, preservatives, emulsifiers, buffers, flavoring agents, or colorants.

The term “pharmaceutically acceptable carrier” includes, for example, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

The terms “individual” or “subject” are used interchangeably herein and refer to any mammal, reptile or bird that may benefit from the present invention. In particular, an individual is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. In particular, the “individual” is a human being.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to infectious diseases, traumatic diseases, inflammatory diseases, cutaneous conditions, endocrine diseases, intestinal diseases, neurological disorders, joint diseases, genetic disorders, autoimmune diseases, and various types of cancer.

The term “infection” refers the invasion of an organism's body tissues by disease-causing agents, their multiplication, and the reaction of host tissues to these organisms and the toxins they produce. An “infectious disease”, also known as transmissible disease or communicable disease, is an illness resulting from such infection. Infections are caused by infectious agents including viruses, viroids, prions, bacteria, nematodes such as parasitic roundworms and pinworms, arthropods such as ticks, mites, fleas, and lice, fungi such as ringworm, and other macroparasites such as tapeworms and other helminths. Infections can be classified by the anatomic location or organ system infected, including: respiratory tract infection, urinary tract infection, skin infection, odontogenic infection (an infection that originates within a tooth or in the closely surrounding tissues), vaginal infections, intra-amniotic infection. In addition, locations of inflammation where infection is the most common cause include pneumonia, meningitis and salpingitis.

“Respiratory tract infection” refers to any infectious diseases involving the respiratory tract. An infection of this type is typically further classified as an upper respiratory tract infection (URI or URTI) or a lower respiratory tract infection (LRI or LRTI). Lower respiratory infections, such as pneumonia, tend to be far more serious conditions than upper respiratory infections, such as the common cold.

“Enveloped viruses” such as orthomyxoviruses, paramyxoviruses, retroviruses, flaviviruses, rhabdoviruses and alphaviruses, are surrounded by a lipid bilayer originating from the host plasma membrane. Enveloped viruses include but are not limited to non-segmented and segmented negative-sense single stranded RNA viruses. Non-segmented negative-sense single stranded RNA viruses include the order of Mononegavirales, comprising the Bornaviridae, Filoviridae, Paramyxoviridae, and Rhabdoviridae families. Segmented negative-sense single stranded RNA viruses comprise the family of Orthomyxoviridae, including the genera Influenza A virus, Influenza B virus, Influenza C virus, Thogotovirus, and Isavirus, the families of Arenaviridae and Bunyaviridae, including the genera Hantavirus, Nairovirus, Orthobunyavirus, Phlebovirus, and Tospovirus. In the context of the present invention a segmented negative-sense single stranded RNA viruses is preferably of the Orthomyxoviridae family, more preferably Influenza A virus, Influenza B virus, Influenza C virus, Thogoto virus, Quarja virus, or Isavirus, in particular Influenza A virus.

Influenza A subtypes include but are not limited to avian and mammal subtypes. Mammal subtypes including human, swine, horse, and bat subtypes. Influenza A subtypes include but are not limited to all subtypes H1N1 to H18N11 such as e.g. H1N1, H1N2, H1N3, H1N8, H1N9, H2N2, H2N3, H2N8, H3N1, H3N2, H3N8, H4N4, H4N6, H4N8, H5N1, H5N2, H5N3, H5N8, H5N9, H6N1, H6N2, H6N4, H6N5, H6N8, H7N1, H7N2, H7N3, H7N4, H7N7, H7N8, H7N9, H8N4, H9N2, H9N8, H10N3, H10N7,H10N8, H10N9, H11N2, H11N6, H11N9, H12N1, H12N3, H12N5, H13N6, H13N8, H14N5, H15N2, H15N8, H16N3, H17N10, and H18N11.

Exemplified these subtypes encompass the following strains:

| Sub-type | Strain |
|---|---|
| H1N1 | • A/Beijing/22808/2009 |
| | • A/Brevig Mission/1/1918 |
| | • A/Brisbane/59/2007 |
| | • A/California/04/2009 |
| | • A/California/07/2009 |
| | • A/England/195/2009 |
| | • A/New Caledonia/20/1999 |
| | • A/New York/18/2009 |
| | • A/Ohio/07/2009 |
| | • A/Ohio/UR06-0091/2007 |
| | • A/Puerto Rico/8/1934 |
| | • A/Puerto Rico/8/34/Mount Sinai |
| | • A/Solomon Islands/3/2006 |
| | • A/Texas/05/2009 |
| | • A/USSR/90/1977 |
| | • A/WSN/1933 |
| H1N2 | • A/swine/Guangxi/13/2006 |
| H1N3 | • A/duck/NZL/160/1976 |
| H2N2 | • A/Ann Arbor/6/1960 |
| | • A/Canada/720/2005 |
| | • A/Guiyang/1/1957 |
| | • A/Japan/305/1957 |
| H3N2 | • A/Aichi/2/1968 |
| | • A/Babo1/36/2005 |
| | • A/Brisbane/10/2007 |
| | • A/California/7/2004 |
| | • A/Hong Kong/1/1968 |
| | • A/Memphis/1/68 |
| | • A/Perth/16/2009 |
| | • A/reassortant/IVR-155 |
| | • A/Victoria/210/2009 |
| | • A/Wisconsin/67/X-161/2005 |
| | • A/Wyoming/03/2003 |
| H4N4 | • A/mallard/duck/Alberta/299/1977 |
| H4N6 | • A/mallard/Ohio/657/2002 |
| | • A/Swine/Ontario/01911-1/99 |
| H4N8 | • A/chicken/Alabama/1/1975 |
| H5N1 | • A/Anhui/1/2005 |
| | • A/bar-headed goose/Qinghai/14/2008 |
| | • A/bar-headed goose/Qinghai/1A/2005 |
| | • A/barnswallow/HongKong/D10-1161/2010 |
| | • A/Cambodia/R0405050/2007 |
| | • A/Cambodia/S1211394/2008 |
| | • A/chicken/Egypt/2253-1/2006 |
| | • A/chicken/India/NIV33487/2006 |
| | • A/chickenNietNam/NCVD-016/2008 |
| | • A/chicken/ Yamaguchi/7/2004 |
| | • A/Common magpie/Hong Kong/2256/2006 |
| | • A/common magpie/Hong Kong/5052/2007 |
| | • A/Duck/Hong Kong/p46/1997 |
| | • A/duck/Hunan/795/2002 |
| | • A/duck/Laos/3295/2006 |
| | • A/Egypt/2321-NAMRU3/2007 |
| | • A/Egypt/3300-NAMRU3/2008 |
| | • A/Egypt/N05056/2009 |
| | • A/goose/Guangdong/1/96 |
| | • A/goose/Guiyang/337/2006 |
| | • A/Hong kong/213/2003 |
| | • A/Hong Kong/483/1997 |
| | • A/Hubei/2011 |
| | • A/Indonesia/5/2005 |
| | • A/Japanese white-eye/Hong Kong/1038/2006 |
| | • A/Thailand/1 (KAN-1) /2004 |
| | • A/turkey/Turkey/1/2005 |
| | • A/Vietnam/UT3141311/2008 |
| | • A/whooper swan/Mongolia/244/2005 |
| | • A/Xinjiang/1/2006 |
| H5N2 | • A/American green-winged teal/California/HKWF609/07 |
| | • A/ostrich/South Africa/AI1091/2006 |
| H5N3 | • A/duck/Hokkaido/167/2007 |
| H5N8 | • A/duck/NY/191255-59/2002 |
| H5N9 | • A/chicken/Italy/22A/1998 |
| H6N1 | • A/northern shoveler/California/HKWF115/2007 |
| H6N4 | • A/chicken/HongKong/17/77 |
| H6N8 | • A/mallard/Ohio/217/1998 |
| H7N2 | • A/ruddy turnstone/New Jersey/563/2006 |
| H7N3 | • A/turkey/Italy/214845/2002 |
| H7N7 | • A/chicken/Netherlands/1/03 |
| | • A/equine/Kentucky/la/1975 |
| | • A/Netherlands/219/2003 |
| H7N8 | • A/mallard/Netherlands/33/2006 |
| H7N9 | • A/Anhui/1/2013 |
| | • A/Hangzhou/1/2013 |
| | • A/Hangzhou/3/2013 |
| | • A/Pigeon/Shanghai/S1069/2013 |
| | • A/Shanghai/1/2013 |
| | • A/Shanghai/4664T/2013 |
| | • A/Zhejiang/1/2013 |
| | • A/Zhejiang/DTID-ZJ1J10/2013 |
| H8N4 | • A/pintail duck/Alberta/114/1979 |
| H9N2 | • A/Chicken/Hong Kong/G9/1997 |
| | • A/duck/Hong Kong/448/78 |
| | • A/Guinea fowl/Hong Kong/WF10/99 |
| | • A/Hong Kong/1073/99 |
| | • A/Hong Kong/35820/2009 |
| H10N3 | • A/duck/Hong Kong/786/1979 |
| | • A/mallard/Minnesota/Sg-00194/2007 |
| H10N9 | • A/duck/HongKong/562/1979 |
| H11N2 | • A/duck/Yangzhou/906/2002 |
| H11N9 | • A/mallard/Alberta/294/1977 |
| H12N1 | • A/mallard duck/Alberta/342/1983 |
| H12N5 | • A/green-winged teal/ALB/199/1991 |
| H13N8 | • A/black-headed gull/Netherlands/1/00 |
| H14N5 | • A/Mallard/Astrakhan(Gurjev)/263/1982 |
| H15N2 | • A/Australian shelduck/Western Australia/1756/1983 |
| H15N8 | • A/duck/AUS/341/1983 |
| H16N3 | • A/black-headed gull/Sweden/5/99 |
| H17N10 | • A/Little yellow shoulder bat/Guatemala/060/2010 |
| H18N11 | • A/flat-faced bat/Peru/033/2010 |
| Influenza B | • B/Brisbane/60/2008 |
| | • B/Florida/4/2006 |
| | • B/Malaysia/2506/2004 |
| | • B/Memphis/13/03 |

### Embodiments

In a first aspect, the present invention relates to an in silico method for identifying compounds which decrease or prevent the binding of the viral RNA-dependent RNA polymerase from the Orthomyxoviridae family or variant thereof, to its ligand comprising the steps of:

(a) constructing a computer model based on the structure coordinates of one or more of the binding site(s) of the viral RNA-dependent RNA polymerase to its ligand;
(b) selecting a potential modulating compound by a method selected from the group consisting of:

(i) modifying the co-crystallised ligand inside the one or more binding site(s),

(ii) filtering and selecting compounds from small molecule databases based on the interaction profile of the co-crystallised ligand with the one or more binding site(s) of the viral RNA-dependent RNA polymerase, and/or based on 3D similarity to the co-crystallised ligand, and

(iii) de novo ligand design of said compound based on the interaction profile of the co-crystallised ligand with the binding site of the viral RNA-dependent RNA polymerase and/or based on 3D similarity to the co-crystallised ligand;

(c) employing computational means to perform a fitting program operation between computer models of the said compound and said one or more binding site(s) in order to provide an energy-minimized configuration of the said compound in the active site; and/or employing computational docking methods to position and place said compounds into the said binding site in order to provide reasonable 3D-arrangements of the chemical entities, said compounds; and
(d) evaluating the results of said fitting operation and optionally said docking methods to quantify the association between the said compound and the one or more binding site(s) model, thereby evaluating the ability of said compound to associate with the said binding site.

In embodiments, the viral RNA-dependent RNA polymerase is the RNA-dependent RNA polymerase of Influenza A, B, C, or D virus or is a variant thereof. In particular, the viral RNA-dependent RNA polymerase is the RNA-dependent RNA polymerase of an Influenza A or B virus.

In embodiments, the one or more binding site(s) are the binding sites of the PA subunit of the RNA-dependent RNA polymerase to its ligand.

In particular embodiments, the PA subunit of the RNA-dependent RNA polymerase of an Influenza A or B virus has an amino acid sequence as set forth in SEQ ID NO: 1, SEQ ID NO 2, or SEQ ID NO: 44, respectively.

In particular, said ligand of the viral RNA-dependent RNA polymerase from the Orthomyxoviridae family or variant thereof, is the cellular Polymerase II (Pol II). In particular, the ligand is the carboxy-terminal domain (CTD) of Pol II.

In particular, said ligand of the PA subunit of the viral RNA-dependent RNA polymerase from the Orthomyxoviridae family or variant thereof, is the cellular Polymerase II (Pol II). In particular, the ligand is the carboxy-terminal domain (CTD) of Pol II.

The in silico method for identifying compounds which decrease or prevent the binding of the viral RNA-dependent RNA polymerase from the Orthomyxoviridae family or variant thereof to its ligand comprises step (a) of constructing a computer model based on the structure coordinates of the one or more binding site(s) of the viral RNA-dependent RNA polymerase, in particular of the binding site(s) of the PA subunit of the RNA-dependent RNA polymerase, to its ligand.

In embodiments, the one or more binding site(s) of the viral RNA-dependent RNA polymerase from the Orthomyxoviridae family or variant thereof, comprises amino acids of the first and/or of the second binding site of the PA subunit of the RNA-dependent RNA polymerase of Influenza A virus (in particular SEQ ID NO: 1 or SEQ ID NO: 44) or Influenza B virus (in particular SEQ ID NO: 2), or comprises amino acids occupying analogous position in the sequence of a PA subunit aligned thereto.

In embodiments, the binding site of the viral RNA-dependent RNA polymerase from the Orthomyxoviridae family or variant thereof, comprises one or more amino acids selected from the group consisting of K630, R633, and E444 of the PA subunit of the RNA-dependent RNA polymerase of Influenza A virus (in particular according to SEQ ID NO: 1); comprises one or more amino acids selected from the group consisting of K635, R638, and E449 of SEQ ID NO: 44; or comprises amino acids occupying analogous position in the sequence of a PA subunit aligned thereto.

In embodiments, the binding site of the viral RNA-dependent RNA polymerase from the Orthomyxoviridae family or variant thereof, comprises amino acids K630, R633, and E444 of the PA subunit of the RNA-dependent RNA polymerase of Influenza A virus (SEQ ID NO: 1); comprises amino acids K635, R638, and E449 of the PA subunit of SEQ ID NO: 44; or comprises amino acids occupying analogous position in the sequence of a PA subunit aligned thereto.

In particular embodiments, the binding site of the RNA-dependent RNA polymerase of Influenza A virus further comprises one or more amino acids selected from the group consisting of K289, R449, and E452 of SEQ ID NO: 1, or comprises one or more amino acids selected from the group consisting of K289, R454 and E457 of SEQ ID NO: 44.

In particular embodiments, the binding site of the RNA-dependent RNA polymerase of Influenza A virus further comprises amino acids K289, R449, and E452 of SEQ ID NO: 1.

In particular embodiments, the binding site of the RNA-dependent RNA polymerase of Influenza A virus further comprises amino acids K289, R454 and E457 of SEQ ID NO: 44.

In particular embodiments, the binding site of the RNA-dependent RNA polymerase of Influenza A virus further comprises amino acids F440 and/or F607 of SEQ ID NO: 1.

In particular embodiments, the binding site of the RNA-dependent RNA polymerase of Influenza A virus further comprises amino acids Y445 and/or F612 of SEQ ID NO: 44.

In particular embodiments, the binding site of the RNA-dependent RNA polymerase of Influenza A virus further comprises one or more amino acid selected from the group consisting of M288, L290, S291, T313, F314, I545, M543, and K554 of SEQ ID NO: 1.

In particular embodiments, the binding site of the RNA-dependent RNA polymerase of Influenza A virus further comprises one or more amino acid selected from the group consisting of L288, L290, S291, T313, F314, L550, M548, and R559 of SEQ ID NO: 44.

In particular embodiments, the binding site of the RNA-dependent RNA polymerase of Influenza A virus further comprises amino acids M288, L290, S291, T313, F314, I545, M543, and K554 of SEQ ID NO: 1.

In particular embodiments, the binding site of the RNA-dependent RNA polymerase of Influenza A virus further comprises amino acid L288, L290, S291, T313, F314, L550, M548, and R559 of SEQ ID NO: 44.

In particular embodiments, the binding site of the RNA-dependent RNA polymerase of Influenza A virus further comprises amino acid G629 of SEQ ID NO: 1.

In particular embodiments, the binding site of the RNA-dependent RNA polymerase of Influenza A virus further comprises amino acid G634 of SEQ ID NO: 44.

In particular embodiments, the binding site of the RNA-dependent RNA polymerase of Influenza A virus comprises amino acids F440, E444, F607, G629, K630, and R633 of the PA subunit of the RNA-dependent RNA polymerase of Influenza A virus (in particular according to SEQ ID NO: 1), comprises amino acids Y445, E449, F612, G634, K635, and R683 according to SEQ ID NO: 44, or comprises amino acids occupying analogous position in the sequence of a PA subunit aligned thereto.

In particular embodiments, the binding site of the RNA-dependent RNA polymerase of Influenza A virus comprises amino acids M288, K289, L290, S291, T313, F314, R449, E452, M543, K554, and I545, of the PA subunit of the RNA-dependent RNA polymerase of Influenza A virus (in particular according to SEQ ID NO: 1), comprises amino acids L288, K289, L290, S291, T313, F314, R454, E457, M548, K559, and L550, of SEQ ID NO: 44, or comprises amino acids occupying analogous position in the sequence of a PA subunit aligned thereto.

In particular embodiments, the binding site of the RNA-dependent RNA polymerase of Influenza A virus comprises amino acids M288, K289, L290, S291, T313, F314, F440, E444, R449, E452, M543, K554, I545, F607, G629, K630, and R633, of the PA subunit of the RNA-dependent RNA polymerase of Influenza A virus (in particular according to SEQ ID NO: 1), comprises amino acids L288, K289, L290, S291, T313, F314, Y445, E449, R454, E457, M548, K559, L550, F612, G634, K635, and R638 of SEQ ID NO: 44, or comprises amino acids occupying analogous position in the sequence of a PA subunit aligned thereto.

In particular embodiments, the binding site of the RNA-dependent RNA polymerase of Influenza A virus comprises amino acids 258-713 of SEQ ID NO: 1, or comprises amino acids 201-716 of SEQ ID NO: 44 and optionally an amino-terminal linker having the amino acid sequence GMGSGMA (SEQ ID NO: 3).

In particular embodiments, said binding site of the RNA-dependent RNA polymerase of Influenza A virus has a structure defined by the structure coordinates as shown in FIG. 11.

In embodiments, the binding site of the viral RNA-dependent RNA polymerase from the Orthomyxoviridae family or variant thereof, comprises one or more amino acids selected from the group consisting of E445, K631, and R634, of the PA subunit of the RNA-dependent RNA polymerase of Influenza B virus (in particular according to SEQ ID NO: 2); or comprises amino acids occupying analogous position in the sequence of a PA subunit aligned thereto.

In embodiments, the binding site of the viral RNA-dependent RNA polymerase from the Orthomyxoviridae family or variant thereof, comprises amino acids E445, K631, and R634, of the PA subunit of the RNA-dependent RNA polymerase of Influenza B virus (in particular according to SEQ ID NO: 2); or comprises amino acids occupying analogous position in the sequence of a PA subunit aligned thereto.

In particular embodiments, the binding site of the RNA-dependent RNA polymerase of Influenza B virus further comprises amino acid Y441 and/or F604 of SEQ ID NO: 2.

In particular embodiments, the binding site of the RNA-dependent RNA polymerase of Influenza B virus further comprises amino acid G630 of SEQ ID NO: 2.

In particular embodiments, the binding site of the RNA-dependent RNA polymerase of Influenza B virus comprises amino Y441, E445, F604, G630, K631, R634 and of the PA subunit of the RNA-dependent RNA polymerase of Influenza B virus (in particular according to SEQ ID NO: 2), or comprises amino acids occupying analogous position in the sequence of a PA subunit aligned thereto.

In particular embodiments, the binding site of the RNA-dependent RNA polymerase of Influenza B virus comprises amino acids 258-722 of SEQ ID NO: 2, and optionally an amino-terminal linker having the amino acid sequence GMGSGMA (SEQ ID NO: 3).

In particular embodiments, said the binding site of the RNA-dependent RNA polymerase of Influenza B virus has structure defined by the structure coordinates as shown in FIG. 12.

In particular embodiments, the one or more binding site(s) of the RNA-dependent RNA polymerase of Influenza A virus or of Influenza B virus are isolated, i.e. do not interact or are not bound by other parts of the RNA-dependent RNA polymerase.

In particular embodiments, the one or more binding site(s) of the PA subunit of the RNA-dependent RNA polymerase of Influenza A virus or of Influenza B virus may further interact with or bind to additional parts of the PA subunit, all or parts of the PB1 subunit and/or all or parts of the PB2 subunit. In particular, the one or more binding site(s) of the PA subunit of the RNA-dependent RNA polymerase of Influenza A virus or of Influenza B virus may bind to the complete heterotrimeric polymerase. In particular, the one or more binding site(s) of the PA subunit of the RNA-dependent RNA polymerase of Influenza A virus or of Influenza B are an integrated part of the complete heterotrimeric polymerase.

The manner of obtaining the structure coordinates as shown e.g. in FIGS. 11 and 12, the interpretation of the coordinates and their utility in understanding the protein structure, as described herein, are commonly understood by the skilled person and by reference to standard texts such as J. Drenth, “Principles of protein X-ray crystallography”, 2nd Ed., Springer Advanced Texts in Chemistry, New York (1999); and G. E. Schulz and R. H. Schirmer, “Principles of Protein Structure”, Springer Verlag, New York (1985). For example, X-ray diffraction data is first acquired, often using cryoprotected (e.g., with 20% to 30% glycerol) crystals frozen to 100 K, e.g., using a beamline at a synchrotron facility or a rotating anode as an X-ray source. Then, the phase problem is solved by a generally known method, e.g., multiwavelength anomalous diffraction (MAD), multiple isomorphous replacement (MIR), single wavelength anomalous diffraction (SAD), or molecular replacement (MR). The sub-structure may be solved using SHELXD (Schneider and Sheldrick, 2002, Acta Crystallogr. D. Biol. Crystallogr. (Pt 10 Pt 2), 1772-1779), phases calculated with SHARP (Vonrhein et al., 2006, Methods Mol. Biol. 364:215-30), and improved with solvent flattening and non-crystallographic symmetry averaging, e.g., with RESOLVE (Terwilliger, 2000, Acta Cryst. D. Biol. Crystallogr. 56:965-972). Model autobuilding can be done, e.g., with ARP/wARP (Perrakis et al., 1999, Nat. Struct. Biol. 6:458-63) and refinement with, e.g. REFMAC (Murshudov, 1997, Acta Crystallogr. D. Biol. Crystallogr. 53: 240-255).

Crystals of the viral RNA-dependent RNA polymerase from the Orthomyxoviridae family or variant thereof can be grown by any method known to the person skilled in the art including, but not limited to, hanging and sitting drop techniques, sandwich-drop, dialysis, and microbatch or microtube batch devices. It would be readily apparent to one of skill in the art to vary the crystallization conditions disclosed above to identify other crystallization conditions that would produce crystals of the viral RNA-dependent RNA polymerase from the Orthomyxoviridae family or variant thereof alone or in complex with a compound. Such variations include, but are not limited to, adjusting pH, protein concentration and/or crystallization temperature, changing the identity or concentration of salt and/or precipitant used, using a different method for crystallization, or introducing additives such as detergents (e.g., TWEEN 20 (monolaurate), LDOA, Brij 30 (4 lauryl ether)), sugars (e.g., glucose, maltose), organic compounds (e.g., dioxane, dimethylformamide), lanthanide ions, or poly-ionic compounds that aid in crystallizations. High throughput crystallization assays may also be used to assist in finding or optimizing the crystallization condition.

Microseeding may be used to increase the size and quality of crystals. In brief, micro-crystals are crushed to yield a stock seed solution. The stock seed solution is diluted in series. Using a needle, glass rod or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

In particular embodiments, the viral RNA-dependent RNA polymerase from the Orthomyxoviridae family or variant thereof, is crystallisable using (i) an aqueous protein solution, i.e. the crystallization solution, with a protein concentration of 5 to 20 mg/ml, e.g. of 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/ml, preferably of 8 to 15 mg/ml, most preferably of 10 to 15 mg/ml in a buffer system such as HEPES or Tris-HCl at concentrations ranging from 10 mM to 3 M, in particular 10 mM to 2 M, in particular 20 mM to 1 M, at pH 3 to pH 9, in particular pH 4 to pH 9, in particular pH 7 to pH 9, and (ii) a precipitant/reservoir solution comprising one or more substances such as sodium formate, ammonium sulphate, lithium sulphate, magnesium acetate, manganese acetate, or ethylene glycol.

Optionally, the protein solution may contain one or more salts such as monovalent salts, e.g., NaCl, KCl, or LiCl, in particular NaCl, at concentrations ranging from 10 mM to 1 M, in particular 20 mM to 500 mM, in particular 50 mM to 200 mM, and/or divalent salts, e.g., MnCl₂, CaCl₂, MgCl₂, ZnCl₂, or CoCl2, in particular MgCl₂ and MnCl₂, at concentrations ranging from 0.1 to 50 mM, in particular 0.5 to 25 mM, in particular 1 to 10 mM or 1 to 5 mM.

In embodiments, the precipitant/reservoir solution comprises sodium formate at concentrations ranging from 0.5 to 2 M, in particular 1 to 1.8 M, a buffer system such as HEPES at concentrations ranging from 10 mM to 1 M, in particular 50 mM to 500 mM, in particular 75 to 150 mM, preferably at pH 4 to 8, in particular pH 5 to 7, and/or ethylene glycol at concentrations ranging from 1% to 20%, in particular 2% to 8%, in particular 2 to 5%.

The viral RNA-dependent RNA polymerase from the Orthomyxoviridae family or variant thereof is preferably 85% to 100% pure, in particular 90% to 100% pure, in particular 95% to 100% pure in the crystallization solution. To produce crystals, the protein solution suitable for crystallization may be mixed with an equal volume of the precipitant solution.

In a particular embodiment, the crystallization medium comprises 0.05 to 2 μl, in pparticular 0.8 to 1.2 μl, of protein solution suitable for crystallization mixed with a similar, in paricular equal volume of precipitant solution comprising 1.0 to 2.0 M sodium formate, 80 to 120 mM HEPES pH 6.5 to pH 7.5, and 2 to 5% glycol.

In a further embodiment, the precipitant solution comprises, preferably essentially consists of or consists of 1.6 M sodium formate, 0.1 M HEPES pH 7.0, and 5% glycol, and the crystallization/protein solution comprises, preferably essentially consists or consists of 10 to 15 mg/ml in 20 mM HEPES pH 7.5, 150 mM NaCl, 2.0 mM MnCl₂, and 2.0 mM MgCl₂.

In another embodiment, the viral RNA-dependent RNA polymerase from the Orthomyxoviridae family or variant thereof, is co-crystallizable with a compound. In particular embodiments, the compound is the natural ligand of the viral RNA-dependent RNA polymerase, in particular CTD of cellular Pol II. In alternative embodiments, the compound modulates, preferably decreases or prevents, the binding of the viral RNA-dependent RNA polymerase to its ligand, in particular to CTD of cellular Pol II. In particular embodiments, the viral RNA-dependent RNA polymerase from the Orthomyxoviridae family or variant thereof, is co-crystallizable with said compound (i) an aqueous protein solution with a concentration of the fragment of the PA subunit and/or the entire polypeptide of 5 to 20 mg/ml, e.g., 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/ml, in particular of 8 to 15 mg/ml, in particular of 10 to 15 mg/ml in a buffer system such as HEPES or Tris-HCl at concentrations ranging from 10 mM to 3 M, in particular 10 mM to 2 M, in particular 20 mM to 1 M, at pH 3 to pH 9, in particular pH 4 to pH 9, in particular pH 7 to pH 9, and (ii) a precipitant/reservoir solution comprising one or more substances such as sodium formate, ammonium sulphate, lithium sulphate, magnesium acetate, manganese acetate, ethylene glycol, or PEG. In particular embodiments, said compound is added to the aqueous protein solution for co-crystallization to a final concentration of between 0.5 and 5 mM, in particular of between 1.5 and 5 mM, i.e. 0.5, 1, 1.5, 2, 2.5, 3, 4.5 or 5 mM.

Optionally, the protein solution may contain one or more salts such as monovalent salts, e.g., NaCl, KC1, or LiCl, in particular NaCl, at concentrations ranging from 10 mM to 1 M, in particular 20 mM to 500 mM, in particular 50 mM to 200 mM, and/or divalent salts, e.g., MnCl2, CaCl2, MgCl2, ZnCl2, or CoCl2, in particular MgCl2 and MnCl2, at concentrations ranging from 0.1 to 50 mM, in particular 0.5 to 25 mM, in particular 1 to 10 mM or 1 to 5 mM.

In particular embodiments, the precipitant/reservoir solution comprises ammonium sulphate at concentrations ranging from 0.1 to 2.5 M, in particular 0.1 to 2.0 M, a buffer system such as Bis-Tris at concentrations ranging from 10 mM to 1 M, in particular 50 mM to 500 mM, in particular 75 to 150 mM, at preferably pH 4 to 7, in particular pH 5 to 6, and/or PEG such as PEG 3350 at concentrations ranging from 1% to 30%, in particular 15% to 30%, in particular 20 to 25%.

In particular embodiments, viral RNA-dependent RNA polymerase from the Orthomyxoviridae family or variant thereof is preferably 85% to 100% pure, more preferably 90% to 100% pure, even more preferably 95% to 100% pure in the protein solution. For co-crystallization, the aqueous protein solution comprising the viral RNA-dependent RNA polymerase from the Orthomyxoviridae family or variant thereof, and the ligand may be mixed with an equal volume of the precipitant solution.

The in silico method for identifying compounds which decrease or prevent the binding of the viral RNA-dependent RNA polymerase from the Orthomyxoviridae family or variant thereof to its ligand further comprises step (b) of selecting a potential modulating compound. Said compound may in particular be selected by

(i) modifying the co-crystallised ligand inside the binding site,

(ii) filtering and selecting compounds from small molecule databases based on the interaction profile of the co-crystallised ligand with the binding site of the viral RNA-dependent RNA polymerase, and/or based on 3D similarity to the co-crystallised ligand, and

(iii) de novo ligand design of said compound based on the interaction profile of the co-crystallised ligand with the binding site of the viral RNA-dependent RNA polymerase and/or based on 3D similarity to the co-crystallised ligand;

The present invention permits the use of molecular design techniques to identify, select, or design compounds that potentially decrease or abolish the binding of the carboxy-terminal domain of the PA subunit of a RNA-dependent RNA polymerase to its ligand, in particular to CTD of cellular Pol II, based on the structure coordinates of the (native) binding site according to FIGS. 1 to 3. Said structure coordinates have been achieved from the carboxy-terminal domain of the PA subunit of the RNA-dependent polymerase according to the present invention which have been co-crystallized with a binding compound, in particular with CTD of cellular Pol II.

Such predictive models are valuable in light of the higher costs associated with the preparation and testing of the many diverse compounds that may possibly bind to the carboxy-terminal fragment of the PA subunit of a RNA-dependent RNA polymerase.

The exact binding region between the PA subunit and its ligand was analysed with the help of computer visualization programs, in particular with computer programs selected from the group consisting of SYBYL-X (SYBYL-X 1.3, Tripos, 1699 South Hanley Rd., St. Louis, Mo., 63144, USA) and Benchware 3D-Explorer (Benchware-3D-Explorer 2.7, Tripos, 1699 South Hanley Rd., St. Louis, Mo., 63144, USA). Thereby two different binding regions are identified as depicted in FIGS. 1 to 3.

For each of the binding sites in the PA-subunit a separate three dimensional computer model was created. This is achieved through the use of commercially available software mentioned above.

The created three dimensional computer models served as input for

(i.) the exact analysis of the interaction profile between the ligand and one or more of the binding sites (in particular by analysing hydrogen bonding, van der Waals interactions, and/or electrostatic interactions)

(ii.) for modifying the co-crystallized ligand in order to improve the binding potential (in particular by increasing the interaction surface between ligand and the respective binding sites and/or decreasing degrees of freedom of the ligand)

(iii.) applying computational docking approaches in order to position compounds into the binding site and evaluating the fit of said compound in one or more of the binding sites, and/or

(iv.) de-novo design of a compound which fits into one or more of the binding sites and is able to interact favourably with said one or more binding sites.

The test compounds mentioned above in point (iii.) may be chosen from molecule libraries of small molecules which are offered from commercial vendors. From these commercial offerings compounds with sub-structural elements may be filtered which can best mimic the co-crystallized ligand, in particular those which mimic the phosphorylated Serine group found in the CTD of the cellular Pol II. This filtering can be done with well-known chemoinformatic tools, in particular with the software suite of SYBY-X (SYBYL-X 1.3, Tripos, 1699 South Hanley Rd., St. Louis, Mo., 63144, USA).

In this screening, the quality of fit of such compounds to the active site may be judged either by shape complementarity or by estimated interaction energy (Meng et al., 1992, J. Comp. Chem. 13:505-524).

Once suitable compounds or fragments have been selected, they can be designed or assembled into a single compound or complex. This manual model building is performed using software such as SYBYL-X (SYBYL-X 1.3, Tripos, 1699 South Hanley Rd., St. Louis, Mo., 63144, USA), MOE (Molecular Operating Environment (MOE), 2013.08; Chemical Computing Group Inc., 1010 Sherbooke St. West, Suite #910, Montreal, QC, Canada, H3A 2R7, 2016.) or Maestro (Schrödinger Release 2016-1: Maestro, version 10.5, Schrödinger, LLC, New York, N.Y., 2016). Useful programs aiding the skilled person in connecting individual compounds or fragments include, for example, (i) LUDI (Bohm, 1992, J. Comp. Aid. Mol. Des. 6:61-78) (ii) Muse Invent (2012, Certara, USA, Inc.); (ii) CAVEAT (Bartlett et al., 1989, in Molecular Recognition in Chemical and Biological Problems, Special Publication, Royal Chem. Soc. 78:182-196; Lauri and Bartlett, 1994, J. Comp. Aid. Mol. Des. 8:51-66; CAVEAT is available from the University of California, Berkley, Calif.), (ii) 3D Database systems such as ISIS (MDL Information Systems, San Leandro, Calif.; reviewed in Martin, 1992, J. Med. Chem. 35:2145-2154), and (iii) HOOK (Eisen et al., 1994, Proteins: Struct., Funct., Genet. 19:199-221; HOOK is available from Molecular Simulations Incorporated, San Diego, Calif.).

Another approach enabled by this invention, is the computational screening/docking of small molecule databases for compounds that can bind in whole or part to the binding site of carboxy-terminal fragment of the PA subunit of a RNA-dependent RNA polymerase. For the computational docking procedure there are several programs available, e.g. FlexX (Rarey et al., J Mol Biol. 1996 Aug. 23; 261(3):470-89), Glide (Friesner et al., J. Med. Chem., 2004, 47, 1739-1749), SurflexDock (Jain et al., J. Computer-Aided Molecular Design. 2007, 21, 281-306.)

Alternatively, a potential inhibitor of the binding of carboxy-terminal fragment of the PA subunit of a RNA-dependent RNA polymerase to its ligand, in particular to CTD of Pol II, may be designed de novo on the basis of the 3D structure of the PA polypeptide fragment according to FIGS. 1 to 3. There are various de novo ligand design methods available to the person skilled in the art. Such methods include (i) LUDI (Bohm, 1992, J. Comp. Aid. Mol. Des. 6:61-78); (ii) Muse Invent (2012, Certara, USA, Inc.); (iii) LEGEND (Nishibata and Itai, Tetrahedron 47:8985-8990; LEGEND is available from Molecular Simulations Incorporated, San Diego, Calif.), (iii) LeapFrog (available from Tripos Associates, St. Louis, Mo.), (iv) SPROUT (Gillet et al., 1993, J. Comp. Aid. Mol. Des. 7:127-153; SPROUT is available from the University of Leeds, UK), (v) GROUPBUILD (Rotstein and Murcko, 1993, J. Med. Chem. 36:1700-1710), and (vi) GROW (Moon and Howe, 1991, Proteins 11:314-328).

In addition, several molecular modelling techniques (hereby incorporated by reference) that may support the person skilled in the art in de novo design and modelling of potential inhibitors of the binding site, have been described and include, for example, Cohen et al., 1990, J. Med. Chem. 33:883-894; Navia and Murcko, 1992, Curr. Opin. Struct. Biol. 2:202-210; Balbes et al., 1994, Reviews in Computational Chemistry, Vol. 5, Lipkowitz and Boyd, Eds., VCH, New York, pp. 37-380; Guida, 1994, Curr. Opin. Struct. Biol. 4:777-781.

A molecule designed or selected as binding to the binding site of the RNA-dependent RNA polymerase may be further computationally optimized so that in its bound state it preferably lacks repulsive electrostatic interaction with the target region. Such non-complementary (e.g., electrostatic) interactions include repulsive charge-charge, dipole-dipole and charge-dipole interactions. Specifically, the sum of all electrostatic interactions between the binding compound and the binding pocket in a bound state, preferably make a neutral or favourable contribution to the enthalpy of binding. Specific computer programs that can evaluate a compound deformation energy and electrostatic interaction are available in the art. Examples of suitable programs include (i) Gaussian 09, Revision E.01, Frisch, M. J.; et al. (2016), AMBER 2016, University of California, San Francisco.); (iii) QUANTA/CHARMM (Molecular Simulations Incorporated, San Diego, Calif.), (iv) OPLS-AA (Jorgensen, 1998, Encyclopedia of Computational Chemistry, Schleyer, Ed., Wiley, New York, Vol. 3, pp. 1986-1989), and (v) Insight II/Discover (Biosysm Technologies Incorporated, San Diego, Calif.). These programs may be implemented, on state of the art computers including hardware enabling 3D visualisation (e.g. NVIDIA Quadro graphics boards, a Silicon Graphics workstation, IRIS 4D/35 or IBM RISC/6000 workstation model 550). Other hardware systems and software packages are known to those skilled in the art.

Once a molecule of interest has been selected or designed, as described above, substitutions may then be made in some of its atoms or side groups in order to improve or modify its binding properties. Generally, initial substitutions are conservative, i.e., the replacement group will approximate the same size, shape, hydrophobicity and charge as the original group. It should, of course, be understood that components known in the art to alter conformation should be avoided. Such substituted chemical compounds may then be analysed for efficiency of fit to the binding site of the carboxy-terminal fragment of the PA subunit of a RNA-dependent RNA polymerase by the same computer methods described in detail above.

The in silico method for identifying compounds which decrease or prevent the binding of the viral RNA-dependent RNA polymerase from the Orthomyxoviridae family or variant thereof to its ligand further comprises step (c) of employing computational means to perform a fitting program operation between computer models of the said compound and the said active site in order to provide an energy-minimized configuration of the said compound in the active site.

The in silico method for identifying compounds which decrease or prevent the binding of the viral RNA-dependent RNA polymerase from the Orthomyxoviridae family or variant thereof to its ligand further comprises step (e) of evaluating the results of said fitting operation and optionally said docking methods to quantify the association between the said compound and the binding site model, thereby evaluating the ability of said compound to associate with said binding site. This evaluation may be achieved (i) based on the output from such docking programs which provide a score reflecting how good a test compound does interact with the binding site, and/or (ii) by visual inspection by the person skilled in the art judging how well the test compound fills the binding site and is adopting a favourable geometry.

In a second aspect, the present invention relates to a method of producing a compound which decreases or prevents the binding of the viral RNA-dependent RNA polymerase from the Orthomyxoviridae family or variant thereof, to its ligand. (in particular to cellular Pol II, in particular to CTD) comprising the steps of

(a) identifying said compound via the method of the first aspect as disclosed above, and

(b) synthesizing said compound, and

(c) optionally formulating said compound or a pharmaceutically acceptable salt thereof with one or more pharmaceutically acceptable excipient(s) and/or carrier(s).

In particular embodiments, the ability of said compound or of a pharmaceutically acceptable salt thereof or of a formulation thereof to decrease or prevent the binding of the carboxy-terminal fragment of the PA subunit of a viral RNA-dependent RNA polymerase from the Orthomyxoviridae family it its ligand, in particular to CTD of cellular Pol II, is tested in vitro or in vivo. Thus, in embodiments, the method of the second aspect further comprises the step of

(c) contacting said compound with the viral RNA-dependent RNA polymerase from the Orthomyxoviridae family, and

(d) determine the ability of said compound to decrease or prevent the binding of viral RNA-dependent RNA polymerase to its ligand, preferably CTD of cellular Pol II.

The quality of fit of such compounds to the binding site may be judged either by shape complementarity or by estimated interaction energy (Meng et al., 1992, J. Comp. Chem. 13:505-524). Methods for synthesizing said compounds are well known to the person skilled in the art.

In particular embodiments, the compound which decreases or prevents the binding of the viral RNA-dependent RNA polymerase to its ligand, in particular to CTD, decreases or completely abolishes said binding. In particular, the compound decreases the binding of the RNA-dependent RNA polymerase to its ligand by 50%, in particular by 60%, in particular by 70%, in particular by 80%, in particular by 90%, and in particular by 100% compared to the binding of the viral RNA-dependent RNA polymerase without said compound, in particular with otherwise the same reaction conditions, i.e., buffer conditions, reaction time and temperature. It is particularly preferred that the compound specifically decreases or prevents the binding of the RNA-dependent RNA polymerase to its ligand, in particular to CTD, but does not decrease or inhibit the binding of other polymerases, in particular of mammalian polymerases, to the same extent, preferably not at all.

The ability of a compound to decrease or prevent the binding of the viral RNA-dependent RNA polymerase from the Orthomyxoviridae family to its ligand, in particular to CTD of cellular Pol II, can easily be assessed. For example, in a first step the purified viral RNA-dependent RNA polymerase from the Orthomyxoviridae family or a variant thereof, is are contacted with its ligand, in particular CTD of cellular Pol II, in presence or absence of varying amounts of the test compound and incubated for a certain period of time, for example, for 5, 10, 15, 20, 30, 40, 60, or 90 minutes. The reaction conditions are chosen such that the purified viral RNA-dependent RNA polymerase from the Orthomyxoviridae family, would bind to its ligand, in particular to CTD of cellular Pol II; without the test compound. In a second step, the mixture is then analysed as to whether the test compound decreases or prevents the binding of the viral RNA-dependent RNA polymerase from the Orthomyxoviridae family or variants thereof, to its ligand, in particular to CTD of cellular Pol II. The analysis of the binding may be performed via any method known in the art, in particular via the one or more of the methods as described in more detail below.

In particular embodiments, the interaction between the viral RNA-dependent RNA polymerase from the Orthomyxoviridae family or variants thereof, and the test compound may be analysed in form of a pull down assay. For example, the viral RNA-dependent RNA polymerase from the Orthomyxoviridae family or variants thereof, may be purified and may be immobilized on a solid surface such as e.g. beads. In one embodiment, the viral RNA-dependent RNA polymerase from the Orthomyxoviridae family or variants thereof, immobilized on beads may be contacted, for example, with (i) another purified protein, polypeptide fragment, or peptide, (ii) a mixture of proteins, polypeptide fragments, or peptides, or (iii) a cell or tissue extract, and binding of proteins, polypeptide fragments, or peptides may be verified by polyacrylamide gel electrophoresis in combination with coomassie staining or Western blotting. Unknown binding partners may be identified by mass spectrometric analysis.

In another embodiment, the interaction between the viral RNA-dependent RNA polymerase from the Orthomyxoviridae family or variants thereof, and a test compound may be analysed in form of an enzyme-linked immunosorbent assay (ELISA)-based experiment. In one embodiment, viral RNA-dependent RNA polymerase from the Orthomyxoviridae family or variants thereof, may be immobilized on the solid surface such as an ELISA plate and contacted with the test compound. Binding of the test compound may be verified, for example, for proteins, polypeptides, peptides, and epitope-tagged compounds by antibodies specific for the test compound or the epitope-tag. These antibodies might be directly coupled to an enzyme or detected with a secondary antibody coupled to said enzyme that—in combination with the appropriate substrates—carries out chemiluminescent reactions (e.g., horseradish peroxidase) or colorimetric reactions (e.g., alkaline phosphatase). In another embodiment, binding of compounds that cannot be detected by antibodies might be verified by labels directly coupled to the test compounds. Such labels may include enzymatic labels, radioisotope or radioactive compounds or elements, fluorescent compounds or metals, chemiluminescent compounds and bioluminescent compounds. In another embodiment, the test compounds might be immobilized on the ELISA plate and contacted with the PA polypeptide fragment or variants thereof according to the invention. Binding of said polypeptide may be verified by an antibody specific to the carboxy-terminal fragment of the PA subunit and chemiluminescence or colorimetric reactions as described above.

In a further embodiment, purified viral RNA-dependent RNA polymerase from the Orthomyxoviridae family or variants thereof, may be incubated with a peptide array and binding of the carboxy-terminal fragment of the PA subunit to specific peptide spots corresponding to a specific peptide sequence may be analysed, for example, by antibodies specific to the viral RNA-dependent RNA polymerase from the Orthomyxoviridae family or variants thereof, antibodies that are directed against an epitope-tag fused to the viral RNA-dependent RNA polymerase from the Orthomyxoviridae family or variants thereof, or by a fluorescence signal emitted by a fluorescent tag coupled to the viral RNA-dependent RNA polymerase from the Orthomyxoviridae family or variants thereof.

In another embodiment, the recombinant host cell expressing the viral RNA-dependent RNA polymerase from the Orthomyxoviridae family or variants thereof, is contacted with a test compound. This may be achieved by co-expression of test proteins or polypeptides and verification of interaction, for example, by fluorescence resonance energy transfer (FRET) or co-immunoprecipitation. In another embodiment, directly labelled test compounds may be added to the medium of the recombinant host cells. The potential of the test compound to penetrate membranes and bind to the viral RNA-dependent RNA polymerase from the Orthomyxoviridae family or variants thereof, may be, for example, verified by immunoprecipitation of said polypeptide and verification of the presence of the label.

In particular embodiments, the above-described methods for identifying compounds which bind to viral RNA-dependent RNA polymerase from the Orthomyxoviridae family or variants thereof, are performed in a high-throughput setting. In a particular embodiment, said methods are carried out in a multi-well microtiter plate as described above using the viral RNA-dependent RNA polymerase from the Orthomyxoviridae family or variants thereof, according to the present invention and labelled test compounds.

In particular embodiments, the test compounds are derived from libraries of synthetic or natural compounds. For instance, synthetic compound libraries are commercially available from Maybridge Chemical Co. (Trevillet, Cornwall, UK), ChemBridge Corporation (San Diego, Calif.), or Aldrich (Milwaukee, Wis.). A natural compound library is, for example, available from TimTec LLC (Newark, Del.). Alternatively, libraries of natural compounds in the form of bacterial, fungal, plant, and animal extracts can be used. Additionally, test compounds can be synthetically produced using combinatorial chemistry either as individual compounds or as mixtures.

In another embodiment, the inhibitory effect of the identified compound on the binding of the viral RNA-dependent RNA polymerase from the Orthomyxoviridae family or variants thereof, to its ligand, in particular CTD of cellular Pol II, may be tested in an in vivo setting. A cell line that is susceptible for Influenza virus infection such as 293T human embryonic kidney cells, Madin-Darby canine kidney cells, or chicken embryo fibroblasts may be infected with an Influenza virus in presence or absence of the identified compound. In a preferred embodiment, the identified compound may be added to the culture medium of the cells in various concentrations. Viral plaque formation may be used as read out for the infectious capacity of the Influenza virus and may be compared between cells that have been treated with the identified compound and cells that have not been treated.

In a further embodiment of the invention, the test compound applied in any of the above described methods is a small molecule. In a particular embodiment, said small molecule is derived from a library, e.g., a small molecule inhibitor library.

In a further embodiment, said test compound is a peptide or protein. In a particular embodiment, said peptide or protein is derived from a peptide or protein library.

In a third aspect, the present invention relates to a compound identifiable by the in silico screening method of the first aspect of the present invention, and/or a compound producible by the production method of the second aspect of the present invention, wherein said compound is able to decrease or to prevent the binding of the viral RNA-dependent RNA polymerase from the Orthomyxoviridae family to its ligand, in particular to CTD of cellular Pol II.

The ability of a compound to decrease or prevent the binding of the viral RNA-dependent RNA polymerase from the Orthomyxoviridae family to its ligand, in particular to CTD of cellular Pol II, can easily be assessed. For example, in a first step the purified viral RNA-dependent RNA polymerase from the Orthomyxoviridae family or a variant thereof, is are contacted with its ligand, in particular CTD of cellular Pol II, in presence or absence of varying amounts of the test compound and incubated for a certain period of time, for example, for 5, 10, 15, 20, 30, 40, 60, or 90 minutes. The reaction conditions are chosen such that the purified viral RNA-dependent RNA polymerase from the Orthomyxoviridae family, would bind to its ligand, in particular to CTD of cellular Pol II; without the test compound. In a second step, the mixture is then analysed as to whether the test compound decreases or prevents the binding of the viral RNA-dependent RNA polymerase from the Orthomyxoviridae family or variants thereof to its ligand, in particular to CTD of cellular Pol II. The analysis of the binding may be performed via any method known in the art, in particular via the one or more of the methods as described in more detail above.

In particular embodiments, said compound may bind to the binding site of the viral RNA-dependent RNA polymerase from the Orthomyxoviridae family or variants thereof, and thereby decrease or prevent the binding of the viral RNA-dependent RNA polymerase from the Orthomyxoviridae family or variants thereof, to its ligand, in particular to CTD of cellular Pol II.

In alternative embodiments, said compound may interact with a different part of the viral RNA-dependent RNA polymerase from the Orthomyxoviridae family and decrease or prevent the binding of the viral RNA-dependent RNA polymerase from the Orthomyxoviridae family to its ligand, preferably to CTD of cellular Pol II, by sterically blocking the binding of the viral RNA-dependent RNA polymerase from the Orthomyxoviridae family to its ligand, in particular CTD of cellular Pol II.

In particular embodiments, said test compound or the ligand of the viral RNA-dependent RNA polymerase from the Orthomyxoviridae family or variants thereof, may comprise a detectable label which provides a signal when bound to the viral RNA-dependent RNA polymerase from the Orthomyxoviridae family or variants thereof, but does not provide a signal if not bound. For example, the test compound may be labelled with fluorescent label which provides a signal when bound to the viral RNA-dependent RNA polymerase from the Orthomyxoviridae family or variants thereof, but does not provide a signal when removed. In further embodiments, test compound may be labelled with fluorescent label and a washing step may be inserted between the incubation step and the analysis step to remove fluorescent unbound test compounds. In particular embodiments, the viral RNA-dependent RNA polymerase from the Orthomyxoviridae family or variants thereof, may be immobilized on a solid surface. In particular, the binding of the test compound to the carboxy-terminal fragment of the PA subunit may be analysed via any of the methods described below with regard to the eighth aspect of the present invention.

Compounds of the present invention can be any agents including, but not restricted to, peptides, peptoids, polypeptides, proteins (including antibodies), lipids, metals, nucleotides, nucleosides, nucleic acids, small organic or inorganic molecules, chemical compounds, elements, saccharides, isotopes, carbohydrates, imaging agents, lipoproteins, glycoproteins, enzymes, analytical probes, polyamines, and combinations and derivatives thereof. The term “small molecules” refers to molecules that have a molecular weight between 50 and about 2,500 Daltons, preferably in the range of 200-800 Daltons. In addition, a test compound according to the present invention may optionally comprise a detectable label. Such labels include, but are not limited to, enzymatic labels, radioisotope or radioactive compounds or elements, fluorescent compounds or metals, chemiluminescent compounds and bioluminescent compounds.

In a fourth aspect, the present invention relates to an antibody directed against the one or more binding site(s) of the RNA-dependent RNA polymerase from a virus belonging to the Orthomyxoviridae family, or variant thereof, to its ligand (in particular to cellular Pol II, in particular to CTD of Pol II).

In embodiments, the viral RNA-dependent RNA polymerase is the RNA-dependent RNA polymerase of Influenza A, B, C, or D virus or is a variant thereof. In particular, the viral RNA-dependent RNA polymerase is the RNA-dependent RNA polymerase of an Influenza A or B virus.

In embodiments, the one or more binding site(s) are the binding sites of the PA subunit of the RNA-dependent RNA polymerase to its ligand.

In particular embodiments, the PA subunit of the RNA-dependent RNA polymerase of an Influenza A or B virus has an amino acid sequence as set forth in SEQ ID NO: 1, SEQ ID NO 2 or SEQ ID NO: 44, respectively.

In particular, said ligand of the viral RNA-dependent RNA polymerase from the Orthomyxoviridae family or variant thereof, is the cellular Polymerase II (Pol II). In particular, the ligand is the carboxy-terminal domain (CTD) of Pol II.

In particular, said ligand of the PA subunit of the viral RNA-dependent RNA polymerase from the Orthomyxoviridae family or variant thereof, is the cellular Polymerase II (Pol II). In particular, the ligand is the carboxy-terminal domain (CTD) of Pol II.

In embodiments, the one or more binding site(s) of the viral RNA-dependent RNA polymerase from the Orthomyxoviridae family or variant thereof, comprises amino acids of the first and/or of the second binding site of the PA subunit of the RNA-dependent RNA polymerase of Influenza A virus (in particular SEQ ID NO: 1 or SEQ ID NO: 44) or Influenza B virus (in particular SEQ ID NO: 2), or comprises amino acids occupying analogous position in the sequence of a PA subunit aligned thereto.

In embodiments, the binding site of the viral RNA-dependent RNA polymerase from the Orthomyxoviridae family or variant thereof, comprises one or more amino acids selected from the group consisting of K630, R633, and E444 of the PA subunit of the RNA-dependent RNA polymerase of Influenza A virus (in particular according to SEQ ID NO: 1); comprises one or more amino acids selected from the group consisting of K635, R638 and E449 of SEQ ID NO: 44; or comprises amino acids occupying analogous position in the sequence of a PA subunit aligned thereto.

In embodiments, the binding site of the viral RNA-dependent RNA polymerase from the Orthomyxoviridae family or variant thereof, comprises amino acids K630, R633, and E444 of the PA subunit of the RNA-dependent RNA polymerase of Influenza A virus (SEQ ID NO: 1); comprises amino acids K635, R638 and E449 of SEQ ID NO: 44; or comprises amino acids occupying analogous position in the sequence of a PA subunit aligned thereto.

In particular embodiments, the binding site of the RNA-dependent RNA polymerase of Influenza A virus further comprises one or more amino acids selected from the group consisting of K289, R449, and E452 of SEQ ID NO: 1, or further comprises one or more amino acids selected from the group consisting of K289, R454 and E457 of SEQ ID NO: 44.

In particular embodiments, the binding site of the RNA-dependent RNA polymerase of Influenza A virus further comprises amino acids K289, R449, and E452 of SEQ ID NO: 1.

In particular embodiments, the binding site of the RNA-dependent RNA polymerase of Influenza A virus further comprises amino acids K289, R454, and E457 of SEQ ID NO: 44.In particular embodiments, the binding site of the RNA-dependent RNA polymerase of Influenza A virus further comprises amino acids F440 and/or F607 of SEQ ID NO: 1.

In particular embodiments, the binding site of the RNA-dependent RNA polymerase of Influenza A virus further comprises amino acids Y445 and/or F612 of SEQ ID NO: 1.

In particular embodiments, the binding site of the RNA-dependent RNA polymerase of Influenza A virus further comprises one or more amino acid selected from the group consisting of M288, L290, S291, T313, F314, I545, M543, and K554 of SEQ ID NO: 1.

In particular embodiments, the binding site of the RNA-dependent RNA polymerase of Influenza A virus further comprises one or more amino acid selected from the group consisting of L288, L290, S291, T313, F314, L550, M548, and R559 of SEQ ID NO: 44.

In particular embodiments, the binding site of the RNA-dependent RNA polymerase of Influenza A virus further comprises amino acids M288, L290, S291, T313, F314, I545, M543, and K554 of SEQ ID NO: 1.

In particular embodiments, the binding site of the RNA-dependent RNA polymerase of Influenza A virus further comprises amino acids L288, L290, S291, T313, F314, L550, M548, and R559 of SEQ ID NO: 44.

In particular embodiments, the binding site of the RNA-dependent RNA polymerase of Influenza A virus further comprises amino acid G629 of SEQ ID NO: 1.

In particular embodiments, the binding site of the RNA-dependent RNA polymerase of Influenza A virus further comprises amino acid G634 of SEQ ID NO: 44.

In particular embodiments, the binding site of the RNA-dependent RNA polymerase of Influenza A virus comprises amino acids F440, E444, F607, G629, K630, and R633 of the PA subunit of the RNA-dependent RNA polymerase of Influenza A virus (in particular according to SEQ ID NO: 1), comprises amino acids Y445, E449, F612, G634, K635, and R683 according to SEQ ID NO: 44, or comprises amino acids occupying analogous position in the sequence of a PA subunit aligned thereto.

In particular embodiments, the binding site of the RNA-dependent RNA polymerase of Influenza A virus comprises amino acids M288, K289, L290, S291, T313, F314, R449, E452, M543, K554, and I545, of the PA subunit of the RNA-dependent RNA polymerase of Influenza A virus (in particular according to SEQ ID NO: 1), comprises amino acids L288, K289, L290, S291, T313, F314, R454, E457, M548, K559, and L550 of SEQ ID NO: 44, or comprises amino acids occupying analogous position in the sequence of a PA subunit aligned thereto.

In particular embodiments, the binding site of the RNA-dependent RNA polymerase of Influenza A virus comprises amino acids M288, K289, L290, S291, T313, F314, F440, E444, R449, E452, M543, K554, I545, F607, G629, K630, and R633, of the PA subunit of the RNA-dependent RNA polymerase of Influenza A virus (in particular according to SEQ ID NO: 1), comprises amino acids L288, K289, L290, S291, T313, F314, Y445, E449, R454, E457, M548, K559, L550, F612, G634, K635, and R638 of SEQ ID NO: 44, or comprises amino acids occupying analogous position in the sequence of a PA subunit aligned thereto.

In particular embodiments, the binding site of the RNA-dependent RNA polymerase of Influenza A virus comprises amino acids 258-713 of SEQ ID NO: 1, or comprises amino acids 201-716 of SEQ ID NO: 44 and optionally an amino-terminal linker having the amino acid sequence GMGSGMA (SEQ ID NO: 3).

In particular embodiments, said binding site of the RNA-dependent RNA polymerase of Influenza A virus has structure defined by the structure coordinates as shown in FIG. 11. In embodiments, the binding site of the viral RNA-dependent RNA polymerase from the Orthomyxoviridae family or variant thereof, comprises one or more amino acids selected from the group consisting of E445, K631, and R634, of the PA subunit of the RNA-dependent RNA polymerase of Influenza B virus (in particular according to SEQ ID NO: 2); or comprises amino acids occupying analogous position in the sequence of a PA subunit aligned thereto.

In embodiments, the binding site of the viral RNA-dependent RNA polymerase from the Orthomyxoviridae family or variant thereof, comprises amino acids E445, K631, and R634, of the PA subunit of the RNA-dependent RNA polymerase of Influenza B virus (in particular according to SEQ ID NO: 2); or comprises amino acids occupying analogous position in the sequence of a PA subunit aligned thereto.

In particular embodiments, the binding site of the RNA-dependent RNA polymerase of Influenza B virus further comprises amino acid Y441 and/or F604 of SEQ ID NO: 2.

In particular embodiments, the binding site of the RNA-dependent RNA polymerase of Influenza B virus further comprises amino acid G630 of SEQ ID NO: 2.

In particular embodiments, the binding site of the RNA-dependent RNA polymerase of Influenza B virus comprises amino Y441, E445, F604, G630, K631, R634 and of the PA subunit of the RNA-dependent RNA polymerase of Influenza B virus (in particular according to SEQ ID NO: 2), or comprises amino acids occupying analogous position in the sequence of a PA subunit aligned thereto.

In particular embodiments, the binding site of the RNA-dependent RNA polymerase of Influenza B virus comprises amino acids 258-722 of SEQ ID NO: 2, and optionally an amino-terminal linker having the amino acid sequence GMGSGMA (SEQ ID NO: 3).

In particular embodiments, said the binding site of the RNA-dependent RNA polymerase of Influenza B virus has structure defined by the structure coordinates as shown in FIG. 12.

In particular embodiments, the one or more binding site(s) of the RNA-dependent RNA polymerase of Influenza A virus or of Influenza B virus are isolated, i.e. do not interact or are not bound by other parts of the RNA-dependent RNA polymerase.

In particular embodiments, the one or more binding site(s) of the PA subunit of the RNA-dependent RNA polymerase of Influenza A virus or of Influenza B virus may further interact with or bind to additional parts of the PA subunit, all or parts of the PB1 subunit and/or all or parts of the PB2 subunit. In particular, the one or more binding site(s) of the PA subunit of the RNA-dependent RNA polymerase of Influenza A virus or of Influenza B virus may bind to the complete heterotrimeric polymerase. In particular, the one or more binding site(s) of the PA subunit of the RNA-dependent RNA polymerase of Influenza A virus or of Influenza B are an integrated part of the complete heterotrimeric polymerase.

The antibody of the present invention may be a monoclonal or polyclonal antibody or portions thereof. Antigen-binding portions may be produced by recombinant DNA techniques or by enzymatic or chemical cleavage of intact antibodies. In some embodiments, antigen-binding portions include Fab, Fab′, F(ab′)₂, Fd, Fv, dAb, and complementarity determining region (CDR) fragments, single-chain antibodies (scFv), chimeric antibodies such as humanized antibodies, diabodies, and polypeptides that contain at least a portion of an antibody that is sufficient to confer specific antigen binding to the polypeptide. The antibody of the present invention is generated according to standard protocols. For example, a polyclonal antibody may be generated by immunizing an animal such as mouse, rat, rabbit, goat, sheep, pig, cattle, or horse with the antigen of interest optionally in combination with an adjuvant such as Freund's complete or incomplete adjuvant, RIBI (muramyl dipeptides), or ISCOM (immunostimulating complexes) according to standard methods well known to the person skilled in the art. The polyclonal antiserum directed against the polypeptide of the first aspect of the present invention is obtained from the animal by bleeding or sacrificing the immunized animal. The serum (i) may be used as it is obtained from the animal, (ii) an immunoglobulin fraction may be obtained from the serum, or (iii) the antibodies specific for the polypeptide of the first aspect of the present invention may be purified from the serum. Monoclonal antibodies may be generated by methods well known to the person skilled in the art. In brief, the animal is sacrificed after immunization and lymph node and/or splenic B cells are immortalized by any means known in the art. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumour suppressor gene. Immortalized cells are screened using the polypeptide of the first aspect of the present invention. Cells that produce antibodies directed against the polypeptide of the first aspect of the present invention, e.g., hybridomas, are selected, cloned, and further screened for desirable characteristics including robust growth, high antibody production, and desirable antibody characteristics. Hybridomas can be expanded (i) in vivo in syngeneic animals, (ii) in animals that lack an immune system, e.g., nude mice, or (iii) in cell culture in vitro. Methods of selecting, cloning, and expanding hybridomas are well known to those of ordinary skill in the art. The skilled person may refer to standard texts such as “Antibodies: A Laboratory Manual”, Harlow and Lane, Eds., Cold Spring Harbor Laboratory Press, Cold Spring Harbor, N.Y. (1990), which is incorporated herein by reference, for support regarding generation of antibodies.

In a fifth aspect, the present invention relates to a nucleic acid encoding an antibody of the fourth aspect of the present invention. The molecular biology methods applied for obtaining such isolated nucleotide fragments are generally known to the person skilled in the art (for standard molecular biology methods see Sambrook et al., Eds., “Molecular Cloning: A Laboratory Manual”, Cold Spring Harbor Laboratory Press, Cold Spring Harbor, N.Y. (1989), which is incorporated herein by reference). For example, RNA can be isolated from Influenza virus infected cells and cDNA generated applying reverse transcription polymerase chain reaction (RT-PCR) using either random primers (e.g., random hexamers of decamers) or primers specific for the generation of the fragments of interest. The fragments of interest can then be amplified by standard PCR using fragment specific primers.

In a sixth aspect, the present invention relates to a vector comprising the nucleic acid of the fifth aspect of the present invention. The person skilled in the art is well aware of techniques used for the incorporation of polynucleotide sequences of interest into vectors (also see Sambrook et al., 1989, supra). Such vectors include any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors may be expression vectors suitable for prokaryotic or eukaryotic expression. Said plasmids may include an origin of replication (ori), a multiple cloning site, and regulatory sequences such as promoter (constitutive or inducible), transcription initiation site, ribosomal binding site, transcription termination site, polyadenylation signal, and selection marker such as antibiotic resistance or auxotrophic marker based on complementation of a mutation or deletion. In one embodiment the polynucleotide sequence of interest is operably linked to the regulatory sequences.

In another embodiment, said vector includes nucleotide sequences coding for epitope-, peptide-, or protein-tags that facilitate purification of polypeptide fragments of interest. Such epitope-, peptide-, or protein-tags include, but are not limited to, hemagglutinin-(HA-), FLAG-, myc-tag, poly-His-tag, glutathione-S-transferase-(GST-), maltose-binding-protein-(MBP-), NusA-, and thioredoxin-tag, or fluorescent protein-tags such as (enhanced) green fluorescent protein ((E)GFP), (enhanced) yellow fluorescent protein ((E)YFP), red fluorescent protein (RFP) derived from Discosoma species (DsRed) or monomeric (mRFP), cyan fluorescence protein (CFP), and the like. In a preferred embodiment, the epitope-, peptide-, or protein-tags can be cleaved off the polypeptide fragment of interest, for example, using a protease such as thrombin, Factor Xa, PreScission, TEV protease, and the like. Preferably, the tag can be cleaved of with a TEV protease. The recognition sites for such proteases are well known to the person skilled in the art. For example, the seven amino acid consensus sequence of the TEV protease recognition site is Glu-X-X-Tyr-X-Gln-Gly/Ser, wherein X may be any amino acid and is in the context of the present invention preferably Glu-Asn-Leu-Tyr-Phe-Gln-Gly (SEQ ID NO: 21). In another embodiment, the vector includes functional sequences that lead to secretion of the polypeptide fragment of interest into the culture medium of the recombinant host cells or into the periplasmic space of bacteria. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

In a seventh aspect, the present invention provides a recombinant host cell comprising said isolated polynucleotide or said recombinant vector. The recombinant host cells may be prokaryotic cells such as archea and bacterial cells or eukaryotic cells such as yeast, plant, insect, or mammalian cells. The person skilled in the art is well aware of methods for introducing said isolated polynucleotide or said recombinant vector into said host cell. For example, bacterial cells can be readily transformed using, for example, chemical transformation, e.g., the calcium chloride method, or electroporation. Yeast cells may be transformed, for example, using the lithium acetate transformation method or electroporation. Other eukaryotic cells can be transfected, for example, using commercially available liposome-based transfection kits such as LipofectamineTM (Invitrogen), commercially available lipid-based transfection kits such as Fugene (Roche Diagnostics), polyethylene glycol-based transfection, calcium phosphate precipitation, gene gun (biolistic), electroporation, or viral infection.

In an eighth aspect, the present invention relates to a pharmaceutical composition producible according to the method of the second aspect of the present invention.

In a ninth aspect, the present invention relates to a pharmaceutical composition comprising the compound of the third aspect of the present invention, an antibody of the fourth aspect of the present invention, a nucleic acid of the fifth aspect of the present invention, or a vector of the sixth aspect of the present invention.

In particular embodiments, the pharmaceutical composition further comprises one or more pharmaceutically acceptable excipient(s) and/or carrier(s).

The pharmaceutical composition contemplated by the present invention may be formulated in various ways well known to one of skill in the art. For example, the pharmaceutical composition of the present invention may be in solid form such as in the form of tablets, pills, capsules (including soft gel capsules), cachets, lozenges, ovules, powder, granules, or suppositories, or in liquid form such as in the form of elixirs, solutions, emulsions, or suspensions.

Solid administration forms may contain excipients such as microcrystalline cellulose, lactose, sodium citrate, calcium carbonate, dibasic calcium phosphate, glycine, and starch (preferably corn, potato, or tapioca starch), disintegrants such as sodium starch glycolate, croscarmellose sodium, and certain complex silicates, and granulation binders such as polyvinylpyrrolidone, hydroxypropylmethyl cellulose (HPMC), hydroxypropylcellulose (HPC), sucrose, gelatine, and acacia. Additionally, lubricating agents such as magnesium stearate, stearic acid, glyceryl behenate, and talc may be included. Solid compositions of a similar type may also be employed as fillers in gelatine capsules. Preferred excipients in this regard include lactose, starch, a cellulose, milk sugar, or high molecular weight polyethylene glycols.

For aqueous suspensions, solutions, elixirs, and emulsions suitable for oral administration the compound may be combined with various sweetening or flavouring agents, colouring matter or dyes, with emulsifying and/or suspending agents and with diluents such as water, ethanol, propylene glycol, and glycerine, and combinations thereof.

The pharmaceutical composition of the present invention may contain release rate modifiers including, for example, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer, and mixtures thereof.

The pharmaceutical composition of the present invention may be in the form of fast dispersing or dissolving dosage formulations (FDDFs) and may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavoring, polyethylene glycol, fumed silica, silicon dioxide, sodium starch glycolate, sodium stearyl fumarate, sorbitol, xylitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized moulds, allowed to cool, and thereby to solidify.

The pharmaceutical composition of the present invention suitable for parenteral administration is best used in the form of a sterile aqueous solution which may contain other substances, for example, enough salts or glucose to make the solution isotonic with blood. The aqueous solutions should be suitably buffered (preferably to a pH of from 3 to 9), if necessary.

The pharmaceutical composition suitable for intranasal administration and administration by inhalation is best delivered in the form of a dry powder inhaler or an aerosol spray from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide, or another suitable gas. The pressurized container, pump, spray or nebulizer may contain a solution or suspension of the active compound, e.g., using a mixture of ethanol and the propellant as the solvent, which may additionally contain a lubricant, e.g., sorbitan trioleate.

In particular embodiments, the pharmaceutical composition is in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation administered in the use of the present invention may be varied or adjusted from about 1 mg to about 1000 mg per m2, preferably about 5 mg to about 150 mg/m2 according to the particular application and the potency of the active component.

In a tenth aspect, the present invention relates to a compound according to the third aspect of the present invention, an antibody of the fourth aspect of the present invention, a nucleic acid of the fifth aspect of the present invention, a vector of the sixth aspect of the present invention, or a pharmaceutical composition of the seventh or eighth aspect of the present invention , for use in treating, ameliorating, or preventing disease conditions caused by viral infections with viruses of the Orthomyxoviridae family.

In particular embodiments said disease condition is caused by a virus selected from the group consisting of Influenza A virus, Influenza B virus, and Influenza C virus.

In particular embodiments, the compound according to the seventh or tenth aspect of the present invention, a pharmaceutical composition according to the eleventh or thirteenth aspect of the present invention, or an antibody according to the twelfth aspect of the present invention, for use in treating, ameliorating, or preventing said disease conditions is administered to an animal patient, in particular a mammalian patient, in particular a human patient, orally, buccally, sublingually, intranasally, via pulmonary routes such as by inhalation, via rectal routes, or parenterally, for example, intracavernosally, intravenously, intra-arterially, intraperitoneally, intrathecally, intraventricularly, intra¬urethrally intrasternally, intracranially, intramuscularly, or subcutaneously, they may be administered by infusion or needleless injection techniques.

In an eleventh aspect, the present invention relates to a method of treating ameliorating, or preventing disease conditions caused by viral infections with viruses of the Orthomyxoviridae family, comprising administering a therapeutically effective amount of the compound according to the third aspect of the present invention, an antibody of the fourth aspect of the present invention, a nucleic acid of the fifth aspect of the present invention, a vector of the sixth aspect of the present invention, or a pharmaceutical composition of the seventh or eighth aspect of the present invention.

In particular embodiments said disease condition is caused by a virus selected from the group consisting of Influenza A virus, Influenza B virus, and Influenza C virus.

In particular embodiments, the a compound according to the third aspect of the present invention, an antibody of the fourth aspect of the present invention, a nucleic acid of the fifth aspect of the present invention, a vector of the sixth aspect of the present invention, or a pharmaceutical composition of the seventh or eighth aspect of the present invention, is administered to an animal patient, in particular a mammalian patient, in particular a human patient, orally, buccally, sublingually, intranasally, via pulmonary routes such as by inhalation, via rectal routes, or parenterally, for example, intracavernosally, intravenously, intra-arterially, intraperitoneally, intrathecally, intraventricularly, intra-urethrally intrasternally, intracranially, intramuscularly, or subcutaneously, they may be administered by infusion or needleless injection techniques.

In particular embodiments, an initial dosage of about 0.05 mg/kg to about 20 mg/kg daily is adminitered. A daily dose range of about 0.05 mg/kg to about 2 mg/kg is preferred, with a daily dose range of about 0.05 mg/kg to about 1 mg/kg being most preferred. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

In particular, the present invention relates to the following aspects:

1. An in silico method for identifying compounds which decrease or prevent the binding of the viral RNA-dependent RNA polymerase from the Orthomyxoviridae family or variant thereof, to its ligand, (in particular to cellular Pol II, in particular to CTD) comprising the steps of

(a) constructing a computer model based on the structure coordinates of the binding site of the viral RNA-dependent RNA polymerase to its ligand;

(b) selecting a potential modulating compound by a method selected from the group consisting of:
(i) modifying the co-crystallised ligand inside the binding site,
(ii) filtering and selecting compounds from small molecule databases based on the interaction profile of the co-crystallised ligand with the binding site of the viral RNA-dependent RNA polymerase, and/or based on 3D similarity to the co-crystallised ligand, and
(iii) de novo ligand design of said compound based on the interaction profile of the co-crystallised ligand with the binding site of the viral RNA-dependent RNA polymerase and/or based on 3D similarity to the co-crystallised ligand;

(c) employing computational means to perform a fitting program operation between computer models of the said compound and said binding site in order to provide an energy-minimized configuration of the said compound in the active site; and/or employing computational docking methods to position and place said compounds into the said binding site in order to provide reasonable 3D-arrangements of the chemical entities, said compounds; and

(d) evaluating the results of said fitting operation and optionally said docking methods to quantify the association between the said compound and the binding site model, thereby evaluating the ability of said compound to associate with the said binding site.

2. The method of aspect 1, wherein the viral RNA-dependent RNA polymerase from the Orthomyxoviridae family is from Influenza A, B, C, or D virus or is a variant thereof, in particular Influenza A.
3. The method of any one of aspects 1 to 2, wherein the binding site comprises amino acids K630, R633, and E444 of the PA subunit of the RNA-dependent RNA polymerase of Influenza A virus (SEQ ID NO: 1); comprises one or more amino acids selected from the group consisting of K635, R638, and E449 of SEQ ID NO: 44; or comprises amino acids occupying analogous position in the sequence of a PA subunit aligned thereto.
4. The method of aspect 3, wherein the binding site further comprises amino acids K289, R449, and E452 of SEQ ID NO: 1, or comprises one or more amino acids selected from the group consisting of K289, R454 and E457 of SEQ ID NO: 44.
5. The method of aspect 3 or 4, wherein the binding site further comprises amino acids F440 and F607 of SEQ ID NO: 1, or comprises amino acids Y445 and F612 of SEQ ID NO: 44.
6. The method of any of aspects 3 to 5, wherein the binding site further comprises one or more amino acid selected from the group consisting of M288, L290, S291, T313, F314, I545, M543, and K554 of SEQ ID NO: 1, or comprises one or more amino acid selected from the group consisting of L288, L290, S291, T313, F314, L550, M548, and R559 of SEQ ID NO: 44.
7. The method of any of aspects 3 to 6, wherein the binding site further comprises amino acid G629 of SEQ ID NO: 1, or comprises amino acid G634 of SEQ ID NO: 44.
8. The method of any of aspects 1 to 7, wherein the binding site comprises amino acids F440, E444, F607, G629, K630, and R633 of SEQ ID NO: 1, comprises amino acids Y445, E449, F612, G634, K635, and R683 according to SEQ ID NO: 44, or comprises amino acids occupying analogous position in the sequence of a PA subunit aligned thereto.
9. The method of any of aspects 1 to 8, wherein the binding site comprises amino acids M288, K289, L290, S291, T313, F314, R449, E452, M543, K554, and I545, of SEQ ID NO: 1, comprises amino acids L288, K289, L290, S291, T313, F314, R454, E457, M548, K559, and L550, of SEQ ID NO: 44, or comprises amino acids occupying analogous position in the sequence of a PA subunit aligned thereto.
10. The method of any of aspects 1 to 9, wherein the binding site of the RNA-dependent RNA polymerase of Influenza A virus comprises amino acids M288, K289, L290, S291, T313, F314, F440, E444, R449, E452, M543, K554, I545, F607, G629, K630, and R633, of SEQ ID NO: 1, comprises amino acids L288, K289, L290, S291, T313, F314, Y445, E449, R454, E457, M548, K559, L550, F612, G634, K635, and R638 of SEQ ID NO: 44, or comprises amino acids occupying analogous position in the sequence of a PA subunit aligned thereto.
11. The method of any of aspects 1 to 10, wherein the binding site comprises amino acids 258-713 of SEQ ID NO: 1, or comprises amino acids 201-716 of SEQ ID NO: 44.
12. The method of any one of aspects 1 to 2, wherein the binding site comprises amino acids E445, K631, and R634, of the PA subunit of the RNA-dependent RNA polymerase of Influenza B virus (SEQ ID NO: 2); or comprises amino acids occupying analogous position in the sequence of a PA subunit aligned thereto.
13. The method of aspect 12, wherein the binding site further comprises amino acids Y441 and F604 of SEQ ID NO: 2.
14. The method of aspect 3 or 4, wherein the binding site further comprises amino acids G630 of SEQ ID NO: 2.
15. The method of any of aspects 12 to 14, wherein the binding site comprises amino acids 258-722 of SEQ ID NO: 2.
16. The method of any one of aspects 1 to 15, wherein said computer model is based on structure coordinates of a crystal which diffracts X-rays to a resolution of 3.5 Å or higher, preferably, 3.0 Å or higher, preferably 2.5 Å or higher.
17. The method of any one of aspects 1 to 16, wherein said computer model is based on the structure coordinates as shown in FIG. 11 or 12.
18. A method of producing a compound which decrease or prevent the binding of the viral RNA-dependent RNA polymerase from the Orthomyxoviridae family or variant thereof, to its ligand, (preferably to cellular Pol II, more preferably to CTD) comprising the steps of

(a) identifying said compound via the method of any of aspects 1 to 9, and

(b) synthesizing said compound and optionally formulating said compound or a pharmaceutically acceptable salt thereof with one or more pharmaceutically acceptable excipient(s) and/or carrier(s).

19. The method of aspect 18 comprising the further step of

(c) contacting said compound with the viral RNA-dependent RNA polymerase from the Orthomyxoviridae family, and

(d) determine the ability of said compound to prevent the binding of viral RNA-dependent RNA polymerase to its ligand, preferably CTD.

20. The method of any of aspects 1 to 19, wherein said test compound is a small molecule.
21. The method of any of aspects 1 to 19, wherein said test compound is a peptide or protein.
22. The method of aspect 21, wherein the test compound is an antibody, preferably directed against the binding site of the viral RNA-dependent RNA polymerase from the Orthomyxoviridae family or variant thereof, to its ligand, (preferably to cellular Pol II, more preferably to CTD).
23. A compound identifiable and/or producible by the method of any one of aspects 1 to 18, wherein said compound is able to decrease or prevent the binding of the viral RNA-dependent RNA polymerase or variant thereof, to its ligand (preferably CTD).
24. An antibody directed against the binding site of the viral RNA-dependent RNA polymerase from the Orthomyxoviridae family or variant thereof, to its ligand, (preferably to cellular Pol II, more preferably to CTD).
25. The antibody of aspect 24, wherein said antibody recognizes a polypeptide of a length between 5 and 15 amino acids of the amino acid sequence as set forth in SEQ ID NO: 1 or SEQ ID NO: 2, wherein the polypeptide comprises one or more amino acid residues selected from the group consisting of K630, R633, and E444 of the PA subunit of the RNA-dependent RNA polymerase of Influenza A virus according to SEQ ID NO: 1; or comprises one or more amino acids residues selected from the group consisting of E445, K631, and R634,of the PA subunit of the RNA-dependent RNA polymerase of Influenza B virus according to SEQ ID NO: 2.
26. A nucleic acid encoding an antibody of any of aspects 24 to 25.
27. A vector comprising the nucleic acid of aspect 26.
28. A recombinant host cell comprising the isolated nucleic acid of aspect 26 or the recombinant vector of aspect 27.
29. A pharmaceutical composition producible according to the method of aspect 18 to 22.
30. A pharmaceutical composition comprising a compound of aspect 23, the antibody of aspect 24-25, the nucleic acid of aspect 26, the vector of aspect 27, or the recombinant host cell of aspect 28.
31. A compound according to aspect 23, the antibody of aspect 24-25, the nucleic acid of aspect 26, or the vector of aspect 27, the recombinant host cell of aspect 28, or the pharmaceutical of aspect 29 or 30, for use in treating, ameliorating, or preventing disease conditions caused by viral infections with viruses of the Orthomyxoviridae family.
32. The compound according to aspect 23, the antibody of aspect 24-25, the nucleic acid of aspect 26, or the vector of aspect 27, the recombinant host cell of aspect 28, or the pharmaceutical of aspect 29 or 30, for use according to aspect 31, wherein said disease condition is caused by a virus selected from the group consisting of Influenza A virus, Influenza B virus, and Influenza C virus.
33. Method of treating ameliorating, or preventing disease conditions caused by viral infections with viruses of the Orthomyxoviridae family, comprising administering a therapeutically effective amount of the compound according to aspect 23, the antibody of aspect 24-25, the nucleic acid of aspect 26, or the vector of aspect 27, the recombinant host cell of aspect 28, or the pharmaceutical of aspect 29 or 30.
34. Method of aspect 33, wherein said disease condition is caused by a virus selected from the group consisting of Influenza A virus, Influenza B virus, and Influenza C virus.

The following figures and examples are merely illustrative of the present invention and should not be construed to limit the scope of the invention as indicated by the appended claims in any way.

### EXAMPLES

The Examples are designed in order to further illustrate the present invention and serve a better understanding. They are not to be construed as limiting the scope of the invention in any way.

### Summary of the Examples

Polymerases from bat influenza A (H17N10) and from human influenza B/Memphis/13/03 as well as respective mutants were expressed and purified as described earlier (Plotch et al. 1981 Cell 23:847-858; Pflug et al. 2014 Nature, 516:355-360). Both polymerases were co-crystallised with a SeP5 28-mer (four heptad repeats) CTD peptide, diffraction data collected at the ESRF and structures determined using standard procedures. Binding assays were performed on wildtype or mutant polymerases by anisotropy measurements with CTD peptides comprising 2- or 4-heptad repeats fluorescently labelled with FAM. In vitro transcription/replication assays were performed with wild-type or mutant polymerases with a fluorescence assay that uses separate short vRNA or cRNA 5′ ends (activator) and 3′ ends (template) with or without capped primer. For minigenome assays HEK293T cells were transfected with pcDNA3 plasmids expressing the polymerase subunits, nucleoprotein and pPoll-encoded luciferase reporter gene in negative polarity, carrying the 5′ and 3′ ends of the nucleoprotein segment. Recombinant viruses were generated by reverse genetics and viral replication efficiency was determined by plaque assay.

### Example 1: Expression and Purification of Recombinant Proteins

The heterotrimeric polymerases from influenza A/little yellow-shouldered bat/Guatemala/060/2010 (H17N10), and from human influenza B/Memphis/13/03 as well as the corresponding mutants were expressed and purified as previously described (Pflug et al. 2014, Nature 516:355-360; Reich, et al. 2014, Nature 516:361-366).

### Example 2: Crystallization, Data Collection and Structure Solution

Bat FluA polymerase-vRNA crystals were produced as previously described (Pflug et al. 2014, Nature 516:355-360) but with the Ser-5-phosphorylated 28-mer CTD peptide (Covalab) added in 1:1 ratio to the polymerase complex. The occupancy of the peptide was enhanced by soaking the crystals with extra peptide before cryo-protection and freezing. Diffraction data were collected on beamline ID29 of the European Synchrotron Radiation Facility (ESRF) and integrated and scaled with the XDS suite (Kabsch 2010, Acta Crystallogr D Biol Crystallogr 66:133-144). The structure was solved by molecular replacement using the original bat FluA polymerase structure (PDB code: 4WSB) and the peptide was manually modelled in the clear difference density (FIG. 1A). FluB polymerase-vRNA complex was co-crystallized with the Ser-5 phosphorylated 28 amino-acid CTD peptide in 1:15 ratio yielding the FluB1 crystal form as previously described (Reich, et al. 2014, Nature 516:361-366). Diffraction data were collected on beamline ID23-1 at the ESRF. The structure was solved by molecular replacement with the model of influenza B structure (PDB code: 4WSA). Both structures were refined using Refmac54 and Phenix5. For the lower resolution FluB structure only TLS parameters were refined. Structure figures were drawn with Pymol (DeLano 2002; PyMOL Molecular Graphics System, available online at http://www.pymol.sourceforge.net). The structure of the co-crystallized vRNA-promoter bound bat FluA polymerase with a four heptadrepeat Ser5-phosphorylated (SeP5) CTD peptide was determined at 2.5 Å resolution. Unambiguous extra electron density for the peptide was clearly observed at two separate sites on the polymerase surface and 16/28 residues could be modelled in the structure (FIG. 1A). The CTD peptide binds to the C-terminal domain of PA (PA-C) (FIG. 2A), distant from the promoter binding site, the polymerase active site and the cap-binding and endonuclease domains, which remain unperturbed. Six residues from one CTD repeat (Y₁ₐS₂ₐP₃ₐT₄ₐSeP₅ₐP₆ₐ, bind to a groove formed by PA helices α16, α20, α21 and the loop connecting helix α15 to α16 (denoted site 1) with an interaction surface of 672 Å² (FIG. 2B). Site 2 accommodates residues from three consecutive repeats (P_{6b}S_{7b}-Y_{1c}S_{2c}P_{3c}T_{4c}SeP_{5c}P_{6c}S_{7c}-Y_{1d}), which upon binding bury a surface area of 1168 Å². The peptide sits on the β319-β320 ribbon and the protruding 550-loop (Pflug et al. 2014, Nature 516:355-360). There is no electron density connecting the sites 1 and 2 peptides although they could plausibly be joined by the missing residues (-S₇ₐ-Y_{1b}-S_{2b}-P_{3b}-T_{4b}-SeP_{5b}-) (FIG. 2B).

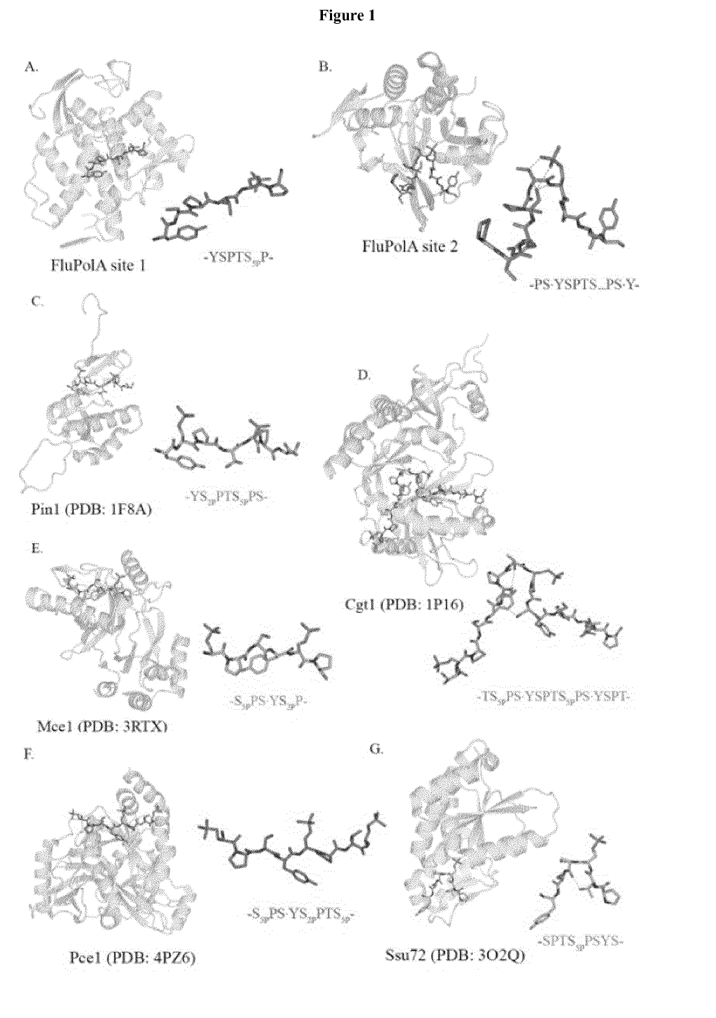

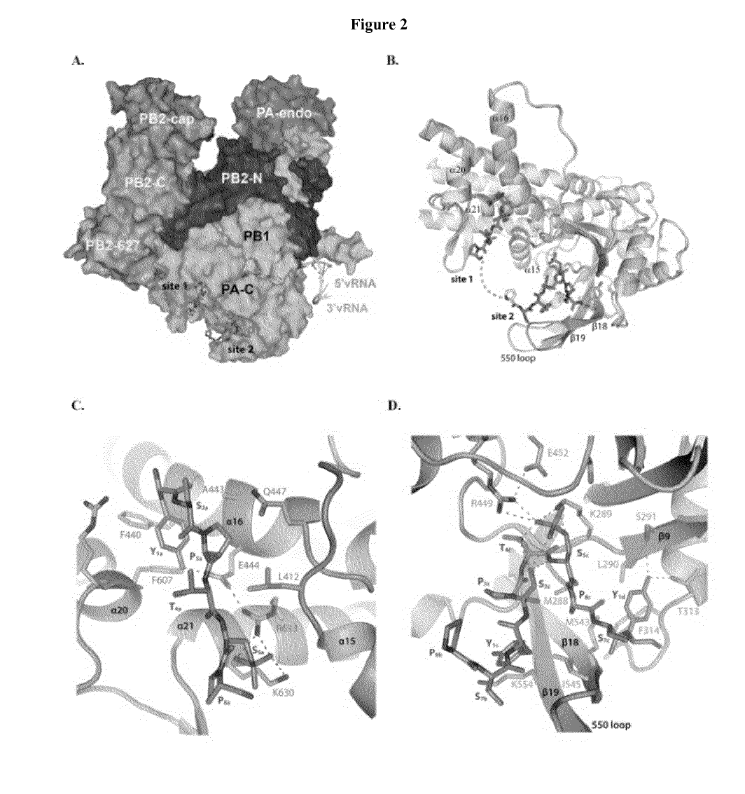

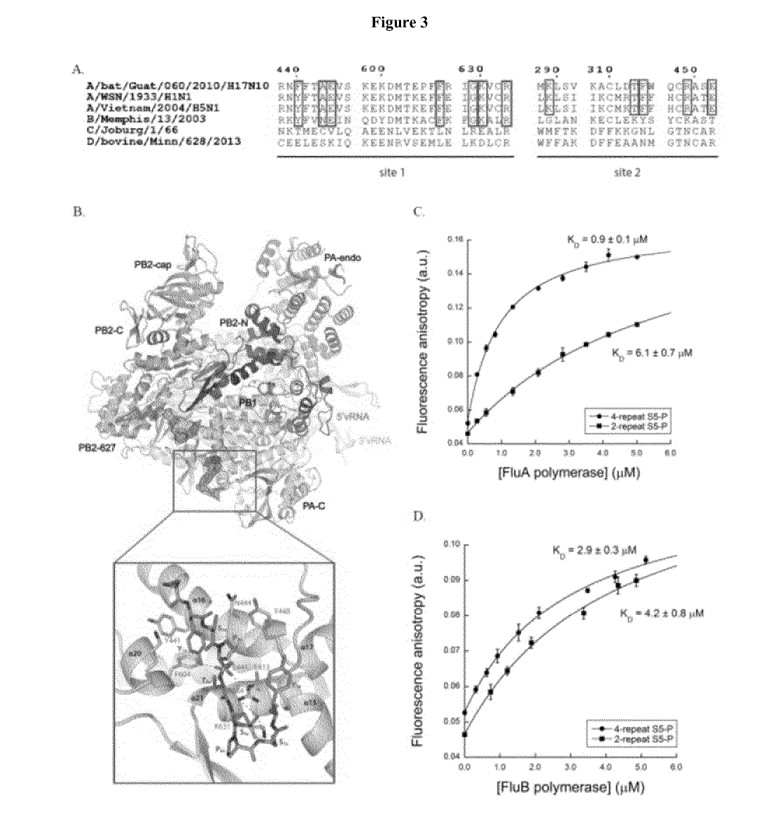

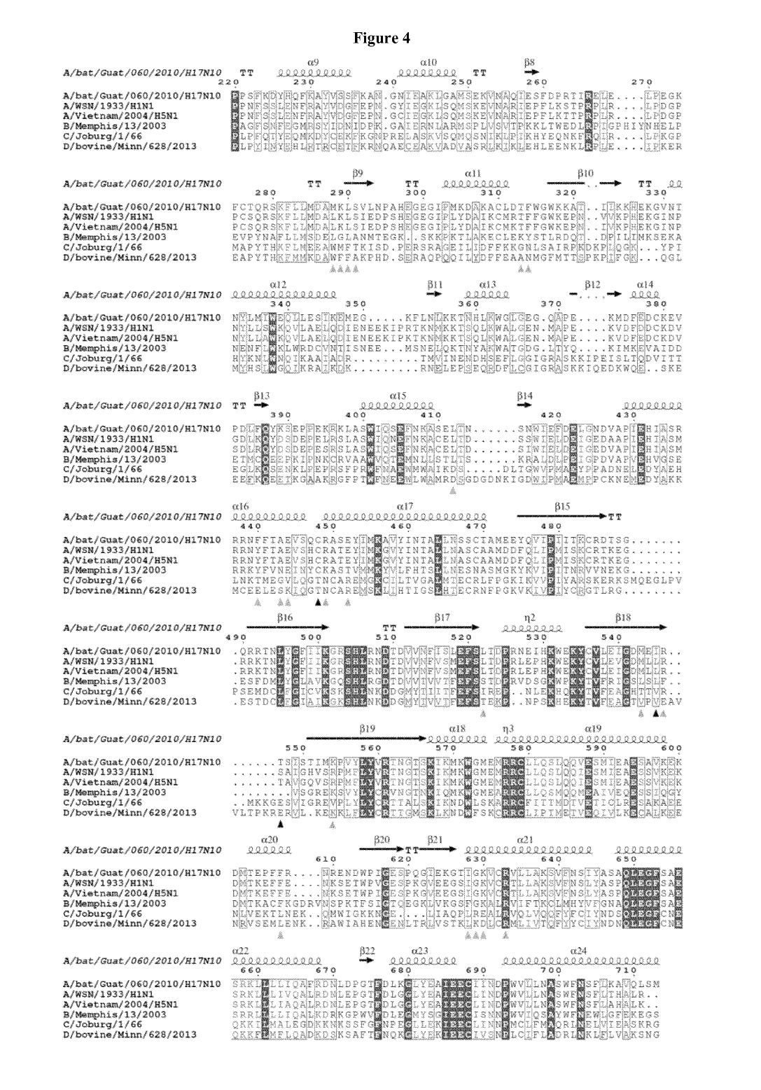

The CTD peptide in site 1 adopts an extended beta-like conformation with alternate residues either interacting with the protein (Y₁ₐ, P₃ₐ, SeP₅ₐ) or pointing into solvent (S₂ₐ, T₄ₐ and P₆ₐ) (FIG. 2C). A similar conformation has previously been observed in other structures of CTD bound to protein partners (FIG. 1B-E), but the specific interactions are different. Y₁ₐ is accommodated in a hydrophobic pocket formed by PA residues F440 and F607 (bat FluA numbering) and its hydroxyl group makes a hydrogen bond with E444. P₃ₐ, is in van der Waals contacts with non-polar residues L412 and A443. The phosphate of SeP₅ₐ is bound by multiple hydrogen bonds in a positively charged pocket formed by K630 and R633, which in turn is positioned interacting with E444. The CTD repeats binding to site 2 form a β-turn, flanked by extended regions, which clamp the 550-loop (FIG. 2B, D). The β18-β19 ribbon and 550-loop are displaced by up to 6 Å compared to the structure without peptide (FIG. 2B). The β-turn is formed by S_{2c}P_{3c}T_{4c}SeP_{5c} and is stabilized by three internal hydrogen bonds between the hydroxyl of S_{2c} and both T_{4c} hydroxyl and SeP_{5c} amide, and between the T_{4c} hydroxyl and the SeP_{5c} phosphate. These interactions preclude the phosphorylation of either S_{2c} or T_{4c} in this configuration, consistent with the known specificity of the polymerase for Ser₅-phosphorylated CTD2. The SeP_{5c} phosphate makes charged hydrogen bonds with basic residues K289 and R449, the latter being stabilized by E452. The two tyrosines (Y_{1c} and Y_{1d}), as well as P_{6c}, are bound in hydrophobic pockets, formed by I545 and the aliphatic sidechain of K554 (for Y_{1c}); M543 and M288 (P6c); M543, L290 and F314 (Y_{1d}). The Y_{1d} hydroxyl also makes two hydrogen bonds to the side-chain of T313 and main-chain amide of S291. Sequence alignments from influenzas A, B, C and D show that all key site 1 residues are highly conserved in all FluA strains, F440 (Y445), E444 (449), F607 (612), G629 (634), K630 (635), R633 (638) in bat (avian/human), and FluB, Y441, E445, F604, G630, K631 and R634, but not in FluC or D (FIG. 3A, FIG. 4). In contrast, key site 2 residues are only conserved in FluA strains. To confirm these observations, we determined the 3.5 Å resolution structure of FluB polymerase co-crystallised with the same 28-mer SeP5 peptide. As predicted, we observed binding in site 1, the mode of interaction being identical to that to bat FluA, with the exception that S₇ₐ-Y_{1b} are additionally observed (FIG. 3B,C). However additional peptide-like difference electron density was also observed extending from close to site 1 (but not connected to it) across the PB2 627 domain (FIG. 3B), which is not observed in the bat Flu structure. These results suggest that site 1 is a universal CTD binding site for all influenza A and B strains, whereas site 2 seems to be FluA specific and there might be an alternative second site in FluB strains.

### Example 3: Fluorescence Anisotropy

To further characterise the interaction of the CTD with FluA and FluB polymerases fluorescence anisotropy binding experiments were performed using fluorophore-labelled peptides with 2 (14-mer) or 4 (28-mer) repeats. Binding assays were performed with CTD peptides, fluorescently labelled with FAM at the N-terminal end and consisting of different number of repeats of the consensus sequence (Y₁S₂P₃T₄S₅P₆S₇). 2- and 4-repeats (14 and 28 amino acids, respectively) Ser-5 phosphorylated peptides and 4-repeats non-phosphorylated peptide were used (Covalab). Peptides were titrated with increasing concentrations of wild-type polymerase or the corresponding mutants in 50 mM HEPES, 150 mM NaCl, 10% glycerol, 5 mM MgCl2, 2 mM tris(2-carboxyethyl)phosphine (TCEP), pH 7.5. The proteins were pre-mixed with the vRNA promoter, 5′-pAGUAGAAACAAGG-3′ (SEQ ID NO: 24) and 3′OH-GCCUGCUUCUGCU-5′ (SEQ ID NO: 25) for bat FluA and 5′-pAGUAGUAACAAGAG-3′ (SEQ ID NO: 26) and 3′OH-CUCUGCUUCUGCU-5′ (SEQ ID NO: 27) for FluB, with one exception (indicated). Fluorescence anisotropy was measured at 23° C. with a fluorescence spectrometer (Photon Technology International). The observed fluorescence anisotropy was plotted. Dissociation constants were obtained by fitting the data to a 1:1 binding model using the following equation:

$${f}_{b } = \frac{( L + P + {K}_{D} - \sqrt{{( L + P + {K}_{D} )}^{2} - 4 LP )} )}{2 L}$$

(fb—fractional concentration of bound peptide, L—total concentration of fluorescent peptide, P—total concentration of protein, KD—dissociation constant). For the displacement assay 0.5 μM FluA polymerase:vRNA complex was incubated with 0.5 μM fluorescently labelled Ser-5 phosphorylated CTD peptide and titrated with increasing amount of non-labelled Ser-5 phosphorylated CTD peptide. The apparent K_{D} was calculated using the same binding model equation.

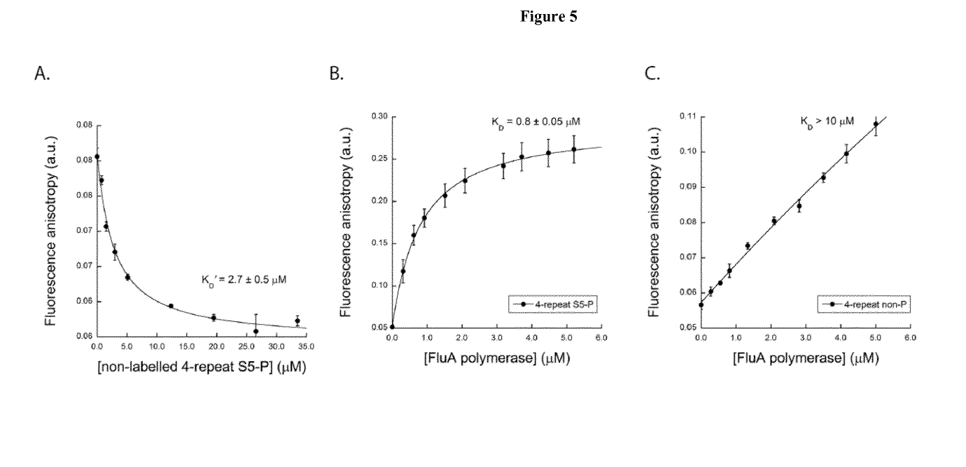

We derived a K_{D} of 0.9 μM for the 28-mer SeP5 peptide binding to the bat FluA polymerase-vRNA promoter complex (FIG. 3C). A similar K_{D} was obtained by displacing bound labelled peptide by an unlabelled peptide (Fig.SA). The binding was independent of vRNA promoter binding (FIG. 5B), consistent with the peptide binding site being on the exterior of the structurally stable core of the polymerase, distant from the vRNA binding site and from flexibly linked peripheral domains 18 and also consistent with previous results showing that vRNA is not required for CTD binding (Engelhardt et al. 2005, Journal of virology 79:5812-5818; Loucaides et al. 2009, Virology 394:154-163). For 14-mer SeP5 and unphosphorylated 28-mer Ser5 peptides we measured KD's of 6.1 and >10 μM, respectively (FIG. 3C, FIG. 5C). This implies a >10-fold higher affinity for Ser5 phosphorylated compared to non-phosphorylated CTD repeats and a tighter binding to four compared to two repeats. The latter result is consistent with the crystal structure, which suggests that four consecutive repeats can bind across both sites and with the expected avidity effect of fusing independent ligands. FluB polymerase did not discriminate between different length Sep5 peptides (K_{D}'s of 2.9 and 4.2 for 28-mer SeP₅ and 14-mer SeP₅, respectively) (FIG. 3D).

### Example 4: In Vitro Polymerase Activity Assays

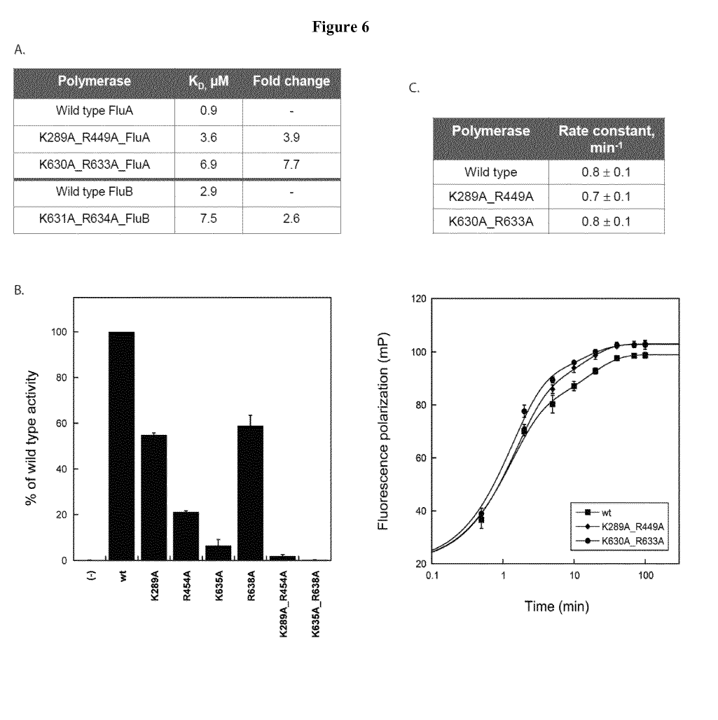

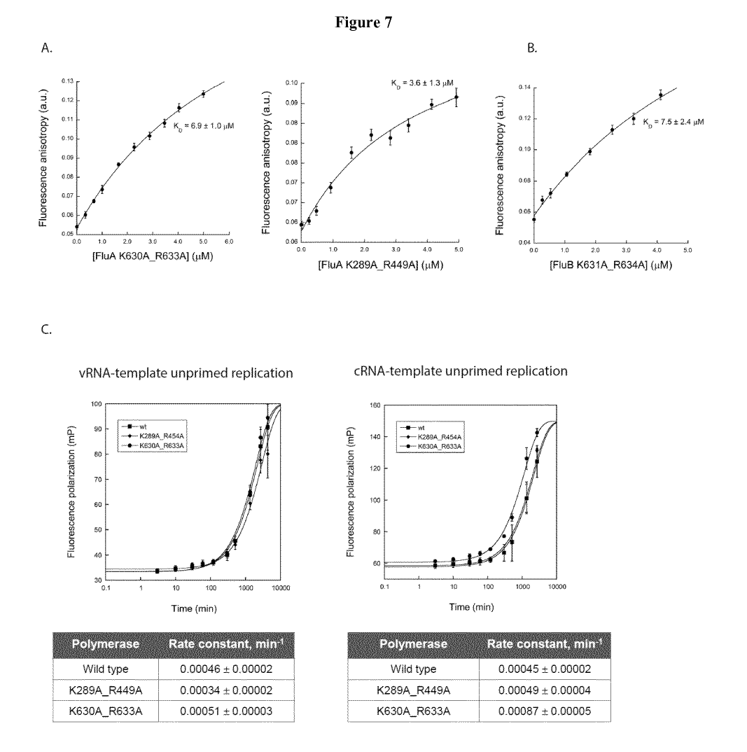

To assess the importance of the CTD interaction for viral replication, we mutated the conserved basic residues forming the positively charged phosphoserine binding sites. We first expressed and purified recombinant bat polymerase with double mutants in either site 1 (K630A+R633A) or site 2 (K289A+R449A). The affinity to the four-repeat SeP5 peptide decreased by around 4-fold and 7.5-fold for the K289A+R449A and K630A+R633A mutants respectively (FIG. 6A, FIG. 7A). The corresponding mutant in FluB (K631A+R634A) had a 2.5-fold decrease in the binding affinity to the CTD (FIG. 6A, FIG. 7B). Polymerase containing all four mutations (K289A, R449A, K630A and R633A) could not be produced recombinantly and studies using fluorescently labelled polymerase subunits expressed in mammalian cells showed that it is not properly localized in the nucleus, unlike the other mutants (data not shown).

Three types of RNA synthesis assays were performed in order to compare the intrinsic enzymatic activity of the FluA polymerase CTD binding mutants: cap-primed transcription-like and unprimed replication-like using v- or cRNA as a template. For each assay, 0.25 μM bat FluA polymerase, pre-mixed with 5′ vRNA (5′-pAGUAGUAACAAGAG-3′) (SEQ ID NO: 28) or 5′ cRNA (5′-pAGCAGAAGCAGAGG-3′) (SEQ ID NO: 29), was added to 0.15 μM fluorescently labelled template 3′ vRNA (5′-FAM-UAUACCUCUGCUUCUGCU-3′) (SEQ ID NO: 30) or cRNA (5′-FAMUACCCUCUUGUUACUACU-3′) (SEQ ID NO: 31). The reactions were performed in 50 mM HEPES, 150 mM NaCl, 10% glycerol, 5 mM MgCl2, 2 mM tris(2-carboxyethyl)phosphine (TCEP), pH 7.5, with 0.025 mM or 0.5 mM NTPs (in the presence or absence of cap-primer, respectively). For the cap-dependent transcription reaction, 0.5 μM capped primer (5′-m7GpppAAUCUAUAAUAG-3′) (SEQ ID NO: 32) was added. The assays were performed at 24° C. Reactions were quenched in NaCl and the fluorescence polarization signal corresponding to the double-stranded product-template duplex was detected using a Clariostar microplate reader (BMG Germany). The obtained time courses for the unprimed replication reactions were fitted to a single exponential equation:

f(t)=−A*e^{-k*t}+B;

or, in the case of cap-dependent transcription, double exponential fit:

f(t)=(−a*e^{-k}^{₁}^{*t}+B)+(−C*e^{-k}^{₂}^{*t}+D),

where A and C are the observed polarization amplitudes, B and D—the final polarization values for the corresponding phases; t is the time and kn—the respective observed rate constants.

### Example 5: Minigenome Assay

Next we assayed the overall effect on the polymerase activity in a cellular environment using a mini-genome assay, where human FluA polymerase and nucleoprotein are expressed together with an RNA encoding a reporter luciferase in negative polarity. The functional reporter protein can only be produced by an actively transcribing ribonucleoprotein complex.

HEK293T cells were seeded in 12-well plates and transfection was performed with XtremeGene transfection reagent. Each well was transfected with 100 ng pcDNA3 expressing nucleoprotein (NP), 10 ng pcDNA3 plasmids expressing PA or corresponding mutants, PB1 and PB2 subunits of the influenza A/Victoria/3/1975(H3N2) polymerase (Ortin et al. 2015, Virology 479-480:532-544), and 100 ng pPoll-NP-Luc, encoding a firefly luciferase reporter gene in negative polarity, flanked by the 5′ and 3′ regions of the NP segment (Palancade et al. 2003, Eur J Biochem 270:3859-3870). Transfection mix without PA was used as a negative control. pRenilla-TK plasmid (Promega) was used to correct for the transfection efficiencies. Cells were lysed 24 hours post-transfection and Firefly and Renilla luciferase activities were measured using Berthold Technologies Centro LB 960 luminometer, according to manufacturer protocol (Promega). Experiments were performed in biological triplicates. For western blot detection of the expression levels of the mutants, 1 μg of PA or corresponding mutants was transfected using polyethylenimine transfection reagent and detected using mouse anti-PA antibody (provided by J. Ortin). Beta-actin antibody (Abeam, UK) was used for normalization of the total protein amount in respective cell lysates.

We observed a drastic decrease in activity of each of the double mutants (site 1: K635A+R638A and site 2: K289A+R454A) to 0.3 and 2% of wild type activity respectively (FIG. 6B). We also produced single alanine mutations of each individual arginine or lysine separately and observed decreases in activity to between 6 and 60% of wild type (FIG. 6B). Interestingly, purified recombinant bat FluA polymerase with the equivalent double mutations showed no difference in activity compared to wild-type in in vitro cap-dependent transcription (FIG. 6C) or unprimed viral replication assays using vRNA or cRNA as template (FIG. 7C), confirming that all intrinsic polymerase enzymatic activities are unaffected by the mutations. These results show that the mutant polymerases are only impaired in the cellular context, presumably due to reduced binding to the Pol II CTD.

### Example 6: Production of Recombinant Viruses by Reverse Genetics

We used reverse genetics to produce recombinant human influenza viruses with the K635A+R638A (site 1) or K289A+R454A (site 2) double mutations in the PA subunit or with the K289A, R454A, K635A or R638A single mutations. HEK-293T cells were grown in complete Dulbecco's modified Eagle's medium (DMEM) supplemented with 10% fetal calf serum (FCS). MDCK cells were grown in modified Eagle's medium supplemented with 5% FCS. Recombinant A/WSN/33 viruses were produced by reverse genetics using a procedure adapted from previous work (Fodor et al. 2003, Journal of virology 77:5017-5020; Eisfeld et al. 2015, Nat Rev Microbiol 13:28-41). The efficiency of reverse genetics was evaluated by titrating the supernatants on MDCK cells using a plaque assay procedure adapted from previous work (Resa-Infante et al. 2011, RNA Biol 8:207-215). Upon plaque-purification and amplification on MDCK cells, viral RNA was extracted using the QIAamp Viral RNA Mini kit (QIAGEN). Reverse-transcription and amplification were performed using the SuperScript One-Step RT-PCR kit (Invitrogen), and oligonucleotides 5′-AGTAGAAACAAGGTACTTTTTTGG-3′ (SEQ ID NO: 33) and 5′-TGCAGGACATTGAGAATGAGG-3′ (SEQ ID NO: 34) or 5′-ACCTCAATTCTGGTTCATCAC-3′ (SEQ ID NO: 35) and 5′-AGCGAAAGCAGGTACTGATCC-3′ (SEQ ID NO: 36) to amplify the 5′ or 3′ section of the PA segment, respectively. The amplification products were purified using a Gel and PCR Cleanup kit (Macherey-Nagel), and were sequenced using internal oligonucleotides.

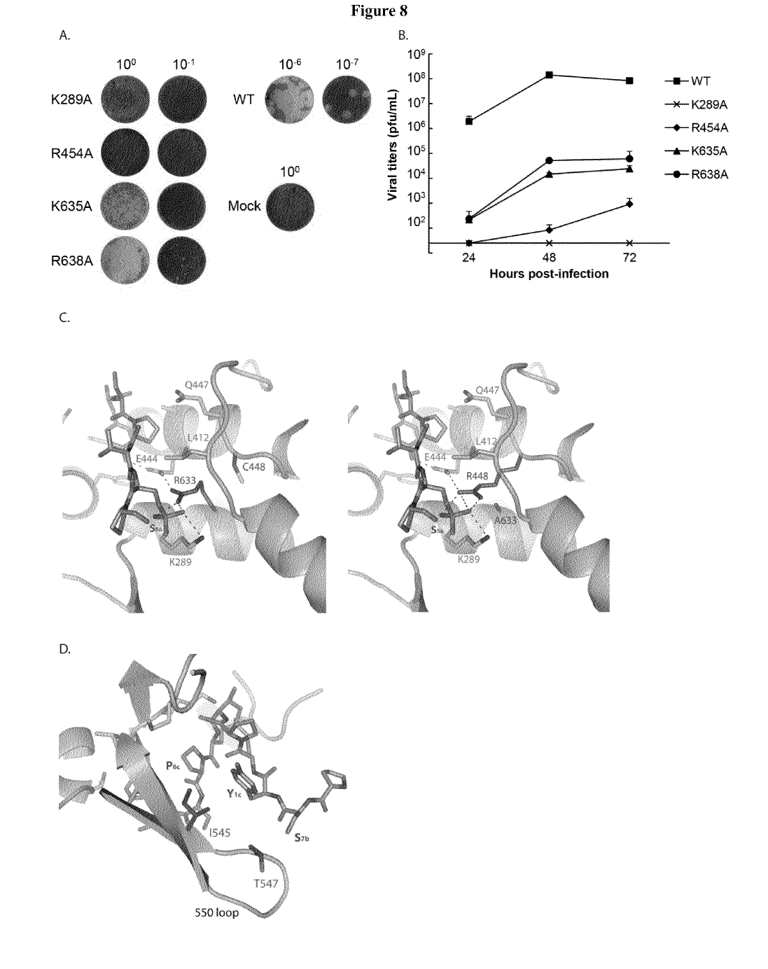

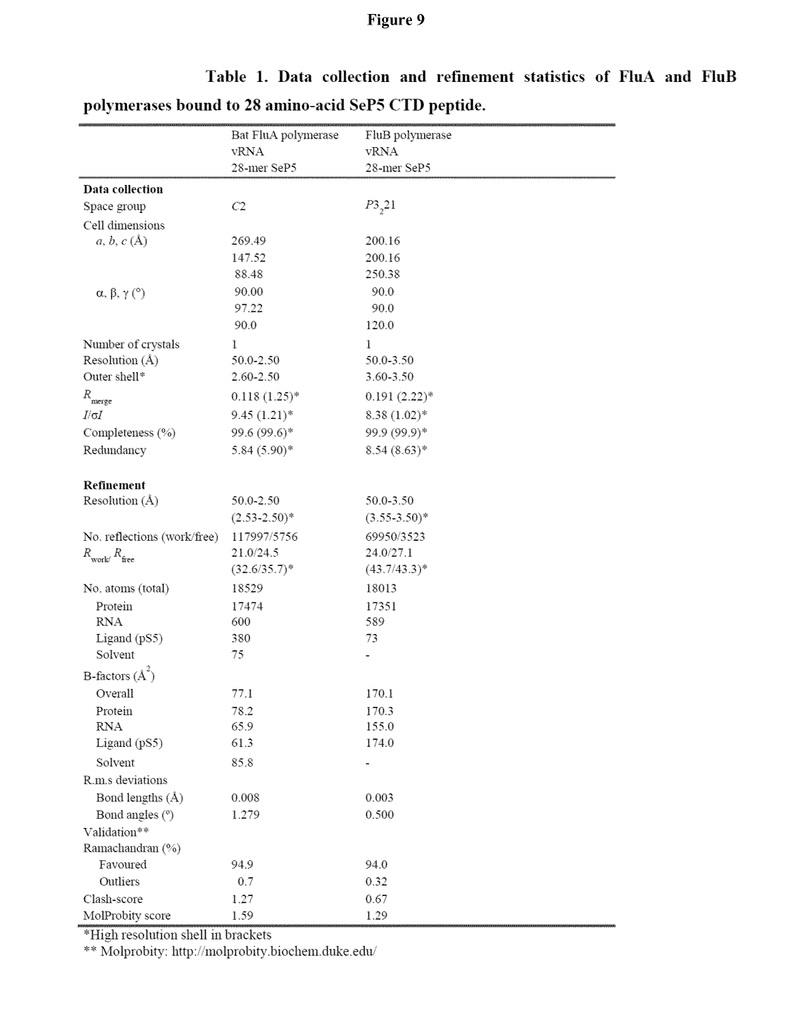

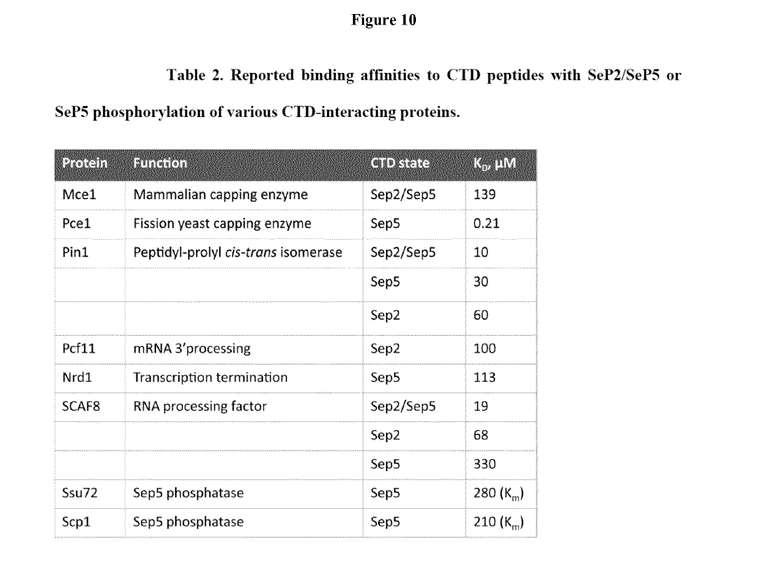

Neither of the double mutant viruses could be rescued. As shown in FIG. 8A, reverse genetics supernatants of the single PA mutants showed titers <103 pfu/ml compared to >107 pfu/ml for the wild-type virus, and smaller plaques (pinpoint plaques in the case of the R454 mutant). For each PA mutation, the virus from a single plaque was purified and amplified on MDCK cells. The resulting viral stocks showed the expected PA sequence and a small plaque phenotype (data not shown). To compare the growth properties of the mutant and wild-type viruses, MDCK cells were infected at a multiplicity of infection of 0.001 and viral titers were determined in the culture supernatant at different time points (FIG. 8B). The K635A and R638A mutants were severely attenuated compared to the wild-type. The R454 mutant was even more attenuated, while the K289A mutant did not grow at detectable levels in these conditions.

### Example 7: Virtual Screening for Compounds Binding to FluPol and Thereby Blocking the Interaction Between the CTD-Domain of Cellular RNA Polymerase II (Pol II) and FluPol

The crystal structure elucidates the existence of at least two distinct binding regions in FluPol which both accommodate parts of the C-terminal domain (CTD) of Pol II. The structural information enabled the analysis of the exact interaction profile of these amino acids of CTD of Pol II with FluPol.

Both interaction sites are analyzed separately and the knowledge was applied for several virtual screening approaches to search for compounds blocking the interaction between the viral and the cellular proteins following both approaches:

1. Ligand-based virtual screen and detecting compounds which can mimic the CTD-amino acids in their bioactive conformation doing important interactions with FluPol.

a. This is done for the binding in site 1

b. This is done for the binding in site 2

2. Structure-based virtual screens:

a. Docking of commercially available compounds into binding site 1 for one part of the CTD repeats. Hereby a bias was included in order to favour compounds with substructures which can best mimic the SeP5 group interacting with the viral protein

b. Docking of commercially available compounds into binding site 2 for one part of the CTD repeats. Hereby a bias was included in order to favour compounds with substructures which can best mimic the SeP5 group interacting with the viral protein.

### CONCLUSION

We have shown that FluA polymerase binds in vitro directly to multiple Ser5 phosphorylated Pol II CTD repeats and that disruption of the two conserved phosphoserine binding sites is highly detrimental to viral infectivity, without affecting the intrinsic RNA synthesis activity of the polymerase.

Modelling based on our CTD bound FluA polymerase structure suggests how the very close spatial proximity of the substitution C453R (C448R in bat FluA) can compensate for the loss of R638 in phosphoserine binding (FIG. 8C). Secondly, it has been shown that the PA substitution L550I is sufficient to impart a low virulence phenotype, with reduced Pol II degradation, to the influenza A/PR/8/34 strain (Llompart et al. 2014, Journal of virology 88:3455-3463; Rolling et al. 2009, Journal of virology 83:6673-6680). PA L550 is highly conserved (99.6%) in human/avian influenza A strains and corresponds to residue I545 in bat FluA, which makes key hydrophobic interactions with CTD residues Tyr_{1c} and Pro_{6c} in CTD binding site 2 (FIG. 2D, 8D). These observations suggest that conservative substitution of this residue can subtly modulate, without eliminating, CTD binding with consequent knock-on effects on polymerase activity, Pol II degradation and virulence. Thirdly, PA residue 552 near the tip of the 550-loop, which is almost exclusively threonine in avian (and bat, T547) or serine in human FluA polymerases is also part of binding site 2, being very close to CTD residue Ser_{7b} (FIG. 8D). The single PA mutation T552S (avian to human signature) is sufficient to 20-fold enhance the activity of an otherwise impaired avian polymerase in human cells (Mehle et al. 2012, Journal of virology 86:1750-1757). Taken together, we conclude that only minor perturbations in CTD binding site residues (e.g. I550L or T552S) can have biologically significant effects. This is consistent with our findings that relatively drastic mutations (e.g. double knockout of both phosphoserine binding basic residues) lead to non-viable virus and even single mutants lead to highly attenuated virus, despite that the in vitro binding of the double mutant polymerases to the CTD is only reduced 4-8 fold (FIG. 6A, FIG. 7A). These considerations point to the need for fine tuning of the affinity of the viral polymerase for Pol II CTD relative to competing host factors. Comparison of the KD for FluA polymerase binding to SeP₅ CTD peptide repeats (0.9 μM) with those for other relevant CTD binding proteins e.g. the mammalian capping enzyme (Mce1), KD ˜139 μM24, Pin1 proline isomerase, KD ˜30 μM (Verdecia et al. 2000, Nat Struct Biot 7:639-643) and Ssu72 Sep5 phosphatase, KM ˜280 μM (Xiang et al. 2010, Nature 467:729-733; Hausmann et al. 2005, The Journal of biological chemistry 280:37681-37688). Table 2 shows that the affinity of the viral polymerase to the CTD is amongst the highest so far reported. This suggests that FluPol can compete strongly for CTD binding and potentially prevent other factors binding, notably those (e.g. Ssu72 and Pinl) which promote transition to the elongation phase.

### Example 8: Construct Design/Cloning of H7N9 Polymerase Core

A co-expression construct for the influenza A/Zhejiang/DTID-ZJU01/2013(H7N9) polymerase core was generated on basis of the commercial baculovirus expression vector pFastBacDual (Thermo Fisher). The sequence encoding for the complete PB1 protein (SEQ ID NO: 41, synthetic, GenBank: AGJ51960.1) was inserted into the PolH-MCS using the restriction sites BamHI and RsrII; and the sequence encoding for residues 1-127 of the protein PB2 (SEQ ID NO: 42, synthetic, GenBank: KJ633805.1) supplemented with a TEV-cleavable poly-histidine tag (SEQ ID NO: 43, GSGSENLYFQGSHHHHHHHH) inserted into the P10-MCS using the restriction sites BbsI and XhoI. The sequence encoding for residues 201-716 of protein PA (SEQ ID NO: 44, synthetic, GenBank: AGJ51952.1) was cloned first into the vector pACEBac via the restriction sites BamHI and EcoRI, then amplified from this construct including PolH promoter and SV40 polA signal and subcloned into the pFastBacDual_PB1_PB2 construct using a unique AvrII restriction site and SLIC cloning technology.

### Example 9: Expression and Purification of Recombinant Proteins

H7N9 core was produced in HighFive insect cells using the baculovirus expression system. Cells were lysed by sonication in buffer A (50 mM Tris-HCl pH8.0, 500 mM NaCl, 10% (v/v) glycerol), cell debris was spun off (30 min, 4° C., 35,000 g) and ammonium sulphate added to the supernatant (0.5 g/ml) to force the protein out of solution. The precipitated protein was collected by centrifugation (30 min, 4° C., 35,000 g), re-suspended in buffer A and a last time cleared by centrifugation (30 min, 4° C., 35,000 g) before subjecting it to immobilized metal ion affinity chromatography (IMAC). Elution fractions containing polymerase protein were pooled and subjected to a digestion with TEV protease (in buffer A supplemented with 5 mM β-mercaptoethanol). After dialyzing the digested protein sample back into buffer A it was passed through an IMAC columns a second time, in order to remove impurities, un-cleaved polymerase protein as well as cleaved polyhistidine tags. The sample was diluted to a salt concentration of 250 mM NaCl prior to loading it on a heparin column (HiPrep Heparin HP, GE Healthcare). The column was first washed with buffer B (50 mM HEPES pH 7.5, 5% (v/v) glycerol, 2 mM TCEP, 150 mM NaCl) and then the protein eluted via a salt gradient plateauing at 1M NaCl. Monomeric and RNA-free polymerase was concentrated to about 9 mg/ml, flash-frozen and stored at −80° C.

### Example 10: Crystallization

H7N9 core typically crystallized within 4-5 days in conditions of 0.1M Tris pH 7.0, 8-13% PEG 8K, 0.2M MgCl2, 0.1 M guanidine hydrochloride; at 4° C. with drop ratios of 1:2-1:3 (protein:well). The crystals are of space-group P212121 (with cell dimensions a˜76.5, b˜144.1, c˜336.2) with two complexes in the asymmetric unit. 5′ vRNA (SEQ ID NO: 45, 5′-pAGUAGUAACAAG) was added in excess of 1.1 over protein for co-crystallization experiments. Ser5 phosphorylated peptides mimicking the C-terminal domain (CTD) of the protein RPB1 of the human RNA polymerase II (a 28mer containing four CTD heptads, Tyr-Ser-Pro-Thr-SerP-Pro-Ser) was soaked into existing crystals at a concentration of ˜2 mM over a period of ˜24 h. Crystals were flash frozen in well solution supplemented with 25% glycerol for data collection at beamlines of the European Synchrotron Radiation Facility.

### CONCLUSION

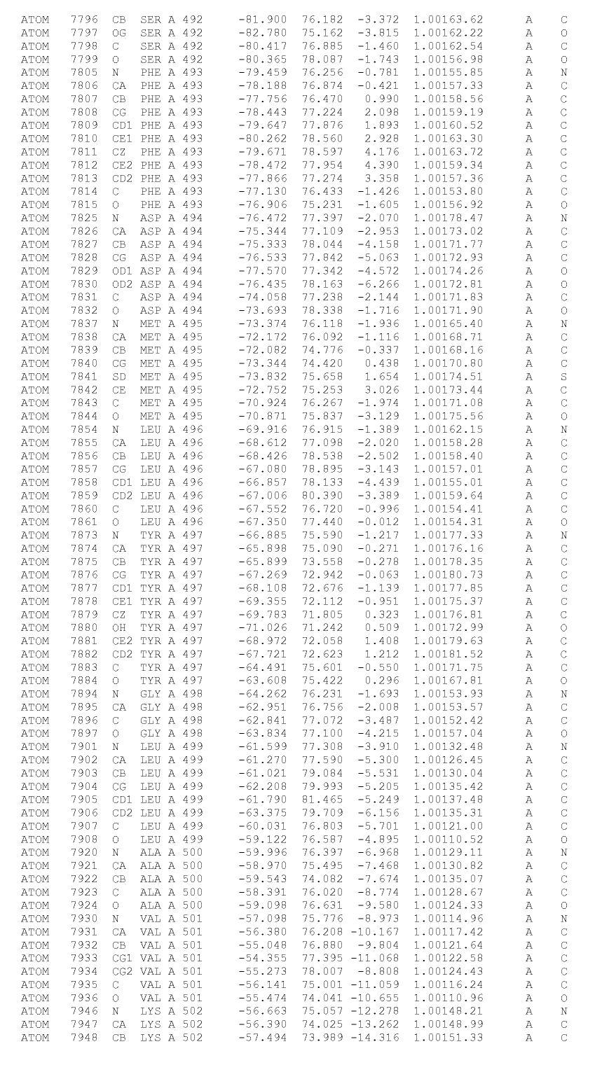

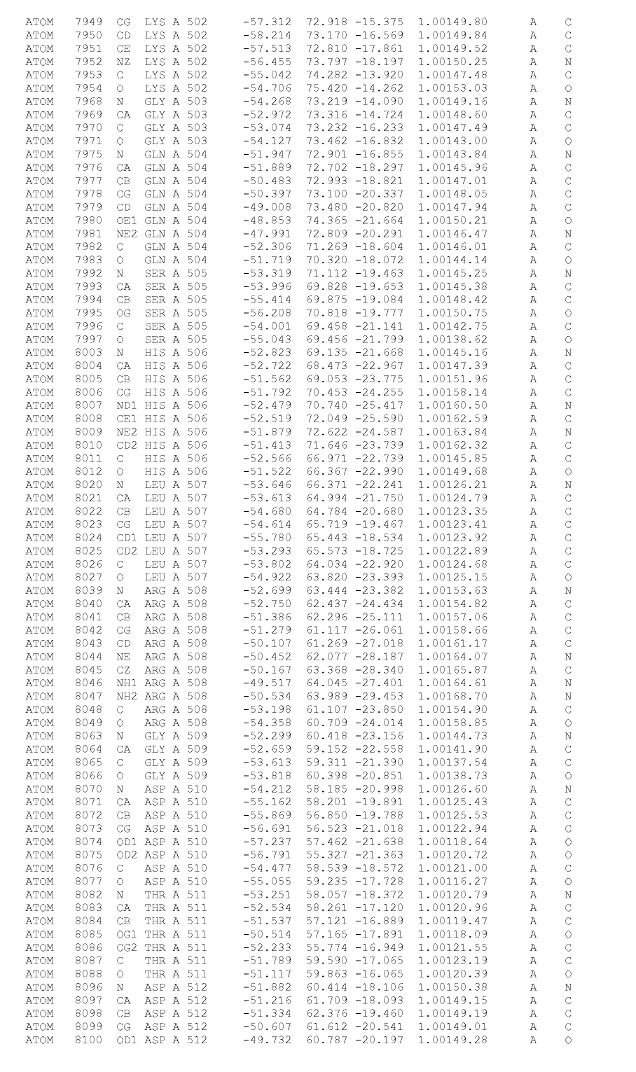

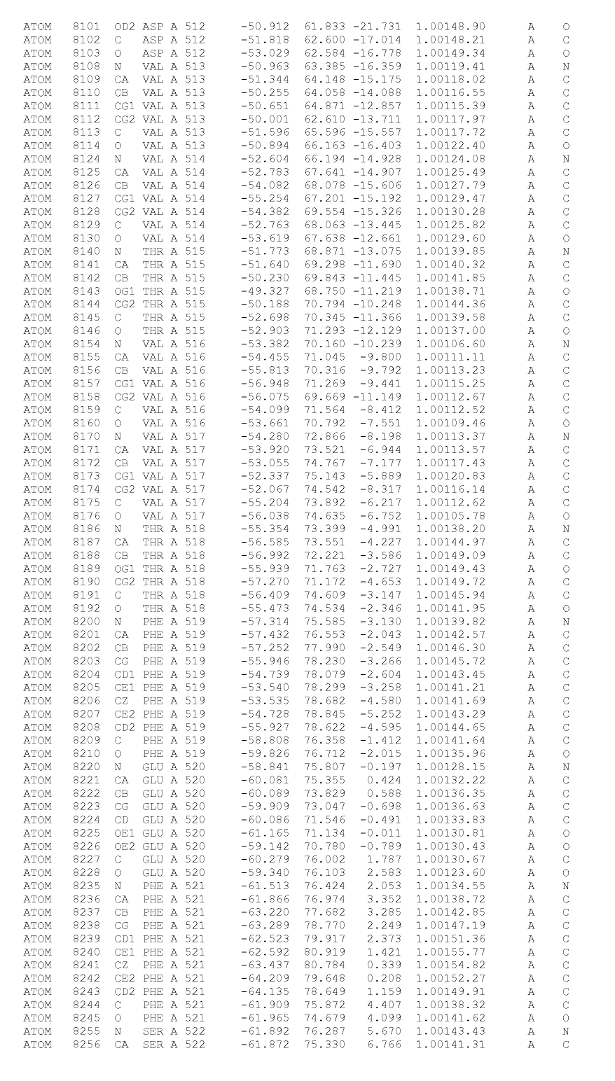

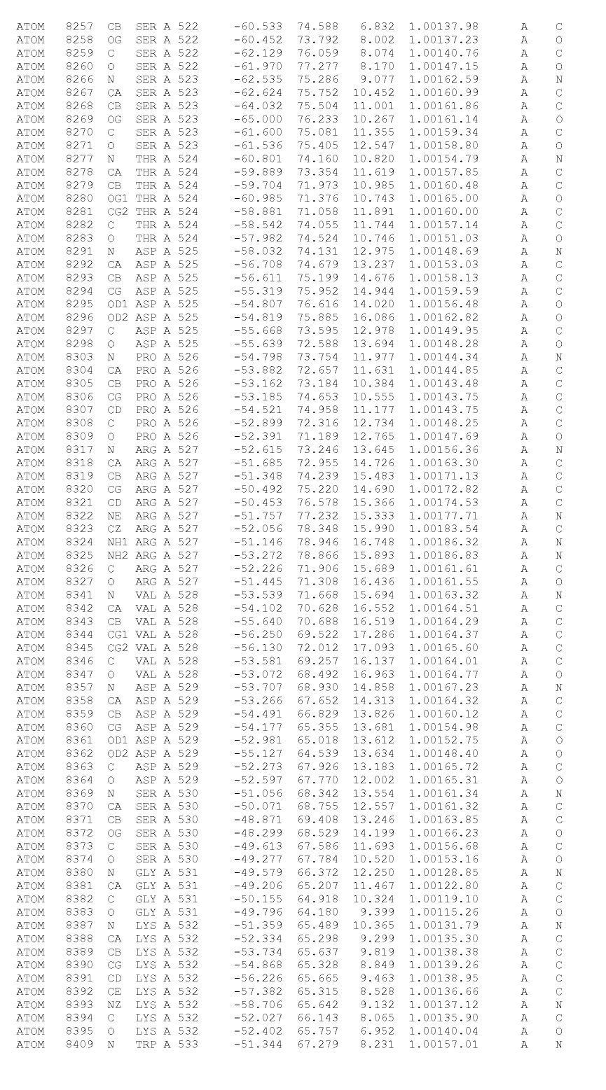

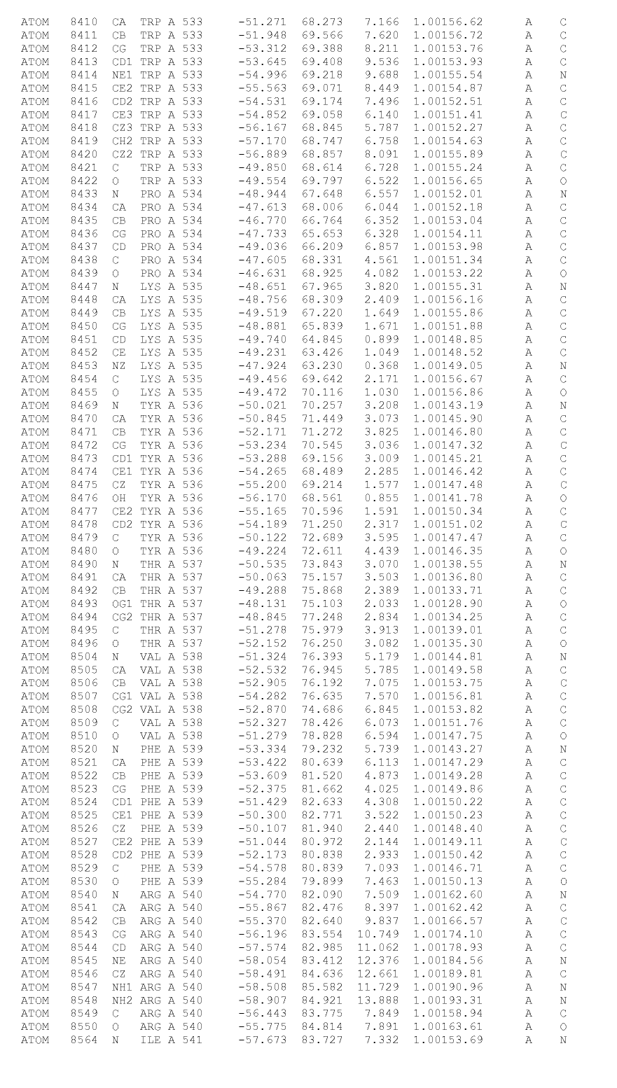

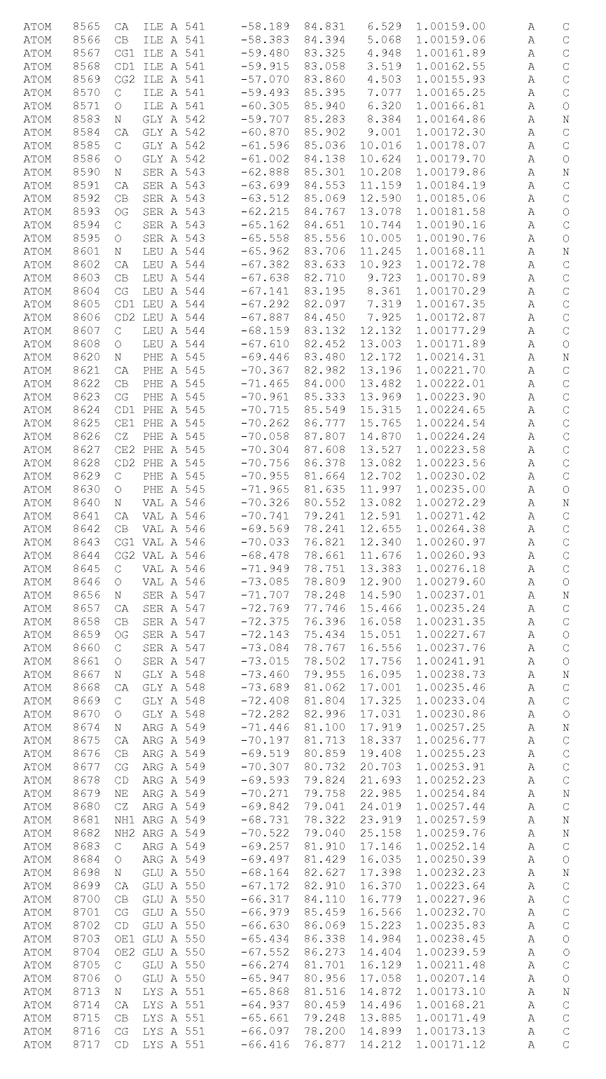

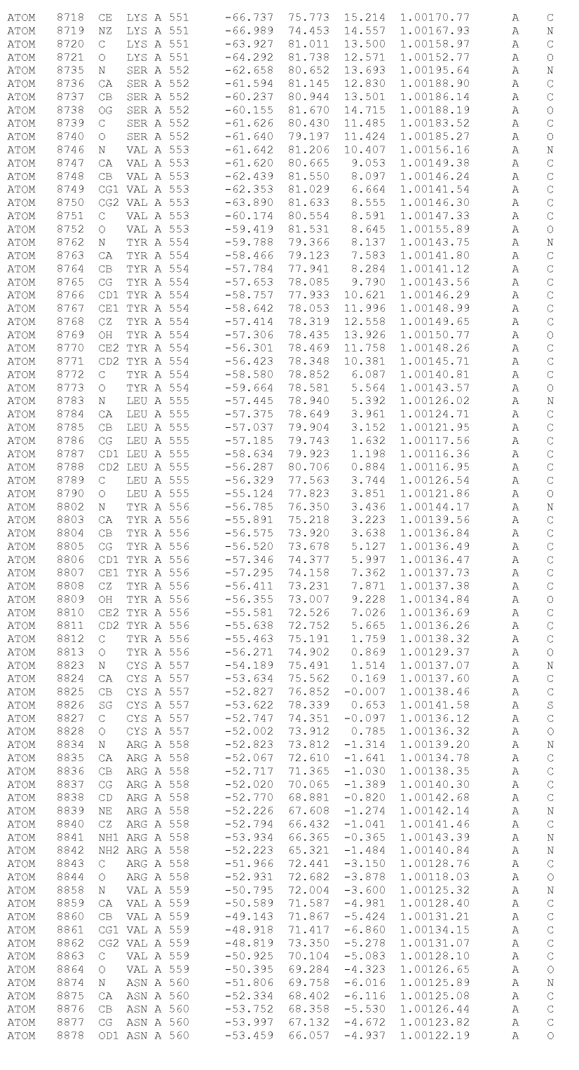

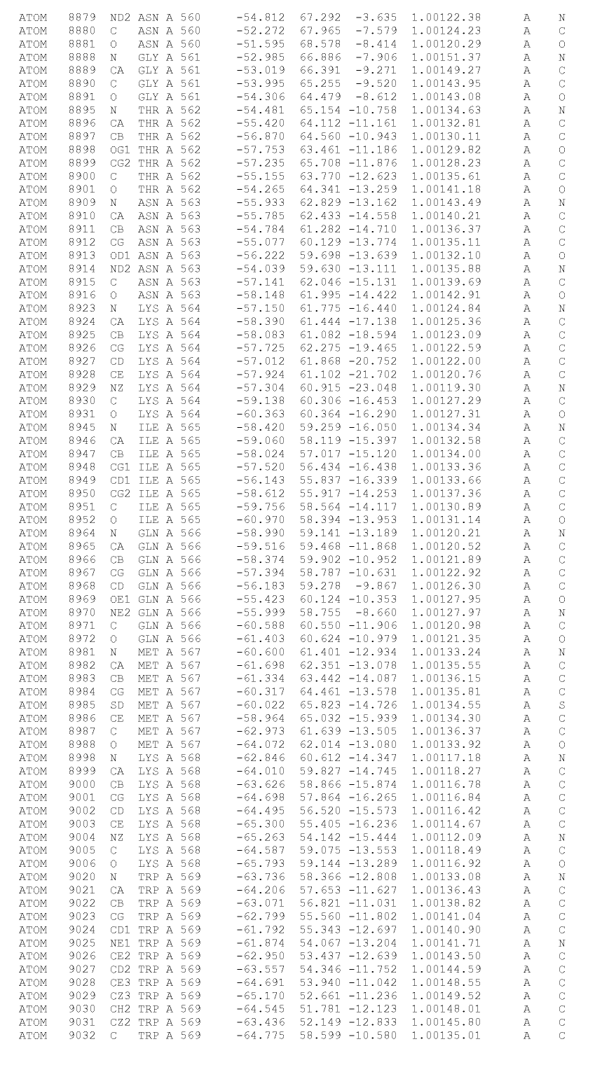

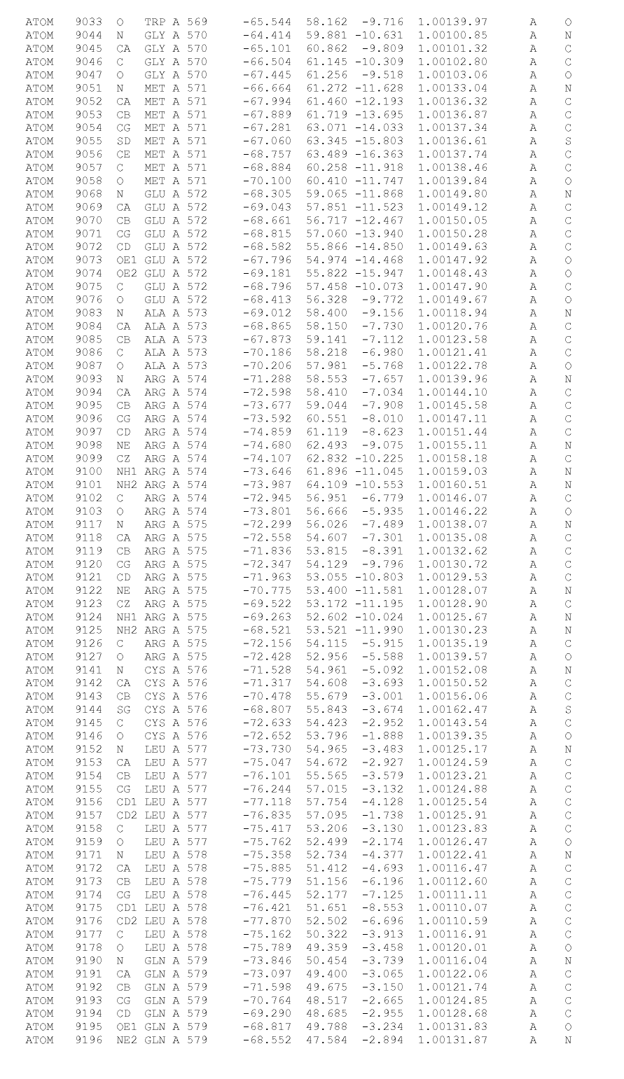

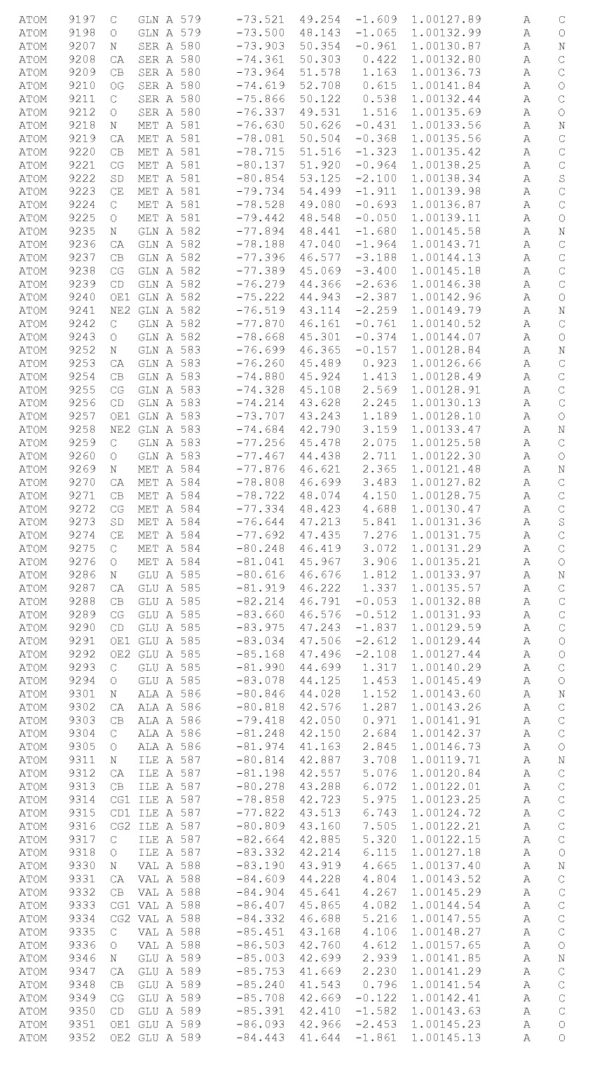

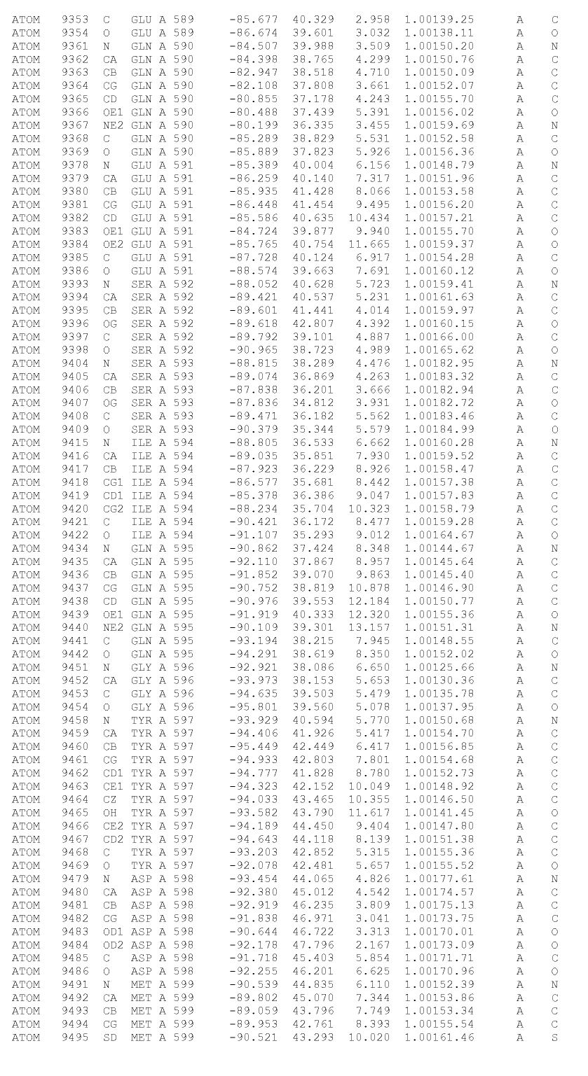

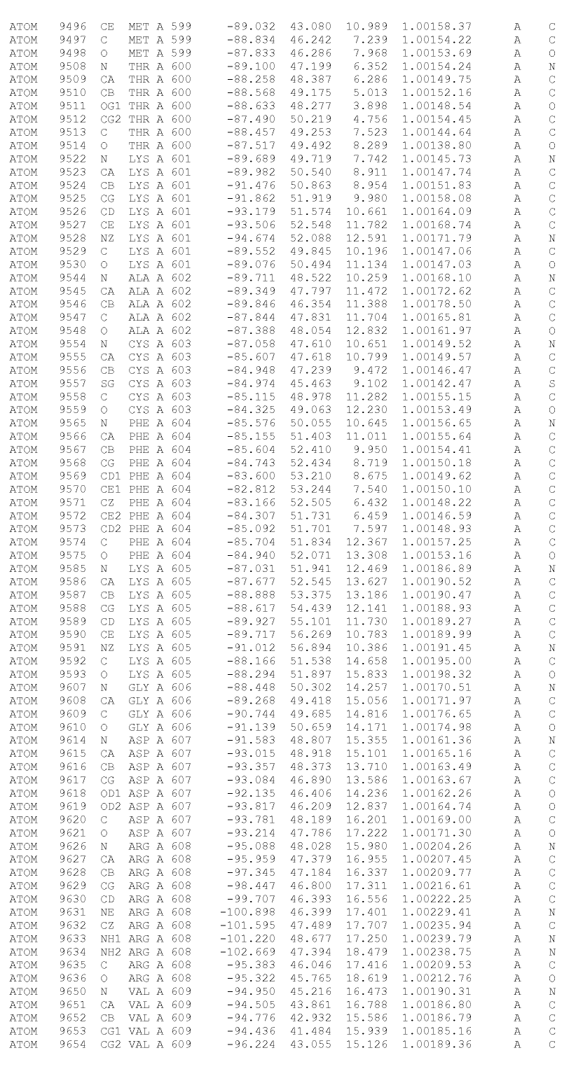

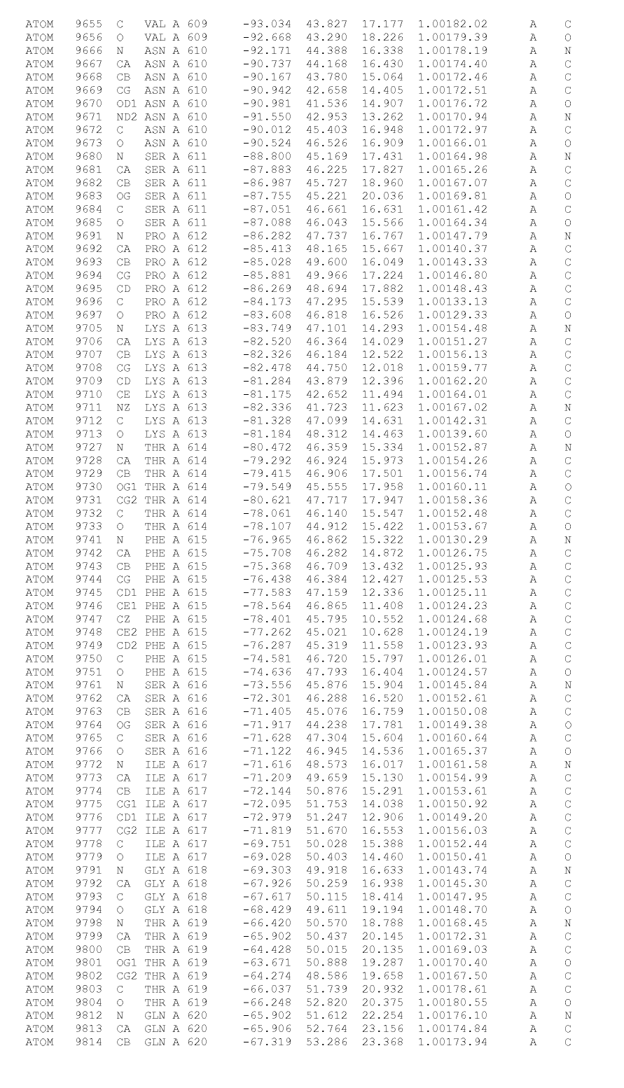

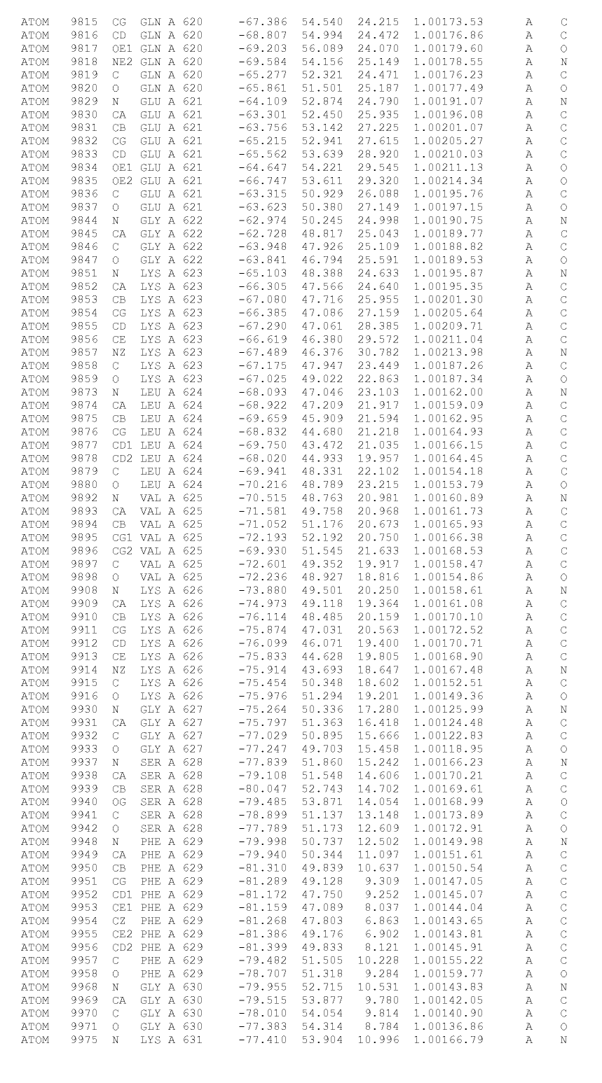

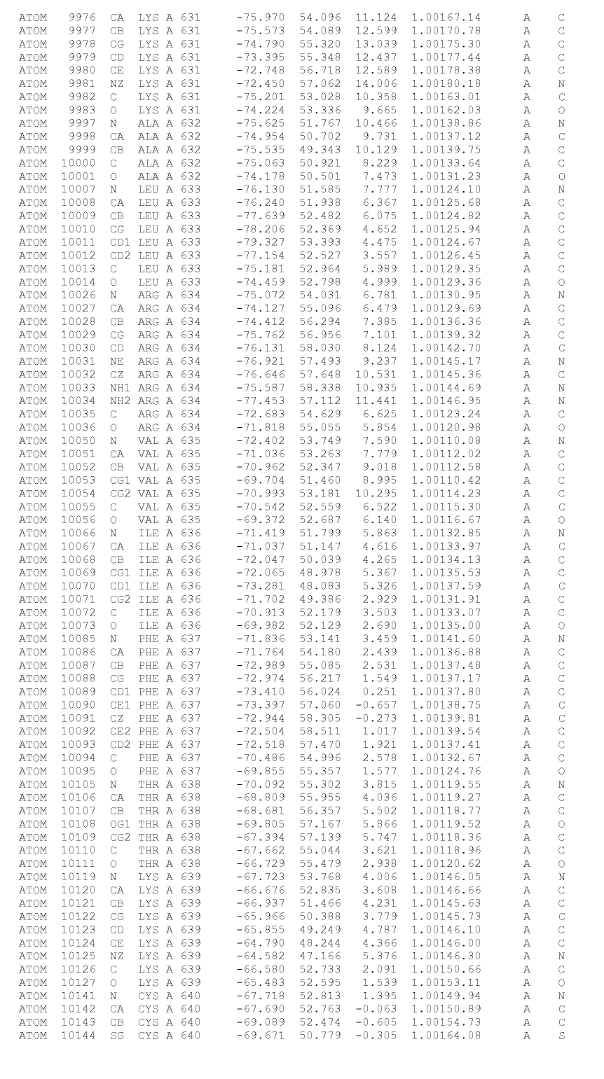

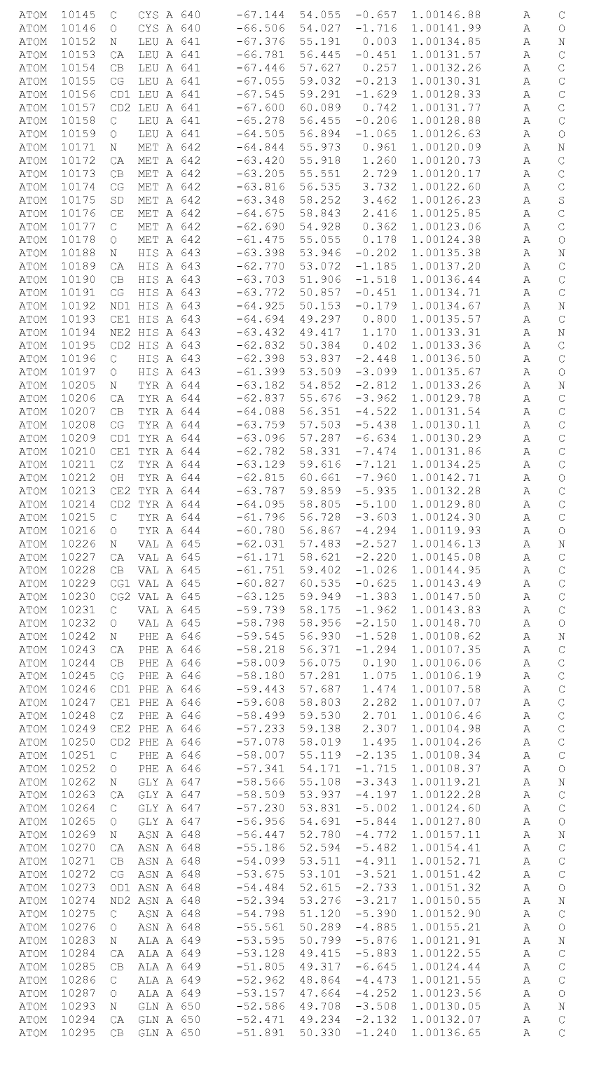

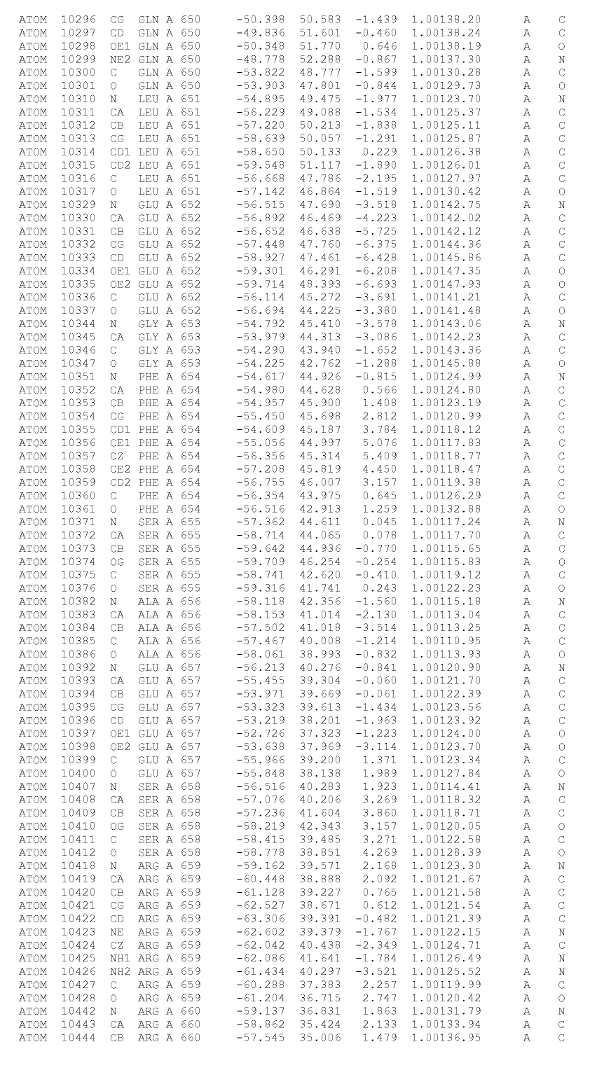

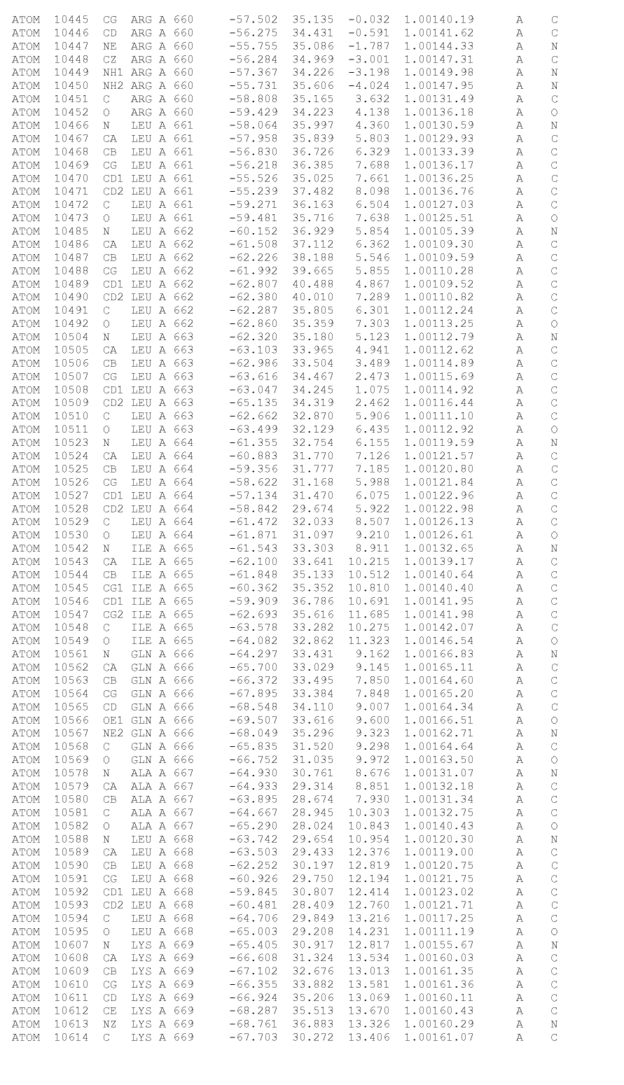

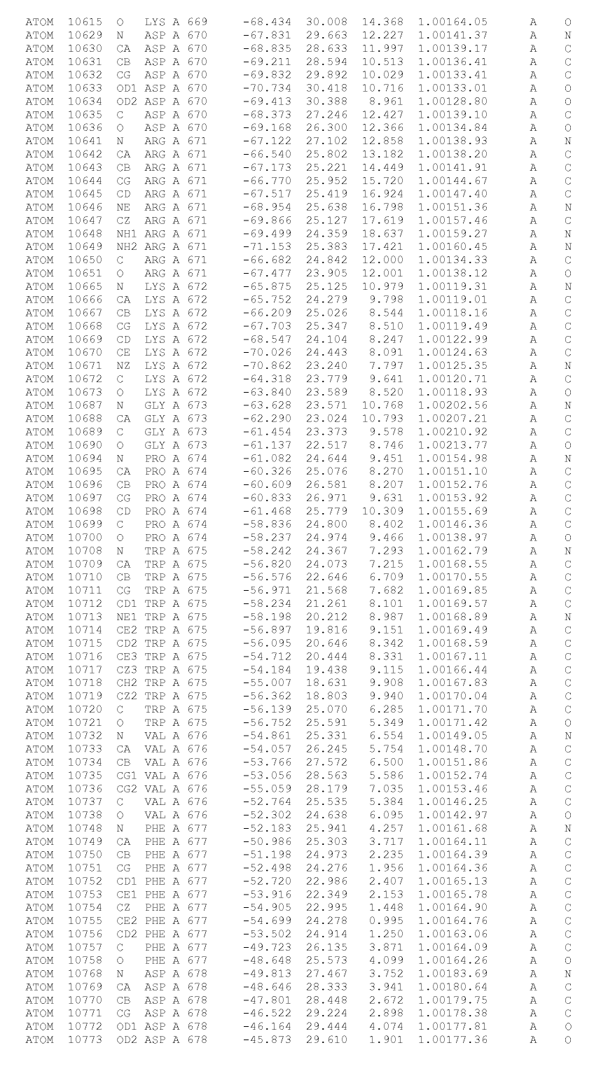

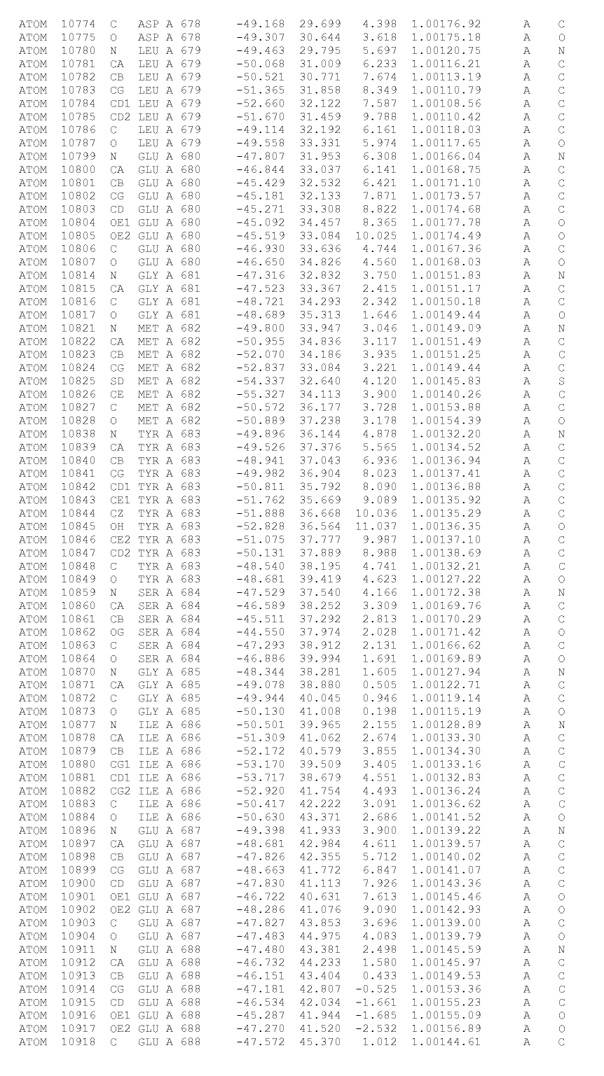

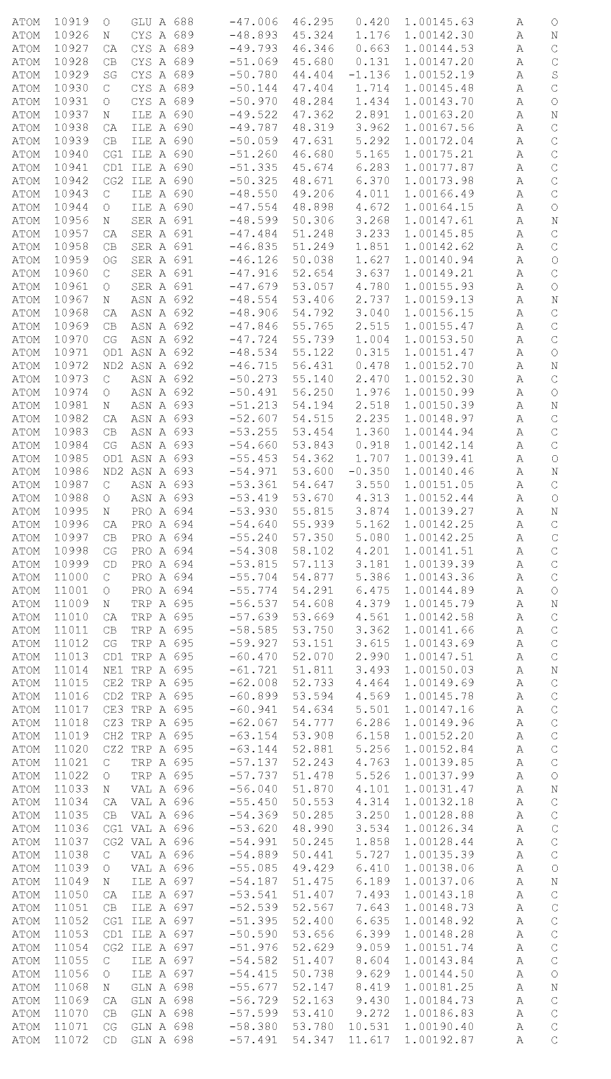

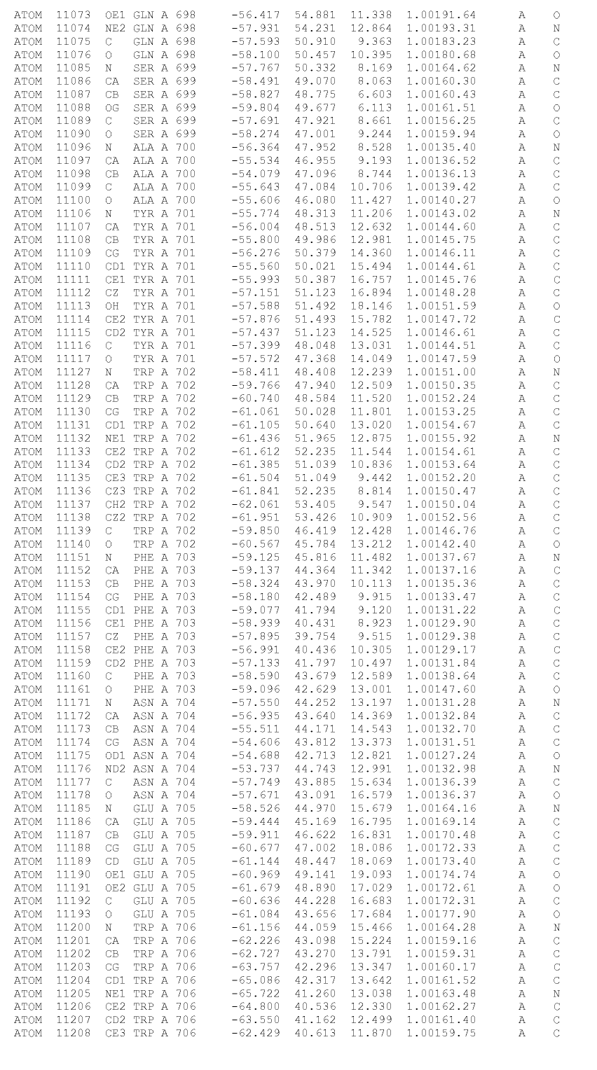

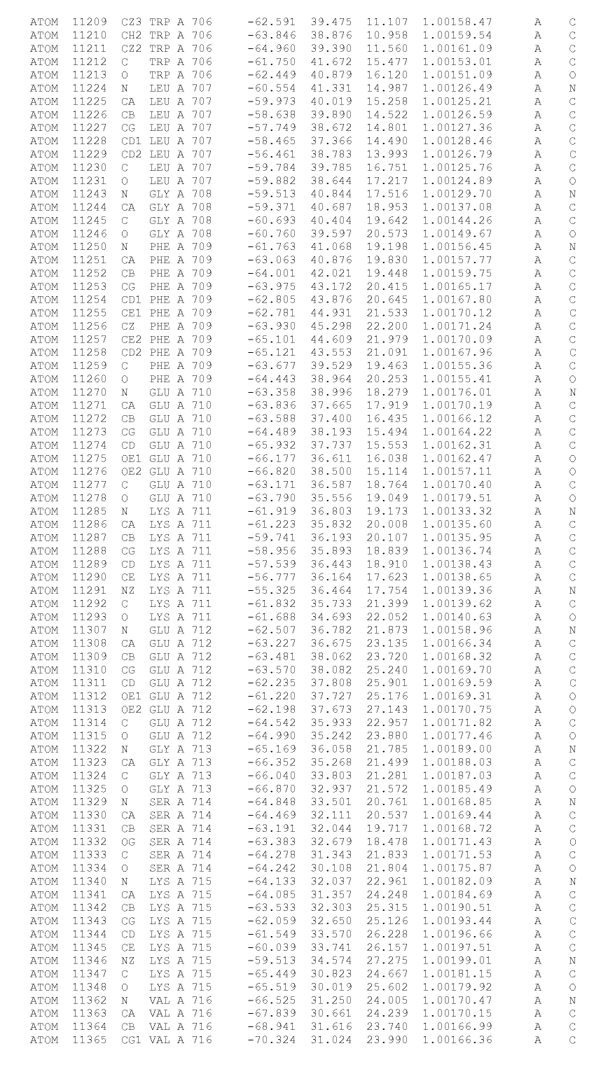

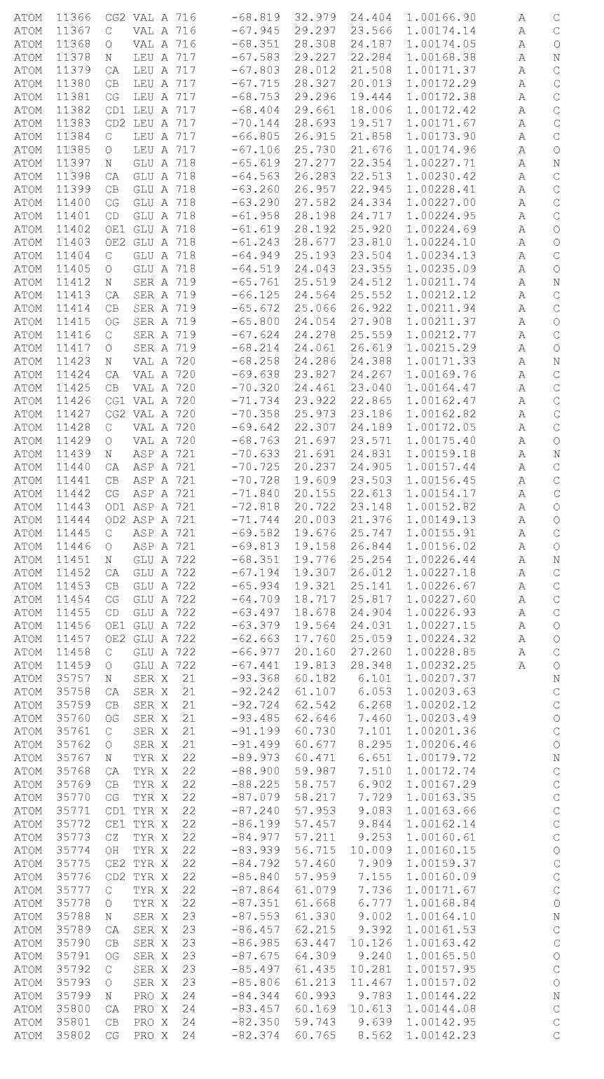

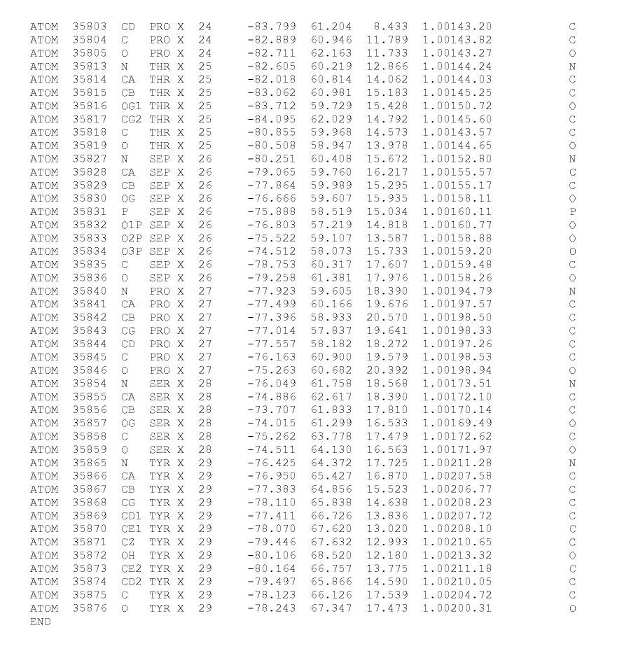

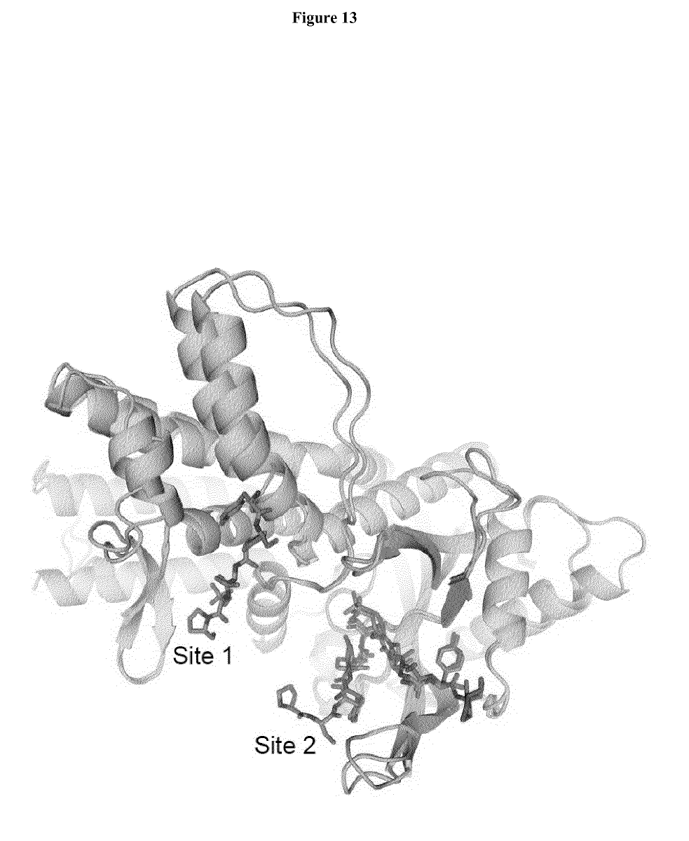

The structure shows CTD peptide binding in site 2 on PA, exactly as observed in the bat polymerase structure, with key basic residues Lys289 and Arg454 interacting with the phosphate of the phosphoserine (FIG. 13, 14). However no binding is observed in site 1, which can either be explained by crystal packing affecting peptide binding, or by the observed distortion in of PA site 1 (FIG. 13) due to the truncation in PB2. The latter is consistent with previous reports that the complete heterotrimer is required for full CTD binding.

These results confirm that the CTD binding site 2 is fully conserved between bat and avian/human influenza A polymerases, as predicted from sequence conservation. Furthermore the high yield of the H7N9 core polymerase construct make this useful for further studies of CTD binding affinity and specificity as well as virtual and biochemical screening for compounds that inhibit CTD binding exclusively in site 2.

1. An in silico method for identifying compounds which decrease or prevent the binding of the viral RNA-dependent RNA polymerase from the Orthomyxoviridae family or variant thereof, to its ligand, comprising the steps of
(a) constructing a computer model based on the structure coordinates of the binding site of the viral RNA-dependent RNA polymerase to its ligand;
(b) selecting a potential modulating compound by a method selected from the group consisting of:
(i) modifying the co-crystallised ligand inside the binding site,
(ii) filtering and selecting compounds from small molecule databases based on the interaction profile of the co-crystallised ligand with the binding site of the viral RNA-dependent RNA polymerase, and/or based on 3D similarity to the co-crystallised ligand, and
(iii) de novo ligand design of said compound based on the interaction profile of the co-crystallised ligand with the binding site of the viral RNA-dependent RNA polymerase and/or based on 3D similarity to the co-crystallised ligand;
(c) employing computational means to perform a fitting program operation between computer models of the said compound and said binding site in order to provide an energy-minimized configuration of the said compound in the active site; and/or employing computational docking methods to position and place said compounds into the said binding site in order to provide reasonable 3D-arrangements of the chemical entities, said compounds; and
(d) evaluating the results of said fitting operation and optionally said docking methods to quantify the association between the said compound and the binding site model, thereby evaluating the ability of said compound to associate with the said binding site.

2. The method of claim 1, wherein the binding site comprises amino acids K630, R633, and E444 of the PA subunit of the RNA-dependent RNA polymerase of Influenza A virus (SEQ ID NO: 1); comprises amino acids K635, R638, and E449 of SEQ ID NO: 44; or comprises amino acids E445, K631, and R634 of the PA subunit of the RNA-dependent RNA polymerase of Influenza B virus (SEQ ID NO: 2), or comprises amino acids occupying analogous position in the sequence of a PA subunit aligned thereto.

3. The method of claim 2, wherein the binding site of the PA subunit of the RNA-dependent RNA polymerase of Influenza A virus further comprises amino acids K289, R449, and E452 of SEQ ID NO: 1, optionally further comprises amino acids F440 and F607, and optionally further comprises one or more amino acid selected from the group consisting of M288, L290, S291, T313, F314, I545, M543, and K554 of SEQ ID NO: 1, optionally further comprises amino acid G629 of SEQ ID NO: 1, or wherein the binding site of the PA subunit of the RNA-dependent RNA polymerase of Influenza A virus further comprises amino acids K289, R454 and E457 of SEQ ID NO: 44, optionally further comprises amino acids Y44 and F612, and optionally further comprises one or more amino acid selected from the group consisting of L288, L290, S291, T313, F314, L550, M548, and R559 of SEQ ID NO: 44, optionally further comprises amino acid G634 of SEQ ID NO: 44, or wherein the binding site of the PA subunit of the RNA-dependent RNA polymerase of Influenza B virus further comprises amino acids Y441 and F604 of SEQ ID NO: 2, and optionally further comprises amino acid G630 of SEQ ID NO: 2.

4. The method of claim 1, wherein the binding site comprises amino acids 258-713 of SEQ ID NO: 1, comprises amino acids 201-716 of SEQ ID NO: 44 or comprises amino acids 258-722 of SEQ ID NO: 2.

5. The method of claim 1, wherein said computer model is based on the structure coordinates as shown in FIG. 11 or 12.

6. A method of producing a compound which decreases or prevents the binding of the viral RNA-dependent RNA polymerase from the Orthomyxoviridae family or variant thereof, to its ligand, comprising the steps of
(a) identifying said compound via the method of claim 1;
(b) synthesizing said compound and optionally formulating said compound or a pharmaceutically acceptable salt thereof with one or more pharmaceutically acceptable excipient(s) and/or carrier(s); and optionally:
(c) contacting said compound with the viral RNA-dependent RNA polymerase from the Orthomyxoviridae family, and
(d) determine the ability of said compound to prevent the binding of viral RNA-dependent RNA polymerase to its ligand.

7. The method of claim 1, wherein said test compound is a small molecule or a peptide or protein.

8. A compound identifiable and/or producible by the method of claim 1, wherein said compound is able to decrease or prevent the binding of the viral RNA-dependent RNA polymerase or variant thereof, to its ligand.

9. An antibody directed against the binding site of the viral RNA-dependent RNA polymerase from the Orthomyxoviridae family or variant thereof, to its ligand.

10. A nucleic acid encoding the antibody of claim 9.

11. A vector comprising the nucleic acid of claim 10.

12. A recombinant host cell comprising the isolated nucleic acid of claim 10.

13. A pharmaceutical composition producible according to the method of claim 6.

14. A pharmaceutical composition comprising the compound of claim 8.

15. A compound according to claim 8, for use in treating, ameliorating, or preventing disease conditions caused by viral infections with viruses of the Orthomyxoviridae family.

16. A recombinant host cell comprising the recombinant vector of claim 11.

17. A pharmaceutical composition comprising the antibody of claim 9.

18. A pharmaceutical composition comprising the nucleic acid of claim 10.

19. A pharmaceutical composition comprising the vector of claim 11.

20. A pharmaceutical composition comprising the recombinant host cell of claim 12.


SEQUENCE LISTING (45 sequences)

SEQ ID NO: 1 - protein, 713 aa, Influenza A virus
MENFVRTNFNPMILERAEKTMKEYGENPQNEGNKFAAISTHMEVCFMYSDFHFIDLEGNTIVKENDDDNAMLKHRFEIIEGQERNIAWTIVNSICNMTENSKPRFLPDLYDYKTNKFIEIGVTRRKVEDYYYEKASKLKGENVYIHIFSFDGEEMATDDEYILDEESRARIKTRLFVLRQELATAGLWDSFRQSEKGEETLEEEFSYPPTFQRLANQSLPPSFKDYHQFKAYVSSFKANGNIEAKLGAMSEKVNAQIESFDPRTIRELELPEGKFCTQRSKFLLMDAMKLSVLNPAHEGEGIPMKDAKACLDTFWGWKKATIIKKHEKGVNTNYLMIWEQLLESIKEMEGKFLNLKKTNHLKWGLGEGQAPEKMDFEDCKEVPDLFQYKSEPPEKRKLASWIQSEFNKASELTNSNWIEFDELGNDVAPIEHIASRRRNFFTAEVSQCRASEYIMKAVYINTALLNSSCTAMEEYQVIPIITKCRDTSGQRRTNLYGFIIKGRSHLRNDTDVVNFISLEFSLTDPRNEIHKWEKYCVLEIGDMEIRTSISTIMKPVYLYVRTNGTSKIKMKWGMEMRRCLLQSLQQVESMIEAESAVKEKDMTEPFFRNRENDWPIGESPQGIEKGTIGKVCRVLLAKSVFNSIYASAQLEGFSAESRKLLLLIQAFRDNLDPGTFDLKGLYEAIEECIINDPWVLLNASWFNSFLKAVQLSM

SEQ ID NO: 2 - protein, 726 aa, Influenza B virus
MDTFITRNFQTTIIQKAKNTMAEFSEDPELQPAMLFNICVHLEVCYVISDMNFLDEEGKAYTALEGQGKEQNLRPQYEVIEGMPRTIAWMVQRSLAQEHGIETPKYLADLFDYKTKRFIEVGITKGLADDYFWKKKEKLGNSMELMIFSYNQDYSLSNESSLDEEGKGRVLSRLTELQAELSLKNLWQVLIGEEDVEKGIDFKLGQTISRLRDISVPAGFSNFEGMRSYIDNIDPKGAIERNLARMSPLVSVTPKKLTWEDLRPIGPHIYNHELPEVPYNAFLLMSDELGLANMTEGKSKKPKTLAKECLEKYSTLRDQTDPILIMKSEKANENFLWKLWRDCVNTISNEEMSNELQKTNYAKWATGDGLTYQKIMKEVAIDDETMCQEEPKIPNKCRVAAWVQTEMNLLSTLTSKRALDLPEIGPDVAPVEHVGSERRKYFVNEINYCKASTVMMKYVLFHTSLLNESNASMGKYKVIPITNRVVNEKGESFDMLYGLAVKGQSHLRGDTDVVTVVTFEFSSTDPRVDSGKWPKYTVFRIGSLFVSGREKSVYLYCRVNGTNKIQMKWGMEARRCLLQSMQQMEAIVEQESSIQGYDMTKACFKGDRVNSPKTFSIGTQEGKLVKGSFGKALRVIFTKCLMHYVFGNAQLEGFSAESRRLLLLIQALKDRKGPWVFDLEGMYSGIEECISNNPWVIQSAYWFNEWLGFEKEGSKVLESVDEIMDE

SEQ ID NO: 3 - protein, 7 aa, Artificial Sequence
  linker peptide
GMGSGMA

SEQ ID NO: 4 - protein, 15 aa, Artificial Sequence
  AviTag
GLNDIFEAQKIEWHE

SEQ ID NO: 5 - protein, 26 aa, Artificial Sequence
  Calmodulin-tag
KRRWKKNFIAVSAANRFKKISSSGAL

SEQ ID NO: 6 - protein, 6 aa, Artificial Sequence
  polyglutamate tag
EEEEEE

SEQ ID NO: 7 - protein, 13 aa, Artificial Sequence
  E-tag
GAPVPYPDPLEPR

SEQ ID NO: 8 - protein, 8 aa, Artificial Sequence
  FLAG-tag
DYKDDDDK

SEQ ID NO: 9 - protein, 9 aa, Artificial Sequence
  HA-tag
YPYDVPDYA

SEQ ID NO: 10 - protein, 6 aa, Artificial Sequence
  His-tag
HHHHHH

SEQ ID NO: 11 - protein, 10 aa, Artificial Sequence
  Myc-tag
EQKLISEEDL

SEQ ID NO: 12 - protein, 15 aa, Artificial Sequence
  S-tag
KETAAAKFERQHMDS

SEQ ID NO: 13 - protein, 38 aa, Artificial Sequence
  SBP-tag
MDEKTTGWRGGHVVEGLAGELEQLRARLEHHPQGQREP

SEQ ID NO: 14 - protein, 13 aa, Artificial Sequence
  Softag 1
SLAELLNAGLGGS

SEQ ID NO: 15 - protein, 8 aa, Artificial Sequence
  Softag 3
TQDPSRVG

SEQ ID NO: 16 - protein, 8 aa, Artificial Sequence
  Strep-tag
WSHPQFEK

SEQ ID NO: 17 - protein, 6 aa, Artificial Sequence
  TC tag
CCPGCC

SEQ ID NO: 18 - protein, 14 aa, Artificial Sequence
  V5 tag
GKPIPNPLLGLDST

SEQ ID NO: 19 - protein, 11 aa, Artificial Sequence
  VSV
YTDIEMNRLGK

SEQ ID NO: 20 - protein, 8 aa, Artificial Sequence
  Xpress tag
DLYDDDDK

SEQ ID NO: 21 - protein, 7 aa, Artificial Sequence
  TEV protease recognition site
ENLYFQG

SEQ ID NO: 22 - protein, 16 aa, Artificial Sequence
  Isopeptag
TDKDMTITFTNKKDAE

SEQ ID NO: 23 - protein, 13 aa, Artificial Sequence
  SpyTag
AHIVMVDAYKPTK

SEQ ID NO: 24 - RNA, 13 nt, Influenza A virus
aguagaaacaagg

SEQ ID NO: 25 - RNA, 13 nt, Influenza A virus
gccugcuucugcu

SEQ ID NO: 26 - RNA, 14 nt, Influenza B virus
aguaguaacaagag

SEQ ID NO: 27 - RNA, 13 nt, Influenza B virus
cucugcuucugcu

SEQ ID NO: 28 - RNA, 14 nt, Influenza A virus
aguaguaacaagag

SEQ ID NO: 29 - RNA, 14 nt, Influenza A virus
agcagaagcagagg

SEQ ID NO: 30 - RNA, 18 nt, Influenza A virus
uauaccucugcuucugcu

SEQ ID NO: 31 - RNA, 18 nt, Influenza A virus
uacccucuuguuacuacu

SEQ ID NO: 32 - RNA, 13 nt, Artificial Sequence
  capped primer
gaaucuauaauag

SEQ ID NO: 33 - DNA, 24 bp, Artificial Sequence
  primer1
agtagaaacaaggtacttttttgg

SEQ ID NO: 34 - DNA, 21 bp, Artificial Sequence
  primer2
tgcaggacattgagaatgagg

SEQ ID NO: 35 - DNA, 21 bp, Artificial Sequence
  primer
acctcaattctggttcatcac

SEQ ID NO: 36 - DNA, 21 bp, Artificial Sequence
  primer
agcgaaagcaggtactgatcc

SEQ ID NO: 37 - protein, 716 aa, Influenza A virus
MEDFVRQCFNPMIVELAEKAMKEYGEDLKIETNKFAAICTHLEVCFMYSDFHFIDEQGESIVVELGDPNALLKHRFEIIEGRDRTIAWTVINSICNTTGAEKPKFLPDLYDYKKNRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKSTPRPLRLPDGPPCSQRSKFLLMDALKLSIEDPSHEGEGIPLYDAIKCMRTFFGWKEPNVVKPHEKGINPNYLLSWKQVLAELQDIENEEKIPRTKNMKKTSQLKWALGENMAPEKVDFDDCKDVGDLKQYDSDEPELRSLASWIQNEFNKACELTDSSWIELDEIGEDAAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEVGDMLLRSAIGHVSRPMFLYVRTNGTSKIKMKWGMEMRRCLLQSLQQIESMIEAESSVKEKDMTKEFFENKSETWPVGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 38 - protein, 716 aa, Influenza A virus
MEDFVRQCFNPMIVELAEKAMKEYGEDPKIETNKFAAICTHLEVCFMYSDFHFIDERSESIIVESGDPNALLKHRFEIIEGRDRTMAWTVVNSICNTTGVEKPKFLPDLYDYKENRFIEIGVTRREVHTYYLEKANKIKSEKTHIHIFSFTGEEMATKADYTLDEESRARIKTRLFTIRQEMASRGLWDSFRQSERGEETIEEKFEITGTMRRLADQSLPPNFSSLENFRAYVDGFEPNGCIEGKLSQMSKEVNARIEPFLKTTPRPLRLPDGPPCSQRSKFLLMDALKLSIEDPSHEGEGIPLYDAIKCMKTFFGWKEPNIVKPHEKGINPNYLLAWKQVLAELQDIENEEKIPKTKNMKKTSQLKWALGENMAPEKVDFEDCKDVSDLRQYDSDEPESRSLASWIQSEFNKACELTDSSWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAVGQVSRPMFLYVRTNGTSKIKMKWGMEMRRCLLQSLQQIESMIEAESSVKEKDMTKEFFENKSETWPIGESPKGVEEGSIGKVCRTLLAKSVFNSLYASPQLEGFSAESRKLLLIAQALRDNLEPGTFDLGGLYEAIEECLINDPWVLLNASWFNSFLAHALK

SEQ ID NO: 39 - protein, 709 aa, Influenza C virus
MSKTFAEIAEAFLEPEAVRIAKEAVEEYGDHERKIIQIGIHFQVCCMFCDEYLSTNGSDRFVLIEGRKRGTAVSLQNELCKSYDLEPLPFLCDIFDREEKQFVEIGITRKADDSYFQSKFGKLGNSCKIFVFSYDGRLDKNCEGPMEEQKLRIFSFLATAADFLRKENMFNEIFLPDNEETIIEMKKGKTFLELRDESVPLPFQTYEQMKDYCEKFKGNPRELASKVSQMQSNIKLPIKHYEQNKFRQIRLPKGPMAPYTHKFLMEEAWMFTKISDPERSRAGEILIDFFKKGNLSAIRPKDKPLQGKYPIHYKNLWNQIKAAIADRTMVINENDHSEFLGGIGRASKKIPEISLTQDVITTEGLKQSENKLPEPRSFPRWFNAEWMWAIKDSDLTGWVPMAEYPPADNELEDYAEHLNKTMEGVLQGTNCAREMGKCILTVGALMTECRLFPGKIKVVPIYARSKERKSMQEGLPVPSEMDCLFGICVKSKSHLNKDDGMYTIITFEFSIREPNLEKHQKYTVFEAGHTTVRMKKGESVIGREVPLYLYCRTTALSKIKNDWLSKARRCFITTMDTVETICLRESAKAEENLVEKTLNEKQMWIGKKNGELIAQPLREALRVQLVQQFYFCIYNDSQLEGFCNEQKKILMALEGDKKNKSSFGFNPEGLLEKIEECLINNPMCLFMAQRLNELVIEASKRGAKFFKTD

SEQ ID NO: 40 - protein, 710 aa, Influenza virus
MSSIIREIAKRFLEQATINIAEEVVREYGDHERTMISVGVHFQACCLISDEYTLEDETTPRYVLLEGLRRQEAISKQNNICSTLGLEPLRNLADIFDRKTRRFLEVGITKRESDEYYQEKFNKIGNDMDIHVFTYEGKYFSNNPNGLEDIQKTRIFTFLSFVSDELRKENMFTEMYVTEEGAPELEMYKSKLFIAMRDESVPLPYINYEHLRTRCETFKRNQAECEAKVADVASRLKIKLEHLEENKLRPLEIPKEREAPYTHKFMMKDAWFFAKPHDSERAQPQQILYDFFEAANMGFMTTSPKPIFGKQGLMYHSLWGQIKRAIKDKRNELEPSEQRDFLCGIGRASKKIQEDKWQESKEEEFKQEETKGAAKRGFPTWFNEEWLWAMRDSGDGDNKIGDWIPMAEMPPCKNEMEDYAKKMCEELESKIQGTNCAREMSKLIHTIGSLHTECRNFPGKVKIVPIYCRGTLRGESTDCLFGIAIKGKSHLNKDDGMYTVVTFEFSTEKPNPSKHEKYTVFEAGTVPVEAVVLTPKRERVLKEKKLFLYCRTTGMSKLKNDWFSKCRRCLIPTMETVEQIVLKECALKEENRVSEMLENKRAWIAHENGENLTRLVSTKLKDLCRMLIVTQFYYCIYNDNQLEGFCNEQKKFLMFLQADKDSKSAFTFNQKGLYEKIEECIVSNPLCIFLADRLNKLFLVAKSNGAKYFE

SEQ ID NO: 41 - protein, 757 aa, Artificial Sequence
  H7N9/PB1_full length
MDVNPTLLFLKVPVQNAISTTFPYTGDPPYSHGTGTGYTMDTVNRTHKYSEKGKWTTNTETGAPQLNPIDGPLPEDNEPSGYAQTDCVLEAMAFLEESHPGIFENSCLETMEIVQQTRVDKLTQGRQTYDWTLNRNQPAATALANTIEVFRSNGLTANESGRLIDFLKDVMDSMDKEEMEITTHFQRKRRVRDNMTKKMVTQRTIGKKKQRLNKRSYLIRALTLNTMTKDAERGKLKRRAIATPGMQIRGFVYFVEALARSICEKLEQSGLPVGGNEKKAKLANVVRKMMTNSQDTELSFTITGDNTKWNENQNPRMFLAMITYITRNQPEWFRNVLSIAPIMFSNKMARLGKGYMFESKSMKLRTQVPAEMLANIDLKYFNKSTREKIEKIRPLLIDGTASLSPGMMMGMFNMLSTVLGVSILNLGQKKYTKTTYWWDGLQSSDDFALIVNAPNHEGIQAGVDRFYRTCKLVGINMSKKKSYINRTGTFEFTSFFYRYGFVANFSMELPSFGVSGINESADMSVGVTVIKNNMINNDLGPATAQMALQLFIKDYRYTYRCHRGDTQIQTRRAFELKKLWEQTRSKAGLLVSDGGPNLYNIRNLHIPEVCLKWELMDEDYQGRLCNPMNPFVSHKEIDSVNNAVVMPAHGPAKSMEYDAVATTHSWIPKRNRSILNTSQRGILEDEQMYQKCCNLFEKFFPSSSYRRPVGISSMVEAMVSRARIDARIDFESGRIKKEEFAEIMKICSTIEELRRQK

SEQ ID NO: 42 - protein, 127 aa, Artificial Sequence
  H7N9/PB2_1-127
MERIKELRDLMSQSRTREILTKTTVDHMAIIKKYTSGRQEKNPALRMKWMMAMKYPITADKRIMEMIPERNEQGQTLWSKTNDAGSDRVMVSPLAVTWWNRNGPTTSTVHYPKVYKTYFEKVERLKH

SEQ ID NO: 43 - protein, 20 aa, Artificial Sequence
  TEV-cleavable poly-histidine tag
GSGSENLYFQGSHHHHHHHH

SEQ ID NO: 44 - protein, 716 aa, Artificial Sequence
  H7N9/PA full length
MEDFVRQCFNPMIVELAEKAMKEYGEDPKIETNKFASICTHLEVCFMYSDFHFIDERGESTIIESGDPNVLLKHRFEIIEGRDRTMAWTVVNSICNTTGVEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRRLADQSLPPNFSSLENFRAYVDGFEPNGCIEGKLSQMSKEVNARIEPFLRTTPRPLRLPDGPPCSQRSKFLLMDALKLSIEDPSHEGEGIPLYDAIKCMKTFFGWKEPNIIKPHEKGINPNYLLTWKQVLAELQDIENEEKIPRTKNMKKTSQLKWALGENMAPEKVDFEDCKDVNDLKQYDSDEPEPRSLACWIQSEFNKACELTDSSWVELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAVGQVSRPMFLYVRTNGTSKIKMKWGMEMRRCLLQSLQQIESMIEAESSVKEKDLTKEFFENKSETWPIGESPKGVEEGSIGKVCRTLLAKSVFNSLYASPQLEGFSAESRKLLLIVQALRDNLEPGTFDLEGLYEAIEECLINDPWVLLNASWFNSFLTHALR

SEQ ID NO: 45 - RNA, 12 nt, Influenza A virus
aguaguaacaag